(19) United States
(12) Patent Application Publication
(10) Pub. No.: US 2025/0305050 A1
(43) Pub. Date: Oct. 2, 2025

(54) Inhibition Of HSD17B13 In The Treatment Of Liver Disease In Patients Expressing The PNPLA3 I148M Variation

(71) Applicant: Regeneron Pharmaceuticals, Inc., Tarrytown, NY (US)

(72) Inventors: Yurong Xin, Tarrytown, NY (US); Jesper Gromada, Tarrytown, NY (US); Xiping Cheng, Tarrytown, NY (US); Frederick Dewey, Tarrytown, NY (US); Tanya Teslovich Dostal, Tarrytown, NY (US); Claudia Schurmann, Tarrytown, NY (US); Aris Baras, Tarrytown, NY (US); Noura Abul-Husn, Tarrytown, NY (US)

(21) Appl. No.: 19/085,403

(22) Filed: Mar. 20, 2025

Related U.S. Application Data
(63) Continuation of application No. 18/325,500, filed on May 30, 2023, now Pat. No. 12,286,677.
Application No. 18/325,500 is a continuation of application No. 17/178,420, filed on Feb. 18, 2021, now Pat. No. 11,702,700.
Application No. 17/178,420 is a continuation of application No. 16/157,503, filed on Oct. 11, 2018, now Pat. No. 10,961,583.
(60) Provisional application No. 62/570,985, filed on Oct. 11, 2017.

(51) Int. Cl. C12Q 1/6883; C12Q 1/6886

(52) U.S. Cl. CPC C12Q 1/6883; C12Q 1/6886; C12Q 2600/106; C12Q 2600/156; C12Q 2600/158

(57) ABSTRACT

The disclosure provides methods of identifying a human subject as a candidate for treating or inhibiting a liver disease by inhibiting HSD17B13. The disclosure also provides methods of treating a subject who is PNPLA3 Ile148Met+ by administering an inhibitor of HSD17B13. The disclosure also provides method of detecting a PNPLA3 Ile148Met variant and functional HSD17B13 in a subject. The disclosure also provides method of identifying a subject having a protective effect against liver disease. The disclosure also provides inhibitors of HSD17B13 for use in the treatment of a liver disease.

### REFERENCE TO A SEQUENCE LISTING

This application includes a Sequence Listing submitted electronically as an XML file named 381204346SEQ, created on Feb. 20, 2025, with a size of 216,235 bytes. The Sequence Listing is incorporated by reference herein.

### FIELD

The disclosure relates generally to the field of precision medicine. More particularly, the disclosure relates to methods of identifying subjects who are patatin like phospholipase domain containing 3 (PNPLA3) Ile148Met positive and have a liver disease or susceptibility to liver disease, and treating such subjects with an inhibitor of hydroxysteroid 17-beta dehydrogenase 13 (HSD17B13).

### BACKGROUND

Various references, including patents, patent applications, accession numbers, technical articles, and scholarly articles are cited throughout the specification. Each reference is incorporated by reference herein, in its entirety and for all purposes.

Chronic liver disease and cirrhosis are leading causes of morbidity and mortality in the United States, accounting for 38,170 deaths (1.5% of total deaths) in 2014 (Kochanek et al., Nat'l. Vital Stat. Rep., 2016, 65, 1-122). The most common etiologies of cirrhosis in the U.S. are alcoholic liver disease, chronic hepatitis C, and nonalcoholic fatty liver disease (NAFLD), together accounting for about 80% of patients awaiting liver transplant between 2004 and 2013 (Wong et al., Gastroenterology, 2015, 148, 547-555). The estimated prevalence of NAFLD in the U.S. is between 19 and 46 percent (Browning et al., Hepatology, 2004, 40, 1387-1395; Lazo et al., Am. J. Epidemiol., 2013, 178, 38-45; and Williams et al., Gastroenterology, 2011, 140, 124-131) and is rising over time (Younossi et al., Clin. Gastroenterol. Hepatol., 2011, 9, 524-530), likely in conjunction with increased rates of obesity, its primary risk factor (Cohen et al., Science, 2011, 332, 1519-1523). While significant advances have been made in the treatment of hepatitis C, there are currently no evidence-based treatments for alcoholic or nonalcoholic liver disease and cirrhosis.

Previous genome wide association studies (GWAS) have identified sequence variations associated with increased risk of chronic liver disease. The most robustly validated association is with a common missense variant in patatin-like phospholipase domain-containing 3, encoded by the gene PNPLA3. This variant (rs738409, p.Ile148Met) was initially found to be associated with an increase in hepatic triglyceride levels (Romeo et al., Nat. Genet., 2008, 40, 1461-5), and subsequently associated with nonalcoholic steatohepatitis (NASH) (Rotman et al., Hepatology, 2010, 52, 894-903; Sookoian et al., J. Lipid Res., 2009, 50, 2111-2116) and cirrhosis (Shen et al., J. Lipid Res., 2015, 56, 167-175). A missense variant in TM6SF2, encoding transmembrane 6 superfamily member 2, also confers increased risk of nonalcoholic fatty liver disease (NAFLD) (Kozlitina et al., Nat. Genet., 2014, 46, 352-6; Liu et al., Nat. Commun., 2014, 5, 4309; and Sookoian et al., Hepatology, 2015, 61, 515-25). Exactly how the variants in PNPLA3 and TM6SF2 contribute to liver disease has yet to be fully elucidated (Smagris et al., J. Biol. Chem., 2016, 291, 10659-76; Mahdessian et al., Proc. Natl. Acad. Sci. USA, 2014, 111, 8913-8; Huang et al., J. Biol. Chem., 2011, 286, 37085-93; and Pirazzi et al., J. Hepatol., 2012, 57, 1276-82). To date, no genetic variants that protect from chronic liver disease have been identified.

### SUMMARY

The present disclosure provides methods for identifying a human subject as a candidate for treating or inhibiting a liver disease, the method comprising: determining whether or not a sample from the subject comprises: i) a first nucleic acid encoding a patatin like phospholipase domain containing 3 (PNPLA3) protein comprising an I148M variation and a second nucleic acid encoding a functional HSD17B13 protein; and/or ii) a PNPLA3 protein comprising an I148M variation and a functional HSD17B13 protein; and identifying the subject as a candidate for treating or inhibiting a liver disease by inhibiting HSD17B13 when both the first and second nucleic acids as defined in i) and/or both of the proteins as defined in ii) are detected.

In some embodiments, the first nucleic acid molecule comprises genomic DNA, mRNA, or a cDNA obtained from mRNA.

In some embodiments, the genomic DNA comprises an ATG codon at the positions corresponding to positions 5107 to 5109 according to SEQ ID NO:31; the mRNA comprises an AUG codon at the positions corresponding to positions 442 to 444 according to SEQ ID NO: 34; the mRNA comprises an AUG codon at the positions corresponding to positions 430 to 432 according to SEQ ID NO:35; the cDNA comprises an ATG codon at the positions corresponding to positions 442 to 444 according to SEQ ID NO:38; or the cDNA comprises an ATG codon at the positions corresponding to positions 430 to 432 according to SEQ ID NO: 39.

In some embodiments, the genomic DNA comprises the nucleotide sequence according to SEQ ID NO:31, or a nucleotide sequence having at least 90% sequence identity to SEQ ID NO:31 and encoding a PNPLA3 protein which comprises the I148M variation; the mRNA comprises the nucleotide sequence according to SEQ ID NO:34, or a nucleotide sequence having at least 90% sequence identity to SEQ ID NO:34 and encoding a PNPLA3 protein which comprises the I148M variation; the mRNA comprises the nucleotide sequence according to SEQ ID NO:35, or a nucleotide sequence having at least 90% sequence identity to SEQ ID NO:35 and encoding a PNPLA3 protein which comprises the I148M variation; the cDNA comprises the nucleotide sequence according to SEQ ID NO:38, or a nucleotide sequence having at least 90% sequence identity to SEQ ID NO:38 and encoding a PNPLA3 protein which comprises the I148M variation; or the cDNA comprises the nucleotide sequence according to SEQ ID NO:39, or a nucleotide sequence having at least 90% sequence identity to SEQ ID NO:39 and encoding a PNPLA3 protein which comprises the I148M variation.

In some embodiments, detecting the first nucleic acid comprises: sequencing at least a portion of the first nucleic acid, wherein the portion comprises the codon which encodes the I148M variation; or hybridizing the first nucleic acid with a probe or primer that specifically hybridizes to a portion of the first nucleic acid, wherein the portion comprises the codon encoding the I148M variation.

In some embodiments, the probe or primer is an allele-specific probe or primer, and wherein the probe or primer optionally comprises a label.

In some embodiments, the methods further comprise determining whether the subject is homozygous or heterozygous for the I148M variation.

In some embodiments, the second nucleic acid comprises genomic DNA, mRNA, or a cDNA obtained from mRNA.

In some embodiments, the genomic DNA comprises an adenine at the position corresponding to position 12,667 according to SEQ ID NO:1; the genomic DNA comprises the nucleotide sequence according to SEQ ID NO:1, or a nucleotide sequence having at least 90% sequence identity to SEQ ID NO:1 and encoding a functional HSD17B13 protein; the mRNA comprises the nucleotide sequence according to SEQ ID NO:3, or a nucleotide sequence having at least 90% sequence identity to SEQ ID NO:3 and encoding a functional HSD17B13 protein; the mRNA comprises the nucleotide sequence according to SEQ ID NO:4 or a nucleotide sequence having at least 90% sequence identity to SEQ ID NO:4 and encoding a functional HSD17B13 protein; the mRNA comprises the nucleotide sequence according to SEQ ID NO:7 or a nucleotide sequence having at least 90% sequence identity to SEQ ID NO:7 and encoding a functional HSD17B13 protein; the mRNA comprises the nucleotide sequence according to SEQ ID NO:11 or a nucleotide sequence having at least 90% sequence identity to SEQ ID NO:11 and encoding a functional HSD17B13 protein; the cDNA comprises the nucleotide sequence according to SEQ ID NO:12 or a nucleotide sequence having at least 90% sequence identity to SEQ ID NO:12 and encoding a functional HSD17B13 protein; the cDNA comprises the nucleotide sequence according to SEQ ID NO:13 or a nucleotide sequence having at least 90% sequence identity to SEQ ID NO:13 and encoding a functional HSD17B13 protein; the cDNA comprises the nucleotide sequence according to SEQ ID NO:16 or a nucleotide sequence having at least 90% sequence identity to SEQ ID NO:16 and encoding a functional HSD17B13 protein; or the cDNA comprises the nucleotide sequence according to SEQ ID NO:20 or a nucleotide sequence having at least 90% sequence identity to SEQ ID NO:20 and encoding a functional HSD17B13 protein.

In some embodiments, detecting the second nucleic acid comprises: sequencing the second nucleic acid; or hybridizing the second nucleic acid with a probe or primer that specifically hybridizes to a portion of the second nucleic acid, wherein the portion comprises the adenine at the position corresponding to position 12,667 according to SEQ ID NO:1.

In some embodiments, the probe or primer is an allele-specific probe or primer, and wherein the probe or primer optionally comprises a label.

In some embodiments, the methods further comprise determining whether the subject is homozygous or heterozygous for the second nucleic acid encoding a functional HSD17B13 protein in the sample.

In some embodiments, the methods further comprise administering an inhibitor of HSD17B13 to the subject.

In some embodiments, the liver disease is an alcoholic liver disease. In some embodiments, the alcoholic liver disease comprises one or more of cirrhosis, steatosis, or hepatocellular carcinoma resulting from alcohol consumption.

In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption.

### BRIEF DESCRIPTION OF THE FIGURES

The accompanying figures, which are incorporated in and constitute a part of this specification, illustrate several aspects and together with the description serve to explain the principles of the present disclosure.

FIG. 1 shows baseline characteristics of sequenced European-ancestry individuals from the discovery and replication cohorts.

FIG. 2 shows single nucleotide variants associated with serum transaminase levels at P<1.0×10⁻⁷ in the discovery cohort.

FIG. 3 shows replication and joint meta-analysis of 35 exome-wide significant single nucleotide variants from the discovery cohort in three separate European-ancestry cohorts.

FIG. 4 shows association of thirteen exome-wide significant and replicating single nucleotide variants with liver disease phenotypes in the discovery cohort.

FIG. 5 shows baseline characteristics of genotyped multi-ethnic cases and controls from the Dallas Liver and Pediatric Liver Studies.

FIG. 6 (panels A and B) shows regional association plots for alanine aminotransferase (ALT; A) and aspartate aminotransferase (AST; B) levels in the GHS discovery cohort in the region around HSD17B13.

FIG. 7 shows the expression of PNPLA3 in homozygous reference (T/T), heterozygous (T/TA), and homozygous alternate (TA/TA) carriers of the HSD17B13 rs72613567 splice variant.

FIG. 8 shows the expression difference of the 63 PNPLA3 rs738409 carriers (C/C and C/G) in the three HSD17B13 rs72613567 genotypes (T/T, T/TA, TA/TA).

FIG. 9 shows an analysis of the genetic interaction between PNPLA3 rs738409 (p.I148M) and HSD17B13 rs72613567.

FIG. 10 (panels A and B) shows HSD17B13 rs72613567: TA mitigates the risk of liver injury associated with PNPLA3 p.I148M.

FIG. 11 (panels A through F) shows raw and residualized ALT levels by PNPLA3 rs738409 (p.I148M) and HSD17B13 rs72613567 genotype.

FIG. 12 (panels A through F) shows raw and residualized AST levels by PNPLA3 rs738409 (p.I148M) and HSD17B13 rs72613567 genotype.

FIG. 13 (panels A through F) show mRNA expression of four additional novel HSD17B13 transcripts (E-H) in homozygous reference (T/T), heterozygous (T/TA), and homozygous alternate (TA/TA) carriers of the HSD17B13 splice variant.

FIG. 14 (panels A and B) shows Manhattan plots (left) and quantile-quantile plots (right) of single nucleotide variant associations with serum transaminase levels in the GHS discovery cohort.

FIG. 15 (panels A and B) shows HSD17B13 rs72613567: TA is associated with reduced risk of alcoholic and nonalcoholic liver disease phenotypes.

FIG. 16 (panels A and B) shows HSD17B13 rs72613567: TA mitigates the risk of liver injury associated with PNPLA3 p.I148M.

FIG. 17 (panels A and B) shows HSD17B13 rs72613567: TA is associated with reduced risk of progression from simple steatosis to steatohepatitis and fibrosis.

FIG. 18 (panels A through G) shows Expression, subcellular localization, and enzymatic activity of a novel HSD17B13 transcript.

FIG. 19 (panels A and B) shows HSD17B13 rs72613567: TA mitigates the risk of alcoholic and nonalcoholic liver disease associated with PNPLA3 1148M. The numbers over each bar represent controls/cases.

Additional advantages of the disclosure will be set forth in part in the description which follows, and in part will be apparent from the description, or can be learned by practice of the embodiments disclosed herein. The advantages of the disclosure will be realized and attained by means of the elements and combinations particularly pointed out in the appended claims. It is to be understood that both the foregoing general description and the following detailed description are exemplary and explanatory only and are not restrictive of the embodiments, as claimed.

### DESCRIPTION

Various terms relating to aspects of disclosure are used throughout the specification and claims. Such terms are to be given their ordinary meaning in the art, unless otherwise indicated. Other specifically defined terms are to be construed in a manner consistent with the definition provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the terms “subject” and “patient” are used interchangeably. A subject may include any animal, including mammals. Mammals include, without limitation, farm animals (e.g., horse, cow, pig), companion animals (e.g., dog, cat), laboratory animals (e.g., mouse, rat, rabbits), and non-human primates. In some embodiments, the subject is a human being.

As used herein, a “nucleic acid,” a “nucleic acid molecule,” a “nucleic acid sequence,” “polynucleotide,” or “oligonucleotide” can comprise a polymeric form of nucleotides of any length, may comprise DNA and/or RNA, and can be single-stranded, double-stranded, or multiple stranded. One strand of a nucleic acid also refers to its complement.

As used herein, the phrase “corresponding to” or grammatical variations thereof when used in the context of the numbering of a given amino acid or nucleic acid sequence or position refers to the numbering of a specified reference sequence when the given amino acid or nucleic acid sequence is compared to the reference sequence (e.g., with the reference sequence herein being the nucleic acid molecule or polypeptide of (functional or transcript behaving as a functional) HSD17B13, for example). In other words, the residue (e.g., amino acid or nucleotide) number or residue (e.g., amino acid or nucleotide) position of a given polymer is designated with respect to the reference sequence rather than by the actual numerical position of the residue within the given amino acid or nucleic acid sequence. For example, a given amino acid sequence can be aligned to a reference sequence by introducing gaps to optimize residue matches between the two sequences. In these cases, although the gaps are present, the numbering of the residue in the given amino acid or nucleic acid sequence is made with respect to the reference sequence to which it has been aligned.

For example, the phrase “nucleic acid molecule encoding an HSD17B13 loss-of-function variant protein which comprises a thymine at the position corresponding to position 12,667 according to SEQ ID NO:2” (and similar phrases) means that, if the nucleic acid sequence of the HSD17B13 genomic DNA being examined is aligned to the nucleotide sequence according to SEQ ID NO:2, the HSD17B13 genomic DNA being examined comprises a thymine at the position that corresponds to position 12,667 of SEQ ID NO:2.

A nucleic acid molecule encoding an HSD17B13 loss-of-function variant protein which comprises a thymine at the position corresponding to position 12,667 according to SEQ ID NO:2, for example, can easily be identified by performing a sequence alignment between the given HSD17B13 protein and the nucleic acid sequence of SEQ ID NO:2. Likewise, a PNPLA3 Ile148Met protein having a methionine at a position corresponding to position 148 according to SEQ ID NO:42, or at a position corresponding to position 144 according to SEQ ID NO: 43 can easily be identified by performing a sequence alignment between the given PNPLA3 protein and the amino acid sequence of SEQ ID NO:42 or SEQ ID NO: 43. A variety of computational algorithms exist that can be used for performing a sequence alignment in order to identify particular nucleic acid molecules and proteins having particular nucleotides or amino acids at the particular position that corresponds to a position of a particular SEQ ID NOs. For example, programs for identifying percent sequence identity can be used to perform a sequence alignment. Percent identity (or percent complementarity) between particular stretches of nucleic acid sequences within nucleic acids or amino acid sequences within polypeptides can be determined using BLAST programs (basic local alignment search tools) and PowerBLAST programs (Altschul et al., J. Mol. Biol., 1990, 215, 403-410; Zhang and Madden, Genome Res., 1997, 7, 649-656) or CLUSTALW software (Sievers et al., 2014, Methods Mol. Biol., 1079, 105-116) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). However, sequences can also be aligned manually. Herein, if reference is made to percent sequence identity, the higher percentages of sequence identity are preferred over the lower ones.

The present disclosure provides methods of identifying a human subject as a candidate for treating or inhibiting a liver disease by inhibiting HSD17B13; methods of treating or inhibiting liver disease comprising administering an inhibitor of HSD17B13; methods of detecting PNPLA3 Ile148Met (also referred to herein as “I148M”) and functional HSD17B13 in a subject; methods of identifying a subject having a protective effect against liver disease; and inhibitors of HSD17B13 for use in the treatment of a liver disease.

The present disclosure provides methods of classifying a human subject as a candidate for treating or inhibiting a liver disease by inhibiting HSD17B13; methods of treating or inhibiting liver disease comprising administering an inhibitor of HSD17B13; methods of detecting PNPLA3 Ile148Met (also referred to herein as “I148M”) and functional HSD17B13 in a subject; methods of classifying a subject having a protective effect against liver disease; and inhibitors of HSD17B13 for use in the treatment of a liver disease.

It has been observed in accordance with the disclosure that a splice variant (rs72613567: TA) in HSD17B13, which encodes 17-beta hydroxysteroid dehydrogenase 13, a hepatic lipid droplet protein, was reproducibly associated with reduced ALT (P=4.2×10⁻¹²) and AST (P=6.2×10⁻¹⁰) levels. It was also observed that this variant was associated with reduced risk of alcoholic and nonalcoholic liver disease (by 38%, 95% confidence interval (CI) 19%-52%; and by 16%, 95% CI 9%-22%, respectively, for each rs72613567: TA allele) and cirrhosis (by 44%, 95% CI 22-59%; and by 26%, 95% CI 12%-38% for alcoholic and nonalcoholic cirrhosis, respectively, for each rs72613567: TA allele) in an allele dosage-dependent manner. The associations were confirmed in two independent cohorts. rs72613567: TA was associated with decreased severity of histological features of nonalcoholic steatohepatitis (NASH) (23% reduction, 95% CI 10%-34% in nonalcoholic steatohepatitis (NASH) for each rs72613567: TA allele among individuals with fatty liver disease), and mitigated liver injury associated with PNPLA3 p.I148M. rs72613567: TA results in a truncated isoform deficient in enzymatic activity against steroid substrates. Thus, a loss-of-function variant in HSD17B13 was associated with reduced risk of alcoholic and nonalcoholic liver disease, and progression from steatosis to NASH. U.S. Patent Application Publication No. US2018/0216084 (corresponding to PCT Publication No. WO 2018/136702) is incorporated herein by reference in its entirety.

The present disclosure provides methods for identifying a human subject as a candidate for treating or inhibiting a liver disease by inhibiting hydroxysteroid 17-beta dehydrogenase 13 (HSD17B13), the method comprising determining whether or not a sample from the subject comprises a first nucleic acid encoding a patatin like phospholipase domain containing 3 (PNPLA3) protein comprising an I148M variation and a second nucleic acid encoding a functional HSD17B13 protein, and/or a PNPLA3 protein comprising an I148M variation and a functional HSD17B13 protein, and identifying the subject as a candidate for treating or inhibiting a liver disease by inhibiting HSD17B13 when both the first and second nucleic acids are detected and/or both of the proteins are detected.

The present disclosure also provides methods of classifying a human subject as a candidate for treating or inhibiting a liver disease by inhibiting HSD17B13; methods of treating or inhibiting liver disease comprising administering an inhibitor of HSD17B13; methods of detecting PNPLA3 Ile148Met (also referred to herein as “I148M”) and functional HSD17B13 in a subject; methods of classifying a subject having a protective effect against liver disease; and inhibitors of HSD17B13 for use in the treatment of a liver disease.

The present disclosure also provides methods of treating or inhibiting liver disease, comprising administering an inhibitor of hydroxysteroid 17-beta dehydrogenase 13 (HSD17B13) to a human liver disease patient expressing a patatin like phospholipase domain containing 3 (PNPLA3) protein comprising an I148M variation such that liver disease is treated or inhibited in the patient.

In the methods described herein, various PNPLA3 and HSD17B13 proteins, and nucleic acid molecules (e.g., genomic DNA, mRNA, and cDNA derived from the mRNA) encoding the same are detected, expressed, or employed. These PNPLA3 and HSD17B13 proteins and nucleic acid molecules encoding the same are described in more detail.

The amino acid sequences for two wild type PNPLA3 proteins are set forth in SEQ ID NO: 40 and SEQ ID NO:41. The wild type PNPLA3 protein having SEQ ID NO:40 is 481 amino acids in length, whereas the wild type PNPLA3 protein having SEQ ID NO:41 is 477 amino acids in length. The wild type PNPLA3 protein having SEQ ID NO:40 has an isoleucine at position 148. The wild type PNPLA3 protein having SEQ ID NO:41 has an isoleucine at position 144.

In some embodiments, a variant PNPLA3 Ile148Met protein comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:42, and comprises a methionine at a position corresponding to position 148 according to SEQ ID NO:42. In some embodiments, the variant PNPLA3 Ile148Met protein comprises an amino acid sequence that has at least about 95%, at least about 96%, at least about 97%, at least about 98%, or at least about 99% sequence identity to the amino acid sequence according to SEQ ID NO:42, and comprises a methionine at a position corresponding to position 148 according to SEQ ID NO:42. In some embodiments, the variant PNPLA3 Ile148Met protein comprises or consists of the amino acid sequence according to SEQ ID NO:42.

In some embodiments, a variant PNPLA3 Ile144Met protein comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:43, and comprises a methionine at a position corresponding to position 144 according to SEQ ID NO:43. In some embodiments, the variant PNPLA3 Ile144Met protein comprises an amino acid sequence that has at least about 95%, at least about 96%, at least about 97%, at least about 98%, or at least about 99% sequence identity to the amino acid sequence according to SEQ ID NO:43, and comprises a methionine at a position corresponding to position 144 according to SEQ ID NO:43. In some embodiments, the variant PNPLA3 Ile144Met protein comprises or consists of the amino acid sequence according to SEQ ID NO:43.

In some embodiments, the variant PNPLA3 Ile148Met and variant PNPLA3 Ile144Met proteins are fragments of the proteins described above, wherein the fragments comprise a methionine at a position corresponding to position 148 according to SEQ ID NO: 42, or comprise a methionine at a position corresponding to position 144 according to SEQ ID NO:43. In some embodiments, the fragments comprise at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 150, or at least about 200 contiguous amino acid residues of the encoded polypeptide (such as the polypeptide having the amino acid sequence of SEQ ID NO:42 or SEQ ID NO:43). In this regard, the longer fragments are preferred over the shorter ones. In some embodiments, the fragments comprise at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 contiguous amino acid residues of the encoded polypeptide. In this regard, the longer fragments are preferred over the shorter ones.

The nucleic acid sequence for a genomic DNA molecule encoding wild type PNPLA3 protein is set forth in SEQ ID NO:30. The wild type PNPLA3 genomic DNA molecule having SEQ ID NO:30 comprises a cytosine at position 5109. The wild type PNPLA3 genomic DNA molecule having SEQ ID NO:30 comprises the codon ATC at the positions 5107 to 5109.

In some embodiments, the variant PNPLA3 genomic DNA molecule comprises or consists of a nucleic acid sequence that encodes a PNPLA3 Ile148Met protein or a PNPLA3 Ile144Met protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:42 or SEQ ID NO:43, respectively, and comprises a methionine at a position corresponding to position 148 according to SEQ ID NO:42 or comprises a methionine at a position corresponding to position 144 according to SEQ ID NO: 43. In some embodiments, the variant PNPLA3 genomic DNA molecule comprises or consists of a nucleic acid sequence that encodes a PNPLA3 Ile148Met protein or a PNPLA3 Ile144Met protein that comprises an amino acid sequence that has at least about 95%, at least about 96%, at least about 97%, at least about 98%, or at least about 99% sequence identity to SEQ ID NO:42 or SEQ ID NO:43, respectively, and comprises a methionine at a position corresponding to position 148 according to SEQ ID NO:42 or comprises a methionine at a position corresponding to position 144 according to SEQ ID NO:43. In some embodiments, the variant PNPLA3 genomic DNA molecule comprises or consists a nucleic acid sequence that encodes a PNPLA3 Ile148Met protein or a PNPLA3 Ile144Met protein that comprises or consists of an amino acid sequence according to SEQ ID NO:42 or SEQ ID NO: 43, respectively.

In some embodiments, the variant PNPLA3 genomic DNA molecule encoding the variant PNPLA3 Ile148Met protein or the variant PNPLA3 Ile144Met protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 31, and comprises a guanine at a position corresponding to position 5109 according to SEQ ID NO:31, or comprises the codon ATG at the positions corresponding to positions 5107 to 5109 according to SEQ ID NO:31. In some embodiments, the variant PNPLA3 genomic DNA molecule encoding the variant PNPLA3 Ile148Met protein or the variant PNPLA3 Ile144Met protein comprises or consists of a nucleic acid sequence that has at least about 95%, at least about 96%, at least about 97%, at least about 98%, or at least about 99% sequence identity to SEQ ID NO:31, and comprises a guanine at a position corresponding to position 5109 according to SEQ ID NO:31, or comprises the codon ATG at the positions corresponding to positions 5107 to 5109 according to SEQ ID NO:31. In some embodiments, the variant PNPLA3 genomic DNA molecule encoding the variant PNPLA3 Ile148Met protein or the variant PNPLA3 Ile144Met protein comprises or consists of the nucleotide sequence according to SEQ ID NO:31.

In some embodiments, the variant PNPLA3 genomic DNA molecules comprise less than the entire genomic DNA sequence. In some embodiments, the variant PNPLA3 genomic DNA molecules comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, at least about 10000, at least about 11000, or at least about 11500 contiguous nucleotides of SEQ ID NO: 31. In some embodiments, the variant PNPLA3 genomic DNA molecules comprise or consist of at least about 1000 to at least about 2000 contiguous nucleotides of SEQ ID NO: 31.

In some embodiments, the variant PNPLA3 genomic DNA molecules comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, or at least about 2500 contiguous nucleotides of SEQ ID NO:31.

The nucleic acid sequences of two wild type PNPLA3 mRNA molecules are set forth in SEQ ID NO:32 and SEQ ID NO:33. The wild type PNPLA3 mRNA molecule having SEQ ID NO: 32 comprises a cytosine at position 444. The wild type PNPLA3 mRNA molecule having SEQ ID NO:32 comprises the codon AUC at the positions 442 to 444. The wild type PNPLA3 mRNA molecule having SEQ ID NO:33 comprises a cytosine at position 432. The wild type PNPLA3 mRNA molecule having SEQ ID NO:33 comprises the codon AUC at the positions 430 to 432.

In some embodiments, the variant PNPLA3 mRNA molecule comprises or consists of a nucleic acid sequence that encodes a PNPLA3 Ile148Met protein or a PNPLA3 Ile144Met protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:42 or SEQ ID NO:43, respectively, and comprises a methionine at a position corresponding to position 148 according to SEQ ID NO:42 or comprises a methionine at a position corresponding to position 144 according to SEQ ID NO:43. In some embodiments, the variant PNPLA3 mRNA molecule comprises or consists of a nucleic acid sequence that encodes a PNPLA3 Ile148Met protein or a PNPLA3 Ile144Met protein that comprises an amino acid sequence that has at least about 95%, at least about 96%, at least about 97%, at least about 98%, or at least about 99% sequence identity to SEQ ID NO:42 or SEQ ID NO:43, respectively, and comprises a methionine at a position corresponding to position 148 according to SEQ ID NO:42 or comprises a methionine at a position corresponding to position 144 according to SEQ ID NO:43. In some embodiments, the variant PNPLA3 mRNA molecule comprises or consists a nucleic acid sequence that encodes a PNPLA3 Ile148Met protein or a PNPLA3 Ile144Met protein that comprises or consists of an amino acid sequence according to SEQ ID NO:42 or SEQ ID NO:43, respectively.

In some embodiments, the variant PNPLA3 mRNA molecule encoding the variant PNPLA3 Ile148Met protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:34, and comprises a guanine at a position corresponding to position 444 according to SEQ ID NO:34, or comprises the codon AUG at the positions corresponding to positions 442 to 444 according to SEQ ID NO:34. In some embodiments, the variant PNPLA3 mRNA molecule encoding the variant PNPLA3 Ile148Met protein comprises or consists of a nucleic acid sequence that has at least about 95%, at least about 96%, at least about 97%, at least about 98%, or at least about 99% sequence identity to SEQ ID NO:34, and comprises a guanine at a position corresponding to position 444 according to SEQ ID NO:34, or comprises the codon AUG at the positions corresponding to positions 442 to 444 according to SEQ ID NO:34. In some embodiments, the variant PNPLA3 mRNA molecule encoding the variant PNPLA3 Ile148Met protein comprises or consists of the nucleotide sequence according to SEQ ID NO:34.

In some embodiments, the variant PNPLA3 mRNA molecule encoding the variant PNPLA3 Ile144Met protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:35, and comprises a guanine at a position corresponding to position 432 according to SEQ ID NO:35, or comprises the codon AUG at the positions corresponding to positions 430 to 432 according to SEQ ID NO:35. In some embodiments, the variant PNPLA3 mRNA molecule encoding the variant PNPLA3 Ile144Met protein comprises or consists of a nucleic acid sequence that has at least about 95%, at least about 96%, at least about 97%, at least about 98%, or at least about 99% sequence identity to SEQ ID NO:35, and comprises a guanine at a position corresponding to position 432 according to SEQ ID NO:35, or comprises the codon AUG at the positions corresponding to positions 430 to 432 according to SEQ ID NO:35. In some embodiments, the variant PNPLA3 mRNA molecule encoding the variant PNPLA3 Ile144Met protein comprises or consists of the nucleotide sequence according to SEQ ID NO:35.

In some embodiments, the variant PNPLA3 mRNA molecule comprises less nucleotides than the entire variant PNPLA3 mRNA sequence. In some embodiments, the variant PNPLA3 mRNA molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, or at least about 600 contiguous nucleotides of SEQ ID NO:34 or SEQ ID NO:35. In some embodiments, the variant PNPLA3 mRNA molecules comprise or consist of at least about 200 to at least about 500 contiguous nucleotides of SEQ ID NO:34 or SEQ ID NO:35. In this regard, the longer mRNA molecules are preferred over the shorter ones. In some embodiments, the variant PNPLA3 mRNA molecules comprise or consist of at least about 50, at least about 60, at least about 70, at least about 80, at least about 90, at least about 100, at least about 200, at least about 300, at least about 400, or at least about 500 contiguous nucleotides of SEQ ID NO:34 or SEQ ID NO:35. In this regard, the longer mRNA molecules are preferred over the shorter ones. In some embodiments, such variant PNPLA3 mRNA molecules include the codon that encodes the methionine at the position that corresponds to position 148 according to SEQ ID NO: 42 or the codon that encodes the methionine at the position that corresponds to position 144 according to SEQ ID NO:43. In some embodiments, such variant PNPLA3 mRNA molecules include the guanine at the position corresponding to position 444 according to SEQ ID NO:34 or the guanine at the position corresponding to position 432 according to SEQ ID NO: 35. In some embodiments, such variant PNPLA3 mRNA molecules include the codon AUG at the positions corresponding to positions 442 to 444 according to SEQ ID NO:34, or the codon AUG at the positions corresponding to positions 430 to 432 according to SEQ ID NO: 35.

The nucleic acid sequences of two wild type PNPLA3 cDNA molecules are set forth in SEQ ID NO:36 and SEQ ID NO:37. The wild type PNPLA3 cDNA molecule having SEQ ID NO: 36 comprises a cytosine at position 444. The wild type PNPLA3 cDNA molecule having SEQ ID NO:36 comprises the codon ATC at positions 442 to 444. The wild type PNPLA3 cDNA molecule having SEQ ID NO:37 comprises a cytosine at position 432. The wild type PNPLA3 cDNA molecule having SEQ ID NO:37 comprises the codon ATC at positions 430 to 432.

In some embodiments, the variant PNPLA3 cDNA molecule comprises or consists of a nucleic acid sequence that encodes a PNPLA3 Ile148Met protein or a PNPLA3 Ile144Met protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:42 or SEQ ID NO:43, respectively, and comprises a methionine at a position corresponding to position 148 according to SEQ ID NO:42 or comprises a methionine at a position corresponding to position 144 according to SEQ ID NO:43. In some embodiments, the variant PNPLA3 cDNA molecule comprises or consists of a nucleic acid sequence that encodes a PNPLA3 Ile148Met protein or a PNPLA3 Ile144Met protein that comprises an amino acid sequence that has at least about 95%, at least about 96%, at least about 97%, at least about 98%, or at least about 99% sequence identity to SEQ ID NO:42 or SEQ ID NO:43, respectively, and comprises a methionine at a position corresponding to position 148 according to SEQ ID NO:42 or comprises a methionine at a position corresponding to position 144 according to SEQ ID NO:43. In some embodiments, the variant PNPLA3 cDNA molecule comprises or consists a nucleic acid sequence that encodes a PNPLA3 Ile148Met protein or a PNPLA3 Ile144Met protein that comprises or consists of an amino acid sequence according to SEQ ID NO:42 or SEQ ID NO:43, respectively.

In some embodiments, the variant PNPLA3 cDNA molecule encoding the variant PNPLA3 Ile148Met protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:38, and comprises a guanine at a position corresponding to position 444 according to SEQ ID NO:38, or comprises the codon ATG at the positions corresponding to positions 442 to 444 according to SEQ ID NO:38. In some embodiments, the variant PNPLA3 cDNA molecule encoding the variant PNPLA3 Ile148Met protein comprises or consists of a nucleic acid sequence that has at least about 95%, at least about 96%, at least about 97%, at least about 98%, or at least about 99% sequence identity to SEQ ID NO:38, and comprises a guanine at a position corresponding to position 444 according to SEQ ID NO:38, or comprises the codon ATG at the positions corresponding to positions 442 to 444 according to SEQ ID NO:38. In some embodiments, the variant PNPLA3 cDNA molecule encoding the variant PNPLA3 Ile148Met protein comprises or consists of the nucleotide sequence according to SEQ ID NO:38.

In some embodiments, the variant PNPLA3 cDNA molecule encoding the variant PNPLA3 Ile144Met protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:39, and comprises a guanine at a position corresponding to position 432 according to SEQ ID NO:39, or comprises the codon ATG at the positions corresponding to positions 430 to 432 according to SEQ ID NO:39. In some embodiments, the variant PNPLA3 cDNA molecule encoding the variant PNPLA3 Ile144Met protein comprises or consists of a nucleic acid sequence that has at least about 95%, at least about 96%, at least about 97%, at least about 98%, or at least about 99% sequence identity to SEQ ID NO:39, and comprises a guanine at a position corresponding to position 432 according to SEQ ID NO:39, or comprises the codon ATG at the positions corresponding to positions 430 to 432 according to SEQ ID NO:39. In some embodiments, the variant PNPLA3 cDNA molecule encoding the variant PNPLA3 Ile144Met protein comprises or consists of the nucleotide sequence according to SEQ ID NO:39.

In some embodiments, the variant PNPLA3 cDNA molecule comprises less nucleotides than the entire variant PNPLA3 cDNA sequence. In some embodiments, the variant PNPLA3 cDNA molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, or at least about 600 contiguous nucleotides of SEQ ID NO:38 or SEQ ID NO:39. In some embodiments, the variant PNPLA3 cDNA molecules comprise or consist of at least about 200 to at least about 500 contiguous nucleotides of SEQ ID NO:38 or SEQ ID NO:39. In this regard, the longer cDNA molecules are preferred over the shorter ones. In some embodiments, the variant PNPLA3 cDNA molecules comprise or consist of at least about 50, at least about 60, at least about 70, at least about 80, at least about 90, at least about 100, at least about 200, at least about 300, at least about 400, or at least about 500 contiguous nucleotides of SEQ ID NO:38 or SEQ ID NO:39. In this regard, the longer cDNA molecules are preferred over the shorter ones. In some embodiments, such variant PNPLA3 cDNA molecules include the codon that encodes the methionine at the position that corresponds to position 148 according to SEQ ID NO: 42 or the codon that encodes the methionine at the position that corresponds to position 144 according to SEQ ID NO:43. In some embodiments, such variant PNPLA3 cDNA molecules include the guanine at the position corresponding to position 444 according to SEQ ID NO:38 or the guanine at the position corresponding to position 432 according to SEQ ID NO: 39. In some embodiments, such variant PNPLA3 cDNA molecules include the codon ATG at the positions corresponding to positions 442 to 444 according to SEQ ID NO:38, or the codon ATG at the positions corresponding to positions 430 to 432 according to SEQ ID NO: 39.

The amino acid sequences for four HSD17B13 isoform proteins associated with the functional HSD17B13 protein are set forth in SEQ ID NO:21 (Isoform A), SEQ ID NO:22 (Isoform B), SEQ ID NO:25 (Isoform E), and SEQ ID NO:29 (Isoform I). The HSD17B13 protein having SEQ ID NO:21 (Isoform A) is 300 amino acids in length. The HSD17B13 protein having SEQ ID NO:22 (Isoform B) is 264 amino acids in length. The HSD17B13 protein having SEQ ID NO: 25 (Isoform E) is 324 amino acids in length. The HSD17B13 protein having SEQ ID NO:29 (Isoform I) is 271 amino acids in length.

In some embodiments, an HSD17B13 isoform protein associated with the functional HSD17B13 protein comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:21 (Isoform A). In some embodiments, the HSD17B13 isoform protein associated with the functional HSD17B13 protein comprises an amino acid sequence that has at least about 95%, at least about 96%, at least about 97%, at least about 98%, or at least about 99% sequence identity to the amino acid sequence according to SEQ ID NO:21 (Isoform A). In some embodiments, the HSD17B13 isoform protein associated with the functional HSD17B13 protein comprises or consists of the amino acid sequence according to SEQ ID NO:21 (Isoform A).

In some embodiments, an HSD17B13 isoform protein associated with the functional HSD17B13 protein comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:22 (Isoform B). In some embodiments, the HSD17B13 isoform protein associated with the functional HSD17B13 protein comprises an amino acid sequence that has at least about 95%, at least about 96%, at least about 97%, at least about 98%, or at least about 99% sequence identity to the amino acid sequence according to SEQ ID NO:22 (Isoform B). In some embodiments, the HSD17B13 isoform protein associated with the functional HSD17B13 protein comprises or consists of the amino acid sequence according to SEQ ID NO:22 (Isoform B).

In some embodiments, an HSD17B13 isoform protein associated with the functional HSD17B13 protein comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:25 (Isoform E). In some embodiments, the HSD17B13 isoform protein associated with the functional HSD17B13 protein comprises an amino acid sequence that has at least about 95%, at least about 96%, at least about 97%, at least about 98%, or at least about 99% sequence identity to the amino acid sequence according to SEQ ID NO:25 (Isoform E). In some embodiments, the HSD17B13 isoform protein associated with the functional HSD17B13 protein comprises or consists of the amino acid sequence according to SEQ ID NO:25 (Isoform E).

In some embodiments, an HSD17B13 isoform protein associated with the functional HSD17B13 protein comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:29 (Isoform I). In some embodiments, the HSD17B13 isoform protein associated with the functional HSD17B13 protein comprises an amino acid sequence that has at least about 95%, at least about 96%, at least about 97%, at least about 98%, or at least about 99% sequence identity to the amino acid sequence according to SEQ ID NO:29 (Isoform I). In some embodiments, the HSD17B13 isoform protein associated with the functional HSD17B13 protein comprises or consists of the amino acid sequence according to SEQ ID NO:29 (Isoform I).

The amino acid sequences for five HSD17B13 isoform proteins associated with the loss-of-function rs72613567 HSD17B13 protein (SEQ ID NO:2) are set forth in SEQ ID NO:23 (Isoform C), SEQ ID NO:24 (Isoform D), SEQ ID NO:26 (Isoform F), SEQ ID NO:27 (Isoform G), and SEQ ID NO:28 (Isoform H). The HSD17B13 protein having SEQ ID NO:23 (Isoform C) is 261 amino acids in length. The HSD17B13 protein having SEQ ID NO:24 (Isoform D) is 274 amino acids in length. The HSD17B13 protein having SEQ ID NO:26 (Isoform F) is 284 amino acids in length. The HSD17B13 protein having SEQ ID NO:27 (Isoform G) is 238 amino acids in length. The HSD17B13 protein having SEQ ID NO:28 (Isoform H) is 298 amino acids in length.

In some embodiments, an HSD17B13 variant protein associated with a loss-of-function comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:23 (Isoform C). In some embodiments, the HSD17B13 variant protein associated with a loss-of-function comprises an amino acid sequence that has at least about 95%, at least about 96%, at least about 97%, at least about 98%, or at least about 99% sequence identity to the amino acid sequence according to SEQ ID NO: 23 (Isoform C). In some embodiments, the HSD17B13 variant protein associated with a loss-of-function comprises or consists of the amino acid sequence according to SEQ ID NO: 23 (Isoform C).

In some embodiments, an HSD17B13 variant protein associated with a loss-of-function comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:24 (Isoform D). In some embodiments, the HSD17B13 variant protein associated with a loss-of-function comprises an amino acid sequence that has at least about 95%, at least about 96%, at least about 97%, at least about 98%, or at least about 99% sequence identity to the amino acid sequence according to SEQ ID NO: 24 (Isoform D). In some embodiments, the HSD17B13 variant protein associated with a loss-of-function comprises an amino acid sequence that comprises or consists of the amino acid sequence according to SEQ ID NO:24 (Isoform D).

In some embodiments, an HSD17B13 variant protein associated with a loss-of-function comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:26 (Isoform F). In some embodiments, the HSD17B13 variant protein associated with a loss-of-function comprises an amino acid sequence that has at least about 95%, at least about 96%, at least about 97%, at least about 98%, or at least about 99% sequence identity to the amino acid sequence according to SEQ ID NO: 26 (Isoform F). In some embodiments, the HSD17B13 variant protein associated with a loss-of-function comprises or consists of the amino acid sequence according to SEQ ID NO: 26 (Isoform F).

In some embodiments, an HSD17B13 variant protein associated with a loss-of-function comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:27 (Isoform G). In some embodiments, the HSD17B13 variant protein associated with a loss-of-function comprises an amino acid sequence that has at least about 95%, at least about 96%, at least about 97%, at least about 98%, or at least about 99% sequence identity to the amino acid sequence according to SEQ ID NO: 27 (Isoform G). In some embodiments, the HSD17B13 variant protein associated with a loss-of-function comprises or consists of the amino acid sequence according to SEQ ID NO: 27 (Isoform G).

In some embodiments, an HSD17B13 variant protein associated with a loss-of-function comprises an amino acid sequence that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:28 (Isoform H). In some embodiments, the HSD17B13 variant protein associated with a loss-of-function comprises an amino acid sequence that has at least about 95%, at least about 96%, at least about 97%, at least about 98%, or at least about 99% sequence identity to the amino acid sequence according to SEQ ID NO: 28 (Isoform H). In some embodiments, the HSD17B13 variant protein associated with a loss-of-function comprises or consists of the amino acid sequence according to SEQ ID NO: 28 (Isoform H).

In some embodiments, the HSD17B13 isoform proteins associated with the functional HSD17B13 protein and the HSD17B13 variant proteins associated with a loss-of-function are fragments of the proteins described above. In some embodiments, the fragments comprise at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 150, or at least about 200 contiguous amino acid residues of the encoded polypeptide (such as the polypeptides having the amino acid sequence of SEQ ID NO:21, SEQ ID NO: 22, SEQ ID NO: 23, SEQ ID NO: 24, SEQ ID NO:25, SEQ ID NO:26, SEQ ID NO:27, SEQ ID NO: 28, or SEQ ID NO:29). In this regard, the longer fragments are preferred over the shorter ones. In some embodiments, the fragments comprise at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 contiguous amino acid residues of the encoded polypeptide. In this regard, the longer fragments are preferred over the shorter ones.

A nucleic acid sequence for the functional HSD17B13 genomic DNA molecule is set forth in SEQ ID NO:1. The functional HSD17B13 genomic DNA molecule having SEQ ID NO:1 comprises an adenine at position 12,667.

In some embodiments, the functional HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 21 (Isoform A). In some embodiments, the functional HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 21 (Isoform A). In some embodiments, the functional HSD17B13 genomic DNA molecule comprises or consists a nucleic acid sequence that encodes an HSD17B13 isoform protein that comprises or consists of an amino acid sequence according to SEQ ID NO:21 (Isoform A).

In some embodiments, the functional HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 22 (Isoform B). In some embodiments, the functional HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 22 (Isoform B). In some embodiments, the functional HSD17B13 genomic DNA molecule comprises or consists a nucleic acid sequence that encodes an HSD17B13 isoform protein that comprises or consists of an amino acid sequence according to SEQ ID NO:22 (Isoform B).

In some embodiments, the functional HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 25 (Isoform E). In some embodiments, the functional HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 25 (Isoform E). In some embodiments, the functional HSD17B13 genomic DNA molecule comprises or consists a nucleic acid sequence that encodes an HSD17B13 isoform protein that comprises or consists of an amino acid sequence according to SEQ ID NO:25 (Isoform E).

In some embodiments, the functional HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 29 (Isoform I). In some embodiments, the functional HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 29 (Isoform I). In some embodiments, the functional HSD17B13 genomic DNA molecule comprises or consists a nucleic acid sequence that encodes an HSD17B13 isoform protein that comprises or consists of an amino acid sequence according to SEQ ID NO:29 (Isoform I).

In some embodiments, the functional HSD17B13 genomic DNA molecule comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 1. In some embodiments, the functional HSD17B13 genomic DNA molecule comprises or consists of a nucleic acid sequence that has at least about 95%, at least about 96%, at least about 97%, at least about 98%, or at least about 99% sequence identity to SEQ ID NO: 1. In some embodiments, the functional HSD17B13 genomic DNA molecule comprises or consists a nucleic acid sequence according to SEQ ID NO:21.

A nucleic acid sequence for the variant HSD17B13 genomic DNA molecule encoding an HSD17B13 variant protein associated with a loss-of-function is set forth in SEQ ID NO:2. The variant HSD17B13 genomic DNA molecule having SEQ ID NO:2 comprises a thymine at position 12,667.

In some embodiments, the variant HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 23 (Isoform C). In some embodiments, the variant HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 23 (Isoform C). In some embodiments, the variant HSD17B13 genomic DNA molecule comprises or consists a nucleic acid sequence that encodes an HSD17B13 isoform protein that comprises or consists of an amino acid sequence according to SEQ ID NO:23 (Isoform C).

In some embodiments, the variant HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 24 (Isoform D). In some embodiments, the variant HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 24 (Isoform D). In some embodiments, the variant HSD17B13 genomic DNA molecule comprises or consists a nucleic acid sequence that encodes an HSD17B13 isoform protein that comprises or consists of an amino acid sequence according to SEQ ID NO:24 (Isoform D).

In some embodiments, the variant HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 26 (Isoform F). In some embodiments, the variant HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 26 (Isoform F). In some embodiments, the variant HSD17B13 genomic DNA molecule comprises or consists a nucleic acid sequence that encodes an HSD17B13 isoform protein that comprises or consists of an amino acid sequence according to SEQ ID NO:26 (Isoform F).

In some embodiments, the variant HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 27 (Isoform G). In some embodiments, the variant HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 27 (Isoform G). In some embodiments, the variant HSD17B13 genomic DNA molecule comprises or consists a nucleic acid sequence that encodes an HSD17B13 isoform protein that comprises or consists of an amino acid sequence according to SEQ ID NO:27 (Isoform G).

In some embodiments, the variant HSD17B13 genomic DNA molecule comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 28 (Isoform H). In some embodiments, the variant HSD17B13 genomic DNA molecule comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 28 (Isoform H). In some embodiments, the variant HSD17B13 genomic DNA molecule comprises or consists a nucleic acid sequence that encodes an HSD17B13 isoform protein that comprises or consists of an amino acid sequence according to SEQ ID NO:28 (Isoform H).

In some embodiments, the variant HSD17B13 genomic DNA molecule comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO: 2. In some embodiments, the variant HSD17B13 genomic DNA molecule comprises or consists of a nucleic acid sequence that has at least about 95%, at least about 96%, at least about 97%, at least about 98%, or at least about 99% sequence identity to SEQ ID NO:2. In some embodiments, the variant HSD17B13 genomic DNA molecule comprises or consists a nucleic acid sequence according to SEQ ID NO:2.

In some embodiments, the functional HSD17B13 genomic DNA and variant HSD17B13 genomic DNA molecules comprise less than the entire genomic DNA sequence. In some embodiments, the functional HSD17B13 genomic DNA and variant HSD17B13 genomic DNA molecules comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, at least about 10000, at least about 11000, or at least about 11500 contiguous nucleotides of SEQ ID NO:1 (functional HSD17B13 genomic DNA) or SEQ ID NO:2 (variant HSD17B13 genomic DNA). In some embodiments, the functional HSD17B13 genomic DNA and variant HSD17B13 genomic DNA molecules comprise or consist of at least about 1000 to at least about 2000 contiguous nucleotides of SEQ ID NO:1 (functional HSD17B13 genomic DNA) or SEQ ID NO:2 (variant HSD17B13 genomic DNA).

In some embodiments, the functional HSD17B13 genomic DNA and variant HSD17B13 genomic DNA molecules comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, or at least about 2500 contiguous nucleotides of SEQ ID NO:1 (functional HSD17B13 genomic DNA) or SEQ ID NO:2 (variant HSD17B13 genomic DNA).

The nucleic acid sequences for four HSD17B13 RNA transcripts encoding isoform proteins associated with the functional HSD17B13 protein are set forth in SEQ ID NO:44 (Transcript A), SEQ ID NO:45 (Transcript B), SEQ ID NO:48 (Transcript E), and SEQ ID NO:52 (Transcript I).

In some embodiments, an HSD17B13 RNA transcript encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:21 (Isoform A). In some embodiments, the HSD17B13 RNA transcript encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:21 (Isoform A). In some embodiments, the HSD17B13 RNA transcript encoding the isoform protein associated with the functional HSD17B13 protein comprises or consists of a nucleic acid sequence that encodes an HSD17B13 isoform protein that comprises or consists of an amino acid sequence according to SEQ ID NO:21 (Isoform A).

In some embodiments, an HSD17B13 RNA transcript encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:22 (Isoform B). In some embodiments, the HSD17B13 RNA transcript encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:22 (Isoform B). In some embodiments, the HSD17B13 RNA transcript encoding the isoform protein associated with the functional HSD17B13 protein comprises or consists of a nucleic acid sequence that encodes an HSD17B13 isoform protein that comprises or consists of an amino acid sequence according to SEQ ID NO:22 (Isoform B).

In some embodiments, an HSD17B13 RNA transcript encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:25 (Isoform E). In some embodiments, the HSD17B13 RNA transcript encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:25 (Isoform E). In some embodiments, the HSD17B13 RNA transcript encoding the isoform protein associated with the functional HSD17B13 protein comprises or consists of a nucleic acid sequence that encodes an HSD17B13 isoform protein that comprises or consists of an amino acid sequence according to SEQ ID NO:25 (Isoform E).

In some embodiments, an HSD17B13 RNA transcript encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:29 (Isoform I). In some embodiments, the HSD17B13 RNA transcript encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:29 (Isoform I). In some embodiments, the HSD17B13 RNA transcript encoding the isoform protein associated with the functional HSD17B13 protein comprises or consists of a nucleic acid sequence that encodes an HSD17B13 isoform protein that comprises or consists of an amino acid sequence according to SEQ ID NO:29 (Isoform I).

In some embodiments, an HSD17B13 RNA transcript encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:44 (Transcript A). In some embodiments, the HSD17B13 RNA transcript encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:44 (Transcript A). In some embodiments, the HSD17B13 RNA transcript encoding the isoform protein associated with the functional HSD17B13 protein comprises or consists of a nucleic acid sequence according to SEQ ID NO: 44 (Transcript A).

In some embodiments, an HSD17B13 RNA transcript encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:45 (Transcript B). In some embodiments, the HSD17B13 RNA transcript encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:45 (Transcript B). In some embodiments, the HSD17B13 RNA transcript encoding the isoform protein associated with the functional HSD17B13 protein comprises or consists of a nucleic acid sequence according to SEQ ID NO: 45 (Transcript B).

In some embodiments, an HSD17B13 RNA transcript encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:48 (Transcript E). In some embodiments, the HSD17B13 RNA transcript encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:48 (Transcript E). In some embodiments, the HSD17B13 RNA transcript encoding the isoform protein associated with the functional HSD17B13 protein comprises or consists of a nucleic acid sequence according to SEQ ID NO: 48 (Transcript E).

In some embodiments, an HSD17B13 RNA transcript encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:52 (Transcript I). In some embodiments, the HSD17B13 RNA transcript encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:52 (Transcript I). In some embodiments, the HSD17B13 RNA transcript encoding the isoform protein associated with the functional HSD17B13 protein comprises or consists of a nucleic acid sequence according to SEQ ID NO: 52 (Transcript I).

The nucleic acid sequences for five HSD17B13 RNA transcripts encoding isoform proteins associated with a loss-of-function are set forth in SEQ ID NO:46 (Transcript C), SEQ ID NO: 47 (Transcript D), SEQ ID NO:49 (Transcript F), SEQ ID NO:50 (Transcript G), and SEQ ID NO: 51 (Transcript H).

In some embodiments, an HSD17B13 RNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:23 (Isoform C). In some embodiments, the HSD17B13 RNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 23 (Isoform C). In some embodiments, the HSD17B13 RNA transcript encoding the isoform protein associated with a loss-of-function comprises or consists of a nucleic acid sequence that encodes an HSD17B13 isoform protein that comprises or consists of an amino acid sequence according to SEQ ID NO:23 (Isoform C).

In some embodiments, an HSD17B13 RNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:24 (Isoform D). In some embodiments, the HSD17B13 RNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 24 (Isoform D). In some embodiments, the HSD17B13 RNA transcript encoding the isoform protein associated with a loss-of-function comprises or consists of a nucleic acid sequence that encodes an HSD17B13 isoform protein that comprises or consists of an amino acid sequence according to SEQ ID NO:24 (Isoform D).

In some embodiments, an HSD17B13 RNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:26 (Isoform F). In some embodiments, the HSD17B13 RNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 26 (Isoform F). In some embodiments, the HSD17B13 RNA transcript encoding the isoform protein associated with a loss-of-function comprises or consists of a nucleic acid sequence that encodes an HSD17B13 isoform protein that comprises or consists of an amino acid sequence according to SEQ ID NO:26 (Isoform F).

In some embodiments, an HSD17B13 RNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:27 (Isoform G). In some embodiments, the HSD17B13 RNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 27 (Isoform G). In some embodiments, the HSD17B13 RNA transcript encoding the isoform protein associated with a loss-of-function comprises or consists of a nucleic acid sequence that encodes an HSD17B13 isoform protein that comprises or consists of an amino acid sequence according to SEQ ID NO:27 (Isoform G).

In some embodiments, an HSD17B13 RNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:28 (Isoform H). In some embodiments, the HSD17B13 RNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 28 (Isoform H). In some embodiments, the HSD17B13 RNA transcript encoding the isoform protein associated with a loss-of-function comprises or consists of a nucleic acid sequence that encodes an HSD17B13 isoform protein that comprises or consists of an amino acid sequence according to SEQ ID NO:28 (Isoform H).

In some embodiments, an HSD17B13 RNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:46 (Transcript C). In some embodiments, the HSD17B13 RNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:46 (Transcript C). In some embodiments, the HSD17B13 RNA transcript encoding the isoform protein associated with a loss-of-function comprises or consists of a nucleic acid sequence according to SEQ ID NO:46 (Transcript C).

In some embodiments, an HSD17B13 RNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:47 (Transcript D). In some embodiments, the HSD17B13 RNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:47 (Transcript D). In some embodiments, the HSD17B13 RNA transcript encoding the isoform protein associated with a loss-of-function comprises or consists of a nucleic acid sequence according to SEQ ID NO:47 (Transcript D).

In some embodiments, an HSD17B13 RNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:49 (Transcript F). In some embodiments, the HSD17B13 RNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:49 (Transcript F). In some embodiments, the HSD17B13 RNA transcript encoding the isoform protein associated with a loss-of-function comprises or consists of a nucleic acid sequence according to SEQ ID NO:49 (Transcript F).

In some embodiments, an HSD17B13 RNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:50 (Transcript G). In some embodiments, the HSD17B13 RNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:50 (Transcript G). In some embodiments, the HSD17B13 RNA transcript encoding the isoform protein associated with a loss-of-function comprises or consists of a nucleic acid sequence according to SEQ ID NO:50 (Transcript G).

In some embodiments, an HSD17B13 RNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:51 (Transcript H). In some embodiments, the HSD17B13 RNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:51 (Transcript H). In some embodiments, the HSD17B13 RNA transcript encoding the isoform protein associated with a loss-of-function comprises or consists of a nucleic acid sequence according to SEQ ID NO:51 (Transcript H).

In some embodiments, the functional HSD17B13 RNA transcripts and variant HSD17B13 RNA transcripts comprise less than the RNA transcript sequence. In some embodiments, the functional HSD17B13 RNA transcripts and variant HSD17B13 RNA transcripts comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or at least about 2500 contiguous nucleotides of SEQ ID NO:44, SEQ ID NO:45, SEQ ID NO:48, or SEQ ID NO:52 (functional HSD17B13 RNA transcripts) or SEQ ID NO:46, SEQ ID NO: 47, SEQ ID NO:49, SEQ ID NO:50, or SEQ ID NO:51 (variant HSD17B13 RNA transcripts). In some embodiments, the functional HSD17B13 RNA transcripts and variant HSD17B13 RNA transcripts comprise less than the RNA transcript sequence. In some embodiments, the functional HSD17B13 RNA transcripts and variant HSD17B13 RNA transcripts comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, or at least about 500 contiguous nucleotides of SEQ ID NO:44, SEQ ID NO:45, SEQ ID NO:48, or SEQ ID NO:52 (functional HSD17B13 RNA transcripts) or SEQ ID NO:46, SEQ ID NO:47, SEQ ID NO:49, SEQ ID NO:50, or SEQ ID NO:51 (variant HSD17B13 RNA transcripts). In some embodiments, the functional HSD17B13 RNA transcripts and variant HSD17B13 RNA transcripts comprise or consist of at least about 1000 to at least about 2000 contiguous nucleotides of SEQ ID NO:44, SEQ ID NO: 45, SEQ ID NO:48, or SEQ ID NO:52 (functional HSD17B13 RNA transcripts) or SEQ ID NO: 46, SEQ ID NO: 47, SEQ ID NO: 49, SEQ ID NO:50, or SEQ ID NO:51 (variant HSD17B13 RNA transcripts).

The nucleic acid sequences for four HSD17B13 cDNA transcripts encoding isoform proteins associated with the functional HSD17B13 protein are set forth in SEQ ID NO:53 (Transcript A), SEQ ID NO:54 (Transcript B), SEQ ID NO:57 (Transcript E), and SEQ ID NO:61 (Transcript I).

In some embodiments, an HSD17B13 cDNA transcript encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:21 (Isoform A). In some embodiments, the HSD17B13 cDNA transcript encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:21 (Isoform A). In some embodiments, the HSD17B13 cDNA transcript encoding the isoform protein associated with the functional HSD17B13 protein comprises or consists of a nucleic acid sequence that encodes an HSD17B13 isoform protein that comprises or consists of an amino acid sequence according to SEQ ID NO:21 (Isoform A).

In some embodiments, an HSD17B13 cDNA transcript encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:22 (Isoform B). In some embodiments, the HSD17B13 cDNA transcript encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:22 (Isoform B). In some embodiments, the HSD17B13 cDNA transcript encoding the isoform protein associated with the functional HSD17B13 protein comprises or consists of a nucleic acid sequence that encodes an HSD17B13 isoform protein that comprises or consists of an amino acid sequence according to SEQ ID NO:22 (Isoform B).

In some embodiments, an HSD17B13 cDNA transcript encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:25 (Isoform E). In some embodiments, the HSD17B13 cDNA transcript encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:25 (Isoform E). In some embodiments, the HSD17B13 cDNA transcript encoding the isoform protein associated with the functional HSD17B13 protein comprises or consists of a nucleic acid sequence that encodes an HSD17B13 isoform protein that comprises or consists of an amino acid sequence according to SEQ ID NO:25 (Isoform E).

In some embodiments, an HSD17B13 cDNA transcript encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:29 (Isoform I). In some embodiments, the HSD17B13 cDNA transcript encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:29 (Isoform I). In some embodiments, the HSD17B13 cDNA transcript encoding the isoform protein associated with the functional HSD17B13 protein comprises or consists of a nucleic acid sequence that encodes an HSD17B13 isoform protein that comprises or consists of an amino acid sequence according to SEQ ID NO:29 (Isoform I).

In some embodiments, an HSD17B13 cDNA transcript encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:53 (Transcript A). In some embodiments, the HSD17B13 cDNA transcript encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:53 (Transcript A). In some embodiments, the HSD17B13 cDNA transcript encoding the isoform protein associated with the functional HSD17B13 protein comprises or consists of a nucleic acid sequence according to SEQ ID NO: 53 (Transcript A).

In some embodiments, an HSD17B13 cDNA transcript encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:54 (Transcript B). In some embodiments, the HSD17B13 cDNA transcript encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:54 (Transcript B). In some embodiments, the HSD17B13 cDNA transcript encoding the isoform protein associated with the functional HSD17B13 protein comprises or consists of a nucleic acid sequence according to SEQ ID NO: 54 (Transcript B).

In some embodiments, an HSD17B13 cDNA transcript encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:57 (Transcript E). In some embodiments, the HSD17B13 cDNA transcript encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:57 (Transcript E). In some embodiments, the HSD17B13 cDNA transcript encoding the isoform protein associated with the functional HSD17B13 protein comprises or consists of a nucleic acid sequence according to SEQ ID NO: 57 (Transcript E).

In some embodiments, an HSD17B13 cDNA transcript encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:61 (Transcript I). In some embodiments, the HSD17B13 cDNA transcript encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:61 (Transcript I). In some embodiments, the HSD17B13 cDNA transcript encoding the isoform protein associated with the functional HSD17B13 protein comprises or consists of a nucleic acid sequence according to SEQ ID NO: 61 (Transcript I).

The nucleic acid sequences for five HSD17B13 cDNA transcripts encoding isoform proteins associated with a loss-of-function are set forth in SEQ ID NO:55 (Transcript C), SEQ ID NO: 56 (Transcript D), SEQ ID NO:58 (Transcript F), SEQ ID NO:59 (Transcript G), and SEQ ID NO: 60 (Transcript H).

In some embodiments, an HSD17B13 cDNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:23 (Isoform C). In some embodiments, the HSD17B13 cDNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 23 (Isoform C). In some embodiments, the HSD17B13 cDNA transcript encoding the isoform protein associated with a loss-of-function comprises or consists of a nucleic acid sequence that encodes an HSD17B13 isoform protein that comprises or consists of an amino acid sequence according to SEQ ID NO:23 (Isoform C).

In some embodiments, an HSD17B13 cDNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:24 (Isoform D). In some embodiments, the HSD17B13 cDNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 24 (Isoform D). In some embodiments, the HSD17B13 cDNA transcript encoding the isoform protein associated with a loss-of-function comprises or consists of a nucleic acid sequence that encodes an HSD17B13 isoform protein that comprises or consists of an amino acid sequence according to SEQ ID NO:24 (Isoform D).

In some embodiments, an HSD17B13 cDNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:26 (Isoform F). In some embodiments, the HSD17B13 cDNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 26 (Isoform F). In some embodiments, the HSD17B13 cDNA transcript encoding the isoform protein associated with a loss-of-function comprises or consists of a nucleic acid sequence that encodes an HSD17B13 isoform protein that comprises or consists of an amino acid sequence according to SEQ ID NO:26 (Isoform F).

In some embodiments, an HSD17B13 cDNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:27 (Isoform G). In some embodiments, the HSD17B13 cDNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 27 (Isoform G). In some embodiments, the HSD17B13 cDNA transcript encoding the isoform protein associated with a loss-of-function comprises or consists of a nucleic acid sequence that encodes an HSD17B13 isoform protein that comprises or consists of an amino acid sequence according to SEQ ID NO:27 (Isoform G).

In some embodiments, an HSD17B13 cDNA transcript encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:28 (Isoform H). In some embodiments, the HSD17B13 cDNA transcript encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 28 (Isoform H). In some embodiments, the HSD17B13 cDNA transcript encoding the isoform protein associated with a loss-of-function comprises or consists of a nucleic acid sequence that encodes an HSD17B13 isoform protein that comprises or consists of an amino acid sequence according to SEQ ID NO:28 (Isoform H).

In some embodiments, an HSD17B13 cDNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:55 (Transcript C). In some embodiments, the HSD17B13 cDNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:55 (Transcript C). In some embodiments, the HSD17B13 cDNA transcript encoding the isoform protein associated with a loss-of-function comprises or consists of a nucleic acid sequence according to SEQ ID NO:55 (Transcript C).

In some embodiments, an HSD17B13 cDNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:56 (Transcript D). In some embodiments, the HSD17B13 cDNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:56 (Transcript D). In some embodiments, the HSD17B13 cDNA transcript encoding the isoform protein associated with a loss-of-function comprises or consists of a nucleic acid sequence according to SEQ ID NO:56 (Transcript D).

In some embodiments, an HSD17B13 cDNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:58 (Transcript F). In some embodiments, the HSD17B13 cDNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:58 (Transcript F). In some embodiments, the HSD17B13 cDNA transcript encoding the isoform protein associated with a loss-of-function comprises or consists of a nucleic acid sequence according to SEQ ID NO:58 (Transcript F).

In some embodiments, an HSD17B13 cDNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:59 (Transcript G). In some embodiments, the HSD17B13 cDNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:59 (Transcript G). In some embodiments, the HSD17B13 cDNA transcript encoding the isoform protein associated with a loss-of-function comprises or consists of a nucleic acid sequence according to SEQ ID NO:59 (Transcript G).

In some embodiments, an HSD17B13 cDNA transcript encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:60 (Transcript H). In some embodiments, the HSD17B13 cDNA transcript encoding an isoform protein associated with a loss-of-function comprises or consists of a nucleic acid sequence that has at least about 95%, at least about 96%, at least about 97%, at least about 98%, or at least about 99% sequence identity to SEQ ID NO:60 (Transcript H). In some embodiments, the HSD17B13 cDNA transcript encoding the isoform protein associated with a loss-of-function comprises or consists of a nucleic acid sequence according to SEQ ID NO:60 (Transcript H).

In some embodiments, the HSD17B13 cDNA transcripts comprise less than the cDNA transcript sequence. In some embodiments, the HSD17B13 cDNA transcripts comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or at least about 2500 contiguous nucleotides of SEQ ID NO:53, SEQ ID NO:54, SEQ ID NO:57, or SEQ ID NO: 61 or SEQ ID NO:55, SEQ ID NO:56, SEQ ID NO:58, SEQ ID NO:59, or SEQ ID NO:60. In some embodiments, the HSD17B13 cDNA transcripts comprise less than the cDNA transcript sequence. In some embodiments, the HSD17B13 cDNA transcripts comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, or at least about 500 contiguous nucleotides of SEQ ID NO:53, SEQ ID NO: 54, SEQ ID NO:57, or SEQ ID NO: 61 or SEQ ID NO:55, SEQ ID NO:56, SEQ ID NO:58, SEQ ID NO: 59, or SEQ ID NO:60. In some embodiments, the HSD17B13 cDNA transcripts comprise or consist of at least about 1000 to at least about 2000 contiguous nucleotides of SEQ ID NO:53, SEQ ID NO:54, SEQ ID NO:57, or SEQ ID NO:61 or SEQ ID NO:55, SEQ ID NO: 56, SEQ ID NO:58, SEQ ID NO:59, or SEQ ID NO:60.

The nucleic acid sequences for four HSD17B13 mRNA molecules encoding isoform proteins associated with the functional HSD17B13 protein are set forth in SEQ ID NO:3 (Transcript A), SEQ ID NO:4 (Transcript B), SEQ ID NO:7 (Transcript E), and SEQ ID NO:11 (Transcript I).

In some embodiments, an HSD17B13 mRNA molecule encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:21 (Isoform A). In some embodiments, the HSD17B13 mRNA molecule encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:21 (Isoform A). In some embodiments, the HSD17B13 mRNA molecule encoding the isoform protein associated with the functional HSD17B13 protein comprises or consists of a nucleic acid sequence that encodes an HSD17B13 isoform protein that comprises or consists of an amino acid sequence according to SEQ ID NO:21 (Isoform A).

In some embodiments, an HSD17B13 mRNA molecule encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:22 (Isoform B). In some embodiments, the HSD17B13 mRNA molecule encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:22 (Isoform B). In some embodiments, the HSD17B13 mRNA molecule encoding the isoform protein associated with the functional HSD17B13 protein comprises or consists of a nucleic acid sequence that encodes an HSD17B13 isoform protein that comprises or consists of an amino acid sequence according to SEQ ID NO:22 (Isoform B).

In some embodiments, an HSD17B13 mRNA molecule encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:25 (Isoform E). In some embodiments, the HSD17B13 mRNA molecule encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:25 (Isoform E). In some embodiments, the HSD17B13 mRNA molecule encoding the isoform protein associated with the functional HSD17B13 protein comprises or consists of a nucleic acid sequence that encodes an HSD17B13 isoform protein that comprises or consists of an amino acid sequence according to SEQ ID NO:25 (Isoform E).

In some embodiments, an HSD17B13 mRNA molecule encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:29 (Isoform I). In some embodiments, the HSD17B13 mRNA molecule encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:29 (Isoform I). In some embodiments, the HSD17B13 mRNA molecule encoding the isoform protein associated with the functional HSD17B13 protein comprises or consists of a nucleic acid sequence that encodes an HSD17B13 isoform protein that comprises or consists of an amino acid sequence according to SEQ ID NO:29 (Isoform I).

In some embodiments, an HSD17B13 mRNA molecule encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:3 (Transcript A). In some embodiments, the HSD17B13 mRNA molecule encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:3 (Transcript A). In some embodiments, the HSD17B13 mRNA molecule encoding the isoform protein associated with the functional HSD17B13 protein comprises or consists of a nucleic acid sequence according to SEQ ID NO: 3 (Transcript A).

In some embodiments, an HSD17B13 mRNA molecule encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:4 (Transcript B). In some embodiments, the HSD17B13 mRNA molecule encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:4 (Transcript B). In some embodiments, the HSD17B13 mRNA molecule encoding the isoform protein associated with the functional HSD17B13 protein comprises or consists of a nucleic acid sequence according to SEQ ID NO: 4 (Transcript B).

In some embodiments, an HSD17B13 mRNA molecule encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:7 (Transcript E). In some embodiments, the HSD17B13 mRNA molecule encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:7 (Transcript E). In some embodiments, the HSD17B13 mRNA molecule encoding the isoform protein associated with the functional HSD17B13 protein comprises or consists of a nucleic acid sequence according to SEQ ID NO: 7 (Transcript E).

In some embodiments, an HSD17B13 mRNA molecule encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:11 (Transcript I). In some embodiments, the HSD17B13 mRNA molecule encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:11 (Transcript I). In some embodiments, the HSD17B13 mRNA molecule encoding the isoform protein associated with the functional HSD17B13 protein comprises or consists of a nucleic acid sequence according to SEQ ID NO: 11 (Transcript I).

The nucleic acid sequences for five HSD17B13 mRNA molecules encoding isoform proteins associated with a loss-of-function are set forth in SEQ ID NO:5 (Transcript C), SEQ ID NO: 6 (Transcript D), SEQ ID NO:8 (Transcript F), SEQ ID NO:9 (Transcript G), and SEQ ID NO: 10 (Transcript H).

In some embodiments, an HSD17B13 mRNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:23 (Isoform C). In some embodiments, the HSD17B13 mRNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 23 (Isoform C). In some embodiments, the HSD17B13 mRNA molecule encoding the isoform protein associated with a loss-of-function comprises or consists of a nucleic acid sequence that encodes an HSD17B13 isoform protein that comprises or consists of an amino acid sequence according to SEQ ID NO:23 (Isoform C).

In some embodiments, an HSD17B13 mRNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:24 (Isoform D). In some embodiments, the HSD17B13 mRNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 24 (Isoform D). In some embodiments, the HSD17B13 mRNA molecule encoding the isoform protein associated with a loss-of-function comprises or consists of a nucleic acid sequence that encodes an HSD17B13 isoform protein that comprises or consists of an amino acid sequence according to SEQ ID NO:24 (Isoform D).

In some embodiments, an HSD17B13 mRNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:26 (Isoform F). In some embodiments, the HSD17B13 mRNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 26 (Isoform F). In some embodiments, the HSD17B13 mRNA molecule encoding the isoform protein associated with a loss-of-function comprises or consists of a nucleic acid sequence that encodes an HSD17B13 isoform protein that comprises or consists of an amino acid sequence according to SEQ ID NO:26 (Isoform F).

In some embodiments, an HSD17B13 mRNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:27 (Isoform G). In some embodiments, the HSD17B13 mRNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 27 (Isoform G). In some embodiments, the HSD17B13 mRNA molecule encoding the isoform protein associated with a loss-of-function comprises or consists of a nucleic acid sequence that encodes an HSD17B13 isoform protein that comprises or consists of an amino acid sequence according to SEQ ID NO:27 (Isoform G).

In some embodiments, an HSD17B13 mRNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:28 (Isoform H). In some embodiments, the HSD17B13 mRNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 28 (Isoform H). In some embodiments, the HSD17B13 mRNA molecule encoding the isoform protein associated with a loss-of-function comprises or consists of a nucleic acid sequence that encodes an HSD17B13 isoform protein that comprises or consists of an amino acid sequence according to SEQ ID NO:28 (Isoform H).

In some embodiments, an HSD17B13 mRNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:5 (Transcript C). In some embodiments, the HSD17B13 mRNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:5 (Transcript C). In some embodiments, the HSD17B13 mRNA molecule encoding the isoform protein associated with a loss-of-function comprises or consists of a nucleic acid sequence according to SEQ ID NO:5 (Transcript C).

In some embodiments, an HSD17B13 mRNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:6 (Transcript D). In some embodiments, the HSD17B13 mRNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:6 (Transcript D). In some embodiments, the HSD17B13 mRNA molecule encoding the isoform protein associated with a loss-of-function comprises or consists of a nucleic acid sequence according to SEQ ID NO:6 (Transcript D).

In some embodiments, an HSD17B13 mRNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:8 (Transcript F). In some embodiments, the HSD17B13 mRNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:8 (Transcript F). In some embodiments, the HSD17B13 mRNA molecule encoding the isoform protein associated with a loss-of-function comprises or consists of a nucleic acid sequence according to SEQ ID NO:8 (Transcript F).

In some embodiments, an HSD17B13 mRNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:9 (Transcript G). In some embodiments, the HSD17B13 mRNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:9 (Transcript G). In some embodiments, the HSD17B13 mRNA molecule encoding the isoform protein associated with a loss-of-function comprises or consists of a nucleic acid sequence according to SEQ ID NO:9 (Transcript G).

In some embodiments, an HSD17B13 mRNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:10 (Transcript H). In some embodiments, the HSD17B13 mRNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:10 (Transcript H). In some embodiments, the HSD17B13 mRNA molecule encoding the isoform protein associated with a loss-of-function comprises or consists of a nucleic acid sequence according to SEQ ID NO:10 (Transcript H).

In some embodiments, the HSD17B13 mRNA molecules comprise less nucleotides than the entire mRNA sequence. In some embodiments, the HSD17B13 mRNA molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, or at least about 900 contiguous nucleotides of SEQ ID NO:3, SEQ ID NO:4, SEQ ID NO: 7, or SEQ ID NO: 11, or SEQ ID NO:5, SEQ ID NO:6, SEQ ID NO:8, SEQ ID NO:9, or SEQ ID NO: 10. In some embodiments, the HSD17B13 mRNA molecules comprise or consist of at least about 200 to at least about 500 contiguous nucleotides of SEQ ID NO:3, SEQ ID NO:4, SEQ ID NO:7, or SEQ ID NO:11, or SEQ ID NO:5, SEQ ID NO:6, SEQ ID NO:8, SEQ ID NO:9, or SEQ ID NO:10. In this regard, the longer mRNA molecules are preferred over the shorter ones. In some embodiments, the HSD17B13 mRNA molecules comprise or consist of at least about 50, at least about 60, at least about 70, at least about 80, at least about 90, at least about 100, at least about 200, at least about 300, at least about 400, or at least about 500 contiguous nucleotides of SEQ ID NO:3, SEQ ID NO:4, SEQ ID NO:7, or SEQ ID NO: 11, or SEQ ID NO: 5, SEQ ID NO: 6, SEQ ID NO: 8, SEQ ID NO:9, or SEQ ID NO:10. In this regard, the longer mRNA molecules are preferred over the shorter ones.

The nucleic acid sequences for four HSD17B13 cDNA molecules encoding isoform proteins associated with the functional HSD17B13 protein are set forth in SEQ ID NO:12 (Transcript A), SEQ ID NO:13 (Transcript B), SEQ ID NO:16 (Transcript E), and SEQ ID NO:20 (Transcript I).

In some embodiments, an HSD17B13 cDNA molecule encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:21 (Isoform A). In some embodiments, the HSD17B13 cDNA molecule encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:21 (Isoform A). In some embodiments, the HSD17B13 cDNA molecule encoding the isoform protein associated with the functional HSD17B13 protein comprises or consists of a nucleic acid sequence that encodes an HSD17B13 isoform protein that comprises or consists of an amino acid sequence according to SEQ ID NO:21 (Isoform A).

In some embodiments, an HSD17B13 cDNA molecule encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:22 (Isoform B). In some embodiments, the HSD17B13 cDNA molecule encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:22 (Isoform B). In some embodiments, the HSD17B13 cDNA molecule encoding the isoform protein associated with the functional HSD17B13 protein comprises or consists of a nucleic acid sequence that encodes an HSD17B13 isoform protein that comprises or consists of an amino acid sequence according to SEQ ID NO:22 (Isoform B).

In some embodiments, an HSD17B13 cDNA molecule encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:25 (Isoform E). In some embodiments, the HSD17B13 cDNA molecule encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:25 (Isoform E). In some embodiments, the HSD17B13 cDNA molecule encoding the isoform protein associated with the functional HSD17B13 protein comprises or consists of a nucleic acid sequence that encodes an HSD17B13 isoform protein that comprises or consists of an amino acid sequence according to SEQ ID NO:25 (Isoform E).

In some embodiments, an HSD17B13 cDNA molecule encoding an isoform protein associated with the functional HSD17B13 protein comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:29 (Isoform I). In some embodiments, the HSD17B13 cDNA molecule encoding an isoform protein associated with the functional HSD17B13 protein comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO:29 (Isoform I). In some embodiments, the HSD17B13 cDNA molecule encoding the isoform protein associated with the functional HSD17B13 protein comprises or consists of a nucleic acid sequence that encodes an HSD17B13 isoform protein that comprises or consists of an amino acid sequence according to SEQ ID NO:29 (Isoform I).

In some embodiments, an HSD17B13 cDNA molecule encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:12 (Transcript A). In some embodiments, the HSD17B13 cDNA molecule encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:12 (Transcript A). In some embodiments, the HSD17B13 cDNA molecule encoding the isoform protein associated with the functional HSD17B13 protein comprises or consists of a nucleic acid sequence according to SEQ ID NO: 12 (Transcript A).

In some embodiments, an HSD17B13 cDNA molecule encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:13 (Transcript B). In some embodiments, the HSD17B13 cDNA molecule encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:13 (Transcript B). In some embodiments, the HSD17B13 cDNA molecule encoding the isoform protein associated with the functional HSD17B13 protein comprises or consists of a nucleic acid sequence according to SEQ ID NO: 13 (Transcript B).

In some embodiments, an HSD17B13 cDNA molecule encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:16 (Transcript E). In some embodiments, the HSD17B13 cDNA molecule encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:16 (Transcript E). In some embodiments, the HSD17B13 cDNA molecule encoding the isoform protein associated with the functional HSD17B13 protein comprises or consists of a nucleic acid sequence according to SEQ ID NO: 16 (Transcript E).

In some embodiments, an HSD17B13 cDNA molecule encoding an isoform protein associated with the functional HSD17B13 protein comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:20 (Transcript I). In some embodiments, the HSD17B13 cDNA molecule encoding an isoform protein associated with the functional HSD17B13 protein comprises or consists of a nucleic acid sequence that has at least about 95%, at least about 96%, at least about 97%, at least about 98%, or at least about 99% sequence identity to SEQ ID NO:20 (Transcript I). In some embodiments, the HSD17B13 cDNA molecule encoding the isoform protein associated with the functional HSD17B13 protein comprises or consists of a nucleic acid sequence according to SEQ ID NO: 20 (Transcript I).

The nucleic acid sequences for five HSD17B13 cDNA molecules encoding isoform proteins associated with a loss-of-function are set forth in SEQ ID NO:14 (Transcript C), SEQ ID NO: 15 (Transcript D), SEQ ID NO:17 (Transcript F), SEQ ID NO:18 (Transcript G), and SEQ ID NO: 19 (Transcript H).

In some embodiments, an HSD17B13 cDNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:23 (Isoform C). In some embodiments, the HSD17B13 cDNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 23 (Isoform C). In some embodiments, the HSD17B13 cDNA molecule encoding the isoform protein associated with a loss-of-function comprises or consists of a nucleic acid sequence that encodes an HSD17B13 isoform protein that comprises or consists of an amino acid sequence according to SEQ ID NO:23 (Isoform C).

In some embodiments, an HSD17B13 cDNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:24 (Isoform D). In some embodiments, the HSD17B13 cDNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 24 (Isoform D). In some embodiments, the HSD17B13 cDNA molecule encoding the isoform protein associated with a loss-of-function comprises or consists of a nucleic acid sequence that encodes an HSD17B13 isoform protein that comprises or consists of an amino acid sequence according to SEQ ID NO:24 (Isoform D).

In some embodiments, an HSD17B13 cDNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:26 (Isoform F). In some embodiments, the HSD17B13 cDNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 26 (Isoform F). In some embodiments, the HSD17B13 cDNA molecule encoding the isoform protein associated with a loss-of-function comprises or consists of a nucleic acid sequence that encodes an HSD17B13 isoform protein that comprises or consists of an amino acid sequence according to SEQ ID NO:26 (Isoform F).

In some embodiments, an HSD17B13 cDNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:27 (Isoform G). In some embodiments, the HSD17B13 cDNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 27 (Isoform G). In some embodiments, the HSD17B13 cDNA molecule encoding the isoform protein associated with a loss-of-function comprises or consists of a nucleic acid sequence that encodes an HSD17B13 isoform protein that comprises or consists of an amino acid sequence according to SEQ ID NO:27 (Isoform G).

In some embodiments, an HSD17B13 cDNA molecule encoding an isoform protein associated with a loss-of-function comprises or consists of a nucleic acid sequence that encodes an HSD17B13 isoform protein that comprises an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:28 (Isoform H). In some embodiments, the HSD17B13 cDNA molecule encoding an isoform protein associated with a loss-of-function comprises or consists of a nucleic acid sequence that encodes an HSD17B13 isoform protein that comprises an amino acid sequence that has at least about 95%, at least about 96%, at least about 97%, at least about 98%, or at least about 99% sequence identity to SEQ ID NO: 28 (Isoform H). In some embodiments, the HSD17B13 cDNA molecule encoding the isoform protein associated with a loss-of-function comprises or consists of a nucleic acid sequence that encodes an HSD17B13 isoform protein that comprises or consists of an amino acid sequence according to SEQ ID NO:28 (Isoform H).

In some embodiments, an HSD17B13 cDNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:14 (Transcript C). In some embodiments, the HSD17B13 cDNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:14 (Transcript C). In some embodiments, the HSD17B13 cDNA molecule encoding the isoform protein associated with a loss-of-function comprises or consists of a nucleic acid sequence according to SEQ ID NO:14 (Transcript C).

In some embodiments, an HSD17B13 cDNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:15 (Transcript D). In some embodiments, the HSD17B13 cDNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:15 (Transcript D). In some embodiments, the HSD17B13 cDNA molecule encoding the isoform protein associated with a loss-of-function comprises or consists of a nucleic acid sequence according to SEQ ID NO:15 (Transcript D).

In some embodiments, an HSD17B13 cDNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:17 (Transcript F). In some embodiments, the HSD17B13 cDNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:17 (Transcript F). In some embodiments, the HSD17B13 cDNA molecule encoding the isoform protein associated with a loss-of-function comprises or consists of a nucleic acid sequence according to SEQ ID NO:17 (Transcript F).

In some embodiments, an HSD17B13 cDNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:18 (Transcript G). In some embodiments, the HSD17B13 cDNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:18 (Transcript G). In some embodiments, the HSD17B13 cDNA molecule encoding the isoform protein associated with a loss-of-function comprises or consists of a nucleic acid sequence according to SEQ ID NO:18 (Transcript G).

In some embodiments, an HSD17B13 cDNA molecule encoding an isoform protein associated with a loss-of-function comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:19 (Transcript H). In some embodiments, the HSD17B13 cDNA molecule encoding an isoform protein associated with a loss-of-function comprises or consists of a nucleic acid sequence that has at least about 95%, at least about 96%, at least about 97%, at least about 98%, or at least about 99% sequence identity to SEQ ID NO:19 (Transcript H). In some embodiments, the HSD17B13 cDNA molecule encoding the isoform protein associated with a loss-of-function comprises or consists of a nucleic acid sequence according to SEQ ID NO:19 (Transcript H).

In some embodiments, the HSD17B13 cDNA molecules comprise less nucleotides than the entire cDNA sequence. In some embodiments, the HSD17B13 cDNA molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, or at least about 900 contiguous nucleotides of SEQ ID NO:12, SEQ ID NO:13, SEQ ID NO: 16, or SEQ ID NO:20 or SEQ ID NO:14, SEQ ID NO: 15, SEQ ID NO:17, SEQ ID NO: 18, or SEQ ID NO:19. In some embodiments, the HSD17B13 cDNA molecules comprise or consist of at least about 200 to at least about 500 contiguous nucleotides of SEQ ID NO:12, SEQ ID NO: 13, SEQ ID NO: 16, or SEQ ID NO:20 or SEQ ID NO: 14, SEQ ID NO: 15, SEQ ID NO: 17, SEQ ID NO: 18, or SEQ ID NO:19. In this regard, the longer cDNA molecules are preferred over the shorter ones. In some embodiments, the HSD17B13 cDNA molecules comprise or consist of at least about 50, at least about 60, at least about 70, at least about 80, at least about 90, at least about 100, at least about 200, at least about 300, at least about 400, or at least about 500 contiguous nucleotides of SEQ ID NO:12, SEQ ID NO:13, SEQ ID NO: 16, or SEQ ID NO:20 or SEQ ID NO: 14, SEQ ID NO:15, SEQ ID NO:17, SEQ ID NO:18, or SEQ ID NO:19. In this regard, the longer cDNA molecules are preferred over the shorter ones.

The probes and primers described herein can be used to hybridize to any of the functional or variant PNPLA3 genomic DNA molecules, mRNA molecules, or cDNA molecules derived from mRNA molecules described herein. The primers can be used, for example, to amplify portions of any of the functional or variant PNPLA3 genomic DNA molecules, mRNA molecules, or cDNA molecules derived from mRNA molecules described herein, so that the amplifications products can be, for example, detected or sequenced.

For example, the probes and primers can be used to hybridize to any of the wild type PNPLA3 genomic DNA molecules described herein, including the wild type PNPLA3 genomic DNA molecule comprising SEQ ID NO:30. The probes and primers can also be used to hybridize to any of the wild type PNPLA3 mRNA molecules described herein, including the wild type PNPLA3 mRNA molecules comprising SEQ ID NO:32 or SEQ ID NO:33. The probes and primers can also be used to hybridize to any of the wild type PNPLA3 cDNA molecules described herein, including the wild type PNPLA3 cDNA molecules comprising SEQ ID NO:36 or SEQ ID NO:37.

The probes and primers can also be used to hybridize to any of the variant PNPLA3 genomic DNA molecules described herein, including the variant PNPLA3 genomic DNA molecule comprising SEQ ID NO:31. The probes and primers can also be used to hybridize to any of the variant PNPLA3 mRNA molecules described herein, including the variant PNPLA3 mRNA molecules comprising SEQ ID NO:34 or SEQ ID NO:35. The probes and primers can also be used to hybridize to any of the variant PNPLA3 cDNA molecules described herein, including the variant PNPLA3 cDNA molecules comprising SEQ ID NO:38 or SEQ ID NO:39.

The probes can be used, for example, to detect any of the functional or variant HSD17B13 genomic DNA molecules, mRNA molecules, or cDNA molecules derived from mRNA molecules described herein. The primers can be used, for example, to amplify portions of any of the functional or variant HSD17B13 genomic DNA molecules, mRNA molecules, or cDNA molecules derived from mRNA molecules described herein, so that the amplifications products can be, for example, detected or sequenced.

For example, the probes and primers can be used to hybridize to any of the functional HSD17B13 genomic DNA molecules described herein, including the functional HSD17B13 genomic DNA molecule comprising SEQ ID NO:1. The probes and primers can also be used to hybridize to any of the functional HSD17B13 RNA transcripts described herein, including the functional HSD17B13 RNA transcripts comprising SEQ ID NO:44, SEQ ID NO: 45, SEQ ID NO:48, or SEQ ID NO:52. The probes and primers can also be used to hybridize to any of the functional HSD17B13 DNA transcripts described herein, including the functional HSD17B13 DNA transcripts comprising SEQ ID NO:53, SEQ ID NO:54, SEQ ID NO: 57, or SEQ ID NO: 61. The probes and primers can also be used to hybridize to any of the functional HSD17B13 mRNA molecules described herein, including the functional HSD17B13 mRNA molecules comprising SEQ ID NO:3, SEQ ID NO:4, SEQ ID NO:7, or SEQ ID NO:11. The probes and primers can also be used to hybridize to any of the functional HSD17B13 cDNA molecules described herein, including the functional HSD17B13 cDNA molecules comprising SEQ ID NO:12, SEQ ID NO:13, SEQ ID NO:16, or SEQ ID NO:20.

The probes and primers can also be used to hybridize to any of the variant HSD17B13 genomic DNA molecules described herein, including the variant HSD17B13 genomic DNA molecule comprising SEQ ID NO:2. The probes and primers can also be used to hybridize to any of the variant HSD17B13 RNA transcripts described herein, including the variant HSD17B13 RNA transcripts comprising SEQ ID NO:46, SEQ ID NO:47, SEQ ID NO:49, SEQ ID NO:50, or SEQ ID NO:51. The probes and primers can also be used to hybridize to any of the variant HSD17B13 DNA transcripts described herein, including the variant HSD17B13 DNA transcripts comprising SEQ ID NO:55, SEQ ID NO:56, SEQ ID NO:58, SEQ ID NO:59, or SEQ ID NO:60. The probes and primers can also be used to hybridize to any of the variant HSD17B13 mRNA molecules described herein, including the variant HSD17B13 mRNA molecules comprising SEQ ID NO:5, SEQ ID NO:6, SEQ ID NO:8, SEQ ID NO:9, or SEQ ID NO: 10. The probes and primers can also be used to hybridize to any of the HSD17B13 cDNA molecules described herein, including the HSD17B13 cDNA molecules comprising SEQ ID NO: 14, SEQ ID NO:15, SEQ ID NO: 17, SEQ ID NO: 18, or SEQ ID NO:19.

In some embodiments, the probes and/or primers described herein comprise a nucleic acid sequence that specifically hybridizes to any of the nucleic acid molecules disclosed herein, or the complement thereof. In some embodiments, the probe or primer specifically hybridizes to any of the nucleic acid molecules disclosed herein under stringent conditions. The present disclosure also provides nucleic acid molecules having nucleic acid sequences that hybridize under moderate conditions to any of the nucleic acid molecules disclosed herein, or the complement thereof.

Appropriate stringency conditions which promote DNA hybridization include, for example, 6× sodium chloride/sodium citrate (SSC) at about 45° C., followed by a wash of 2×SSC at 50° C. (see, also Current Protocols in Molecular Biology, John Wiley & Sons, N.Y. (1989), 6.3.1-6.3.6). Typically, stringent conditions for hybridization and detection will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for longer probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In hybridization reactions, specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 1984, 138, 267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≥90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1° C., 2° C., 3° C., or 4° C. lower than the thermal melting point (Tₘ); moderately stringent conditions can utilize a hybridization and/or wash at 6° C., 7° C., 8° C., 9° C., or 10° C. lower than the thermal melting point (Tₘ); low stringency conditions can utilize a hybridization and/or wash at 11° C., 12° C., 13° C., 14° C., 15° C., or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used.

The probes described herein can be linked or fused to a label to aid in detection. The label can be directly detectable (e.g., fluorophore) or indirectly detectable (e.g., hapten, enzyme, or fluorophore quencher). Such labels can be detectable by spectroscopic, photochemical, biochemical, immunochemical, or chemical means. Such labels include, for example, radiolabels that can be measured with radiation-counting devices; pigments, dyes or other chromogens that can be visually observed or measured with a spectrophotometer; spin labels that can be measured with a spin label analyzer; and fluorescent labels (e.g., fluorophores), where the output signal is generated by the excitation of a suitable molecular adduct and that can be visualized by excitation with light that is absorbed by the dye or can be measured with standard fluorometers or imaging systems. The label can also be, for example, a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term “label” can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The probe or primer can comprise any suitable length, non-limiting examples of which include at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides in length. In some embodiments, the probe or primer comprises at least about 18 nucleotides in length to about 25 nucleotides in length. The probe or primer can comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides in length. In some embodiments, the probe or primer is from about 18 to about 30 nucleotides in length. Alternately, in some embodiments, the probe comprises or consists of at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 nucleotides.

In some embodiments, the probes and/or primers can hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to any of the wild type PNPLA3 or HSD17B13 nucleic acid molecules or variant PNPLA3 or HSD17B13 nucleic acid molecules described herein.

In some embodiments, the probe or primer comprises DNA. In some embodiments, the probe or primer comprises RNA.

The probes and primers described herein can also be alteration-specific probes and alteration-specific primers. The alteration-specific probe or alteration-specific primer can comprise a nucleic acid sequence which is complementary to and/or hybridizes, or specifically hybridizes, to a nucleic acid sequence encoding a variant PNPLA3 protein but which is not complementary to and/or hybridizes, or specifically hybridizes, to a nucleic acid sequence encoding a wild type PNPLA3 protein. In this context, “specifically hybridizes” means that the probe or primer (e.g., the alteration-specific probe or alteration-specific primer) does not hybridize to a nucleic acid molecule encoding a wild type PNPLA3 protein. Herein, the term “specifically hybridizes” means that the probe or primer exclusively hybridizes to the indicated nucleic acid molecule and not to another nucleic acid molecule. Accordingly, a probe or primer which specifically hybridizes to a nucleic acid molecule encoding a PNPLA3 protein comprising the I148M variation does not hybridize to a nucleic acid molecule encoding a PNPLA3 protein which does not comprise the I148M variation. The alteration-specific probe or alteration-specific primer can also comprise a nucleic acid sequence which is complementary to and/or hybridizes, or specifically hybridizes, to a nucleic acid sequence encoding a wild type PNPLA3 protein but which is not complementary to and/or hybridizes, or specifically hybridizes, to a nucleic acid sequence encoding a variant PNPLA3 protein. In this context, “specifically hybridizes” means that the probe or primer (e.g., the alteration-specific probe or alteration-specific primer) does not hybridize to a nucleic acid molecule encoding a variant PNPLA3 protein.

The alteration-specific probe or alteration-specific primer can also comprise a nucleic acid sequence which is complementary to and/or hybridizes, or specifically hybridizes, to a nucleic acid sequence encoding a variant HSD17B13 protein but which is not complementary to and/or hybridizes, or specifically hybridizes, to a nucleic acid sequence encoding a functional HSD17B13 protein. In this context, “specifically hybridizes” means that the probe or primer (e.g., the alteration-specific probe or alteration-specific primer) does not hybridize to a nucleic acid molecule encoding a functional HSD17B13 protein. For example, in this context “specifically hybridizes” means that the probe or primer does not hybridize to a nucleic acid molecule encoding a non-active/loss of function HSD17B13 protein. The alteration-specific probe or alteration-specific primer can also comprise a nucleic acid sequence which is complementary to and/or hybridizes, or specifically hybridizes, to a nucleic acid sequence encoding a functional HSD17B13 protein but which is not complementary to and/or hybridizes, or specifically hybridizes, to a nucleic acid sequence encoding a variant HSD17B13 protein. In this context, “specifically hybridizes” means that the probe or primer (e.g., the alteration-specific probe or alteration-specific primer) does not hybridize to a nucleic acid molecule encoding a variant HSD17B13 protein.

In some embodiments, the alteration-specific probe or alteration-specific primer comprises a nucleic acid sequence which is complementary to and/or hybridizes, or specifically hybridizes, to a portion of a PNPLA3 nucleic acid sequence that comprises a methionine at a position corresponding to position 148 according to SEQ ID NO:42, or comprises a methionine at a position corresponding to position 144 according to SEQ ID NO: 43. In some embodiments, the alteration-specific probe or alteration-specific primer comprises a nucleic acid sequence which is complementary to and/or hybridizes, or specifically hybridizes, to the portion of the variant PNPLA3 genomic DNA that comprises an ATG codon at the positions corresponding to positions 5107 to 5109 according to SEQ ID NO: 31. In some embodiments, the alteration-specific probe or alteration-specific primer comprises a nucleic acid sequence which is complementary to and/or hybridizes, or specifically hybridizes, to the portion of the variant PNPLA3 mRNA that comprises an AUG codon at the positions corresponding to positions 442 to 444 according to SEQ ID NO:34. In some embodiments, the alteration-specific probe or alteration-specific primer comprises a nucleic acid sequence which is complementary to and/or hybridizes, or specifically hybridizes, to the portion of the variant PNPLA3 mRNA that comprises an AUG codon at the positions corresponding to positions 430 to 432 according to SEQ ID NO:35. In some embodiments, the alteration-specific probe or alteration-specific primer comprises a nucleic acid sequence which is complementary to and/or hybridizes, or specifically hybridizes, to the portion of the variant PNPLA3 cDNA that comprises an ATG codon at the positions corresponding to positions 442 to 444 according to SEQ ID NO:38. In some embodiments, the alteration-specific probe or alteration-specific primer comprises a nucleic acid sequence which is complementary to and/or hybridizes, or specifically hybridizes, to the portion of the variant PNPLA3 cDNA that comprises an ATG codon at the positions corresponding to positions 430 to 432 according to SEQ ID NO:39.

In some embodiments, the alteration-specific probe or alteration-specific primer comprises a nucleic acid sequence which is complementary to and/or hybridizes, or specifically hybridizes, to the portion of the HSD17B13 genomic DNA that comprises an adenine at a position corresponding to position 12,667 according to SEQ ID NO:1. In some embodiments, the alteration-specific probe or alteration-specific primer comprises a nucleic acid sequence which is complementary to and/or hybridizes, or specifically hybridizes, to the portion of the HSD17B13 genomic DNA that comprises a thymine at a position corresponding to position 12,667 according to SEQ ID NO:2.

In some embodiments, the portion of the nucleic acid molecule to which the probe or primer is hybridized comprises from about 10 to about 200, from about 10 to about 150, from about 10 to about 100, from about 10 to about 50, from about 10 to about 40, from about 10 to about 30, or from about 10 to about 20 nucleotides, and comprises the codon corresponding to the position containing codon encoding the particular variation (e.g., I148M of PNPLA3 or the portion of the variant HSD17B13 protein that is different from the corresponding wild type HSD17B13 protein). In some preferred embodiments, the portion of the nucleic acid molecule to which the probe or primer is hybridized comprises from about 10 to about 50, from about 10 to about 40, from about 10 to about 30, or from about 10 to about 20 nucleotides, and comprises the codon corresponding to the position containing codon encoding the particular variation (e.g., I148M of PNPLA3 or the portion of the variant HSD17B13 protein that is different from the corresponding wild type HSD17B13 protein).

The kits described herein can comprise detection and/or amplification assay reagents that can be used for detecting and/or amplifying any of the wild type PNPLA3 and/or HSD17B13 nucleic acid molecules described herein and/or any of the variant PNPLA3 and/or HSD17B13 nucleic acid molecules described herein. In some embodiments, the kits for such detection and/or amplification can contain any of the reagents (e.g., probes and primers) described herein. In some embodiments, a basic kit can comprise a container having at least one probe or primer or at least two probes or primers, such as alteration-specific probes or alteration-specific primers, for a locus in any of the nucleic acid molecules disclosed herein. A kit can also optionally comprise instructions for use. A kit can also comprise other optional kit components, such as, for example, one or more of an allelic ladder directed to each of the loci amplified, a sufficient quantity of enzyme for amplification, amplification buffer to facilitate the amplification, divalent cation solution to facilitate enzyme activity, dNTPs for strand extension during amplification, loading solution for preparation of the amplified material for electrophoresis, genomic DNA as a template control, a size marker to insure that materials migrate as anticipated in the separation medium, and a protocol and manual to educate the user and limit error in use. The amounts of the various reagents in the kits also can be varied depending upon a number of factors, such as the optimum sensitivity of the process. It is within the scope of these teachings to provide test kits for use in manual applications or test kits for use with automated sample preparation, reaction set-up, detectors or analyzers. In some embodiments, the kits comprise at least one labeled probe (e.g., alteration-specific probe) for detection. In some embodiments, any of the kits disclosed herein can further comprise products and reagents required to carry out an annealing reaction, and instructions.

The present disclosure provides methods for detecting the presence of any of the wild type PNPLA3 proteins described herein. The present disclosure also provides methods for detecting the presence of any of the variant PNPLA3 proteins described herein. The present disclosure also provides methods for detecting the presence of any of the wild type PNPLA3 nucleic acid molecules described herein (e.g., genomic DNA molecules, mRNA molecules, and cDNA molecules) described herein. The present disclosure also provides methods for detecting the presence of any of the variant PNPLA3 nucleic acid molecules described herein (e.g., genomic DNA molecules, mRNA molecules, and cDNA molecules) described herein.

The present disclosure also provides methods for detecting the presence of any of the functional HSD17B13 proteins described herein. The present disclosure also provides methods for detecting the presence of any of the variant HSD17B13 proteins described herein. The present disclosure also provides methods for detecting the presence of any of the functional HSD17B13 nucleic acid molecules described herein (e.g., genomic DNA molecules, RNA transcripts, cDNA transcripts, mRNA molecules, and cDNA molecules) described herein. The present disclosure also provides methods for detecting the presence of any of the variant HSD17B13 nucleic acid molecules described herein (e.g., genomic DNA molecules, RNA transcripts, cDNA transcripts, mRNA molecules, and cDNA molecules) described herein.

In some embodiments of any of the methods described herein, a functional HSD17B13 protein, or nucleic acid molecule encoding the same, is detected or sought to be detected in a subject or patient. In some embodiments, the subject or patient comprises a functional HSD17B13 protein. In some embodiments, the functional HSD17B13 protein is one of the functional HSD17B13 proteins described herein (which can be encoded by any of the nucleic acid molecules described herein encoding the same). In some embodiments, a functional HSD17B13 protein has at least 90%, at least 80%, at least 70%, at least 60%, at least 50%, at least 40%, at least 30%, at least 20%, at least 10%, at least 5%, or at least 1% of the biological activity of the HSD17B13 protein having the amino acid sequence according to SEQ ID NO:40. In some embodiments, a functional HSD17B13 protein has at least 90%, at least 80%, at least 70%, at least 60%, or at least 50% of the biological activity of the HSD17B13 protein having the amino acid sequence according to SEQ ID NO:40. In some embodiments, a functional HSD17B13 protein has at least 90%, at least 80%, at least 70%, at least 60%, at least 50%, at least 40%, at least 30%, at least 20%, at least 10%, at least 5%, or at least 1% of the biological activity of HSD17B13 protein having SEQ ID NO:40. In some embodiments, a functional HSD17B13 protein has at least 70%, at least 60%, at least 50%, at least 40%, at least 30%, at least 20%, at least 10%, at least 5%, or at least 1% of the biological activity of HSD17B13 protein having SEQ ID NO:40. In some embodiments, a functional HSD17B13 protein has at least 50%, at least 40%, at least 30%, at least 20%, at least 10%, at least 5%, or at least 1% of the biological activity of HSD17B13 protein having SEQ ID NO:40. In some embodiments, a functional HSD17B13 protein has at least 30%, at least 20%, at least 10%, at least 5%, or at least 1% of the biological activity of HSD17B13 protein having SEQ ID NO:40. In some embodiments, a functional HSD17B13 protein has at least 90% of the biological activity of HSD17B13 protein having SEQ ID NO:40. In some embodiments, a functional HSD17B13 protein has at least 80% of the biological activity of HSD17B13 protein having SEQ ID NO:40. In some embodiments, a functional HSD17B13 protein has at least 70% of the biological activity of HSD17B13 protein having SEQ ID NO:40. In some embodiments, a functional HSD17B13 protein has at least 60% of the biological activity of HSD17B13 protein having SEQ ID NO:40. In some embodiments, a functional HSD17B13 protein has at least 50% of the biological activity of HSD17B13 protein having SEQ ID NO:40. In some embodiments, a functional HSD17B13 protein has at least 40% of the biological activity of HSD17B13 protein having SEQ ID NO:40. In some embodiments, a functional HSD17B13 protein has at least 30% of the biological activity of HSD17B13 protein having SEQ ID NO:40. In some embodiments, a functional HSD17B13 protein has at least 20% of the biological activity of HSD17B13 protein having SEQ ID NO:40. In some embodiments, a functional HSD17B13 protein has at least 10% of the biological activity of HSD17B13 protein having SEQ ID NO:40. In some embodiments, a functional HSD17B13 protein has at least 5% of the biological activity of HSD17B13 protein having SEQ ID NO:40. In some embodiments, a functional HSD17B13 protein has at least 1% of the biological activity of HSD17B13 protein having SEQ ID NO:40. In some embodiments, the activity of an HSD17B13 protein (e.g., functionality) can be determined by, for example, performing an oxidoreductase activity assay.

It is understood that gene sequences within a population and mRNAs and proteins encoded by such genes can vary due to polymorphisms such as single-nucleotide polymorphisms. The sequences provided herein are only exemplary sequences. Other sequences for the variant PNPLA3 and HSD17B13 genomic DNA, mRNA, cDNA, and polypeptide are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting a variant PNPLA3 nucleic acid molecule, preliminary processing designed to isolate or enrich the sample for the genomic DNA can be employed. A variety of techniques can be used for this purpose. When detecting the level of variant PNPLA3 mRNA, different techniques can be used enrich the biological sample with mRNA. Various methods to detect the presence or level of an mRNA or the presence of a particular variant genomic DNA locus can be used.

In some embodiments, the presence or absence of a particular PNPLA3 protein or HSD17B13 protein (e.g., functional or variant) is detected by sequencing at least a portion of the protein to determine whether the protein comprises an amino acid sequence encoding any of the variant PNPLA3 proteins or HSD17B13 proteins (e.g., functional or variant) described herein. In some embodiments, the presence or absence of a particular PNPLA3 protein or HSD17B13 protein (e.g., functional or variant) is detected by performing an immunoassay, such as an ELISA, to determine whether any of the variant PNPLA3 proteins or HSD17B13 proteins (e.g., functional or variant) described herein are present in the sample.

In some embodiments, the portion of the protein sequenced comprises from about 5 to about 100, from about 5 to about 50, from about 5 to about 40, from about 5 to about 30, from about 5 to about 20, or from about 5 to about 10 amino acids, and comprises the position corresponding to the position containing the variation (e.g., I148M of PNPLA3 or the portion of the variant HSD17B13 protein that is different from the corresponding wild type HSD17B13 protein). In some preferred embodiments, the portion of the protein sequenced comprises from about 5 to about 20, or from about 5 to about 10 amino acids, and comprises the position corresponding to the position containing the variation (e.g., I148M of PNPLA3 or the portion of the variant HSD17B13 protein that is different from the corresponding wild type HSD17B13 protein).

Illustrative non-limiting examples of protein sequencing techniques include, but are not limited to, mass spectrometry and Edman degradation. Illustrative examples of immunoassays include, but are not limited to, immunoprecipitation, Western blot, immunohistochemistry, ELISA, immunocytochemistry, flow cytometry, and immuno-PCR. Polyclonal or monoclonal antibodies detectably labeled using various techniques (e.g., calorimetric, fluorescent, chemiluminescent, or radioactive) are suitable for use in the immunoassays.

In some embodiments, the presence or absence of a particular PNPLA3 nucleic acid molecule or HSD17B13 nucleic acid molecule (e.g., functional or variant genomic DNA, mRNA, cDNA, RNA transcript, or cDNA transcript) is detected by sequencing at least a portion of the nucleic acid molecule to determine whether the nucleic acid molecule comprises a nucleic acid sequence according to any of the variant PNPLA3 nucleic acid molecules or HSD17B13 nucleic acid molecules (e.g., functional or variant) described herein.

In some embodiments, the portion of the nucleic acid molecule sequenced comprises from about 10 to about 200, from about 10 to about 150, from about 10 to about 100, from about 10 to about 50, from about 10 to about 40, from about 10 to about 30, or from about 10 to about 20 nucleotides, and comprises the codon corresponding to the position containing codon encoding the particular variation (e.g., I148M of PNPLA3 or the portion of the variant HSD17B13 protein that is different from the corresponding wild type HSD17B13 protein). In some preferred embodiments, the portion of the nucleic acid molecule sequenced comprises from about 10 to about 50, from about 10 to about 40, from about 10 to about 30, or from about 10 to about 20 nucleotides, and comprises the codon corresponding to the position containing codon encoding the particular variation (e.g., I148M of PNPLA3 or the portion of the variant HSD17B13 protein that is different from the corresponding wild type HSD17B13 protein).

In some embodiments, the methods of detecting the presence or absence of any of the particular PNPLA3 nucleic acid molecules or HSD17B13 nucleic acid molecules (e.g., any of the functional or variant genomic DNA molecules, mRNA molecules, cDNA molecules, RNA transcripts, or cDNA transcripts) described herein in a subject, comprise: performing an assay on a biological sample obtained from the subject, which assay determines whether a nucleic acid molecule in the biological sample comprises any of the particular PNPLA3 nucleic acid molecules or HSD17B13 nucleic acid molecules (e.g., any of the functional or variant genomic DNA molecules, mRNA molecules, cDNA molecules, RNA transcripts, or cDNA transcripts) described herein. In some embodiments, the biological sample comprises a cell or cell lysate. Such methods can further comprise, for example, obtaining a biological sample from the subject, optionally reverse transcribing the mRNA into cDNA, and performing the assay. Such assays can comprise, for example, determining the identity of particular positions of the particular nucleic acid molecules described herein.

For example, the assay can comprise the use of alteration-specific probes or alteration-specific primers that comprise a nucleic acid sequence which is complementary to and/or hybridizes, or specifically hybridizes, to a portion of a PNPLA3 nucleic acid sequence that comprises a methionine at a position corresponding to position 148 according to SEQ ID NO:42, or comprises a methionine at a position corresponding to position 144 according to SEQ ID NO:43, or a portion adjacent thereto. In some embodiments, the assay can comprise the use of alteration-specific probes or alteration-specific primers that comprise a nucleic acid sequence which is complementary to and/or hybridizes, or specifically hybridizes, to the portion of the variant PNPLA3 genomic DNA that comprises an ATG codon at the positions corresponding to positions 5107 to 5109 according to SEQ ID NO:31, or a portion adjacent thereto. In some embodiments, the assay can comprise the use of alteration-specific probes or alteration-specific primers that comprise a nucleic acid sequence which is complementary to and/or hybridizes, or specifically hybridizes, to the portion of the variant PNPLA3 mRNA that comprises an AUG codon at the positions corresponding to positions 442 to 444 according to SEQ ID NO:34, or a portion adjacent thereto. In some embodiments, the assay can comprise the use of alteration-specific probes or alteration-specific primers that comprise a nucleic acid sequence which is complementary to and/or hybridizes, or specifically hybridizes, to the portion of the variant PNPLA3 mRNA that comprises an AUG codon at the positions corresponding to positions 430 to 432 according to SEQ ID NO:35, or a portion adjacent thereto. In some embodiments, the assay can comprise the use of alteration-specific probes or alteration-specific primers that comprise a nucleic acid sequence which is complementary to and/or hybridizes, or specifically hybridizes, to the portion of the variant PNPLA3 cDNA that comprises an ATG codon at the positions corresponding to positions 442 to 444 according to SEQ ID NO:38, or a portion adjacent thereto. In some embodiments, the assay can comprise the use of alteration-specific probes or alteration-specific primers that comprise a nucleic acid sequence which is complementary to and/or hybridizes, or specifically hybridizes, to the portion of the variant PNPLA3 cDNA that comprises an ATG codon at the positions corresponding to positions 430 to 432 according to SEQ ID NO:39, or a portion adjacent thereto.

In some embodiments, the assay can comprise the use of alteration-specific probes or alteration-specific primers that comprise a nucleic acid sequence which is complementary to and/or hybridizes, or specifically hybridizes, to the portion of the HSD17B13 genomic DNA that comprises an adenine at a position corresponding to position 12,667 according to SEQ ID NO:1, or a portion adjacent thereto. In some embodiments, the assay can comprise the use of alteration-specific probes or alteration-specific primers that comprise a nucleic acid sequence which is complementary to and/or hybridizes, or specifically hybridizes, to the portion of the HSD17B13 genomic DNA that comprises a thymine at a position corresponding to position 12,667 according to SEQ ID NO:2, or a portion adjacent thereto.

In some embodiments, the assay comprises: sequencing at least a portion of the nucleic acid molecules described herein present in the biological sample from the subject, wherein the portion sequenced includes the positions disclosed herein. For example, the portion sequenced can be a portion of a PNPLA3 nucleic acid sequence that comprises a methionine at a position corresponding to position 148 according to SEQ ID NO:42, or comprises a methionine at a position corresponding to position 144 according to SEQ ID NO: 43. In some embodiments, the portion sequenced can be portion of the variant PNPLA3 genomic DNA that comprises an ATG codon at the positions corresponding to positions 5107 to 5109 according to SEQ ID NO:31. In some embodiments, the portion sequenced can be a portion of the variant PNPLA3 mRNA that comprises an AUG codon at the positions corresponding to positions 442 to 444 according to SEQ ID NO:34. In some embodiments, the portion sequenced can be a portion of the variant PNPLA3 mRNA that comprises an AUG codon at the positions corresponding to positions 430 to 432 according to SEQ ID NO: 35. In some embodiments, the portion sequenced can be a portion of the variant PNPLA3 cDNA that comprises an ATG codon at the positions corresponding to positions 442 to 444 according to SEQ ID NO:38. In some embodiments, the portion sequenced can be a portion of the variant PNPLA3 cDNA that comprises an ATG codon at the positions corresponding to positions 430 to 432 according to SEQ ID NO:39.

In some embodiments, the portion sequenced can be a portion of the HSD17B13 genomic DNA that comprises an adenine at a position corresponding to position 12,667 according to SEQ ID NO:1. In some embodiments, the portion sequenced can be a portion of the HSD17B13 genomic DNA that comprises a thymine at a position corresponding to position 12,667 according to SEQ ID NO:2.

In some embodiments, the assay comprises: a) contacting the biological sample with a primer (or alteration-specific primer) hybridizing to the regions adjacent to the portions of the nucleic acid molecules identified herein (e.g., adjacent to a portion of a PNPLA3 nucleic acid sequence that comprises a methionine at a position corresponding to position 148 according to SEQ ID NO:42, or comprises a methionine at a position corresponding to position 144 according to SEQ ID NO:43; adjacent to a portion of the variant PNPLA3 genomic DNA that comprises an ATG codon at the positions corresponding to positions 5107 to 5109 according to SEQ ID NO:31; adjacent to a portion of the variant PNPLA3 mRNA that comprises an AUG codon at the positions corresponding to positions 442 to 444 according to SEQ ID NO:34; adjacent to a portion of the variant PNPLA3 mRNA that comprises an AUG codon at the positions corresponding to positions 430 to 432 according to SEQ ID NO:35; adjacent to a portion of the variant PNPLA3 cDNA that comprises an ATG codon at the positions corresponding to positions 442 to 444 according to SEQ ID NO:38; adjacent to a portion of the variant PNPLA3 cDNA that comprises an ATG codon at the positions corresponding to positions 430 to 432 according to SEQ ID NO:39; adjacent to a portion of the HSD17B13 genomic DNA that comprises an adenine at a position corresponding to position 12,667 according to SEQ ID NO:1; or adjacent to a portion of the HSD17B13 genomic DNA that comprises a thymine at a position corresponding to position 12,667 according to SEQ ID NO:2); b) extending the primer at least through the position of the nucleic acid molecules corresponding to nucleotide positions beyond the altered site (e.g., the portion of a PNPLA3 nucleic acid sequence that comprises a methionine at a position corresponding to position 148 according to SEQ ID NO: 42, or comprises a methionine at a position corresponding to position 144 according to SEQ ID NO: 43; the portion of the variant PNPLA3 genomic DNA that comprises an ATG codon at the positions corresponding to positions 5107 to 5109 according to SEQ ID NO:31; the portion of the variant PNPLA3 mRNA that comprises an AUG codon at the positions corresponding to positions 442 to 444 according to SEQ ID NO:34; the portion of the variant PNPLA3 mRNA that comprises an AUG codon at the positions corresponding to positions 430 to 432 according to SEQ ID NO:35; the portion of the variant PNPLA3 cDNA that comprises an ATG codon at the positions corresponding to positions 442 to 444 according to SEQ ID NO:38; the portion of the variant PNPLA3 cDNA that comprises an ATG codon at the positions corresponding to positions 430 to 432 according to SEQ ID NO:39; the portion of the HSD17B13 genomic DNA that comprises an adenine at a position corresponding to position 12,667 according to SEQ ID NO:1; or the portion of the HSD17B13 genomic DNA that comprises a thymine at a position corresponding to position 12,667 according to SEQ ID NO: 2); and c) determining whether the extension product of the primer comprises the nucleic acid sequence of any of the variant or wild type PNPLA3 or HSD17B13 nucleic acid molecules described herein.

In some embodiments, only PNPLA3 genomic DNA is analyzed. In some embodiments, only PNPLA3 mRNA is analyzed. In some embodiments, only PNPLA3 cDNA obtained from PNPLA3 mRNA is analyzed. In some embodiments, only HSD17B13 genomic DNA is analyzed. In some embodiments, only HSD17B13 mRNA is analyzed. In some embodiments, only HSD17B13 cDNA obtained from HSD17B13 mRNA is analyzed. In some embodiments, only HSD17B13 RNA transcripts is analyzed. In some embodiments, only HSD17B13 cDNA obtained from HSD17B13 RNA transcripts is analyzed.

In some embodiments, the assay comprises contacting the biological sample with a primer or probe that specifically hybridizes to any of the particular variant PNPLA3 nucleic acid molecules or variant HSD17B13 nucleic acid molecules (e.g., any of the variant genomic DNA molecules, mRNA molecules, cDNA molecules, RNA transcripts, or cDNA transcripts) described herein and not the corresponding functional nucleic acid molecules under stringent conditions, and determining whether hybridization has occurred.

In some embodiments, the assay comprises contacting the biological sample with a primer or probe that specifically hybridizes to any of the particular variant PNPLA3 nucleic acid molecules (e.g., any of the variant genomic DNA molecules, mRNA molecules, cDNA molecules, RNA transcripts, or cDNA transcripts) or nucleic acid molecules encoding a functional HSD17B13 protein (e.g., any of the genomic DNA molecules, mRNA molecules, cDNA molecules, RNA transcripts, or cDNA transcripts encoding a functional HSD17B13 protein) described herein and not to the corresponding nucleic acid molecules encoding wild type PNPLA3 or variant HSD17B13, respectively, under stringent conditions, and determining whether hybridization has occurred.

In some embodiments, the assay comprises RNA sequencing (RNA-Seq). In some embodiments, the assays also comprise reverse transcribing mRNA into cDNA via the reverse transcriptase polymerase chain reaction (RT-PCR).

Such probes and primers can hybridize specifically to a target sequence under high stringency hybridization conditions. Probes and primers may have complete nucleic acid sequence identity of contiguous nucleotides with the target sequence, although probes differing from the target nucleic acid sequence and that retain the ability to specifically detect and/or identify a target nucleic acid sequence may be designed by conventional methods. Accordingly, probes and primers can share at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity or complementarity to the target nucleic acid molecule.

When a probe is hybridized with a nucleic acid molecule in a biological sample under conditions that allow for the binding of the probe to the nucleic acid molecule, this binding can be detected and allow for an indication of the presence of the particular variant or wild type PNPLA3 or variant or functional HSD17B13 locus or the presence or the level of the particular variant or wild type PNPLA3 or variant or functional HSD17B13 mRNA or cDNA in the biological sample. Such identification of a bound probe has been described. The specific probe may comprise a sequence of at least about 80%, from about 80% to about 85%, from about 85% to about 90%, from about 90% to about 95%, and from about 95% to about 100% identical (or complementary) to a specific region of a variant or wild type PNPLA3 or variant or functional HSD17B13 gene. The specific probe may comprise a sequence of at least about 80%, from about 80% to about 85%, from about 85% to about 90%, from about 90% to about 95%, and from about 95% to about 100% identical (or complementary) to a specific region of a variant or wild type PNPLA3 or variant or functional HSD17B13 mRNA. The specific probe may comprise a sequence of at least about 80%, from about 80% to about 85%, from about 85% to about 90%, from about 90% to about 95%, and from about 95% to about 100% identical (or complementary) to a specific region of a variant or wild type PNPLA3 or variant or functional HSD17B13 cDNA.

In some embodiments, to determine whether a particular nucleic acid complement of a biological sample comprises a nucleic acid sequence encoding a particular functional or variant PNPLA3 protein or HSD17B13 protein, the biological sample may be subjected to a nucleic acid amplification method using a primer pair that includes a first primer derived from the 5′ flanking sequence adjacent to positions encoding a site of interest (e.g., any of the positions described herein), and a second primer derived from the 3′ flanking sequence adjacent to positions encoding the same site of interest, to produce an amplicon that is diagnostic for the presence of the particular functional or variant PNPLA3 protein or HSD17B13 protein. For example, with regard to PNPLA3 the amplicon may comprise a nucleotide sequence encoding the position which corresponds to position 148 according to SEQ ID NO: 42. With regard to HSD17B13 the amplicon may comprise a nucleotide sequence which corresponds to positions 5107 to 5109 according to SEQ ID NO: 31. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions encoding the site of interest and at least 1, 2, 3, 4, 5, 6, 7, 8, 9, 10, or more nucleotides on each side of positions encoding the site of interest. Similar amplicons can be generated from the mRNA and/or cDNA sequences.

Representative methods for preparing and using probes and primers are described, for example, in Molecular Cloning: A Laboratory Manual, 2nd Ed., Vol. 1-3, ed. Sambrook et al., Cold Spring Harbor Laboratory Press, Cold Spring Harbor, N.Y. 1989 (hereinafter, “Sambrook et al., 1989”); Current Protocols in Molecular Biology, ed. Ausubel et al., Greene Publishing and Wiley-Interscience, New York, 1992 (with periodic updates) (hereinafter, “Ausubel et al., 1992”); and Innis et al., PCR Protocols: A Guide to Methods and Applications, Academic Press: San Diego, 1990). PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using guidelines.

Any nucleic acid hybridization or amplification or sequencing method can be used to specifically detect the presence of the functional or variant PNPLA3 or HSD17B13 gene locus and/or the level of functional or variant PNPLA3 or HSD17B13 mRNA or cDNA produced from mRNA. In some embodiments, the nucleic acid molecule can be used either as a primer to amplify a region of the functional or variant PNPLA3 or HSD17B13 nucleic acid or the nucleic acid molecule can be used as a probe that specifically hybridizes, for example, under stringent conditions, to a nucleic acid molecule comprising the functional or variant PNPLA3 or HSD17B13 gene locus or a nucleic acid molecule comprising a functional or variant PNPLA3 or HSD17B13 mRNA or cDNA produced from mRNA.

A variety of techniques are available in the art including, for example, nucleic acid sequencing, nucleic acid hybridization, and nucleic acid amplification. Illustrative examples of nucleic acid sequencing techniques include, but are not limited to, chain terminator (Sanger) sequencing and dye terminator sequencing.

Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

Any method can be used for detecting either the non-amplified or amplified polynucleotides including, for example, Hybridization Protection Assay (HPA), quantitative evaluation of the amplification process in real-time, and determining the quantity of target sequence initially present in a sample, but which is not based on a real-time amplification.

Also provided are methods for identifying nucleic acids which do not necessarily require sequence amplification and are based on, for example, the methods of Southern (DNA: DNA) blot hybridizations, in situ hybridization (ISH), and fluorescence in situ hybridization (FISH) of chromosomal material. Southern blotting can be used to detect specific nucleic acid sequences. In such methods, nucleic acid that is extracted from a sample is fragmented, electrophoretically separated on a matrix gel, and transferred to a membrane filter. The filter bound nucleic acid is subject to hybridization with a labeled probe complementary to the sequence of interest. Hybridized probe bound to the filter is detected. In any such methods, the process can include hybridization using any of the probes described or exemplified herein.

In hybridization techniques, stringent conditions can be employed such that a probe or primer will specifically hybridize to its target. In some embodiments, a polynucleotide primer or probe under stringent conditions will hybridize to its target sequence (e.g., the functional or variant PNPLA3 or HSD17B13 locus, functional or variant PNPLA3 or HSD17B13 mRNA, or functional or variant PNPLA3 or HSD17B13 cDNA) to a detectably greater degree than to other sequences (e.g., the corresponding functional or variant PNPLA3 or HSD17B13 locus, functional or variant PNPLA3 or HSD17B13 mRNA, or functional or variant PNPLA3 or HSD17B13 cDNA), such as, at least 2-fold, at least 3-fold, at least 4-fold, or more over background, including over 10-fold over background. In some embodiments, a polynucleotide primer or probe under stringent conditions will hybridize to its target sequence to a detectably greater degree than to other sequences by at least 2-fold. In some embodiments, a polynucleotide primer or probe under stringent conditions will hybridize to its target sequence to a detectably greater degree than to other sequences by at least 3-fold. In some embodiments, a polynucleotide primer or probe under stringent conditions will hybridize to its target sequence to a detectably greater degree than to other sequences by at least 4-fold. In some embodiments, a polynucleotide primer or probe under stringent conditions will hybridize to its target sequence to a detectably greater degree than to other sequences by over 10-fold over background. Stringent conditions are sequence-dependent and will be different in different circumstances. By controlling the stringency of the hybridization and/or washing conditions, target sequences that are 100% complementary to the probe can be identified (homologous probing). Alternately, stringency conditions can be adjusted to allow some mismatching in sequences so that lower degrees of identity are detected (heterologous probing).

In some embodiments, the detecting step comprises: amplifying at least a portion of the nucleic acid molecule that encodes a site of interest (e.g., any of the positions described herein); labeling the nucleic acid molecule with a detectable label; contacting the labeled nucleic acid with a support comprising a probe, wherein the probe comprises a nucleic acid sequence which hybridizes under stringent conditions to a nucleic acid sequence encoding the a site of interest (e.g., any of the positions described herein); and detecting the detectable label.

In some embodiments, the detecting step comprises: amplifying at least a portion of the nucleic acid molecule that encodes a PNPLA3 or HSD17B13 protein, wherein the amplified nucleic acid molecule encodes an amino acid sequence which comprises a site of interest (e.g., any of the positions described herein); labeling the nucleic acid molecule with a detectable label; contacting the labeled nucleic acid with a support comprising a probe, wherein the probe comprises a nucleic acid sequence which hybridizes under stringent conditions to a nucleic acid sequence encoding a site of interest (e.g., any of the positions described herein); and detecting the detectable label. Any of the nucleic acid molecules disclosed herein can be amplified. For example, any of the genomic DNA, cDNA, or mRNA molecules disclosed herein can be amplified. In some embodiments, the nucleic acid molecule is mRNA and the method further comprises reverse-transcribing the mRNA into a cDNA prior to the amplifying step.

In some embodiments, the detecting step comprises: contacting the nucleic acid molecule that encodes a PNPLA3 or HSD17B13 protein with a probe comprising a detectable label, wherein the probe comprises a nucleic acid sequence which hybridizes under stringent conditions to a nucleic acid sequence encoding the variant PNPLA3 or HSD17B13 protein, and detecting the detectable label. In some embodiments, the detecting step comprises: contacting the nucleic acid molecule that encodes a PNPLA3 or HSD17B13 protein with a probe comprising a detectable label, wherein the probe comprises a nucleic acid sequence which hybridizes under stringent conditions to a nucleic acid sequence encoding a site of interest (e.g., any of the positions described herein), and detecting the detectable label. In some embodiments, the nucleic acid molecule is present within a cell obtained from the human subject, such that the detection is according to an in situ hybridization technique.

Other assays that can be used in the methods disclosed herein include, for example, reverse transcription polymerase chain reaction (RT-PCR) or quantitative RT-PCR (qRT-PCR). Yet other assays that can be used in the methods disclosed herein include, for example, RNA sequencing (RNA-Seq) followed by detection of the presence and quantity of variant mRNA or cDNA in the biological sample.

In some embodiments, the detecting step comprises amplifying at least a portion of the nucleic acid molecule that encodes a particular PNPLA3 or HSD17B13 protein, labeling the amplified nucleic acid molecule with a detectable label, contacting the labeled nucleic acid molecule with a support comprising a probe, wherein the probe comprises a nucleic acid sequence which specifically hybridizes, including, for example, under stringent conditions, to a nucleic acid sequence encoding the particular PNPLA3 or HSD17B13 protein, and detecting the detectable label. In some embodiments, the detecting step comprises amplifying at least a portion of the nucleic acid molecule that encodes a particular PNPLA3 or HSD17B13 protein, labeling the amplified nucleic acid molecule with a detectable label, contacting the labeled nucleic acid molecule with a support comprising a probe, wherein the probe comprises a nucleic acid sequence which specifically hybridizes, including, for example, under stringent conditions, to a nucleic acid sequence encoding a site of interest (e.g., the portion of a PNPLA3 nucleic acid sequence that encodes a methionine at a position corresponding to position 148 according to SEQ ID NO:42, or encodes a methionine at a position corresponding to position 144 according to SEQ ID NO:43; the portion of the variant PNPLA3 genomic DNA that comprises an ATG codon at the positions corresponding to positions 5107 to 5109 according to SEQ ID NO:31; the portion of the variant PNPLA3 mRNA that comprises an AUG codon at the positions corresponding to positions 442 to 444 according to SEQ ID NO:34; the portion of the variant PNPLA3 mRNA that comprises an AUG codon at the positions corresponding to positions 430 to 432 according to SEQ ID NO:35; the portion of the variant PNPLA3 cDNA that comprises an ATG codon at the positions corresponding to positions 442 to 444 according to SEQ ID NO:38; the portion of the variant PNPLA3 cDNA that comprises an ATG codon at the positions corresponding to positions 430 to 432 according to SEQ ID NO:39; the portion of the HSD17B13 genomic DNA that comprises an adenine at a position corresponding to position 12,667 according to SEQ ID NO: 1; or the portion of the HSD17B13 genomic DNA that comprises a thymine at a position corresponding to position 12,667 according to SEQ ID NO:2), and detecting the detectable label. If the nucleic acid includes mRNA, the method may further comprise reverse-transcribing the mRNA into a cDNA prior to the amplifying step. In some embodiments, the determining step comprises contacting the nucleic acid molecule that encodes a particular PNPLA3 or HSD17B13 protein with a probe comprising a detectable label and detecting the detectable label.

The disclosure provides methods for identifying a human subject as a candidate for treating or inhibiting a liver disease by inhibiting hydroxysteroid 17-beta dehydrogenase 13 (HSD17B13), the method comprising determining whether or not a sample from the subject comprises a first nucleic acid encoding a patatin like phospholipase domain containing 3 (PNPLA3) protein comprising an I148M variation and a second nucleic acid encoding a functional HSD17B13 protein, and/or a PNPLA3 protein comprising an I148M variation and a functional HSD17B13 protein, and identifying the subject as a candidate for treating or inhibiting a liver disease by inhibiting HSD17B13 when both the first and second nucleic acids are detected and/or both of the proteins are detected. In some embodiments, the subject is obese. In some embodiments, the subject has a fatty liver. In some embodiments, the first nucleic acid molecule comprises genomic DNA. In some embodiments, the genomic DNA comprises an ATG codon at the positions corresponding to positions 5107 to 5109 according to SEQ ID NO:31. In some embodiments, the genomic DNA comprises the nucleotide sequence according to SEQ ID NO:31. In some embodiments, the first nucleic acid molecule comprises mRNA. In some embodiments, the mRNA comprises an AUG codon at the positions corresponding to positions 442 to 444 according to SEQ ID NO:34. In some embodiments, the mRNA comprises the nucleotide sequence according to SEQ ID NO:34. In some embodiments, the mRNA comprises an AUG codon at the positions corresponding to positions 430 to 432 according to SEQ ID NO:35. In some embodiments, the mRNA comprises the nucleotide sequence according to SEQ ID NO:35. In some embodiments, the first nucleic acid molecule comprises a cDNA obtained from mRNA. In some embodiments, the cDNA comprises an ATG codon at the positions corresponding to positions 442 to 444 according to SEQ ID NO:38. In some embodiments, the cDNA comprises the nucleotide sequence according to SEQ ID NO:38. In some embodiments, the cDNA comprises an ATG codon at the positions corresponding to positions 430 to 432 according to SEQ ID NO:39. In some embodiments, the cDNA comprises the nucleotide sequence according to SEQ ID NO: 39. In some embodiments, detecting the first nucleic acid comprises sequencing at least a portion of the first nucleic acid and the portion comprises the codon which encodes the I148M variation. In some embodiments, detecting the first nucleic acid comprises hybridizing the first nucleic acid with a probe or primer that specifically hybridizes to a portion of the first nucleic acid, wherein the portion comprises the codon encoding the I148M variation. In some embodiments, the probe or primer is an allele-specific probe or primer. In some embodiments, the probe or primer comprises a label. In some embodiments, the methods further comprise determining whether the subject is homozygous or heterozygous for the I148M variation.

In some embodiments, the second nucleic acid comprises genomic DNA. In some embodiments, the genomic DNA comprises an adenine at a position corresponding to position 12,667 according to SEQ ID NO:1. In some embodiments, the genomic DNA comprises SEQ ID NO:1. In some embodiments, the second nucleic acid molecule comprises mRNA. In some embodiments, the mRNA comprises SEQ ID NO:3. In some embodiments, the mRNA comprises SEQ ID NO:4. In some embodiments, the mRNA comprises SEQ ID NO: 7. In some embodiments, the mRNA comprises SEQ ID NO:11. In some embodiments, the second nucleic acid molecule comprises cDNA obtained from mRNA. In some embodiments, the cDNA comprises SEQ ID NO:12. In some embodiments, the cDNA comprises SEQ ID NO:13. In some embodiments, the cDNA comprises SEQ ID NO:16. In some embodiments, the cDNA comprises SEQ ID NO:20. In some embodiments, detecting the second nucleic acid comprises sequencing the second nucleic acid. In some embodiments, detecting the second nucleic acid comprises hybridizing the second nucleic acid with a probe or primer that specifically hybridizes to the second nucleic acid. In some embodiments, the probe or primer is an allele-specific probe or primer. In some embodiments, the probe or primer comprises a label. In some embodiments, the methods further comprise determining whether the subject is homozygous or heterozygous for the second nucleic acid encoding a functional HSD17B13 protein in the sample.

The present disclosure provides methods of identifying a subject who is a candidate for HSD17B13 inhibition, the method comprising determining whether or not a sample from the subject comprises a nucleic acid encoding a PNPLA3 Ile148Met variant or PNPLA3 Ile144Met variant. The present disclosure also provides methods for identifying a human subject as a candidate for treating or inhibiting a liver disease by inhibiting HSD17B13, the method comprising determining whether or not a sample from the subject comprises a first nucleic acid encoding a PNPLA3 protein comprising an I148M variation and a second nucleic acid encoding a functional HSD17B13 protein, and/or a PNPLA3 protein comprising an I148M variation and a functional HSD17B13 protein, and identifying the subject as a candidate for treating or inhibiting a liver disease by inhibiting HSD17B13 when both the first and second nucleic acids are detected and/or when both proteins are detected.

The present disclosure also provides methods of classifying a subject who is a candidate for HSD17B13 inhibition, the method comprising determining whether or not a sample from the subject comprises a nucleic acid encoding a PNPLA3 Ile148Met variant or PNPLA3 Ile144Met variant. The present disclosure also provides methods for classifying a human subject as a candidate for treating or inhibiting a liver disease by inhibiting HSD17B13, the method comprising determining whether or not a sample from the subject comprises a first nucleic acid encoding a PNPLA3 protein comprising an I148M variation and a second nucleic acid encoding a functional HSD17B13 protein, and/or a PNPLA3 protein comprising an I148M variation and a functional HSD17B13 protein, and classifying the subject as a candidate for treating or inhibiting a liver disease by inhibiting HSD17B13 when both the first and second nucleic acids are detected and/or when both proteins are detected.

The variant PNPLA3 Ile148Met variant or PNPLA3 Ile144Met variant can be any of the variant PNPLA3 Ile148Met variants and PNPLA3 Ile144Met variants described herein. The variant PNPLA3 Ile148Met variant or PNPLA3 Ile144Met variant can be detected by any of the methods described herein. In some embodiments, the methods further comprise determining whether the subject is homozygous or heterozygous for the variant PNPLA3 Ile148Met variant or PNPLA3 Ile144Met variant. In some embodiments, the subject is homozygous for the variant PNPLA3 Ile148Met variant or PNPLA3 Ile144Met variant. In some embodiments, the subject is heterozygous for the variant PNPLA3 Ile148Met variant or PNPLA3 Ile144Met variant. In some embodiments, the subject is homozygous for the variant PNPLA3 Ile148Met variant. In some embodiments, the subject is heterozygous for the variant PNPLA3 Ile148Met variant. In some embodiments, the subject is homozygous for the variant PNPLA3 Ile144Met variant. In some embodiments, the subject is heterozygous for the variant PNPLA3 Ile144Met variant.

In preferred embodiments, the subject does not comprise any genes encoding loss of function variations in the HSD17B13 protein. It is believed that loss of function variations in the HSD17B13 protein, including those described or exemplified herein, confer a liver disease-protective effect and it is further believed that this protective effect is enhanced in the presence of the variant PNPLA3 Ile148M variation. Thus, it is believed that subjects (e.g., subjects comprising the I148M variation in PNPLA3) in whom both copies of the genes (from each chromosome) encoding the HSD17B13 protein encode a loss of function variation are unlikely to benefit from HSD17B13 inhibition therapy. Nevertheless, it is believed that subjects who express at least a partially functional HSD17B13 protein will benefit from HSD17B13 inhibition therapy. Thus, the methods may comprise classifying the status of the gene (in one or both chromosomes) encoding HSD17B13, including whether the gene encodes a loss of function variation in the HSD17B13 protein, as well as whether the subject is homozygous or heterozygous.

In some embodiments, the methods further comprise detecting the presence of a nucleic acid molecule or gene encoding a functional HSD17B13 protein in a sample from the subject. The nucleic acid molecule can encode any of the functional HSD17B13 proteins described herein. The HSD17B13 nucleic acid molecule can be detected by any of the methods described herein. In some embodiments, the methods further comprise determining whether the subject is homozygous or heterozygous for a gene encoding a functional HSD17B13 protein. In some embodiments, the subject is homozygous for a gene encoding a functional HSD17B13 protein. In some embodiments, the subject is heterozygous for a gene encoding a functional HSD17B13.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A form for a solid-state substrate is a microtiter dish, such as a standard 96-well type. In some embodiments, a multi-well glass slide can be employed that normally contain one array per well. This feature allows for greater control of assay reproducibility, increased throughput and sample handling, and ease of automation. In some embodiments, the support is a microarray.

In some embodiments, the methods further comprises determining whether the subject is obese. In some embodiments, a subject is obese if their body mass index (BMI) is over 30 kg/m². Obesity is can be a characteristic of a subject having or at risk of developing a liver disease. In some embodiments, the methods further comprises determining whether the subject has a fatty liver. A fatty liver can be a characteristic of a subject having or at risk of developing a liver disease. In some embodiments, the methods further comprises determining whether the subject is obese and has a fatty liver.

In some embodiments, the methods further comprise administering an inhibitor of HSD17B13 to the subject. Methods of administering an inhibitor of HSD17B13 to the subject are described herein.

In some embodiments, the nucleic acid molecule encoding the variant PNPLA3 Ile148Met protein or PNPLA3 Ile144Met protein is any of the nucleic acid molecules described herein. In some embodiments, the nucleic acid molecule encoding the variant PNPLA3 Ile148Met protein or PNPLA3 Ile144Met protein is genomic DNA. In some embodiments, the genomic DNA encoding the variant PNPLA3 Ile148Met protein or PNPLA3 Ile144Met protein is any of the genomic DNA molecules described herein. In some embodiments, the nucleic acid molecule encoding the variant PNPLA3 Ile148Met protein is genomic DNA. In some embodiments, the genomic DNA comprises an ATG codon at the positions corresponding to positions 5107 to 5109 according to SEQ ID NO:31. In some embodiments, the genomic DNA comprises the nucleotide sequence according to SEQ ID NO: 31. In some embodiments, the genomic DNA encoding the variant PNPLA3 protein is detected by nucleic acid sequencing or hybridization of a probe, as described herein. In some embodiments, the genomic DNA encoding the variant PNPLA3 protein that comprises the ATG codon is detected by nucleic acid sequencing or hybridization of a probe, as described herein.

In some embodiments, the nucleic acid molecule encoding the variant PNPLA3 Ile148Met protein or PNPLA3 Ile144Met protein is mRNA. In some embodiments, the mRNA encoding the variant PNPLA3 Ile148Met protein or PNPLA3 Ile144Met protein is any of the mRNA molecules described herein. In some embodiments, the nucleic acid molecule encoding the variant PNPLA3 Ile148Met protein is mRNA. In some embodiments, the mRNA comprises an AUG codon at the positions corresponding to positions 442 to 444 according to SEQ ID NO:34. In some embodiments, the mRNA comprises the nucleotide sequence according to SEQ ID NO:34. In some embodiments, the mRNA comprises an AUG codon at the positions corresponding to positions 430 to 432 according to SEQ ID NO:35. In some embodiments, the mRNA comprises the nucleotide sequence according to SEQ ID NO:35. In some embodiments, the mRNA encoding the variant PNPLA3 protein is detected by nucleic acid sequencing or hybridization of a probe, as described herein. In some embodiments, the mRNA encoding the variant PNPLA3 protein that comprises the AUG codon is identified by nucleic acid sequencing or hybridization of a probe, as described herein.

In some embodiments, the nucleic acid molecule encoding the variant PNPLA3 Ile148Met protein or PNPLA3 Ile144Met protein is cDNA. In some embodiments, the cDNA encoding the variant PNPLA3 Ile148Met protein or PNPLA3 Ile144Met protein is any of the cDNA molecules described herein. In some embodiments, the nucleic acid molecule encoding a PNPLA3 Ile148Met protein is cDNA. In some embodiments, the cDNA comprises an ATG codon at the positions corresponding to positions 442 to 444 according to SEQ ID NO: 38. In some embodiments, the cDNA comprises the nucleotide sequence according to SEQ ID NO:38. In some embodiments, the cDNA comprises an ATG codon at the positions corresponding to positions 430 to 432 according to SEQ ID NO:39. In some embodiments, the cDNA comprises the nucleotide sequence according to SEQ ID NO:39. In some embodiments, the cDNA encoding the variant PNPLA3 protein is identified by nucleic acid sequencing or hybridization of a probe. In some embodiments, the cDNA encoding the variant PNPLA3 protein that comprises the ATG codon is identified by nucleic acid sequencing or hybridization of a probe, as described herein.

In some embodiments, the nucleic acid molecule encoding the functional HSD17B13 protein is any of the nucleic acid molecules described herein. In some embodiments, the nucleic acid molecule encoding the functional HSD17B13 protein is genomic DNA. In some embodiments, the genomic DNA encoding the functional HSD17B13 protein is any of the genomic DNA molecules described herein. In some embodiments, the genomic DNA comprises an adenine at a position corresponding to position 12,667 according to SEQ ID NO:1. In some embodiments, the genomic DNA comprises SEQ ID NO:1. In some embodiments, the presence of the functional HSD17B13 genomic DNA is determined by nucleic acid sequencing or hybridization of a probe, as described herein.

In some embodiments, the nucleic acid molecule encoding the functional HSD17B13 protein is mRNA. In some embodiments, the mRNA encoding the functional HSD17B13 protein is any of the mRNA molecules described herein. In some embodiments, the functional HSD17B13 nucleic acid molecule is mRNA. In some embodiments, the mRNA comprises SEQ ID NO:3. In some embodiments, the mRNA comprises SEQ ID NO:4. In some embodiments, the mRNA comprises SEQ ID NO:7. In some embodiments, the mRNA comprises SEQ ID NO:11. In some embodiments, the presence of the functional HSD17B13 mRNA is determined by nucleic acid sequencing or hybridization of a probe, as described herein.

In some embodiments, the nucleic acid molecule encoding the functional HSD17B13 protein is cDNA. In some embodiments, the cDNA encoding the functional HSD17B13 protein is any of the cDNA molecules described herein. In some embodiments, the cDNA comprises SEQ ID NO:12. In some embodiments, the cDNA comprises SEQ ID NO: 13. In some embodiments, the cDNA comprises SEQ ID NO:16. In some embodiments, the cDNA comprises SEQ ID NO:20. In some embodiments, the presence of the functional HSD17B13 cDNA is determined by nucleic acid sequencing or hybridization of a probe, as described herein.

In some embodiments, the methods further comprising obtaining the sample from the subject. In some embodiments, the subject who is a candidate for HSD17B13 inhibition has a liver disease or is susceptible to developing a liver disease. In some embodiments, the liver disease is a chronic liver disease. In some preferred embodiments, the chronic liver disease is nonalcoholic fatty liver disease (NAFLD), alcoholic liver disease (ALD), non-alcoholic steatohepatitis (NASH), cirrhosis, steatosis, or hepatocellular carcinoma. In some preferred embodiments, the chronic liver disease is nonalcoholic fatty liver disease (NAFLD), alcoholic liver disease (ALD), non-alcoholic steatohepatitis (NASH), cirrhosis, or steatosis. In some embodiments, the liver disease is an alcoholic liver disease. In some embodiments, the alcoholic liver disease comprises one or more of cirrhosis, steatosis, or hepatocellular carcinoma resulting from alcohol consumption. In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption.

The present disclosure also provides methods of detecting a PNPLA3 Ile148Met variant, or a PNPLA3 Ile144Met variant, and functional HSD17B13 in a subject comprising: detecting the presence of a PNPLA3 Ile148Met protein, or a nucleic acid molecule encoding a PNPLA3 Ile148Met protein, in a sample from the subject, or detecting the presence of a PNPLA3 Ile144Met protein, or a nucleic acid molecule encoding a PNPLA3 Ile144Met protein, in a sample from the subject; and detecting the presence of a functional HSD17B13 protein, or a nucleic acid molecule encoding a functional HSD17B13 protein, in a sample from the subject. The variant PNPLA3 Ile148Met variant protein or nucleic acid molecule can be any of the variant PNPLA3 Ile148Met variant proteins or nucleic acid molecules described herein. The variant PNPLA3 Ile144Met variant protein or nucleic acid molecule can be any of the variant PNPLA3 Ile144Met variant proteins or nucleic acid molecules described herein. The functional HSD17B13 protein or nucleic acid molecule can be any of the functional HSD17B13 proteins or nucleic acid molecules described herein.

In some embodiments, the methods further comprise determining whether the subject is homozygous or heterozygous for the variant PNPLA3 Ile148Met variant or PNPLA3 Ile144Met variant. In some embodiments, the subject is homozygous for the variant PNPLA3 Ile148Met variant or PNPLA3 Ile144Met variant. In some embodiments, the subject is heterozygous for the variant PNPLA3 Ile148Met variant or PNPLA3 Ile144Met variant. In some embodiments, the subject is homozygous for the variant PNPLA3 Ile148Met variant. In some embodiments, the subject is heterozygous for the variant PNPLA3 Ile148Met variant. In some embodiments, the subject is homozygous for the variant PNPLA3 Ile144Met variant. In some embodiments, the subject is heterozygous for the variant PNPLA3 Ile144Met variant.

In some embodiments, the methods further comprise determining whether the subject is homozygous or heterozygous for functional HSD17B13. In some embodiments, the subject is homozygous for functional HSD17B13. In some embodiments, the subject is heterozygous for functional HSD17B13.

In some embodiments, the presence of a functional HSD17B13 protein is detected in the sample. The functional HSD17B13 protein can be any of the functional HSD17B13 proteins described herein. In some embodiments, the functional HSD17B13 protein comprises an amino acid sequence according to SEQ ID NO:21, SEQ ID NO:22, SEQ ID NO:25, or SEQ ID NO:29. In some embodiments, the functional HSD17B13 protein is detected by amino acid sequencing or immunoassay, as described herein.

In some embodiments, the presence of a functional HSD17B13 nucleic acid molecule is detected in the sample. The functional HSD17B13 nucleic acid molecule can be any of the functional HSD17B13 nucleic acid molecules described herein. In some embodiments, the functional HSD17B13 nucleic acid molecule is genomic DNA. The functional HSD17B13 genomic DNA molecule can be any of the functional HSD17B13 genomic DNA molecules described herein. In some embodiments, the genomic DNA comprises an adenine at a position corresponding to position 12,667 according to SEQ ID NO: 1. In some embodiments, the genomic DNA comprises SEQ ID NO:1. In some embodiments, the genomic DNA is detected by nucleic acid sequencing or hybridization of a probe, as described herein.

In some embodiments, the functional HSD17B13 nucleic acid molecule is mRNA. The functional HSD17B13 mRNA molecule can be any of the functional HSD17B13 mRNA molecules described herein. In some embodiments, the mRNA comprises SEQ ID NO:3. In some embodiments, the mRNA comprises SEQ ID NO:4. In some embodiments, the mRNA comprises SEQ ID NO:7. In some embodiments, the mRNA comprises SEQ ID NO:11. In some embodiments, the mRNA is detected by nucleic acid sequencing or hybridization of a probe, as described herein.

In some embodiments, the functional HSD17B13 nucleic acid molecule is cDNA. The functional HSD17B13 cDNA molecule can be any of the functional HSD17B13 cDNA molecules described herein. In some embodiments, the cDNA comprises SEQ ID NO:12. In some embodiments, the cDNA comprises SEQ ID NO:13. In some embodiments, the cDNA comprises SEQ ID NO:16. In some embodiments, the cDNA comprises SEQ ID NO:20. In some embodiments, the cDNA is detected by nucleic acid sequencing or hybridization of a probe, as described herein.

In some embodiments, the presence of a PNPLA3 Ile148Met protein or PNPLA3 Ile144Met protein is detected in the sample. The variant PNPLA3 Ile148Met protein or PNPLA3 Ile144Met protein can be any of the variant PNPLA3 Ile148Met proteins or PNPLA3 Ile144Met proteins described herein. In some embodiments, the variant PNPLA3 Ile148Met protein comprises a methionine at a position corresponding to position 148 according to SEQ ID NO:42, or comprises a methionine at a position corresponding to position 144 according to SEQ ID NO:43. In some embodiments, the variant PNPLA3 Ile148Met protein comprises a methionine at a position corresponding to position 148 according to SEQ ID NO: 42. In some embodiments, the variant PNPLA3 protein in the sample comprises the amino acid sequence according to SEQ ID NO:42. In some embodiments, the variant PNPLA3 Ile148Met protein comprises a methionine at a position corresponding to position 144 according to SEQ ID NO:43. In some embodiments, the variant PNPLA3 protein in the sample comprises the amino acid sequence according to SEQ ID NO:43. In some embodiments, the variant PNPLA3 Ile148Met protein or PNPLA3 Ile144Met protein is detected by amino acid sequencing or immunoassay, as described herein.

In some embodiments, the presence of a nucleic acid molecule encoding a PNPLA3 Ile148Met protein or PNPLA3 Ile148Met protein is detected in the sample. The nucleic acid molecule encoding a PNPLA3 Ile148Met protein or PNPLA3 Ile148Met protein can be any of the nucleic acid molecules encoding a PNPLA3 Ile148Met protein or PNPLA3 Ile148Met protein described herein.

In some embodiments, the nucleic acid molecule encoding a PNPLA3 Ile148Met protein or PNPLA3 Ile144Met protein is genomic DNA. The genomic DNA encoding a PNPLA3 Ile148Met protein or PNPLA3 Ile144Met protein can be any of the genomic DNA molecules described herein. In some embodiments, the genomic DNA comprises an ATG codon at the positions corresponding to positions 5107 to 5109 according to SEQ ID NO:31. In some embodiments, the genomic DNA comprises the nucleotide sequence according to SEQ ID NO: 31. In some embodiments, the genomic DNA encoding the variant PNPLA3 protein that comprises the ATG codon is detected by nucleic acid sequencing or hybridization of a probe, as described herein.

In some embodiments, the nucleic acid molecule encoding a PNPLA3 Ile148Met protein or PNPLA3 Ile148Met protein is mRNA. The mRNA molecule encoding a PNPLA3 Ile148Met protein or PNPLA3 Ile148Met protein can be any of the mRNA molecules described herein. In some embodiments, the mRNA comprises an AUG codon at the positions corresponding to positions 442 to 444 according to SEQ ID NO:34. In some embodiments, the mRNA comprises the nucleotide sequence according to SEQ ID NO:34. In some embodiments, the mRNA comprises an AUG codon at the positions corresponding to positions 430 to 432 according to SEQ ID NO:35. In some embodiments, the mRNA comprises the nucleotide sequence according to SEQ ID NO:35. In some embodiments, the mRNA encoding the variant PNPLA3 protein that comprises the AUG codon is identified by nucleic acid sequencing or hybridization of a probe, as described herein.

In some embodiments, nucleic acid molecule encoding a PNPLA3 Ile148Met protein or PNPLA3 Ile144Met protein is cDNA. The cDNA encoding a PNPLA3 Ile148Met protein or PNPLA3 Ile144Met protein can be any of the cDNA molecules described herein. In some embodiments, the cDNA comprises an ATG codon at the positions corresponding to positions 442 to 444 according to SEQ ID NO:38. In some embodiments, the cDNA comprises the nucleotide sequence according to SEQ ID NO:38. In some embodiments, the cDNA comprises an ATG codon at the positions corresponding to positions 430 to 432 according to SEQ ID NO:39. In some embodiments, the cDNA comprises the nucleotide sequence according to SEQ ID NO:39. In some embodiments, the cDNA encoding the variant PNPLA3 protein that comprises the ATG codon is identified by nucleic acid sequencing or hybridization of a probe, as described herein.

In some embodiments, the method further comprises obtaining the sample from the subject.

The present disclosure also provides methods of identifying a subject having a protective effect against liver disease, comprising: detecting the presence of a PNPLA3 Ile148Met variant or PNPLA3 Ile144Met variant in a sample from the subject; and detecting the presence of an HSD17B13 loss-of-function variant in a sample from the subject. The present disclosure also provides methods of classifying a subject having a protective effect against liver disease, comprising: detecting the presence of a PNPLA3 Ile148Met variant or PNPLA3 Ile144Met variant in a sample from the subject; and detecting the presence of an HSD17B13 loss-of-function variant in a sample from the subject. The variant PNPLA3 Ile148Met variant and PNPLA3 Ile144Met variant can be any of the variant PNPLA3 Ile148Met variants and PNPLA3 Ile144Met variants described herein. The HSD17B13 loss-of-function variant can be any of the HSD17B13 loss-of-function variants described herein.

In some embodiments, the methods further comprise determining whether the subject is homozygous or heterozygous for the variant PNPLA3 Ile148Met variant or PNPLA3 Ile144Met variant. In some embodiments, the subject is homozygous for the variant PNPLA3 Ile148Met variant or PNPLA3 Ile144Met variant. In some embodiments, the subject is heterozygous for the variant PNPLA3 Ile148Met variant or PNPLA3 Ile144Met variant. In some embodiments, the subject is homozygous for the variant PNPLA3 Ile148Met variant. In some embodiments, the subject is heterozygous for the variant PNPLA3 Ile148Met variant. In some embodiments, the subject is homozygous for the variant PNPLA3 Ile144Met variant. In some embodiments, the subject is heterozygous for the variant PNPLA3 Ile144Met variant.

In some embodiments, the methods further comprise determining whether the subject is homozygous or heterozygous for functional HSD17B13. In some embodiments, the subject is homozygous for functional HSD17B13. In some embodiments, the subject is heterozygous for functional HSD17B13.

In some embodiments, the variant PNPLA3 Ile148Met variant or PNPLA3 Ile144Met variant is detected in the subject by detecting a PNPLA3 Ile148Met protein, or a nucleic acid molecule encoding a PNPLA3 Ile148Met protein, in a sample from the subject, or detecting a PNPLA3 Ile144Met protein, or a nucleic acid molecule encoding a PNPLA3 Ile144Met protein, in a sample from the subject; and the HSD17B13 loss-of-function is detected in the subject by detecting an HSD17B13 loss-of-function variant protein, or a nucleic acid molecule encoding an HSD17B13 loss-of-function variant protein, in a sample from the subject. In some embodiments, the variant PNPLA3 Ile148Met variant or PNPLA3 Ile144Met variant is detected in the subject by detecting a PNPLA3 Ile148Met protein, or a nucleic acid molecule encoding a PNPLA3 Ile148Met protein, in a sample from the subject; and the HSD17B13 loss-of-function is detected in the subject by detecting an HSD17B13 loss-of-function variant protein, or a nucleic acid molecule encoding an HSD17B13 loss-of-function variant protein, in a sample from the subject.

In some embodiments, the presence of a PNPLA3 Ile148Met protein or PNPLA3 Ile144Met protein is detected in the sample. The variant PNPLA3 Ile148Met protein and PNPLA3 Ile144Met protein can be any of the variant PNPLA3 Ile148Met proteins and PNPLA3 Ile144Met proteins described herein. In some embodiments, the variant PNPLA3 Ile148Met protein comprises a methionine at a position corresponding to position 148 according to SEQ ID NO:42, or comprises a methionine at a position corresponding to position 144 according to SEQ ID NO:43. In some embodiments, the variant PNPLA3 Ile148Met protein comprises a methionine at a position corresponding to position 148 according to SEQ ID NO:42. In some embodiments, the variant PNPLA3 protein in the sample comprises the amino acid sequence according to SEQ ID NO:42. In some embodiments, the variant PNPLA3 Ile148Met protein comprises a methionine at a position corresponding to position 144 according to SEQ ID NO:43. In some embodiments, the variant PNPLA3 protein in the sample comprises the amino acid sequence according to SEQ ID NO: 43. In some embodiments, the variant PNPLA3 Ile148Met protein is detected by amino acid sequencing or immunoassay, as described herein.

In some embodiments, the presence of a nucleic acid molecule encoding a PNPLA3 Ile148Met protein or PNPLA3 Ile144Met protein is detected in the sample. The nucleic acid molecule encoding the variant PNPLA3 Ile148Met protein or PNPLA3 Ile144Met protein can be any of the nucleic acid molecules encoding the variant PNPLA3 Ile148Met proteins and PNPLA3 Ile144Met proteins described herein.

In some embodiments, wherein the nucleic acid molecule encoding a PNPLA3 Ile148Met protein or PNPLA3 Ile144Met protein is genomic DNA. The genomic DNA encoding the variant PNPLA3 Ile148Met protein or PNPLA3 Ile144Met protein can be any of the variant PNPLA3 Ile148Met proteins and PNPLA3 Ile144Met proteins described herein. In some embodiments, the genomic DNA comprises an ATG codon at the positions corresponding to positions 5107 to 5109 according to SEQ ID NO:31. In some embodiments, the genomic DNA comprises the nucleotide sequence according to SEQ ID NO:31. In some embodiments, the genomic DNA encoding the variant PNPLA3 protein that comprises the ATG codon is detected by nucleic acid sequencing or hybridization of a probe.

In some embodiments, the nucleic acid molecule encoding the variant PNPLA3 Ile148Met protein or PNPLA3 Ile144Met protein is mRNA. The mRNA molecule encoding the variant PNPLA3 Ile148Met proteins and PNPLA3 Ile144Met proteins can be any of the variant PNPLA3 Ile148Met proteins and PNPLA3 Ile144Met proteins described herein. In some embodiments, the mRNA comprises an AUG codon at the positions corresponding to positions 442 to 444 according to SEQ ID NO:34. In some embodiments, the mRNA comprises the nucleotide sequence according to SEQ ID NO:34. In some embodiments, the mRNA comprises an AUG codon at the positions corresponding to positions 430 to 432 according to SEQ ID NO:35. In some embodiments, the mRNA comprises the nucleotide sequence according to SEQ ID NO:35. In some embodiments, the mRNA encoding the variant PNPLA3 protein that comprises the AUG codon is identified by nucleic acid sequencing or hybridization of a probe, as described herein.

In some embodiments, the nucleic acid molecule encoding a PNPLA3 Ile148Met protein or PNPLA3 Ile148Met protein is cDNA. The cDNA encoding the variant PNPLA3 Ile148Met protein and PNPLA3 Ile148Met protein can be any of the variant PNPLA3 Ile148Met proteins and PNPLA3 Ile148Met proteins described herein. In some embodiments, the cDNA comprises an ATG codon at the positions corresponding to positions 442 to 444 according to SEQ ID NO:38. In some embodiments, the cDNA comprises the nucleotide sequence according to SEQ ID NO:38. In some embodiments, the cDNA comprises an ATG codon at the positions corresponding to positions 430 to 432 according to SEQ ID NO:39. In some embodiments, the cDNA comprises the nucleotide sequence according to SEQ ID NO:39. In some embodiments, the cDNA encoding the variant PNPLA3 protein that comprises the ATG codon is identified by nucleic acid sequencing or hybridization of a probe, as described herein.

In some embodiments, the presence of an HSD17B13 loss-of-function variant protein is detected in the sample. The HSD17B13 loss-of-function variant can be any of the HSD17B13 loss-of-function variant proteins described herein. In some embodiments, the HSD17B13 loss-of-function variant protein comprises an amino acid sequence according to SEQ ID NO:23. In some embodiments, the HSD17B13 loss-of-function variant protein comprises an amino acid sequence according to SEQ ID NO:24. In some embodiments, the HSD17B13 loss-of-function variant protein comprises an amino acid sequence according to SEQ ID NO:26. In some embodiments, the HSD17B13 loss-of-function variant protein comprises an amino acid sequence according to SEQ ID NO:27. In some embodiments, the HSD17B13 loss-of-function variant protein comprises an amino acid sequence according to SEQ ID NO:28. In some embodiments, the HSD17B13 loss-of-function variant protein is detected by amino acid sequencing or immunoassay, as described herein.

In some embodiments, the presence of a nucleic acid molecule encoding an HSD17B13 loss-of-function variant protein is detected in the sample. The nucleic acid molecule encoding an HSD17B13 loss-of-function variant protein can be any of the nucleic acid molecules encoding the HSD17B13 loss-of-function variant protein described herein.

In some embodiments, the nucleic acid molecule encoding the HSD17B13 loss-of-function variant protein is genomic DNA. The genomic DNA molecule encoding the HSD17B13 loss-of-function variant protein can be any of the HSD17B13 loss-of-function variant proteins described herein. In some embodiments, the genomic DNA encoding an HSD17B13 loss-of-function variant protein which comprises a thymine at a position corresponding to position 12,667 according to SEQ ID NO:2. In some embodiments, the genomic DNA encoding an HSD17B13 loss-of-function variant protein comprises SEQ ID NO: 2. In some embodiments, the genomic DNA encoding the HSD17B13 loss-of-function variant protein is detected by nucleic acid sequencing or hybridization of a probe, as described herein.

In some embodiments, the nucleic acid molecule encoding the HSD17B13 loss-of-function variant protein is mRNA. The mRNA molecule encoding the HSD17B13 loss-of-function variant protein can be any of the mRNA molecules encoding the HSD17B13 loss-of-function variant proteins described herein. In some embodiments, the mRNA encoding an HSD17B13 loss-of-function variant protein comprises SEQ ID NO:5. In some embodiments, the mRNA encoding an HSD17B13 loss-of-function variant protein comprises SEQ ID NO:6. In some embodiments, the mRNA encoding an HSD17B13 loss-of-function variant protein comprises SEQ ID NO:8. In some embodiments, the mRNA encoding an HSD17B13 loss-of-function variant protein comprises SEQ ID NO:9. In some embodiments, the mRNA encoding an HSD17B13 loss-of-function variant protein comprises SEQ ID NO:10. In some embodiments, the mRNA encoding the HSD17B13 loss-of-function variant protein is detected by nucleic acid sequencing or hybridization of a probe, as described herein.

In some embodiments, the nucleic acid molecule encoding the HSD17B13 loss-of-function variant protein is cDNA. The cDNA molecules encoding the HSD17B13 loss-of-function variant protein can be any of the cDNA molecules encoding the HSD17B13 loss-of-function variant protein described herein. In some embodiments, the cDNA encoding an HSD17B13 loss-of-function variant protein comprises SEQ ID NO:5. In some embodiments, the cDNA encoding an HSD17B13 loss-of-function variant protein comprises SEQ ID NO:6. In some embodiments, the cDNA encoding an HSD17B13 loss-of-function variant protein comprises SEQ ID NO:8. In some embodiments, the cDNA encoding an HSD17B13 loss-of-function variant protein comprises SEQ ID NO:9. In some embodiments, the cDNA encoding an HSD17B13 loss-of-function variant protein comprises SEQ ID NO:10. In some embodiments, the cDNA encoding the HSD17B13 loss-of-function variant protein is detected by nucleic acid sequencing or hybridization of a probe, as described herein.

In some embodiments, the methods further comprises obtaining the sample from the subject. In some embodiments, the liver disease is a chronic liver disease. In some embodiments, the chronic liver disease is nonalcoholic fatty liver disease (NAFLD), alcoholic liver disease (ALD), non-alcoholic steatohepatitis (NASH), cirrhosis, steatosis, or hepatocellular carcinoma. In some embodiments, the liver disease is an alcoholic liver disease. In some embodiments, the alcoholic liver disease comprises one or more of cirrhosis, steatosis, or hepatocellular carcinoma resulting from alcohol consumption. In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption.

The present disclosure also provides any of the methods described herein further comprising administering to the subject an inhibitor of HSD17B13. In some embodiments, the inhibitor of HSD17B13 comprises a functional polypeptide, an antisense DNA, RNA, an siRNA, or an shRNA that hybridizes to the endogenous HSD17B13 genomic DNA or mRNA and decreases expression of HSD17B13 polypeptide in a cell in the subject. In some embodiments, the HSD17B13 inhibitor can also inhibit one or more additional members of the short-chain dehydrogenases/reductases (SDR) family, of which HSD17B13 is a member. Such other members include, but are not limited to, HSD17B1, HSD17B2, HSD17B3, HSD17B4, HSD17B6, HSD17B7, HSD17B8, HSD17B10, HSD17B11, HSD17B12, HSD17B13, HSD17B14, HSD11B1, HSD11B2, HSD3B1, HSD3B2, and HSD3B7, as well as close homologs dehydrogenase/reductase 3 (DHRS3) and retinol dehydrogenase 10 (RDH10). In some embodiments, the inhibitor of HSD17B13 is administered to inhibit liver disease in the subject. In some embodiments, the inhibitor of HSD17B13 is administered to treat liver disease in the subject. In some embodiments, the liver disease is a chronic liver disease. In some embodiments, the chronic liver disease is one or more of nonalcoholic fatty liver disease (NAFLD), alcoholic liver disease (ALD), non-alcoholic steatohepatitis (NASH), cirrhosis, steatosis, or hepatocellular carcinoma. In some embodiments, the liver disease is an alcoholic liver disease. In some embodiments, the alcoholic liver disease comprises one or more of cirrhosis, steatosis, or hepatocellular carcinoma resulting from alcohol consumption. In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption. In some embodiments, the subject is homozygous for the gene encoding the I148M variation. In some embodiments, the subject is heterozygous for the gene encoding the I148M variation. In some embodiments, the subject further is homozygous for the gene encoding the functional HSD17B13 protein. In some embodiments, the subject further is heterozygous for the gene encoding the functional HSD17B13 protein and a gene encoding a loss of function variant of HSD17B13.

The disclosure also provides methods of treating or inhibiting liver disease, comprising administering an inhibitor of hydroxysteroid 17-beta dehydrogenase 13 (HSD17B13) to a human liver disease patient expressing a patatin like phospholipase domain containing 3 (PNPLA3) protein comprising an I148M variation such that liver disease is treated or inhibited in the patient. In some embodiments, the liver disease is a chronic liver disease. In some embodiments, the liver disease is one or more of nonalcoholic fatty liver disease (NAFLD), alcoholic liver disease (ALD), non-alcoholic steatohepatitis (NASH), cirrhosis, steatosis, or hepatocellular carcinoma. In some embodiments, the liver disease is an alcoholic liver disease. In some embodiments, the alcoholic liver disease comprises one or more of cirrhosis, steatosis, or hepatocellular carcinoma resulting from alcohol consumption. In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption. In some embodiments, the patient is obese. In some embodiments, the patient has a fatty liver. In some embodiments, the patient has been determined to express the variant PNPLA3 protein (e.g., a PNPLA3 protein comprising the I148M or 1144M variation) by detection of the variant PNPLA3 protein in a sample from the subject. In some embodiments, the variant PNPLA3 protein comprises a methionine at a position corresponding to position 148 according to SEQ ID NO:42. In some embodiments, the variant PNPLA3 protein in the sample comprises the amino acid sequence according to SEQ ID NO:42. In some embodiments, the variant PNPLA3 protein is detected by amino acid sequencing or by an immunoassay. In some embodiments, the subject has been determined to express the variant PNPLA3 protein by detection of a nucleic acid molecule encoding the variant PNPLA3 protein (e.g., a variant PNPLA3 nucleic acid molecule encoding a PNPLA3 protein comprising the I148M or 1144M variation) in a sample from the subject. In some embodiments, the variant PNPLA3 protein comprises a methionine at a position corresponding to position 148 according to SEQ ID NO:42. In some embodiments, the variant PNPLA3 protein comprises the amino acid sequence according to SEQ ID NO:42. In some embodiments, the nucleic acid molecule encoding the variant PNPLA3 protein comprises genomic DNA, mRNA, or cDNA obtained from mRNA. In some embodiments, the nucleic acid molecule comprises genomic DNA comprising an ATG codon at the positions corresponding to positions 5107 to 5109 according to SEQ ID NO:31. In some embodiments, the genomic DNA comprises the nucleotide sequence according to SEQ ID NO:31. In some embodiments, the nucleic acid molecule comprises mRNA comprising an AUG codon at the positions corresponding to positions 442 to 444 according to SEQ ID NO:34. In some embodiments, the mRNA comprises the nucleotide sequence according to SEQ ID NO:34. In some embodiments, the nucleic acid molecule comprises mRNA comprising an AUG codon at the positions corresponding to positions 430 to 432 according to SEQ ID NO:35. In some embodiments, the mRNA comprises the nucleotide sequence according to SEQ ID NO:35. In some embodiments, the nucleic acid molecule comprises cDNA obtained from mRNA, the cDNA comprising an ATG codon at the positions corresponding to positions 442 to 444 according to SEQ ID NO:38. In some embodiments, the cDNA comprises the nucleotide sequence according to SEQ ID NO:38. In some embodiments, the nucleic acid molecule comprises cDNA obtained from mRNA, the cDNA comprising an ATG codon at the positions corresponding to positions 430 to 432 according to SEQ ID NO:39. In some embodiments, the cDNA comprises the nucleotide sequence according to SEQ ID NO:39. In some embodiments, the nucleic acid is detected by sequencing at least a portion of the nucleic acid, the portion encoding the I148M variation. In some embodiments, the nucleic acid is detected by hybridization of a probe or primer that specifically hybridizes to a portion of the nucleic acid, wherein the portion comprises the codon encoding the I148M variation. In some embodiments, the probe or primer is an allele-specific probe or primer. In some embodiments, the probe or primer comprises a label. In some embodiments, the patient is homozygous for a gene encoding the variant PNPLA3 protein. In some embodiments, the patient is heterozygous for a gene encoding the variant PNPLA3 protein. In some embodiments, patient is homozygous for a gene encoding a functional HSD17B13 protein. In some embodiments, the patient is heterozygous for a gene encoding a functional HSD17B13 protein. In some embodiments, the patient is heterozygous for the gene encoding the functional HSD17B13 protein and a gene encoding a loss of function variant of HSD17B13.

Inhibitors of HSD17B13 can be used as described herein for treatment of a liver disease in a human subject having a PNPLA3 protein comprising an I148M variation and having a functional HSD17B13 protein. In some embodiments, the human subject has been tested positive for a PNPLA3 protein comprising an I148M variation and for a functional HSD17B13 protein. In some embodiments, the treatment comprises determining whether or not the human subject has a PNPLA3 protein comprising an I148M variation and a functional HSD17B13 protein. In some embodiments, the human subject has been identified as being a candidate for treating or inhibiting a liver disease by inhibiting HSD17B13 by using any of the method as defined herein. In some embodiments, the variant PNPLA3 protein comprises a methionine at the position corresponding to position 148 according to SEQ ID NO: 42. In some embodiments, the variant PNPLA3 protein comprises the amino acid sequence according to SEQ ID NO:42, or an amino acid sequence having at least 90% sequence identity to SEQ ID NO:42 and comprising the I148M variation. In some embodiments, the variant PNPLA3 protein comprises a methionine at the position corresponding to position 144 according to SEQ ID NO:43. In some embodiments, the variant PNPLA3 protein comprises the amino acid sequence according to SEQ ID NO:43, or an amino acid sequence having at least 90% sequence identity to SEQ ID NO:43 and comprising the 1144M variation. In some embodiments, the nucleic acid molecule encoding the variant PNPLA3 protein is genomic DNA. In some embodiments, the genomic DNA comprises an ATG codon at the positions corresponding to positions 5107 to 5109 according to SEQ ID NO:31. In some embodiments, the genomic DNA comprises the nucleotide sequence according to SEQ ID NO:31, or a nucleotide sequence having at least 90% sequence identity to SEQ ID NO:31 and encoding a PNPLA3 protein which comprises the I148M variation. In some embodiments, the nucleic acid molecule encoding the variant PNPLA3 protein is mRNA. In some embodiments, the mRNA comprises an AUG codon at the positions corresponding to positions 442 to 444 according to SEQ ID NO:34. In some embodiments, the mRNA comprises the nucleotide sequence according to SEQ ID NO:34, or a nucleotide sequence having at least 90% sequence identity to SEQ ID NO:34 and encoding a PNPLA3 protein which comprises the I148M variation. In some embodiments, the mRNA comprises an AUG codon at the positions corresponding to positions 430 to 432 according to SEQ ID NO:35. In some embodiments, the mRNA comprises the nucleotide sequence according to SEQ ID NO:35, or a nucleotide sequence having at least 90% sequence identity to SEQ ID NO:35 and encoding a PNPLA3 protein which comprises the 1144M variation. In some embodiments, the nucleic acid molecule encoding the variant PNPLA3 protein is cDNA. In some embodiments, the cDNA comprises an ATG codon at the positions corresponding to positions 442 to 444 according to SEQ ID NO:38. In some embodiments, the cDNA comprises the nucleotide sequence according to SEQ ID NO:38, or a nucleotide sequence having at least 90% sequence identity to SEQ ID NO:38 and encoding a PNPLA3 protein which comprises the I148M variation. In some embodiments, the cDNA comprises an ATG codon at the positions corresponding to positions 430 to 432 according to SEQ ID NO: 39. In some embodiments, the cDNA comprises the nucleotide sequence according to SEQ ID NO:39, or a nucleotide sequence having at least 90% sequence identity to SEQ ID NO: 39 and encoding a PNPLA3 protein which comprises the 1144M variation. In some embodiments, the liver disease is a chronic liver disease. In some embodiments, the chronic liver disease is nonalcoholic fatty liver disease (NAFLD), alcoholic liver disease (ALD), non-alcoholic steatohepatitis (NASH), cirrhosis, steatosis, or hepatocellular carcinoma. In some embodiments, the liver disease is an alcoholic liver disease. In some embodiments, the alcoholic liver disease comprises one or more of cirrhosis, steatosis, or hepatocellular carcinoma resulting from alcohol consumption. In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption. In some embodiments, the human subject is homozygous or heterozygous for functional HSD17B13.

In some embodiments, inhibitors of HSD17B13 reduce or inhibit HSD17B13 gene expression or the function of HSD17B13 protein. Inhibitors of HSD17B13 include, but are not limited to, naturally occurring and synthetic ligands, antagonists, agonists, antibodies, peptides, cyclic peptides, nucleic acids, functional polynucleotides, small organic molecules, and the like. Functional polynucleotides are nucleic acid molecules that have a specific function, such as binding a target molecule or catalyzing a specific reaction. Examples of functional polynucleotides include, but are not limited to, antisense molecules, aptamers, ribozymes, and triplex forming molecules. The functional polynucleotides can act as inhibitors of a specific activity possessed by a target molecule. Antisense molecules are designed to interact with a target nucleic acid molecule through either canonical or non-canonical base pairing. The interaction of the antisense molecule and the target molecule is designed to promote the destruction of the target molecule through, for example, RNase-H-mediated RNA-DNA hybrid degradation. Alternately, the antisense molecule is designed to interrupt a processing function that normally would take place on the target molecule, such as transcription or replication. Antisense molecules can be designed based on the sequence of the target molecule. Numerous methods for optimization of antisense efficiency by identifying the most accessible regions of the target molecule exist. Exemplary methods include, but are not limited to, in vitro selection experiments and DNA modification studies using DMS and DEPC. Antisense molecules generally bind the target molecule with a dissociation constant (kd) less than or equal to about 10-6, less than or equal to about 10-8, less than or equal to about 10-10, or less than or equal to about 10-12. A representative sample of methods and techniques which aid in the design and use of antisense molecules, and antisense molecules, can be found in the following non-limiting list of U.S. Patents and applications: U.S. Pat. Nos. 5,135,917; 5,294,533; 5,627,158; 5,641,754; 5,691,317; 5,780,607; 5,786,138; 5,849,903; 5,856,103; 5,919,772; 5,955,590; 5,990,088; 5,994,320; 5,998,602; 6,005,095; 6,007,995; 6,013,522; 6,017,898; 6,018,042; 6,025,198; 6,033,910; 6,040,296; 6,046,004; 6,046,319; 6,057,437; and U.S. Ser. No. 62/645,941 filed Mar. 21, 2018, each of which is incorporated herein by reference in its entirety. Examples of antisense molecules include, but are not limited to, antisense RNAs, small interfering RNAs (siRNAs), and short hairpin RNAs (shRNAs). For example, the antisense RNAs, siRNAs, or shRNAs can be designed to target a region unique of the HSD17B13 genomic DNA or mRNA. In some embodiments, the inhibitor of HSD17B13 is an antisense molecule. In some embodiments, the inhibitor of HSD17B13 is an shRNA molecule. In some embodiments, the inhibitor of HSD17B13 is an siRNA molecule.

In any of the methods described herein, administration of an inhibitor of HSD17B13 can result in the reduction or elimination of particular characteristics of liver disease. In some embodiments, the characteristics of liver disease include, but are not limited to inflammation and fibrosis.

The present disclosure also provides methods of treating a subject who is PNPLA3 Ile148Met positive (i.e., “PNPLA3 Ile148Met+”) or PNPLA3 Ile144Met positive (i.e., “PNPLA3 Ile144Met+”), comprising administering an inhibitor of HSD17B13 to the subject. The present disclosure also provides methods of treating or inhibiting liver disease comprising administering an inhibitor of HSD17B13 to a human liver disease patient expressing a PNPLA3 protein comprising an I148M variation such that liver disease is treated or inhibited in the patient.

The variant PNPLA3 Ile148Met positive or PNPLA3 Ile144Met positive subject can have any of the variant PNPLA3 proteins described herein. In some embodiments, the subject is also homozygous or heterozygous for functional HSD17B13. In some embodiments, the subject is homozygous for functional HSD17B13. In some embodiments, the subject is heterozygous for functional HSD17B13. In some embodiments, the subject is homozygous for the HSD17B13 loss-of-function variant. The subject can have any of the functional HSD17B13 proteins described herein.

In some embodiments, the subject who is PNPLA3 Ile148Met+ or PNPLA3 Ile144Met+ has been determined to be PNPLA3 Ile148Met+ or PNPLA3 Ile144Met+ by detection of a PNPLA3 protein in a sample from the subject, wherein the variant PNPLA3 protein comprises a methionine at a position corresponding to position 148 according to SEQ ID NO:42, or comprises a methionine at a position corresponding to position 144 according to SEQ ID NO:43. The variant PNPLA3 Ile148Met positive or PNPLA3 Ile144Met positive subject can have any of the variant PNPLA3 proteins described herein.

In some embodiments, the subject who is PNPLA3 Ile148Met+ has been determined to be PNPLA3 Ile148Met+ by detection of a PNPLA3 protein in a sample from the subject, wherein the variant PNPLA3 protein comprises a methionine at a position corresponding to position 148 according to SEQ ID NO:42. In some embodiments, the variant PNPLA3 protein in the sample comprises the amino acid sequence according to SEQ ID NO: 42.

In some embodiments, the subject who is PNPLA3 Ile144Met+ has been determined to be PNPLA3 Ile144Met+ by detection of a PNPLA3 protein in a sample from the subject, wherein the variant PNPLA3 protein comprises a methionine at a position corresponding to position 144 according to SEQ ID NO:43. In some embodiments, the variant PNPLA3 protein in the sample comprises the amino acid sequence according to SEQ ID NO: 43.

In some embodiments, the variant PNPLA3 protein that comprises the methionine at the position corresponding to position 148 according to SEQ ID NO:42, or that comprises the methionine at the position corresponding to position 144 according to SEQ ID NO:43 is identified by amino acid sequencing or immunoassay as described herein.

In some embodiments, the subject who is PNPLA3 Ile148Met+ or PNPLA3 Ile144Met+ has been determined to be PNPLA3 Ile148Met+ or PNPLA3 Ile144Met+ by detection of a nucleic acid molecule encoding a PNPLA3 protein in a sample from the subject, wherein the variant PNPLA3 protein comprises a methionine at a position corresponding to position 148 according to SEQ ID NO:42, or comprises a methionine at a position corresponding to position 144 according to SEQ ID NO:43. The variant PNPLA3 Ile148Met positive or PNPLA3 Ile144Met positive subject can have any of the variant PNPLA3 nucleic acid molecules described herein.

In some embodiments, the subject who is PNPLA3 Ile148Met+ has been determined to be PNPLA3 Ile148Met+ by detection of a nucleic acid molecule encoding a PNPLA3 protein in a sample from the subject, wherein the variant PNPLA3 protein comprises a methionine at a position corresponding to position 148 according to SEQ ID NO: 42. In some embodiments, the variant PNPLA3 protein in the sample comprises the amino acid sequence according to SEQ ID NO:42.

In some embodiments, the subject who is PNPLA3 Ile144Met+ has been determined to be PNPLA3 Ile144Met+ by detection of a nucleic acid molecule encoding PNPLA3 protein in a sample from the subject, wherein the variant PNPLA3 protein comprises a methionine at a position corresponding to position 144 according to SEQ ID NO: 43. In some embodiments, the variant PNPLA3 protein in the sample comprises the amino acid sequence according to SEQ ID NO:43.

In some embodiments, the nucleic acid molecule encoding the variant PNPLA3 protein is genomic DNA, mRNA, or cDNA derived from mRNA.

In some embodiments, the genomic DNA comprises an ATG codon at the positions corresponding to positions 5107 to 5109 according to SEQ ID NO:31. In some embodiments, the genomic DNA comprises the nucleotide sequence according to SEQ ID NO:31. In some embodiments, the genomic DNA encoding the variant PNPLA3 protein that comprises the ATG codon is identified by nucleic acid sequencing or hybridization of a probe, as described herein.

In some embodiments, the mRNA comprises an AUG codon at the positions corresponding to positions 442 to 444 according to SEQ ID NO:34. In some embodiments, the mRNA comprises the nucleotide sequence according to SEQ ID NO:34. In some embodiments, the mRNA comprises an AUG codon at the positions corresponding to positions 430 to 432 according to SEQ ID NO:35. In some embodiments, the mRNA comprises the nucleotide sequence according to SEQ ID NO:35. In some embodiments, the mRNA encoding the variant PNPLA3 protein that comprises the AUG codon is identified by nucleic acid sequencing or hybridization of a probe, as described herein.

In some embodiments, the cDNA comprises an ATG codon at the positions corresponding to positions 442 to 444 according to SEQ ID NO:38. In some embodiments, the cDNA comprises the nucleotide sequence according to SEQ ID NO:38. In some embodiments, the cDNA comprises an ATG codon at the positions corresponding to positions 430 to 432 according to SEQ ID NO:39. In some embodiments, the cDNA comprises the nucleotide sequence according to SEQ ID NO:39. In some embodiments, the cDNA encoding the variant PNPLA3 protein that comprises the ATG codon is identified by nucleic acid sequencing or hybridization of a probe, as described herein.

Administration of the inhibitor of HSD17B13 can be by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

In some embodiments, the subject has a liver disease or is susceptible to developing a liver disease. In some embodiments, the liver disease is a chronic liver disease. In some embodiments, the chronic liver disease is nonalcoholic fatty liver disease (NAFLD), alcoholic liver disease (ALD), non-alcoholic steatohepatitis (NASH), cirrhosis, steatosis, or hepatocellular carcinoma. In some embodiments, the liver disease is an alcoholic liver disease. In some embodiments, the alcoholic liver disease comprises one or more of cirrhosis, steatosis, or hepatocellular carcinoma resulting from alcohol consumption. In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption.

The present disclosure also provides methods for treating a patient with a liver disease therapeutic agent, wherein the patient is suffering from a liver disease. The methods comprise determining whether or not a sample from the subject comprises: i) a first nucleic acid encoding a PNPLA3 protein comprising an I148M variation and a second nucleic acid encoding a functional HSD17B13 protein, and/or ii) a PNPLA3 protein comprising an I148M variation and a functional HSD17B13 protein. In some embodiments, the methods comprise determining whether or not a sample from the subject comprises a first nucleic acid encoding a PNPLA3 protein comprising an I148M variation and a second nucleic acid encoding a functional HSD17B13 protein. In some embodiments, the methods comprise determining whether or not a sample from the subject comprises a PNPLA3 protein comprising an I148M variation and a functional HSD17B13 protein.

In some embodiments, this determination is carried out by obtaining or having obtained a biological sample from the patient. In some embodiments, the methods further comprise performing or having performed a genotyping assay on the biological sample to determine if the patient has a first nucleic acid encoding a PNPLA3 protein comprising an I148M variation and a second nucleic acid encoding a functional HSD17B13 protein. In some embodiments, the methods further comprise performing or having performed an assay on the biological sample to determine if the patient has a PNPLA3 protein comprising an I148M variation and a functional HSD17B13 protein.

In some embodiments, when the patient has a nucleic acid encoding a PNPLA3 protein comprising an I148M variation and/or has a PNPLA3 protein comprising an I148M variation, and has a nucleic acid encoding a functional HSD17B13 protein and/or has a functional HSD17B13 protein, then the method further comprises administering an inhibitor of HSD17B13 to the patient. In some embodiments, when the patient has a nucleic acid encoding a PNPLA3 protein comprising an I148M variation and/or has a PNPLA3 protein comprising an I148M variation, and has a nucleic acid encoding a functional HSD17B13 protein and/or has a functional HSD17B13 protein, then the method further comprises administering an inhibitor of HSD17B13 to the patient and administering a liver disease therapeutic agent to the patient. In some embodiments, when the patient has a nucleic acid encoding a PNPLA3 protein comprising an I148M variation and/or has a PNPLA3 protein comprising an I148M variation, but does not have a nucleic acid encoding a functional HSD17B13 protein and/or has a functional HSD17B13 protein, then the method further comprises administering a liver disease therapeutic agent to the patient.

Examples of liver disease therapeutic agents include, but are not limited to, Disulfiram, Naltrexone, Acamprosate, Prednisone, Prednisone, Azathioprine, Penicillamine, Trientine, Deferoxamine, Ciprofloxacin, Norofloxacin, Ceftriaxone, Ofloxacin, Amoxicillin-clavulanate, Phytonadione, Bumetanide, Furosemide, Hydrochlorothiazide, Chlorothiazide, Amiloride, Triamterene, Spironolactone, Octreotide, Atenolol, Metoprolol, Nadolol, Propranolol, Timolol, and Carvedilol.

Additional examples of liver disease therapeutic agents (e.g., for use in chronic hepatitis C treatment) include, but are not limited to, ribavirin, paritaprevir, simeprevir (Olysio), grazoprevir, ledipasvir, ombitasvir, elbasvir, daclatasvir (Daklinza), dasabuvir, ritonavir, sofosbuvir, velpatasvir, voxilaprevir, glecaprevir, pibrentasvir, peginterferon alfa-2a, peginterferon alfa-2b, and interferon alfa-2b.

Additional examples of liver disease therapeutic agents (e.g., for use in nonalcoholic fatty liver disease) include, but are not limited to, weight loss inducing agents such as orlistat or sibutramine; insulin sensitizing agents such as thiazolidinediones (TZDs), metformin, and meglitinides; lipid lowering agents such as statins, fibrates, and omega-3 fatty acids; antioxidants such as, vitamin E, betaine, N-Acetyl-cysteine, lecithin, silymarin, and beta-carotene; anti TNF agents such as pentoxifylline; probiotics, such as VSL #3; and cytoprotective agents such as ursodeoxycholic acid (UDCA). Other suitable treatments include ACE inhibitors/ARBs, oligofructose, and Incretin analogs.

Additional examples of liver disease therapeutic agents (e.g., for use in NASH) include, but are not limited to, obeticholic acid (Ocaliva®), Selonsertib, Elafibranor, Cenicriviroc, GR_MD_02, MGL_3196, IMM124E, arachidyl amido cholanoic acid (Aramchol™), GS0976, Emricasan, Volixibat, NGM282, GS9674, Tropifexor, MN_001, LMB763, BI_1467335, MSDC_0602, PF_05221304, DF102, Saroglitazar, BMS986036, Lanifibranor, Semaglutide, Nitazoxanide, GRI_0621, EYP001, VK2809, Nalmefene, LIK066, MT_3995, Elobixibat, Namodenoson, Foralumab, SAR425899, Sotagliflozin, EDP_305, Isosabutate, Gemcabene, TERN_101, KBP_042, PF_06865571, DUR928, PF_06835919, NGM313, BMS_986171, Namacizumab, CER_209, ND_L02_s0201, RTU_1096, DRX_065, IONIS_DGAT2Rx, INT_767, NC_001, Seladepar, PXL770, TERN_201, NV556, AZD2693, SP_1373, VK0214, Hepastem, TGFTX4, RLBN1127, GKT_137831, RYI_018, CB4209-CB4211, and JH_0920.

The present disclosure also provides inhibitors of HSD17B13 for use in the manufacture of a medicament for the treatment of liver disease in a human subject who is PNPLA3 Ile148Met positive or PNPLA3 Ile144Met positive and who is also homozygous or heterozygous for functional HSD17B13. In some embodiments, the subject is homozygous for PNPLA3 Ile148Met variant or PNPLA3 Ile144Met variant. In some embodiments, the subject is heterozygous for PNPLA3 Ile148Met variant or PNPLA3 Ile144Met variant. In some embodiments, the subject is homozygous for functional HSD17B13. In some embodiments, the subject is heterozygous for functional HSD17B13.

In some embodiments, the inhibitor of HSD17B13 is for use in the treatment of a liver disease in a human subject having a PNPLA3 protein comprising a methionine at a position corresponding to position 148 according to SEQ ID NO:42, or a nucleic acid molecule encoding a PNPLA3 protein comprising a methionine at a position corresponding to position 148 according to SEQ ID NO:42, or comprising a methionine at a position corresponding to position 144 according to SEQ ID NO:43, or a nucleic acid molecule encoding a PNPLA3 protein comprising a methionine at a position corresponding to position 148 according to SEQ ID NO:43. In some embodiments, the variant PNPLA3 protein comprises a methionine at a position corresponding to position 148 according to SEQ ID NO: 42. In some embodiments, the variant PNPLA3 protein comprises the amino acid sequence according to SEQ ID NO:42. In some embodiments, the variant PNPLA3 protein comprises a methionine at a position corresponding to position 144 according to SEQ ID NO: 43. In some embodiments, the variant PNPLA3 protein comprises the amino acid sequence according to SEQ ID NO:43.

In some embodiments, the nucleic acid molecule encoding the variant PNPLA3 protein is genomic DNA. In some embodiments, the genomic DNA comprises an ATG codon at the positions corresponding to positions 5107 to 5109 according to SEQ ID NO:31. In some embodiments, the genomic DNA comprises the nucleotide sequence according to SEQ ID NO: 31.

In some embodiments, the nucleic acid molecule encoding the variant PNPLA3 protein is mRNA. In some embodiments, the mRNA comprises an AUG codon at the positions corresponding to positions 442 to 444 according to SEQ ID NO:34. In some embodiments, the mRNA comprises the nucleotide sequence according to SEQ ID NO:34. In some embodiments, the mRNA comprises an AUG codon at the positions corresponding to positions 430 to 432 according to SEQ ID NO:35. In some embodiments, the mRNA comprises the nucleotide sequence according to SEQ ID NO:35.

In some embodiments, nucleic acid molecule encoding the variant PNPLA3 protein is cDNA. In some embodiments, the cDNA comprises an ATG codon at the positions corresponding to positions 442 to 444 according to SEQ ID NO:38. In some embodiments, the cDNA comprises the nucleotide sequence according to SEQ ID NO:38. In some embodiments, the cDNA comprises an ATG codon at the positions corresponding to positions 430 to 432 according to SEQ ID NO:39. In some embodiments, the cDNA comprises the nucleotide sequence according to SEQ ID NO:39.

In some embodiments, the liver disease is a chronic liver disease. In some embodiments, the chronic liver disease is nonalcoholic fatty liver disease (NAFLD), alcoholic liver disease (ALD), non-alcoholic steatohepatitis (NASH), cirrhosis, steatosis, or hepatocellular carcinoma. In some embodiments, the liver disease is an alcoholic liver disease. In some embodiments, the alcoholic liver disease comprises one or more of cirrhosis, steatosis, or hepatocellular carcinoma resulting from alcohol consumption. In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption.

In some embodiments, the human subject is homozygous or heterozygous for functional HSD17B13. In some embodiments, the subject is homozygous for functional HSD17B13. In some embodiments, the subject is heterozygous for functional HSD17B13.

In any of the methods described herein, a probe or primer or an alteration-specific probe or an alteration-specific primer can be specifically complementary to or specifically hybridize with a single nucleic acid species. For example, a probe or primer or an alteration-specific probe or an alteration-specific primer specifically complementary to or specifically hybridizing with a nucleic acid molecule for HSD17B13 transcript A, transcript B, transcript E, or transcript I (e.g., any of the mRNA, cDNA, RNA transcript, or cDNA transcript for functional HSD17B13 described herein) is not complementary to or does not hybridize with any of the nucleic acid molecules for a variant HSD17B13 (e.g., any of the mRNA, cDNA, RNA transcripts, or cDNA transcripts for variants C, D, F, G, H of HSD17B13).

The present disclosure also provides an inhibitor of HSD17B13 for use in the treatment of a liver disease in a human subject having a PNPLA3 protein comprising an I148M variation and having a functional HSD17B13 protein. In some embodiments, the human subject has been tested positive for a PNPLA3 protein comprising an I148M variation and for a functional HSD17B13 protein. In some embodiments, the treatment comprises determining whether or not the human subject has a PNPLA3 protein comprising an I148M variation and a functional HSD17B13 protein. In some embodiments, the human subject has been identified as being a candidate for treating or inhibiting a liver disease by inhibiting HSD17B13 by using any of the methods as defined herein.

In some embodiments, the variant PNPLA3 protein comprises a methionine at the position corresponding to position 148 according to SEQ ID NO:42. In some embodiments, the variant PNPLA3 protein comprises the amino acid sequence according to SEQ ID NO:42, or an amino acid sequence having at least 90% sequence identity to SEQ ID NO:42 and comprising the I148M variation. In some embodiments, the nucleic acid molecule encoding the variant PNPLA3 protein is genomic DNA. In some embodiments, the genomic DNA comprises an ATG codon at the positions corresponding to positions 5107 to 5109 according to SEQ ID NO:31. In some embodiments, the genomic DNA comprises the nucleotide sequence according to SEQ ID NO:31, or a nucleotide sequence having at least 90% sequence identity to SEQ ID NO:31 and encoding a PNPLA3 protein which comprises the I148M variation. In some embodiments, the nucleic acid molecule encoding the variant PNPLA3 protein is mRNA. In some embodiments, the mRNA comprises an AUG codon at the positions corresponding to positions 442 to 444 according to SEQ ID NO:34. In some embodiments, the mRNA comprises the nucleotide sequence according to SEQ ID NO:34, or a nucleotide sequence having at least 90% sequence identity to SEQ ID NO:34 and encoding a PNPLA3 protein which comprises the I148M variation. In some embodiments, the mRNA comprises an AUG codon at the positions corresponding to positions 430 to 432 according to SEQ ID NO:35. In some embodiments, the mRNA comprises the nucleotide sequence according to SEQ ID NO:35, or a nucleotide sequence having at least 90% sequence identity to SEQ ID NO:35 and encoding a PNPLA3 protein which comprises the I148M variation. In some embodiments, the nucleic acid molecule encoding the variant PNPLA3 protein is cDNA. In some embodiments, the cDNA comprises an ATG codon at the positions corresponding to positions 442 to 444 according to SEQ ID NO:38. In some embodiments, the cDNA comprises the nucleotide sequence according to SEQ ID NO:38, or a nucleotide sequence having at least 90% sequence identity to SEQ ID NO:38 and encoding a PNPLA3 protein which comprises the I148M variation. In some embodiments, the cDNA comprises an ATG codon at the positions corresponding to positions 430 to 432 according to SEQ ID NO: 39. In some embodiments, the cDNA comprises the nucleotide sequence according to SEQ ID NO:39, or a nucleotide sequence having at least 90% sequence identity to SEQ ID NO: 39 and encoding a PNPLA3 protein which comprises the I148M variation.

In some embodiments, the liver disease is a chronic liver disease. In some embodiments, the chronic liver disease is nonalcoholic fatty liver disease (NAFLD), alcoholic liver disease (ALD), non-alcoholic steatohepatitis (NASH), cirrhosis, steatosis, or hepatocellular carcinoma. In some embodiments, the liver disease is an alcoholic liver disease. In some embodiments, the alcoholic liver disease comprises one or more of cirrhosis, steatosis, or hepatocellular carcinoma resulting from alcohol consumption. In some embodiments, the liver disease is a non-alcoholic liver disease. In some embodiments, the non-alcoholic liver disease comprises nonalcoholic fatty liver disease (NAFLD) or non-alcoholic steatohepatitis (NASH). In some embodiments, the non-alcoholic liver disease comprises one or more of cirrhosis, steatosis, or hepatocellular carcinoma not caused by alcohol consumption.

In some embodiments, the human subject is homozygous or heterozygous for functional HSD17B13.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The nucleotide and amino acid sequences recited herein are shown using standard letter abbreviations for nucleotide bases, and one-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

The following examples are provided to describe the embodiments in greater detail. They are intended to illustrate, not to limit, the claimed embodiments.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of what the inventors regard as their subject matter. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Genetic Interaction Between PNPLA3 rs738409 (p.I148M) and HSD17B13 Rs72613567—Study Design

In this study, exome sequencing was used to identify variants associated with serum alanine aminotransferase (ALT) and aspartate aminotransferase (AST) levels, which are markers of hepatocyte injury, in the DiscovEHR human genetics study, a cohort that links exome sequence data to electronic health records (EHR), and in three additional studies. The associations between implicated genetic variants and clinical diagnoses of chronic liver disease in DiscovEHR and two independent cohorts was also studied. The association between one of these variants and the histopathological severity of liver disease in an independent cohort of bariatric surgery patients who underwent liver biopsy was also studied.

### Study Design and Participants

Human genetics studies were conducted using genomic DNA samples and data from six cohorts. These studies included two Regeneron Genetics Center and the Geisinger Health System (GHS) DiscovEHR study populations originating from the first 50,726 adult consented participants from the MyCode® Community Health Initiative of GHS20. The GHS discovery cohort consisted of 46,544 European individuals recruited from outpatient primary care and specialty clinics between 2007 and 2016, excluding all those recruited to the bariatric surgery cohort. The GHS bariatric surgery cohort consisted of 2,644 European individuals who had been referred for bariatric surgery. Replication studies of associations with liver transaminases were performed in the Dallas Heart Study and the Penn Medicine Biobank, which included 1,357 and 8,527 individuals of European ancestry, respectively. Replication studies of the associations with chronic liver disease included 517 individuals from the Dallas Liver Study (DLS) and 439 individuals from the Dallas Pediatric Liver Study (DPLS). Full study descriptions and clinical phenotype and disease definitions are described the Methods section in the Supplementary Appendix.

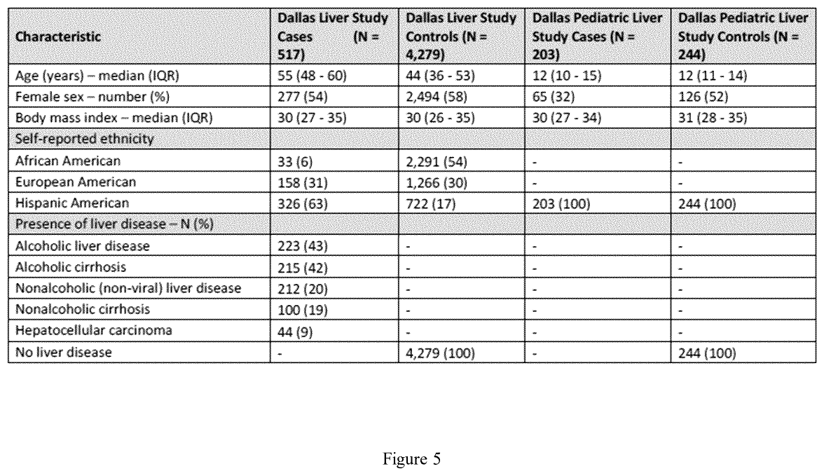

Baseline characteristics of genotyped multi-ethnic cases and controls from the Dallas Liver and Pediatric Liver Studies are shown in FIG. 5.

### Sample Preparation, Sequencing, and Genotyping

DNA sample preparation and whole exome sequencing for the participants in the DiscovEHR study, the Dallas Heart Study, and the Penn Medicine Biobank were performed at the Regeneron Genetics as previously described (Dewey et al., Science, 2016, In Press). HSD17B13 rs72613567 was genotyped by Taqman assay (and verified by Sanger sequencing in 5 individuals of each genotype) in the Dallas Liver Study and Dallas Pediatric Liver Study.

### Clinical Measurements and Chronic Liver Disease Definitions in the Discovery Cohort

Clinical laboratory measurements for alanine aminotransferase (ALT) and aspartate aminotransferase (AST) were extracted from EHRs of participants from the GHS discovery cohort and bariatric surgery cohort. Median ALT and AST values were calculated for all participants with two or more measurements, and were log 10-transformed to normalize the distribution prior to association analyses.

International Classification of Diseases, Ninth Revision (ICD-9) disease diagnosis codes were extracted from EHRs and collapsed into clinical disease categories for non-viral, nonalcoholic (ICD-9 571.40, 571.41, 571.49, 571.5, 571.8, 571.9) or alcoholic (ICD-9 571.0, 571.1, 571.2, 571.3) liver disease case definitions. Additional case definitions based on single diagnosis codes included: alcoholic cirrhosis (ICD-9 571.2), nonalcoholic cirrhosis (ICD-9 571.5), and HCC (ICD-9 155.0). For these case definitions, a common control group without liver disease (“no liver disease”) was defined as participants with no case criteria or single-encounter or problem-list diagnosis code indicating any type of liver disease.

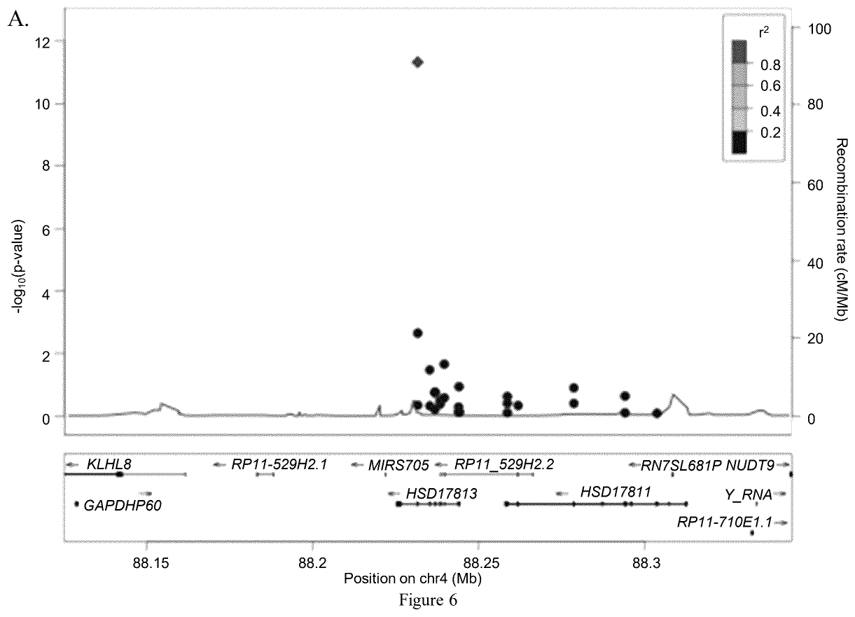

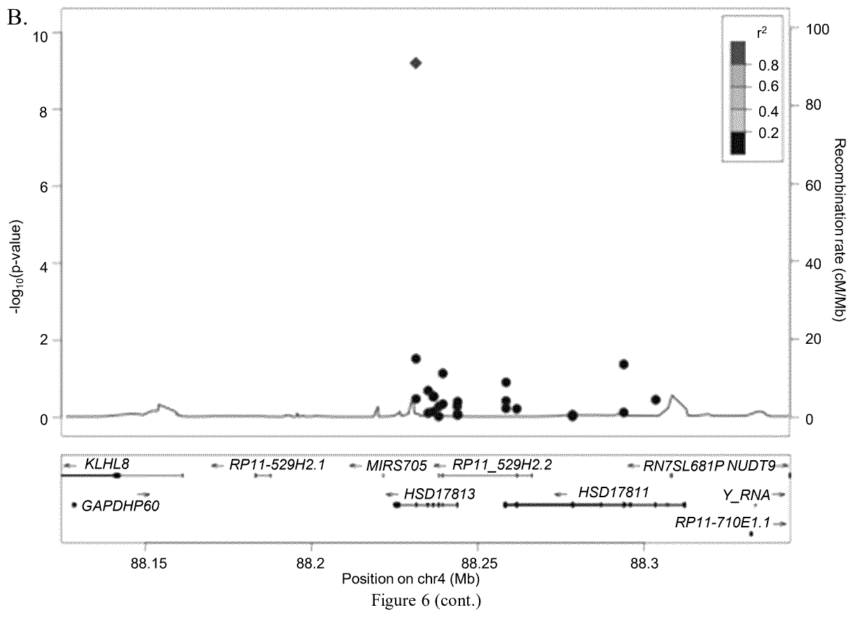

Regional association plots for alanine aminotransferase (ALT; A) and aspartate aminotransferase (AST; B) levels in the GHS discovery cohort in the region around HSD17B13 are shown in FIG. 6 (panels A and B). Purple diamonds indicate the splice variant rs72613567. Each circle indicates a single nucleotide variant with the color of the circle indicating the linkage disequilibrium (r2 calculated in the DiscovEHR cohort) between that variant and rs72613567. Blue lines indicate estimated recombination rates in HapMap. The bottom portion of the panels show the relative position and the transcribed strand of each gene in the locus. There were no significant associations between AST or ALT and coding or splice region variants in the neighboring gene HSD17B11 (most significant P-values 1.4×10⁻¹ and 4.3×10⁻² for ALT and AST, respectively).

### Liver Histopathologic Phenotype Definitions in the Bariatric Surgery Cohort

The GHS bariatric surgery cohort consisted of 2,644 individuals of European descent, with intra-operative liver biopsy specimens available from 2,391 of these individuals. Liver biopsy specimens were formalin-fixed and stained with hematoxylin and eosin for histology, and Masson's trichrome stain for assessment of fibrosis, as previously described (Gerhard et al., Patient Saf. Surg., 2011, 5, 1). Histologic diagnoses were determined by hepatopathologists using previously established criteria (Brunt et al., Am. J. Gastroenterol., 1999, 94, 2467-74). Histologic diagnoses were used to defined the following phenotypes: 1) Normal: no evidence of steatosis, NASH, or fibrosis; 2) Simple steatosis: Steatosis (regardless of grade) with no evidence of NASH or fibrosis; 3) NASH: Any presence of lobular inflammation or hepatocyte ballooning (regardless of grade), or any presence of fibrosis (regardless of stage).

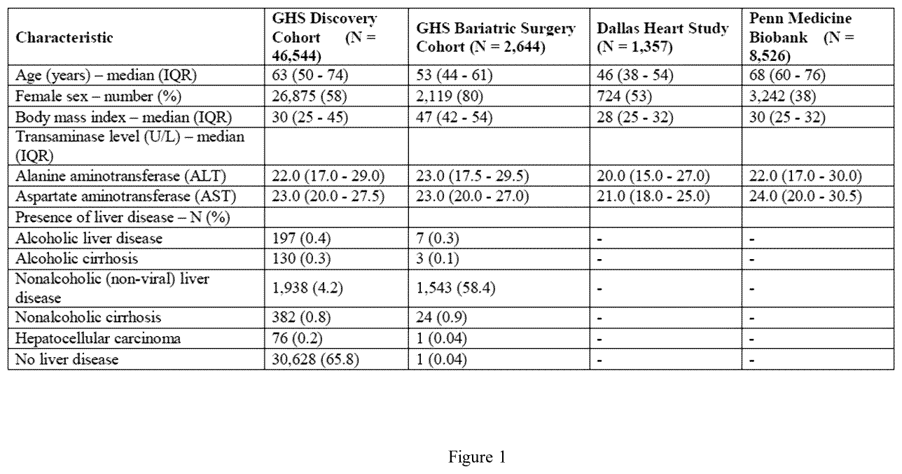

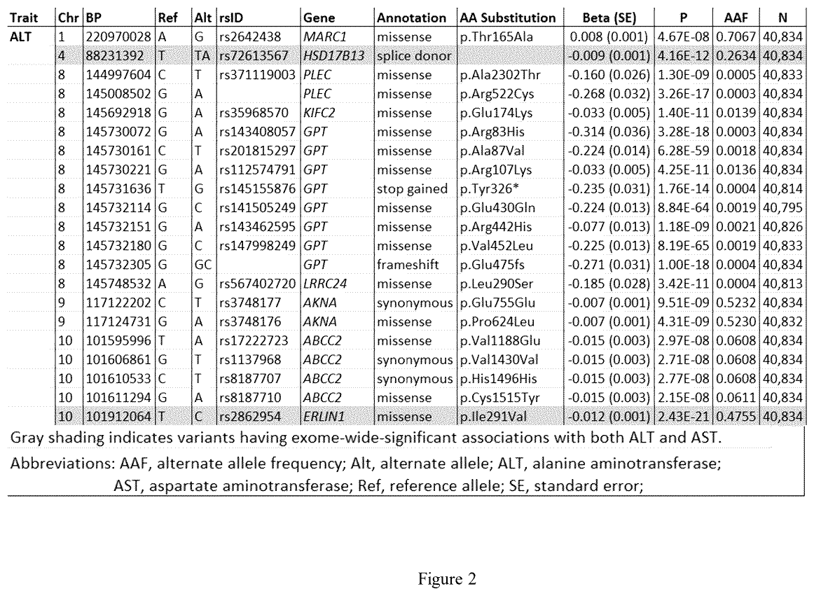

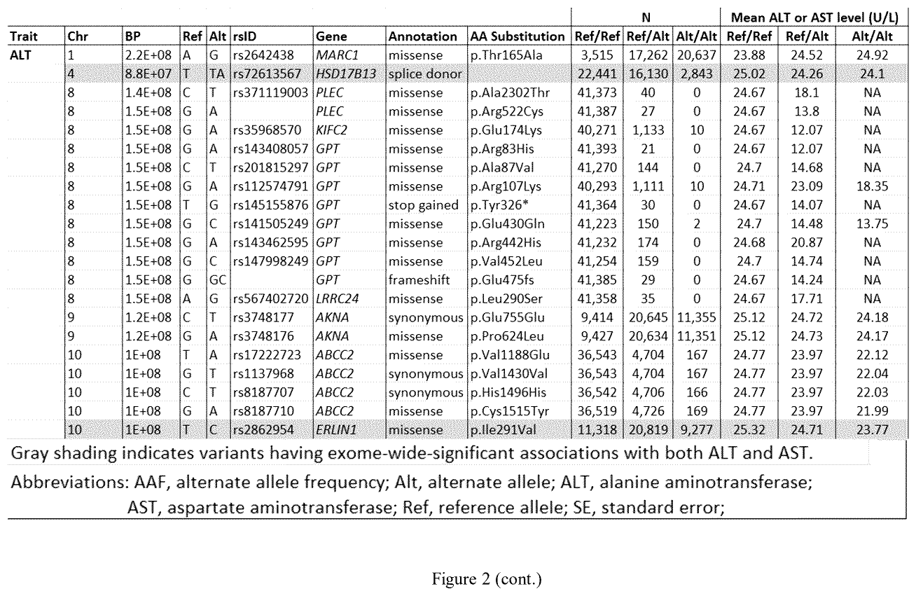

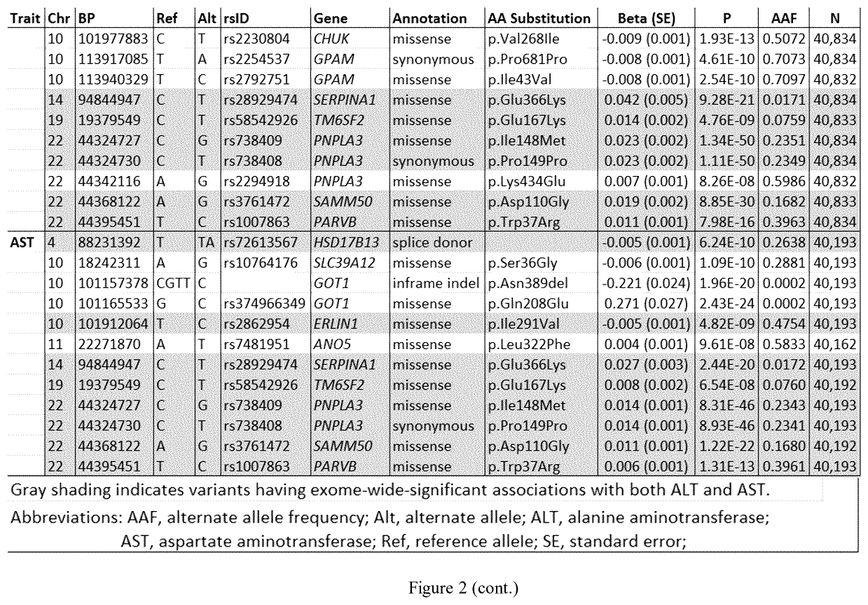

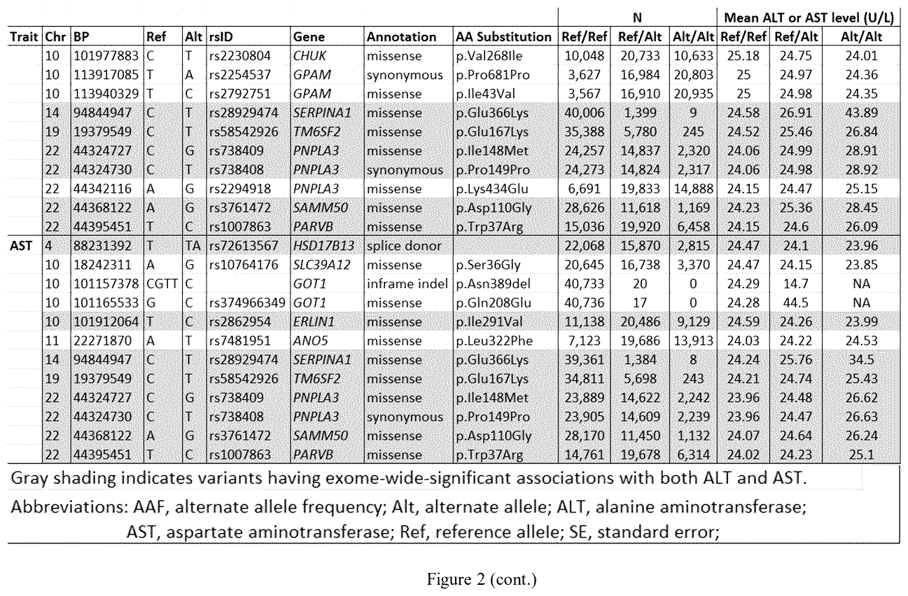

Baseline characteristics of sequenced European-ancestry individuals from the discovery and replication cohorts are shown in FIG. 1. Single nucleotide variants associated with serum transaminase levels at P<1.0×10⁻⁷ in the discovery cohort are shown in FIG. 2.

### DNA Sample Preparation and Sequencing

In brief, exome capture was performed using NimbleGen probes according to the manufacturer's recommended protocol (Roche NimbleGen). The captured DNA was PCR amplified and quantified by qRT-PCR (Kapa Biosystems). The multiplexed samples were sequenced using 75 bp paired-end sequencing on an Illumina v4 HiSeq 2500 to a coverage depth sufficient to provide greater than 20× haploid read depth of over 85% of targeted bases in 96% of samples (approximately 80× mean haploid read depth of targeted bases). Raw sequence data from each Illumina Hiseq 2500 run were uploaded to the DNAnexus platform (Reid et al., BMC Bioinformatics, 2014, 15, 30) for sequence read alignment and variant identification. In brief, raw sequence data were converted from BCL files to sample-specific FASTQ-files, which were aligned to the human reference build GRCh37.p13 with BWA-mem (Li et al., Bioinformatics, 2009, 25, 1754-60). Single nucleotide variants (SNV) and insertion/deletion (indel) sequence variants were identified using the Genome Analysis Toolkit (Mckenna et al., Genome Res., 2010, 20, 1297-303).

### Exome-wide Association Analysis of Liver Enzymes and Chronic Liver Disease Phenotypes

502,219 biallelic variants with missing data rate<1%, Hardy-Weinberg equilibrium P-value>1.0×10⁻⁶, and minor allele frequency>0.1%, were examined for association with transaminase levels. Log₁₀-transformed median ALT and AST were adjusted for age, age², sex, BMI, and the first four principal components of ancestry. To account for relatedness among study participants, a genetic relatedness matrix was fit as a random-effects covariate. Both principal components and the genetic relatedness matrix were constructed from 39,858 non-MHC markers in approximate linkage equilibrium and with minor allele frequency>0.1%. A linear mixed models was used as implemented in the GCTA package (Yang et al., Am. J. Hum. Genet., 2011, 88, 76-82) to test for association between trait residuals and single nucleotide variants. All P-values reported in the text correspond to the allelic model.

Replication of associations in the GHS discovery cohort was attempted in three separate European-ancestry cohorts: the GHS bariatric surgery cohort, the Dallas Heart Study, and the Penn Medicine Biobank (described above). ALT and AST measures from the GHS bariatric surgery cohort and from Penn Medicine Biobank were log 10-transformed and adjusted for age, age², sex, BMI, and the first four principal components of ancestry. Genetic relatedness matrices were included as random-effects covariates, and analysis was performed using linear mixed models in GCTA. In the Dallas Heart study, log 10-transformed ALT and AST measures were adjusted for age, age², sex, BMI, and the first ten principal components of ancestry, and analysis was performed using linear regression implemented in PLINK. Summary statistics for the three replication cohorts were meta-analyzed using METAL (Willer et al., Bioinformatics, 2010, 26, 2190-1) (replication meta-analysis). Summary statistics for the discovery cohort and the three replication cohorts were meta-analyzed similarly (joint meta-analysis).

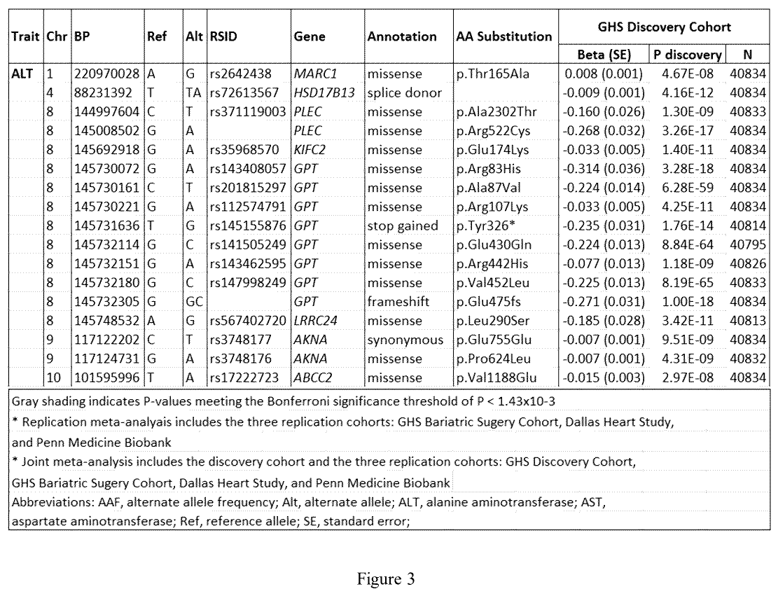

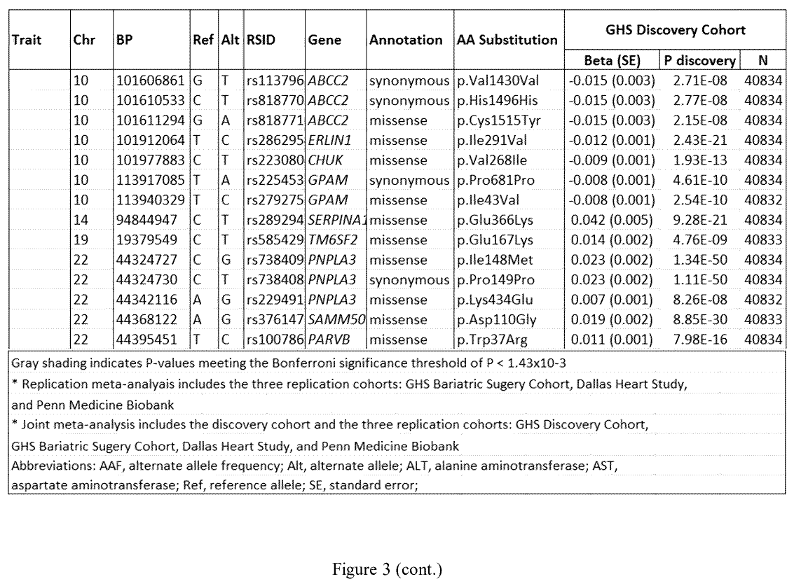

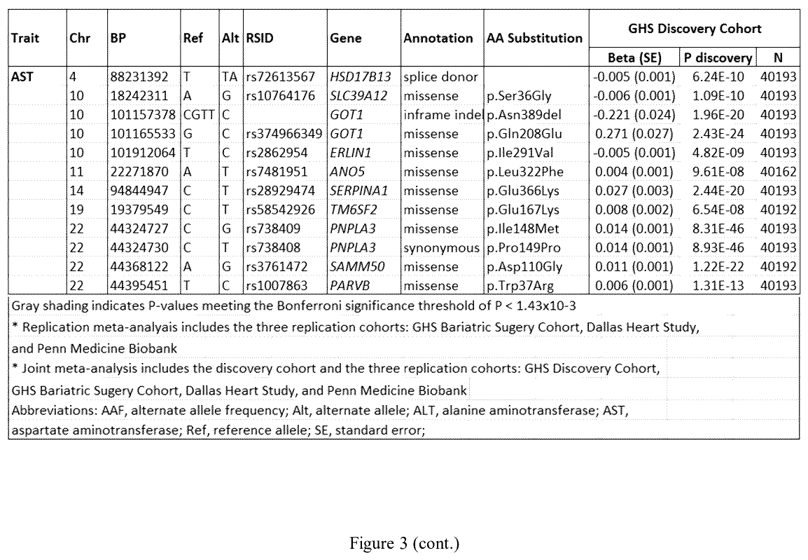

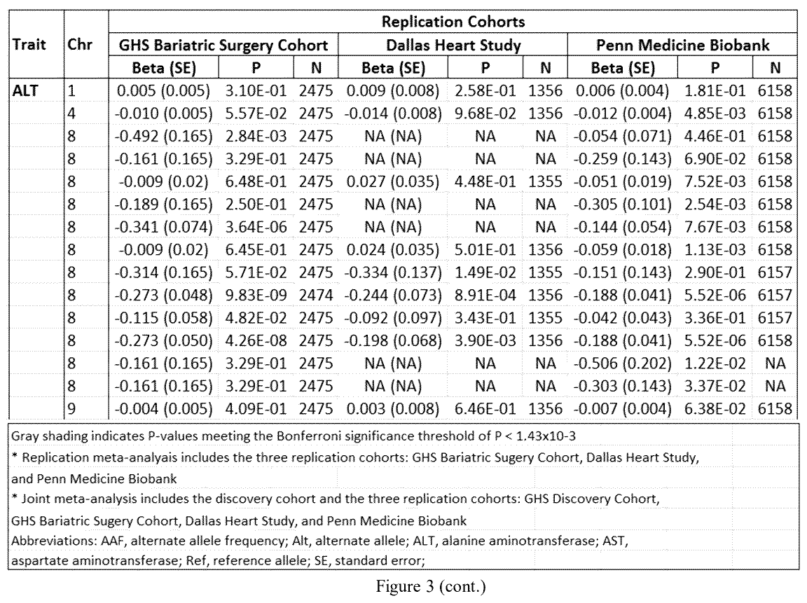

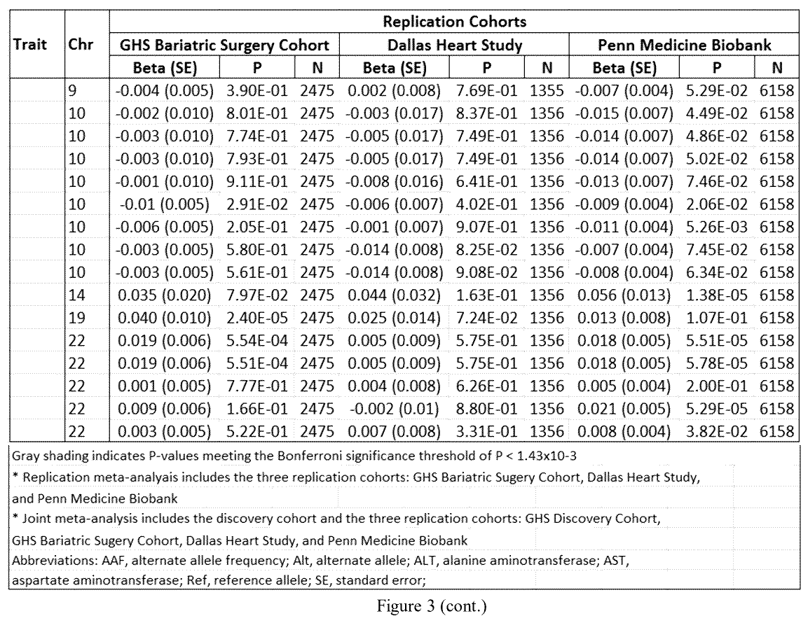

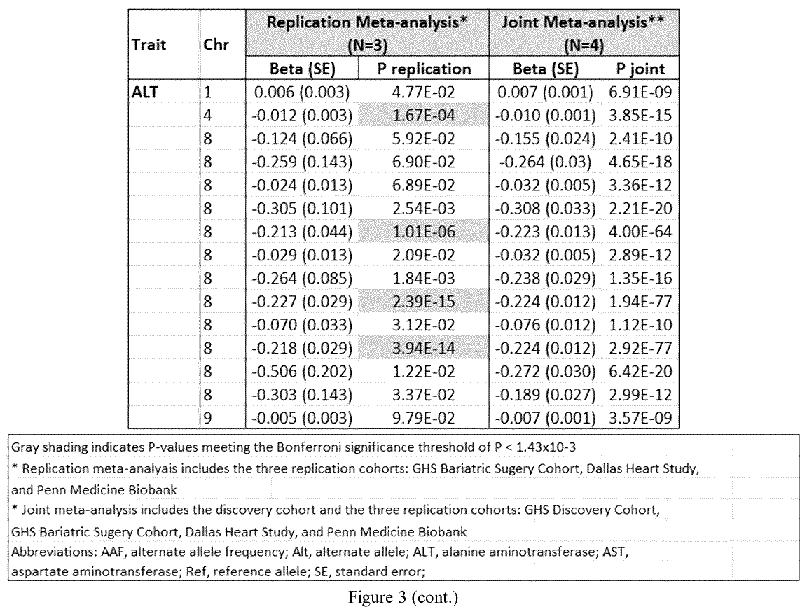

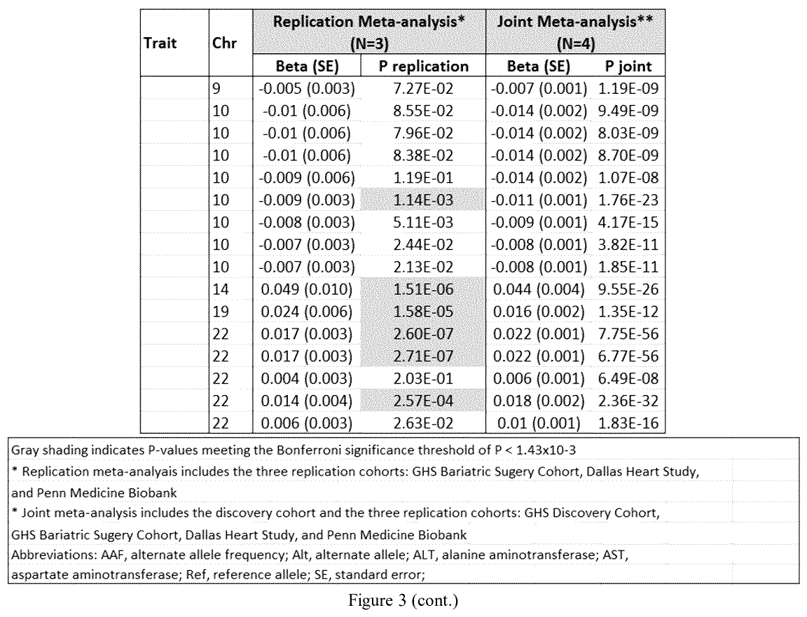

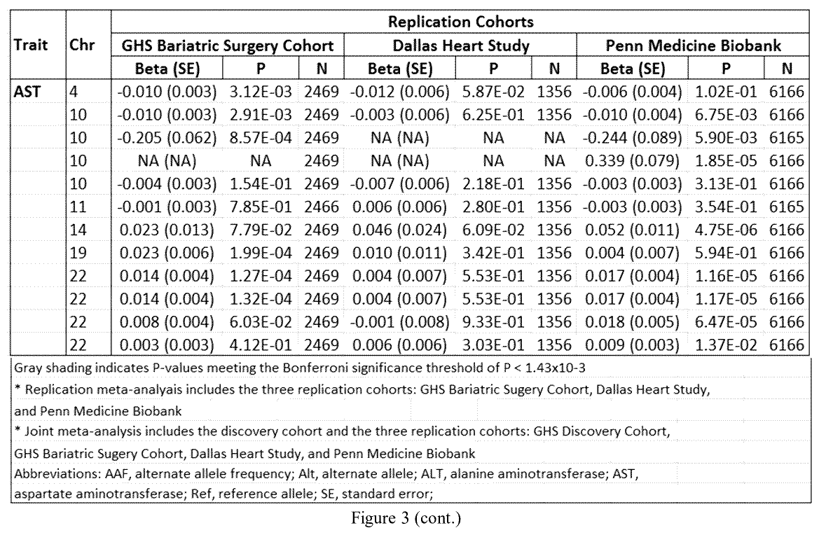

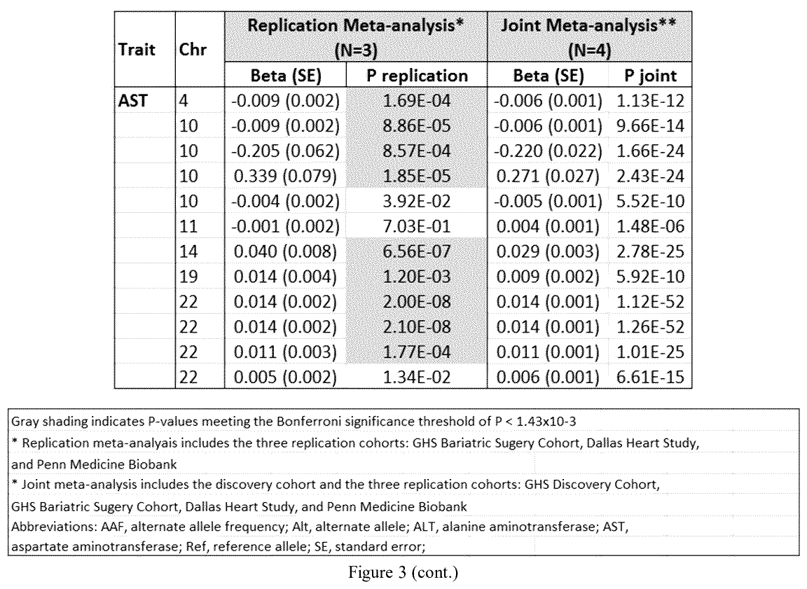

Replication and joint meta-analysis of 35 exome-wide significant single nucleotide variants from the discovery cohort in three separate European-ancestry cohorts is shown in FIG. 3.

For variants with exome wide significant associations with transaminases (p<1×10⁻⁷) in the GHS discovery cohort, association analyses and meta-analysis were performed, as described herein, in the European-ancestry replication studies described herein. A Bonferroni significance threshold determined by the number of variants tested was used to define replicated associations. Meta-analysis of discovery and replication studies was also performed. All P-values reported in the text correspond to the allelic model.

Transaminase-associated single nucleotide variants was also examined for associations with chronic liver disease phenotypes (defined and analyzed as described herein). A Bonferroni significance threshold determined by the number of variants and broad chronic liver disease categories tested was used to determine significance of associations. Replicated novel variants were also examined for association with histopathologically defined liver phenotypes from the GHS bariatric surgery cohort.

Association Analysis with Chronic Liver Disease Phenotypes

Thirteen significant and replicated single nucleotide variants from the liver enzyme ExWAS were analyzed for associations with chronic liver disease phenotypes defined from the GHS discovery cohort, as described above. A Bonferroni significance threshold of P<0.05/26 (P<1.92×10⁻³) was used to account for the thirteen variants and two broad chronic liver disease categories (alcoholic and nonalcoholic) tested. The HSD17B13 rs72613567 variant was further tested for association with histopathologically defined liver phenotypes from the GHS bariatric surgery cohort, as described above. Odds ratios were estimated with the use of Firth's penalized likelihood method of logistic regression after adjustment for age, age², sex, BMI, and the first four principal components of ancestry. Genotypic odds ratios were estimated for HSD17B13 rs72613567 using the same covariates.

Odds ratios for liver disease in the DLS were estimated by logistic regression, adjusted for age, age², sex, BMI, and self-reported ethnicity. Participants from the Dallas Heart Study with available rs72613567 genotypes were used as normal controls (n=4,279). Odds ratios in the DPLS were estimated by logistic regression.

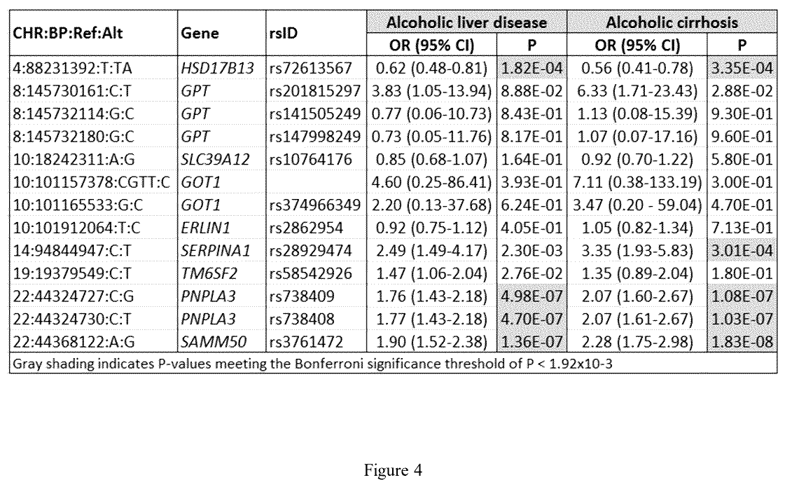

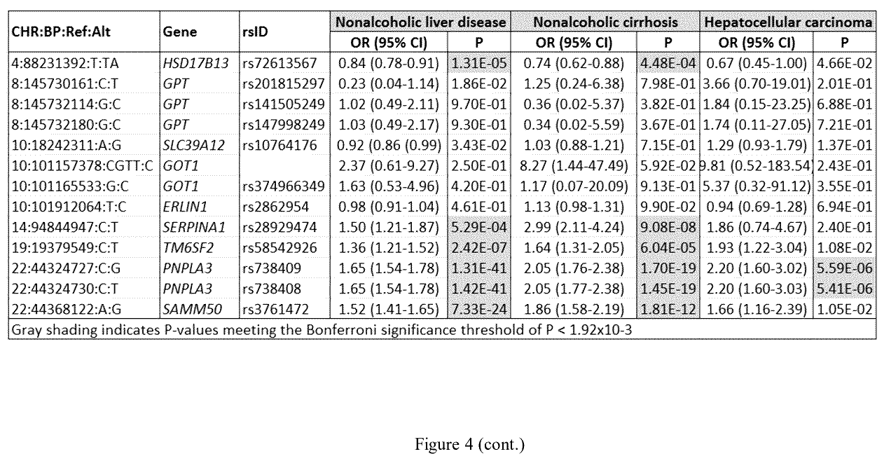

Association of thirteen exome-wide significant and replicating single nucleotide variants with liver disease phenotypes in the discovery cohort is shown in FIG. 4.

Genetic Interaction Between PNPLA3 rs738409 (p.I148M) And HSD17B13 rs72613567—Analysis

To evaluate the combined effect of PNPLA3 rs738409 and HSD17B13 rs72613567, association analyses for quantitative (ALT and AST) and binary (nonalcoholic liver disease and alcoholic liver disease) traits were conducted using linear and logistic regression, respectively, modeling main effects for both genetic variants as well as an interaction term, assuming an additive genetic model. All models were adjusted for age, age², sex, BMI, and the first four principal components of ancestry. Statistical analyses were performed using the glm function in base R.

### Software

Genetic association analyses were performed using GCTA software, version 1.25.0 (Yang et al., Am. J. Hum. Genet., 2011, 88, 76-82) and PLINK, version 1.9.0. Quantile-quantile and Manhattan plots were generated using R software, version 3.2.1 (R Project for Statistical Computing). Regional association plots were generated using LocusZoom (Pruim et al., Bioinformatics, 2010, 26, 2336-7).

### RNA Sequencing Studies

RNA quality and concentration was evaluated by running total RNA on an Agilent RNA Nano Bioanalyzer chip; all samples had an RNA integrity number (RIN) greater than 8. Polyadenlylated RNA transcripts were isolated using two rounds of enrichment with oligo (dT) 25 beads (Thermo Fisher Scientific). Samples were purified and concentrated with RNAclean XP beads (Beckman Coulter) and heat-fragmented to approximately 140 base pairs. First-strand synthesis was completed with SuperScript Ill reverse transcriptase (Thermo Fisher Scientific) using random hexamers; dTTP was replaced with dUTP during second-strand synthesis. Samples were processed according to the standard DNA library preparation method referenced above for exomes with the addition of a uracil DNA-glycosylase step to generate strand-specific sequencing libraries. Samples were pooled and sequenced using 75 bp paired-end sequencing on an Illumina v4 HiSeq 2500.

### Identification and Validation of Novel HSD17B13 Transcripts

Reads were mapped to the Human.B38 using ArrayStudio® software (OmicSoft®, Cary, NC) allowing two mismatches. Two approaches were employed to identify novel HSD17B13 transcripts. Novel exon junctions were discovered based on Gencode v24 using ArrayStudio. De novo transcript assembly was performed using Trinity (v2.2.0) in default setting. Custom gene models were built to incorporate novel transcripts of HSD17B13, and transcript quantification was estimated by read alignment to the custom gene model. Protein sequence alignment of all identified HSD17B13 isoforms was determined. RT-PCR was performed on total RNA from human liver samples using the SuperScript™ One-Step RT-PCR System with Platinum™ Taq DNA Polymerase (Thermofisher). Each 50 μL RT-PCR reaction contained 1× Reaction Mix, 500 nM each forward and reverse primers (PST516: ATGAACATCATCCTAGAAATCCTTC; SEQ ID NO:62) and PST517: ATCATGCATACATCTCTGGCT GGAG; SEQ ID NO:63), 1 μL of RT/Platinum Taq, and 75 ng RNA. Cycling conditions were: one cycle of 45° C. for 30 minutes; one cycle of 94° C. for 2 minutes; 40 cycles of 94° C. for 20 seconds, 53° C. for 30 seconds, and 72° C. for 90 seconds; one cycle of 72° C. for 5 minutes; then a 10° C. hold. Products were purified using the QIAquick PCR Purification Kit (Qiagen) and submitted for direct Sanger sequencing using the primer DE002 (ATCAGAACTTC AGGCCTTGG; SEQ ID NO:64). To identify the B and C transcripts, the RT-PCR products were run out on a 2% agarose gel stained with SYBR GoldSYBR® Gold Nucleic Acid Gel Stain (Thermofisher), and bands of the expected molecular weight were excised and purified using the QIAquick Gel Extraction Kit (Qiagen), then subjected to cloning with the TOPO® TA Cloning Kit (Thermofisher). Sequencing of the TOPO clones was performed using, M13F and M13R sequencing primers. Sequence analysis was performed using the Sequencher DNA analysis software (Gene Codes Corporation).

Full-length HSD17B13 transcripts were amplified directly from 50 ng of total RNA with the SuperScript III One-step RT-PCR System with Platinum Taq High Fidelity (Thermo Fisher Scientific) using gene-specific primers in the first (GCAAAGCCATGAACATCATCC; SEQ ID NO: 65) and last exons (TCTTGATGTAGTGGGAGTCGGATT; SEQ ID NO:66) to generate an amplicon of about 2.2 kb (maximum predicted size transcript). Amplicons were verified on an Agilent Bioanalyzer. PacBio-compatible barcoded adapters were ligated to the amplicons and cleaned with PacBio PB beads (Pacific Biosciences). Libraries were pooled in equal amounts and sequenced on one SMRT cell for 180 minutes on the PacBio RSII platform. The data was demultiplexed using PacBio software smrtanalysis v2.3 tool labelzmw and then analyzed with ConsensusTools AmpliconAnalysis. Resulting amplicons were compared to HSD17B13 RefSeq genes to determine isoform and genotype status.

### Subcellular Localization of HSD17B13 Isoforms

HepG2 cells were infected with lentivirus carrying the HSD17B13 A and D transcripts, stable cell lines were selected, and HSD17B13 isoforms, lipid droplets, and endoplasmic reticulum were visualized using immunofluorescence. Briefly, HepG2 cells were cultured in Eagle's Minimum Essential Medium supplemented with 10% fetal bovine serum. HSD17B13 transcripts A and D were sub-cloned into Myc-DDK backbone lentivirus constructs, and lentivirus were generated. HepG2 cells were infected with lentivirus carrying the HSD17B13 transcripts. Stable cell lines expressing each HSD17B13 transcript were selected with 1-3 mg/ml Geneticin G-418 sulfate in complete culture medium for two weeks. Following fixation, HSD17B13 isoforms were detected with mouse anti-Myc antibody. Lipid droplets were labeled with BODIPY FL dye (Sigma). Lipid coat protein and endoplasmic reticulum were labeled with rabbit anti-PLIN antibody (Sigma) and rabbit anti-calnexin antibody (Cell Signaling Technology). Secondary antibodies for immunofluorescence were Alexa Fluor 488 donkey anti-rabbit IgG and Alexa Fluor 594 donkey anti-mouse IgG (Jackson ImmunoResearch).

### Quantification of HSD171B3 Protein Expression in Human Liver Biopsy Tissue

Human liver and cell pellet samples were homogenized in ice-cold 1× RIPA lysis buffer (EMD Millipore) in the presence of protease and phosphatase inhibitor mixtures (Thermo-Fisher). Supernatant was collected and used for protein concentration using BCA protein assay (Thermo-Fisher). Human tissue lysates were loaded at 30 μg/well and stable cell lines were loaded 9 μg/well and separated on SDS/PAGE gels (Bio-Rad) and transferred to PVDF membranes (Bio-Rad). The membranes were blocked for 1 hour with 5% (wt/vol) milk in 1× TBS supplemented with 0.1% Tween20 (Bio-Rad). Membranes were incubated with antibody at 4° C. overnight against HSD17B13 (1:200, Thermo-Fisher) and B-Actin (1:500, Cell Signaling Technology). Bound antibody was detected using HRP-conjugated anti-rabbit antibody (1:10,000, Jackson ImmunoResearch) and enhanced using chemiluminescence reagent (Thermo-Fisher). Band intensities were quantified using Image J software.

In vitro and Cellular Characterization of HSD17B13 Enzymatic Activity

Recombinant human HSD17B13 protein was purified from E. coli (Genscript) transformed with plasmid DNA harboring HSD17B13 transcript A or transcript D. The HSD17B13 variants contained a 10×His tag at the C terminus and were purified from soluble fraction using a Ni²⁺ affinity purification. Enzymatic activity was determined through measurement of NADH production using the NAD (P) H-Glo Detection System (Promega). Reactions were performed for 3 hours at 25° C. in 0.2 M tris-HCl, pH 7.5, 0.5 mM NAD⁺, 75 UM of substrate (Sigma) and 500 ng purified enzyme in a final volume of 100 μL. After incubation, 20 μL of the reaction was combined with 20 μL luciferase reagent (Promega), incubated at room temperature for 1 hour and read on an Envision Plate Reader (Perkin Elmer).

HEK293 cells overexpressing HSD17B13 transcript A, transcript D or green fluorescent protein (GFP, control) were used to investigate the activity of HSD17B13 against estradiol in a cell-based assay. Estradiol was fed to each cell type. After 48 hours, the media was collected and the concentration of estradiol and its converted product estrone were identified and quantified by LC-MS. Hydroxyestradiol (metabolite from estradiol) and hydroxyestrone (metablolite from estrone) were identified by LC-MS.

### Example 2: Gene Expression Analysis of HSD17B13 and PNPLA3 in 66 Human Liver Samples

Gene expression of HSD17B13 and PNPLA3 were analyzed with 66 human liver samples. All the samples were from control donors without steatosis, lobular inflammation, or fibrosis. The distribution of HSD17B13 rs72613567 (T/T, T/TA, and TA/TA) and PNPLA3 rs738409 (C/C, C/G, and G/G) genotypes is shown in Table 1.

| Genotype | C/C | C/G | G/G | ND |
|---|---|---|---|---|
| T/T | 12 | 8 | 1 | 0 |
| T/TA | 15 | 12 | 0 | 2 |
| TA/TA | 12 | 4 | 0 | 0 |

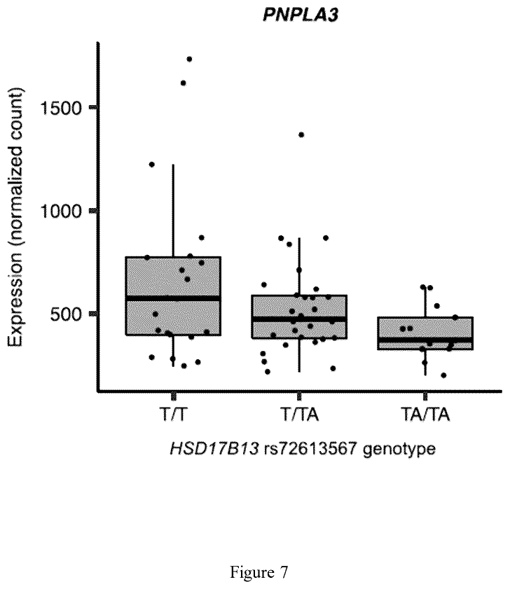

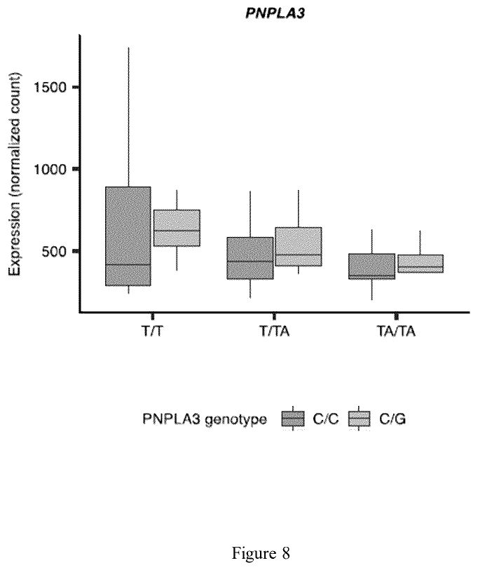

The expression of PNPLA3 was significantly reduced in homozygous alternate carriers of the HSD17B13 rs72613567 splice variant (see, FIG. 7). mRNA expression is displayed in FPKM units. A 1.6-fold decrease compared to T/T with FDR 0.0071 was observed. The variant PNPLA3 C/C carries with the HSD17B13 TA/TA genotype had significantly decreased expression when compared with HSD17B13 T/T carries: 1.7-fold (FDR 0.017) decrease. The variant PNPLA3 C/G carriers with TA/TA genotype showed decrease in expression but not statistically significant (1.4-fold, FDR 1). FIG. 8 shows the expression difference of the 63 PNPLA3 rs738409 carriers (C/C and C/G, see Table 1) in the three HSD17B13 rs72613567 genotypes (T/T, T/TA, TA/TA).

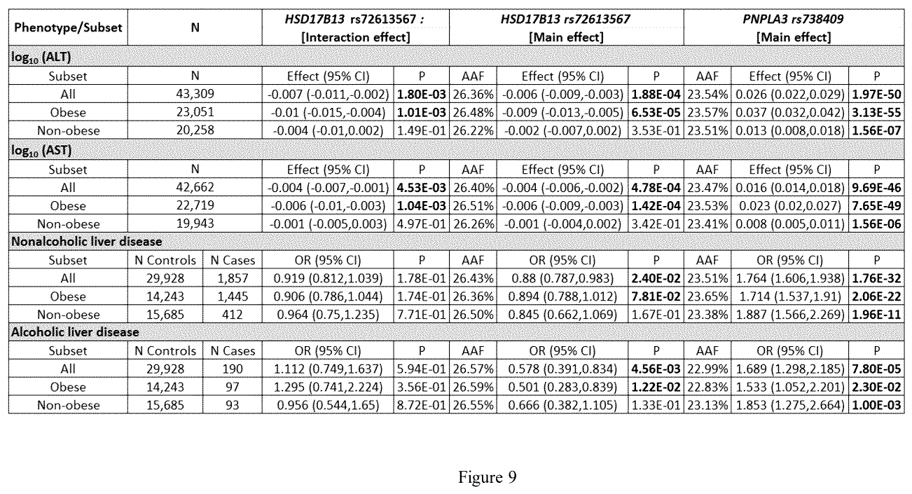

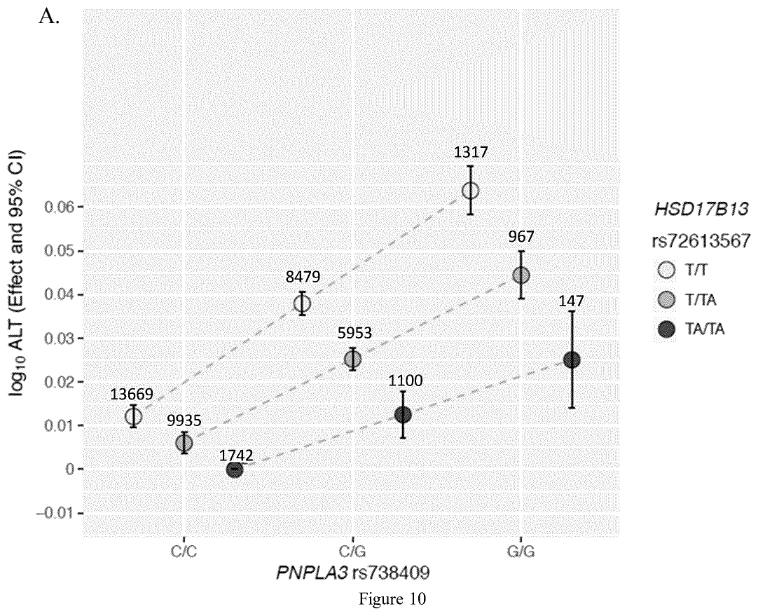

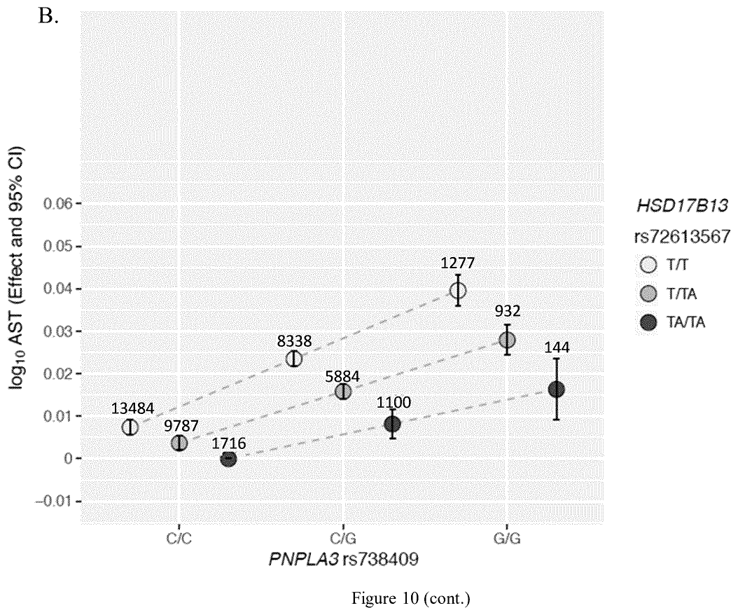

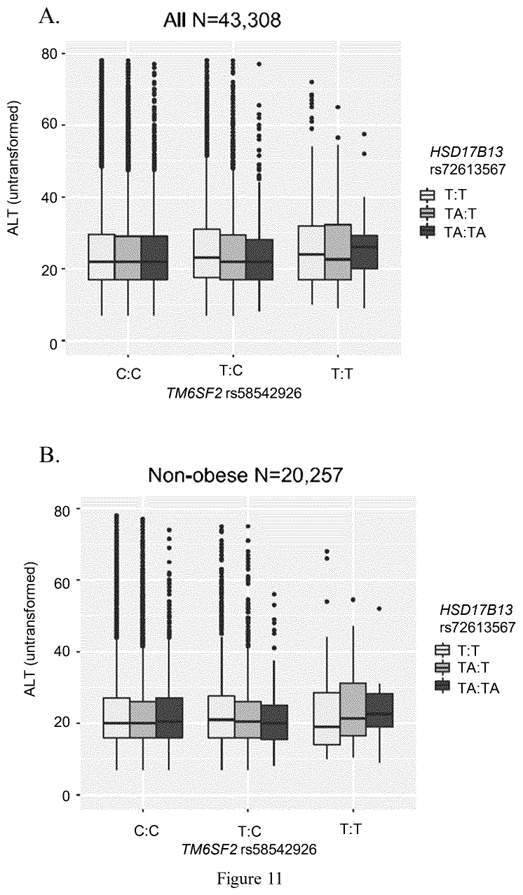

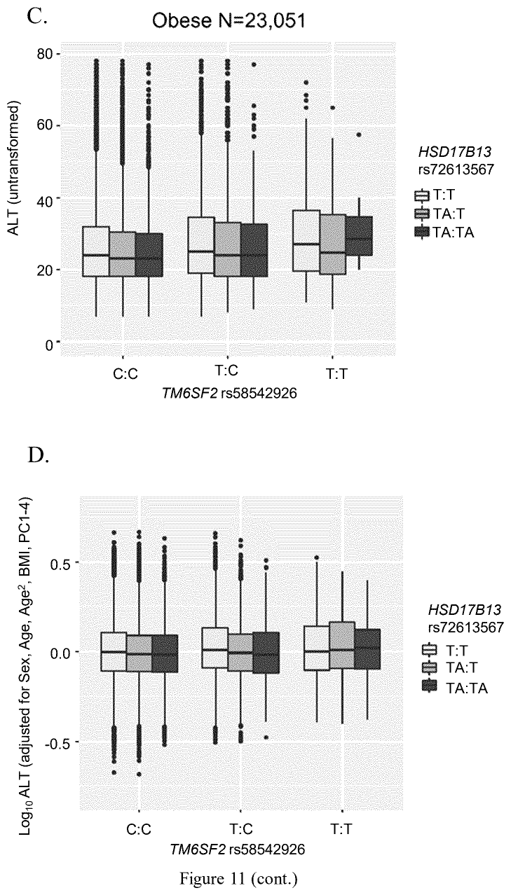

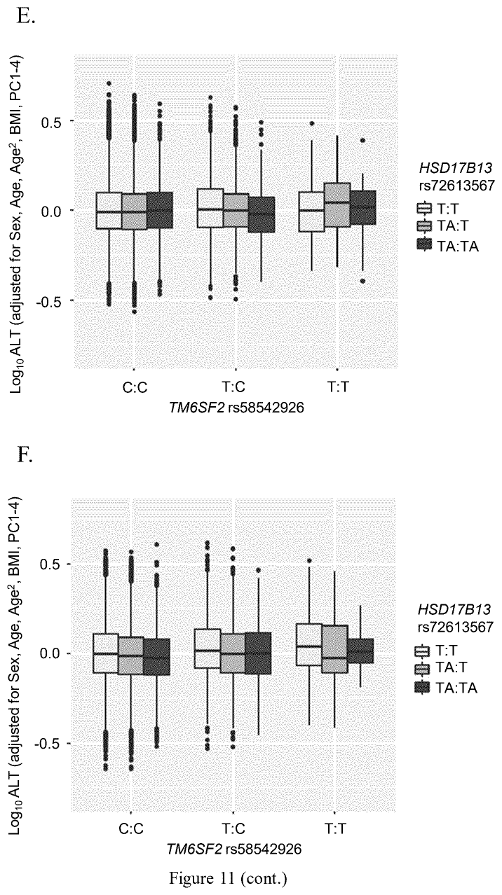

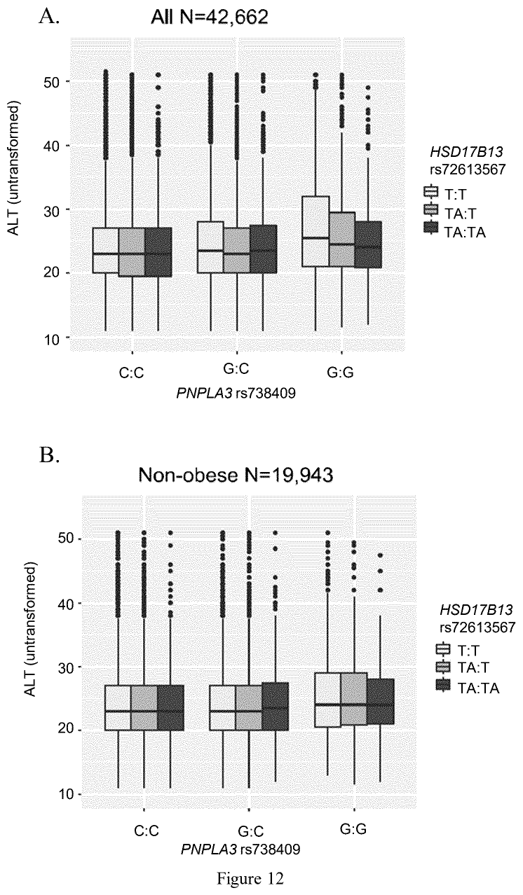

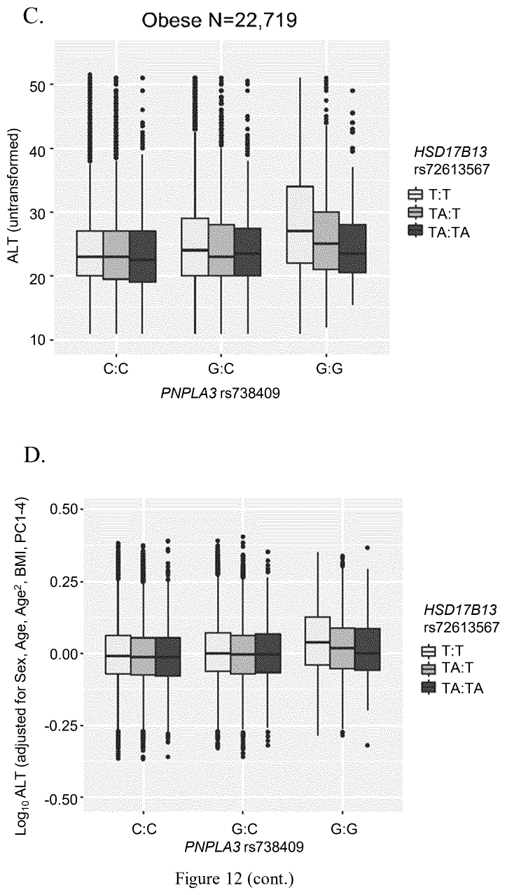

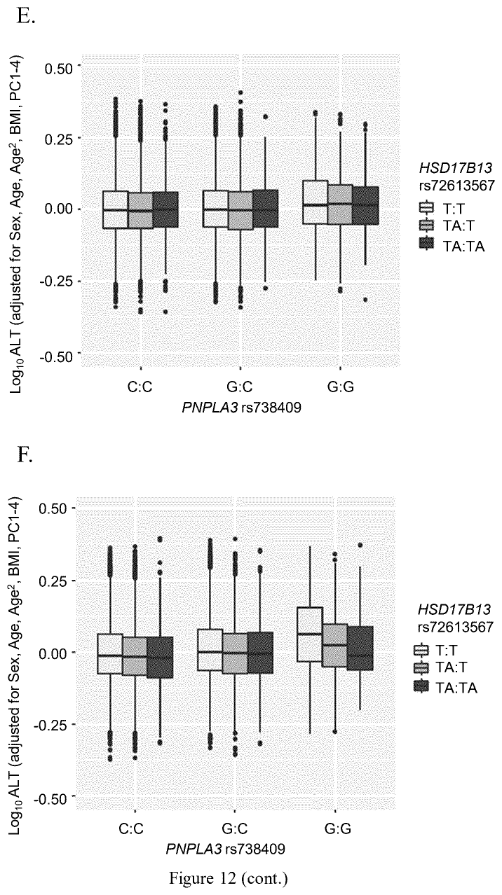

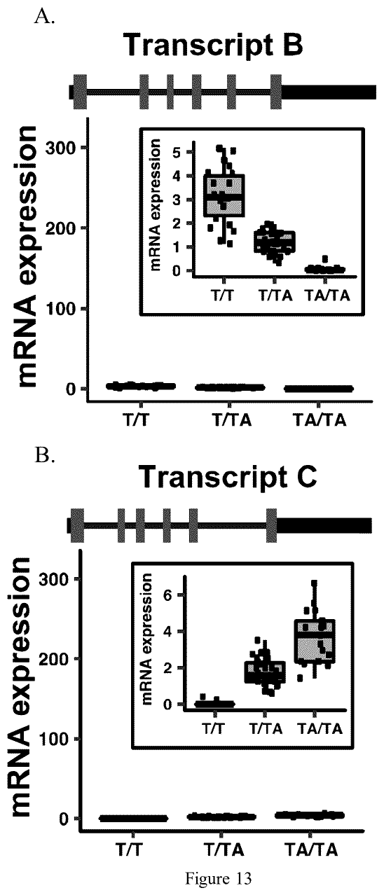

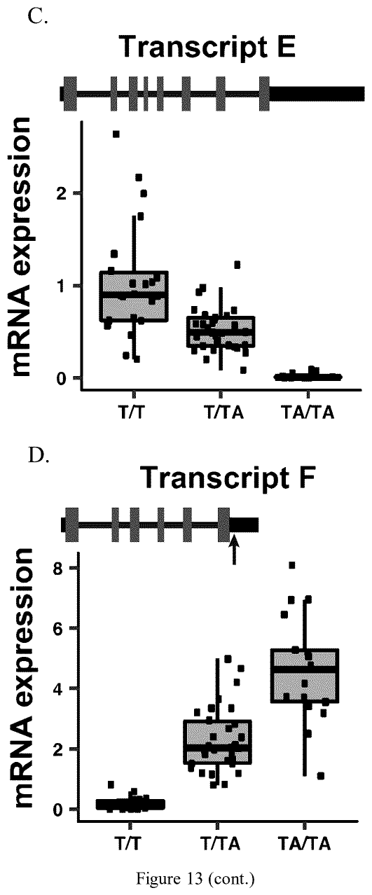

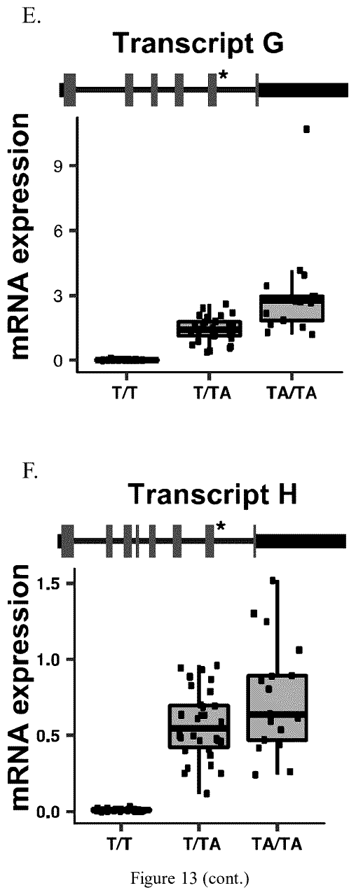

The variant PNPLA3 p.I148M variant is the most well validated genetic risk factor for NAFLD, and the 148M allele exists in homozygous state in 5-25% of individuals, depending on ancestry. To understand whether the HSD17B13 rs72613567: TA modifies the risk of liver injury associated with PNPLA3 p.I148M, analyses of interaction between the two variants in association with ALT, AST, and chronic liver disease phenotypes in DiscovEHR was performed. These analyses were performed in all participants, as well as in obese (body mass index (BMI)>30 kg/m²) and non-obese (BMI≤30 kg/m²) subpopulations. There was nominally significant interaction between HSD17B13 rs72613567: TA and PNPLA3 p.I148M in association analyses of ALT (P=1.8×10⁻³ for interaction) and AST (P=4.5×10⁻³ for interaction) levels; these associations were primarily driven by associations in obese individuals (see, FIG. 9). In these analyses, the rs72613567: TA allele mitigated the allele dosage-dependent associations of PNPLA3 148M allele with increased ALT and AST (see, FIG. 10). Referring to FIG. 10, panel A shows the association of HSD17B13 rs72613567 with ALT in individuals with each PNPLA3 p.I148M genotype, and panel B shows the association of HSD17B13 rs72613567 with AST in individuals with each PNPLA3 p.I148M genotype. Effect estimates (beta and 95% Cl) were calculated using linear regression, with adjustment for age, age², sex, BMI, and four principal components of ancestry. FIG. 11 (panels A through F) show raw and residualized ALT levels by PNPLA3 rs738409 (p.I148M) and HSD17B13 rs72613567 genotype. Residuals were calculated by linear regression adjusted for age, age², sex, BMI, and four principal components 1-4. FIG. 12 (panels A through F) show raw and residualized AST levels by PNPLA3 rs738409 (p.I148M) and HSD17B13 rs72613567 genotype. Residuals were calculated by linear regression adjusted for age, age², sex, BMI, and four principal components 1-4. FIG. 13 (panels A through F) show mRNA expression of four additional novel HSD17B13 transcripts (E-H) in homozygous reference (T/T), heterozygous (T/TA), and homozygous alternate (TA/TA) carriers of the HSD17B13 splice variant. Coding regions in gene models are indicated in red and untranslated regions in black. Transcripts E and H contain an additional exon between exons 3 and 4. Transcript F involves read-through from exon 6 to intron 6. The blue arrow indicates the A insertion from rs72613567. Transcript G lacks exon 2. The asterisk in transcripts G and H illustrates insertion of G at the 3′-end of exon 6, which leads to premature truncation of the protein (similar to transcript D). Transcripts are differentially expressed according to HSD17B13 genotype, as shown in the box plots. mRNA expression is displayed in FPKM units.

These data suggest the HSD17B13 rs72613567: TA variant mitigates the risk of liver injury in individuals genetically predisposed to steatotic liver disease by the variant PNPLA3 p.I148M variant. This finding may suggest an important subpopulation for therapeutic modulation of HSD17B13-individuals heterozygous or homozygous for the variant PNPLA3 148M allele.

### Example 3: Gene Expression Analysis of HSD17B13 and PNPLA3 in 66 Human Liver Samples

Association of Exonic Variants with Aspartate and Alanine Aminotransferases

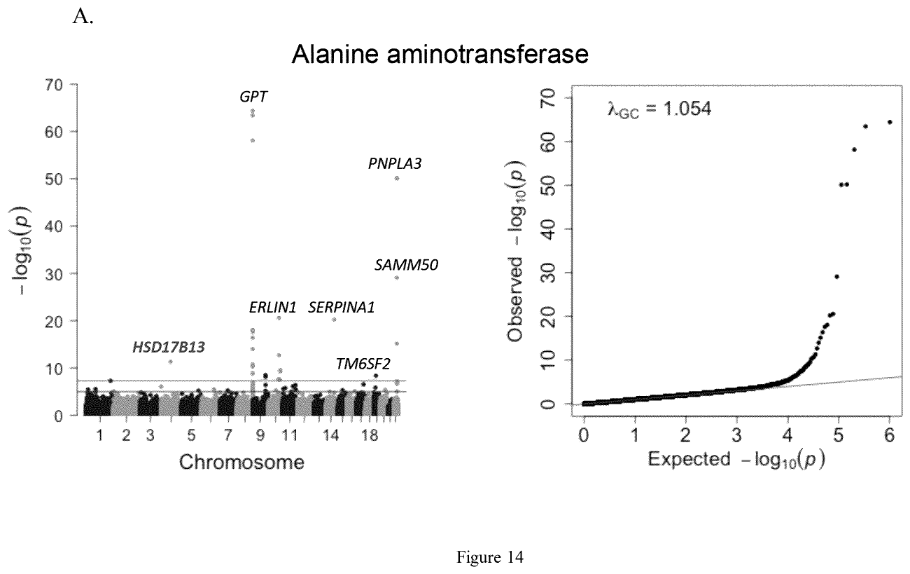

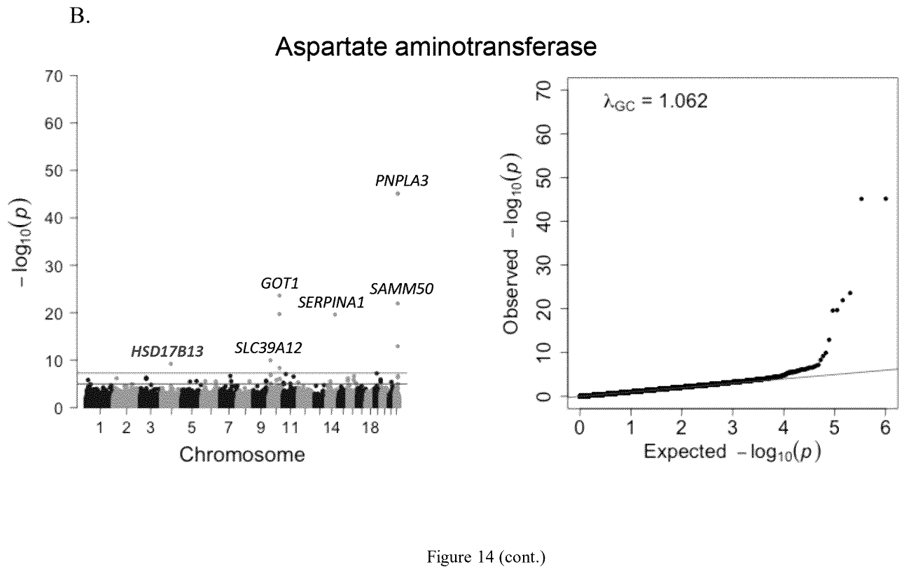

502,219 biallelic single genetic variants were examined for association with serum ALT or AST levels in 46,544 individuals of European descent from the DiscovEHR study (“GHS discovery cohort”; basic demographics in FIG. 1). A total of 35 variants in 19 genes were found to be associated with ALT or AST at P<1.0×10⁻⁷ (see, FIG. 14 and FIG. 2). Referring to FIG. 14, Manhattan plots (left) and quantile-quantile plots (right) of single nucleotide variant associations with serum transaminase levels in the GHS discovery cohort are shown. There were 31 variants in 16 genes significantly associated with alanine aminotransferase (ALT) levels at P<1.0×10⁻⁷ (see, Panel A). There were 12 variants in 10 genes significantly associated with aspartate aminotransferase (AST) levels at P<1.0×10⁻⁷ (see, Panel B). All significant associations are shown in FIG. 2. There were thirteen variants in nine genes (indicated here by their gene name), including HSD17B13, that remained significantly associated with ALT or AST in a replication meta-analysis of three separate European-ancestry cohorts (see, FIG. 3). The association tests were well calibrated, as shown by exome-wide quantile-quantile plots and genomic control lambda values.

Replication studies were performed in three cohorts of European-ancestry individuals: 1) bariatric surgery patients (n=2,644) from DiscovEHR (“GHS bariatric surgery cohort”); 2) 1,357 individuals from the Dallas Heart Study; and 3) 8,526 individuals from the Penn Medicine Biobank. In meta-analysis of the replication cohorts, thirteen variants in nine genes were significantly associated with serum levels of ALT or AST (Bonferroni significance threshold of P<1.43×10⁻³ for 35 variants tested (see, FIG. 3). These included variants that were previously reported to be associated with elevated transaminase levels, such as PNPLA3 (Romeo et al., Nat. Genet., 2008, 40, 1461-5), TM6SF2 (Kozlitina et al., Nat. Genet. 2014, 46, 352-6), SERPINA1 (Brantly et al., Am. J. Med., 1988, 84, 13-31), SAMM50 (Kitamoto et al., Hum. Genet., 2013, 132, 783-92), and ERLIN1 (Feitosa et al., Atherosclerosis, 2013, 228, 175-80). SERPINA1 encodes alpha-1-antitrypsin, whose functional deficiency causes liver disease; the association with SAMM50 is mediated via linkage disequilibrium with variation in PNPLA3, and ERLIN1 has been implicated in liver fat deposition. Variants that were not previously reported to be associated with liver disease were also identified. These included several variants in GPT and GOT1, the genes encoding ALT and AST, respectively, and SLC39A12, which encodes solute carrier family 39 member 12.

A reproducible association between a variant in HSD17B13, the gene encoding hydroxysteroid 17-beta dehydrogenase 13, an uncharacterized member of the 17-beta hydroxysteroid dehydrogenase family, and decreased levels of ALT (discovery P=4.2×10⁻¹², replication P=1.7×10⁻⁴) and AST (discovery P=6.2×10⁻¹⁰, replication P=1.7×10⁻⁴, see, FIG. 3) was also identified. The associated variant, rs72613567, is an insertion of an adenine adjacent to the donor splice site of exon six (TA allele), and had an allele frequency of 26.0% in the GHS discovery cohort. Previously, Chambers, et al identified a nearby locus at 4q22 (rs6834314) associated with ALT levels (Chambers et al., Nat. Genet., 2011, 43, 1131-8); rs72613567 has not heretofore been reported to be associated with transaminase levels. HSD17B13 is 30 kb upstream of HSD17B11, another member of the same gene family. No exome-wide significant associations were observed between coding or splice variants in HSD17B11 and transaminase levels in the discovery cohort (see, FIG. 6) or in the joint meta-analysis of the discovery cohort and three replication cohorts. Furthermore, linkage disequilibrium of rs72613567 with variants in HSD17B11 was modest across all ancestry groups (r²<0.4 with all ascertained variants in HSD17B11 in all ancestry groups; data not shown). Collectively, these findings suggest HSD17B13 as the gene in the genomic region that is most likely to be functionally related to transaminase levels.

Association of Exonic Variants with Clinical Diagnoses of Chronic Liver Disease

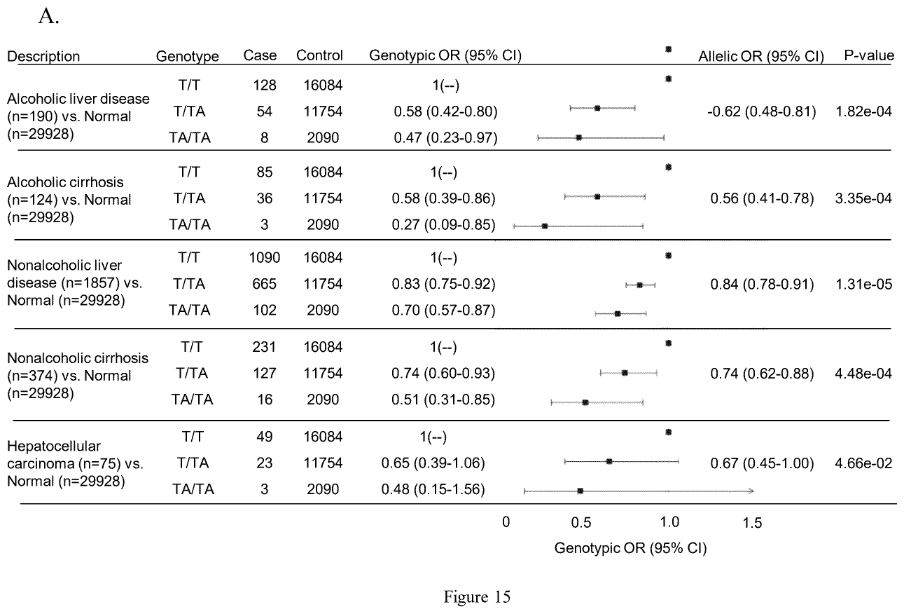

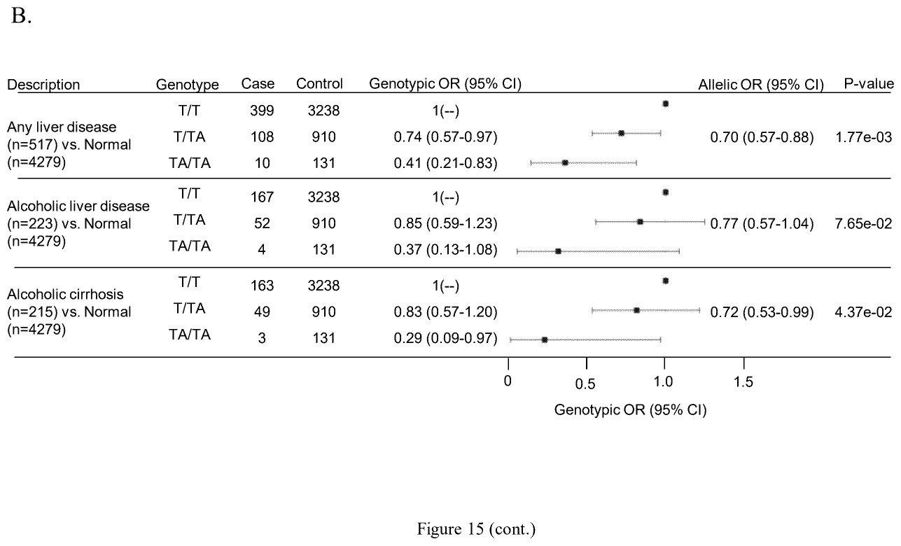

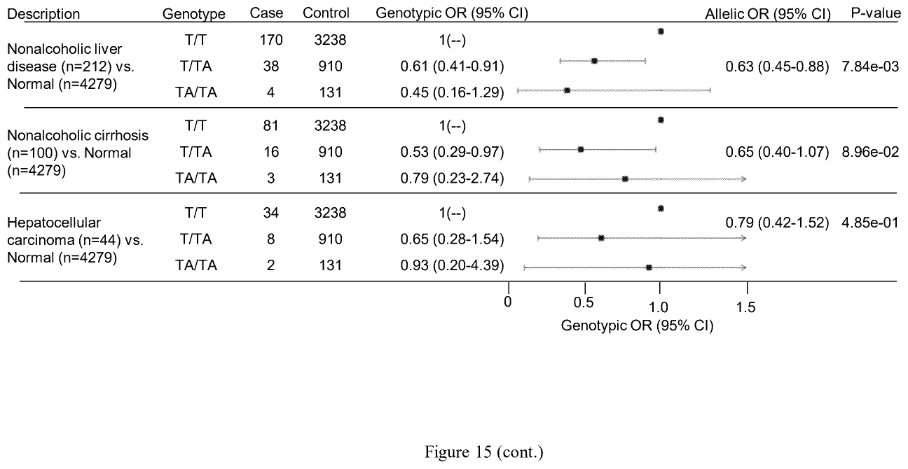

The relationship between the thirteen transaminase-associated variants in the nine genes found in the discovery and replication cohorts and chronic liver disease, including alcoholic and nonalcoholic (non-viral) liver disease, as well as the most advanced forms of chronic liver disease: alcoholic cirrhosis, nonalcoholic cirrhosis, and hepatocellular carcinoma (HCC), was also analyzed. Using a Bonferroni significance threshold of P<1.92×10⁻³ for the thirteen variants tested, significant associations were found between six variants in five genes (HSD17B13, SERPINA1, TM6SF2, PNPLA3, and SAMM50) and chronic liver disease phenotypes (see, FIG. 4). The SERPINA1, TM6SF2, PNPLA3, and SAMM50 associations confirm previously reported associations. In the discovery cohort, HSD17B13 rs72613567: TA was associated with lower odds of all EHR-derived categories of both alcoholic and nonalcoholic liver disease in an allele dosage-dependent manner (see, FIG. 15, panel A): all categories of alcoholic liver disease, heterozygous odds ratio (ORₕₑₜ) (95% confidence interval) 0.58 (0.42-0.80), homozygous OR (ORₕₒₘ) 0.47 (0.23-0.97), allelic OR (OR_{allelic}) 0.62 (0.48-0.81), P=1.8×10⁻⁴; all categories of nonalcoholic liver disease, ORₕₑₜ 0.83 (0.75-0.92), ORₕₒₘ 0.70 (0.57-0.87), OR_{allelic} 0.84 (0.78-0.91), P=1.3×10⁻⁵. HSD17B13 rs72613567: TA was also associated with lower odds alcoholic and nonalcoholic cirrhosis, with 42% and 73% lower odds of alcoholic cirrhosis for heterozygotes and homozygotes, respectively, (ORₕₑₜ 0.58 (0.39-0.86), ORₕₒₘ 0.27 (0.09-0.85), OR_{allelic} 0.56 (0.41-0.78), P=3.4×10⁻⁴) and 26% and 49% lower odds of nonalcoholic cirrhosis for heterozygotes and homozygotes, respectively (ORₕₑₜ 0.74 (0.60-0.93), ORₕₒₘ 0.51 (0.31-0.85), OR_{allelic} 0.74 (0.62-0.88), P=4.5×10⁻⁴). HSD17B13 rs72613567: TA was also nominally associated with lower odds of HCC.

These findings were confirmed and extended in the multi-ethnic Dallas Liver Study (DLS) and the Dallas Pediatric Liver Study (DPLS) (see, FIG. 5). In the DLS, the TA allele was associated with lower odds of any liver disease in an allele-dosage dependent manner (ORₕₑₜ 0.74 (0.57-0.97), ORₕₒₘ 0.41 (0.21-0.83), OR_{allelic} 0.70 (0.5-0.88), P=1.8×10⁻³, see FIG. 15, panel B). Similar effects were observed across EHR-derived liver disease subtypes, including protective associations with advanced, cirrhotic forms of alcoholic (OR_{allelic} 0.72 (0.53-0.99), P=4.4×10⁻²) and nonalcoholic (OR_{allelic} 0.65 (0.40-1.07), P=9.0×10⁻²) liver disease. In subset analyses of individuals grouped by self-reported ethnicity, the association with liver disease was significant in Hispanic Americans (n=326 cases and 722 controls, OR_{allelic} 0.51 (0.35-0.74), P=4.0×10⁻⁴); similar numerical trends, which did not achieve statistical significance, were also noted in the African American (n=33 cases and 2,291 controls, OR_{allelic} 0.74 (0.25-2.47), P=0.67) and European American (n=158 cases and 1,266 controls, OR_{allelic} 0.87 (0.65-1.15), P=0.32) subsets of the DLS. In the DPLS, a separate study of Hispanic American pediatric liver disease patients and obese controls, the TA allele was also associated with lower odds of liver disease (OR_{allelic} 0.61 (0.37-0.99), P=4.6×10⁻²). Thus, HSD17B13 rs72613567: TA was associated with reduced odds of multiple forms of chronic liver disease, including cirrhosis, in adults and children in three independent populations.

Referring to FIG. 15, HSD17B13 rs72613567: TA is associated with reduced risk of alcoholic and nonalcoholic liver disease phenotypes is shown. In the GHS discovery cohort, HSD17B13 rs72613567 was associated with lower odds of nonalcoholic and alcoholic liver disease, cirrhosis, and hepatocellular carcinoma in an allele dosage-dependent manner (see, Panel A). Odds ratios were calculated using logistic regression, with adjustment for age, age², sex, BMI, and principal components of ancestry. Genotypic odds ratios for heterozygous (Het OR) and homozygous (Hom OR) carriers are also shown. In the Dallas Liver Study, HSD17B13 rs72613567 was associated with lower odds of any liver disease in an allele dosage-dependent manner (see, Panel B). Similar allele dosage-dependent effects were observed across liver disease subtypes. Odds ratios were calculated using logistic regression, with adjustment for age, age², sex, BMI, and self-reported ethnicity.

Genetic Interaction Between PNPLA3 rs738409 (p.I148M) and HSD17B13 rs72613567

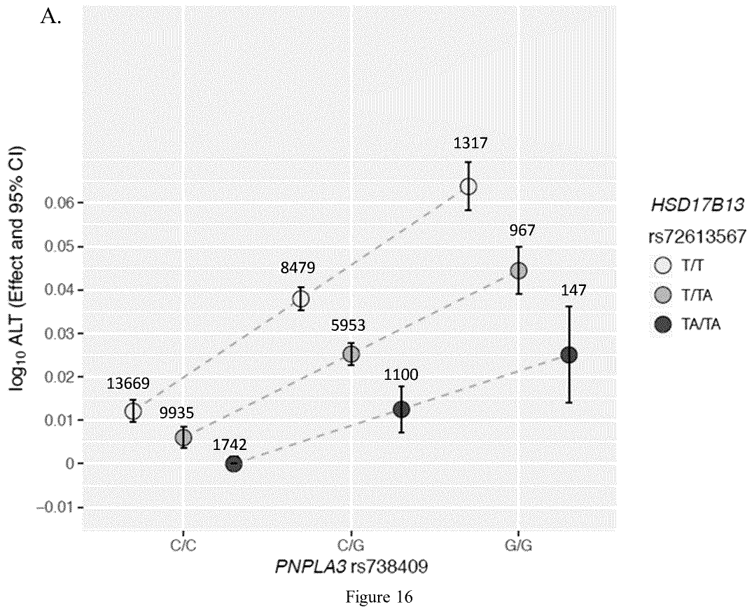

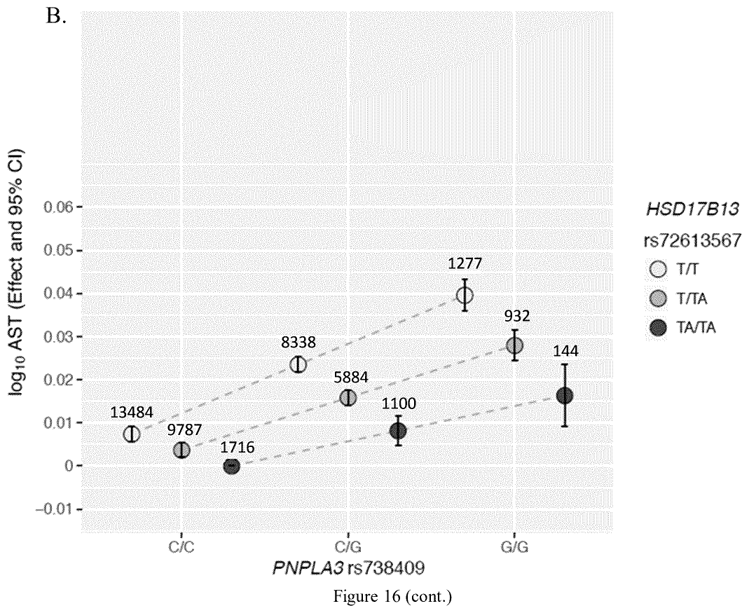

The variant PNPLA3 p.I148M variant is the most well validated genetic risk factor for NAFLD, and the 148M allele exists in homozygous state in 5-25% of individuals, depending on ancestry. To understand whether the HSD17B13 rs72613567: TA modifies the risk of liver injury associated with PNPLA3 p.I148M, analyses of interaction between the two variants in association with ALT, AST, and chronic liver disease phenotypes in DiscovEHR was performed. These analyses were performed in all participants, as well as in obese (body mass index [BMI]≥30 kg/m²) and non-obese (BMI<30 kg/m²) subpopulations. There was nominally significant interaction between HSD17B13 rs72613567: TA and PNPLA3 p.I148M in association analyses of ALT (P=1.8×10⁻³ for interaction) and AST (P=4.5×10⁻³ for interaction) levels; these associations were primarily driven by associations in obese individuals (see, FIG. 9). In these analyses, the rs72613567: TA allele mitigated the allele dosage-dependent associations of PNPLA3 148M allele with increased ALT and AST (see, FIG. 16, FIG. 11, and FIG. 12). RNA sequencing-based expression analysis revealed that HSD17B13 rs72613567: TA was associated with decreased PNPLA3 mRNA expression in an allele dosage-dependent manner (see, FIG. 7). These data suggest the HSD17B13 rs72613567: TA variant mitigates the risk of liver injury in individuals genetically predisposed to steatotic liver disease by the variant PNPLA3 p.I148M variant.

Referring to FIG. 16, HSD17B13 rs72613567: TA mitigates the risk of liver injury associated with PNPLA3 p.I148M is shown. Association of HSD17B13 rs72613567 with ALT in individuals with each PNPLA3 p.I148M genotype (see, Panel A). Association of HSD17B13 rs72613567 with AST in individuals with each PNPLA3 p.I148M genotype (see, Panel B). Effect estimates (beta and 95% Cl) were calculated using linear regression, with adjustment for age, age², sex, BMI, and four principal components of ancestry. The P values for interaction between HSD17B13 rs72613567: TA and PNPLA3 p.I148M in association analyses of ALT and AST levels were P=1.8×10⁻³ and P=4.5×10⁻³, respectively.

Association of HSD17B13 rs72613567: TA with Liver Pathology

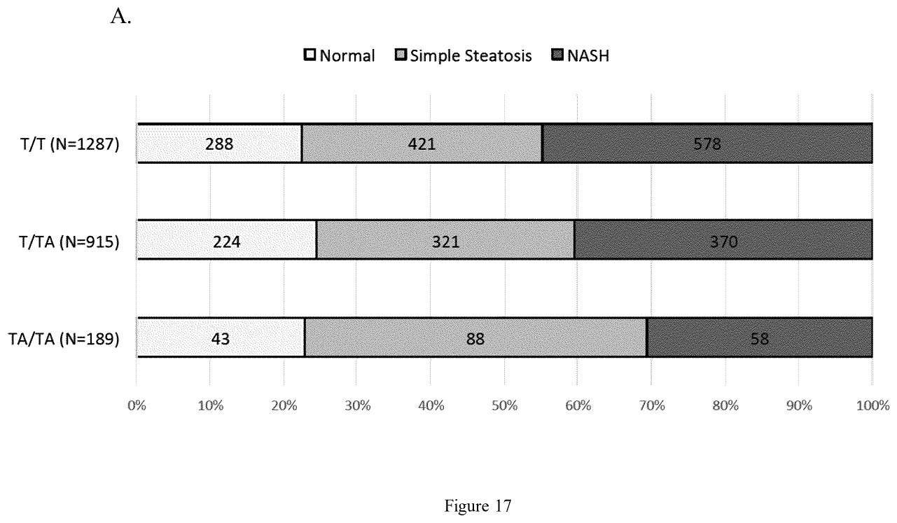

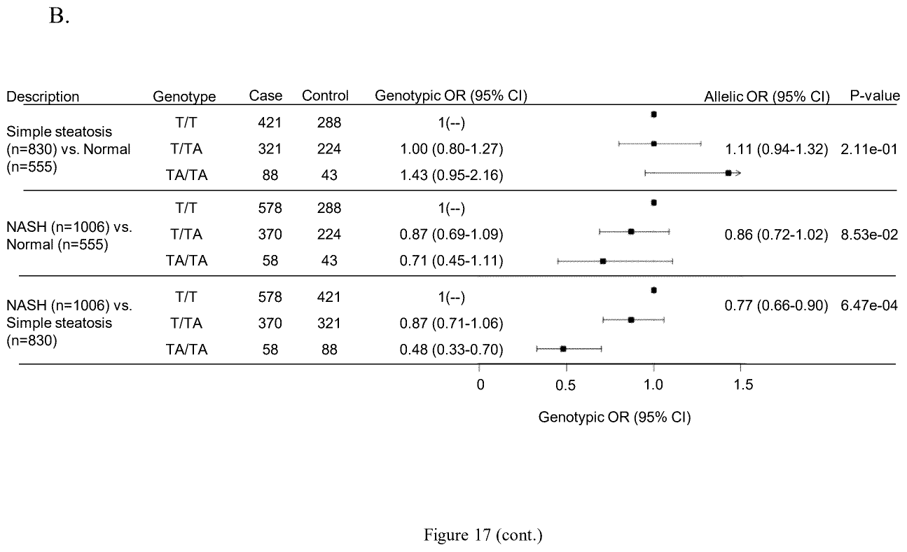

NAFLD describes a disease spectrum ranging from liver fat accumulation without evidence of significant inflammation (simple steatosis), to more clinically impactful NASH. To confirm the association between the HSD17B13 rs72613567: TA and EHR-derived liver disease diagnoses codes, and to further understand its association with histopathological progression of steatosis to NASH, tests of association in the GHS bariatric surgery cohort were performed. In this cohort of 2,391 of the whole exome sequenced individuals assessed by liver biopsy at the time of bariatric surgery, a total of 555 (23%) individuals had no evidence of steatosis, steatohepatitis, or fibrosis (“normal”), 830 (35%) had simple steatosis, and 1006 (42%) had NASH. When comparing prevalence of normal liver, simple steatosis, and NASH by genotype, it was observed that the prevalence of normal liver did not appear to differ by genotype (23%, 24%, and 23% for T/T, T/TA, and TA/TA carriers, respectively, P=0.5 by Chi-squared test for trend in proportions), but that the prevalence of NASH decreased (45%, 40%, and 31% for T/T, T/TA, and TA/TA carriers, respectively, P=1.6×10⁻⁴) and that of simple steatosis increased (33%, 35%, and 47% for T/T, T/TA, and TA/TA carriers, respectively, P=1.1×10⁻³) with each TA allele (see, FIG. 17, Panel A). Among individuals with steatosis, the TA allele was associated with statistically significantly lower odds of NASH, as compared to simple steatosis, in an allele dosage-dependent manner (ORₕₑₜ 0.87 (0.71-1.06), ORₕₒₘ 0.48 (0.33-0.70), OR_{allelic} 0.77 (0.66-0.90), P=6.5×10⁻⁴) (see, FIG. 17, Panel B). Altogether, these data suggest a role for HSD17B13 in mediating NAFLD progression from simple steatosis to more advanced stages of NASH and fibrosis.

Referring to FIG. 17, HSD17B13 rs72613567: TA is associated with reduced risk of progression from simple steatosis to steatohepatitis and fibrosis is shown. Prevalence of histopathologically-characterized liver disease according to HSD17B13 rs72613567 genotype in 2,391 individuals with liver biopsies from the GHS bariatric surgery cohort (see, Panel A). The prevalence of normal liver did not appear to differ by genotype (P=0.5 by Chi-squared test for trend in proportions), but the prevalence of NASH decreased (P=1.6×10⁻⁴) and that of simple steatosis increased (P=1.1×10⁻³) with each TA allele. In the GHS bariatric surgery cohort, HSD17B13 rs72613567 was associated with 13% and 52% lower odds of NASH in heterozygous and homozygous TA carriers, respectively (see, Panel B). Odds ratios were calculated using logistic regression, with adjustment for age, age², sex, BMI, and principal components of ancestry. Genotypic odds ratios for heterozygous (Het OR) and homozygous (Hom OR) carriers are also shown.

Effect of rs72613567: TA on HSD17B13 mRNA and HSD17B13 Protein Expression

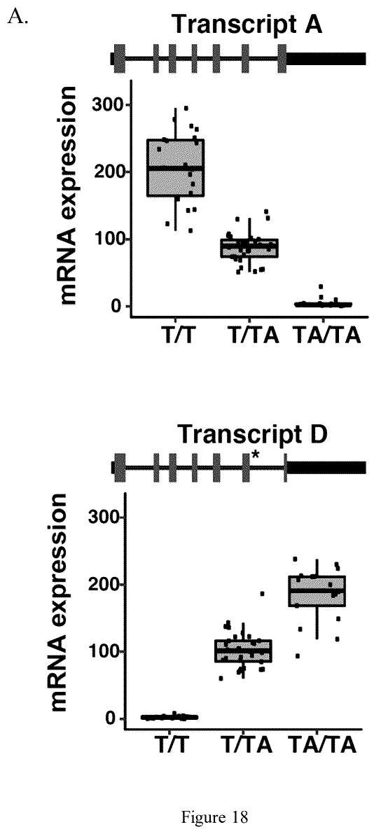

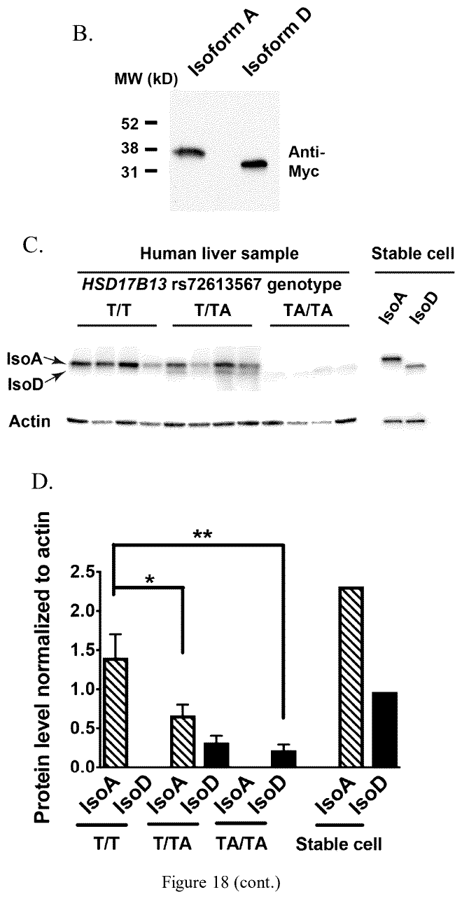

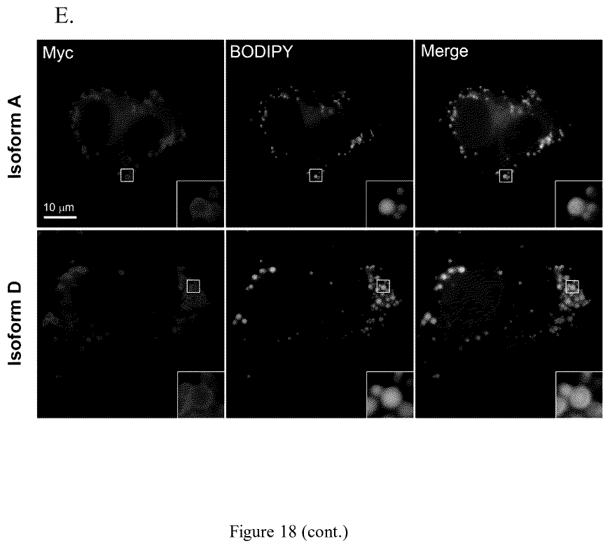

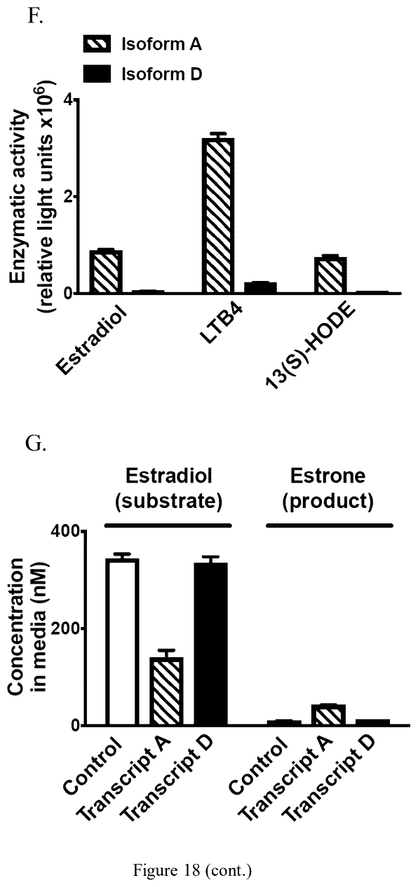

The effect of the HSD17B13 rs72613567: TA allele on expression of known and novel transcripts of the gene was examined. RNA sequencing was used to assess HSD17B13 mRNA expression in histologically normal liver samples from 22 T/T homozygous, 30 T/TA heterozygous, and 17 TA/TA homozygous carriers of the HSD17B13 rs72613567 splice variant. In addition to the two HSD17B13 transcripts, A and B, two novel transcripts were identified: transcript C, which lacked exon 6, and transcript D which contained an insertion of a guanine nucleotide at the 3′ end of exon 6, which would be predicted to result in premature truncation of the protein. Four additional transcripts (E-H) were expressed at very low levels (see, FIG. 13). The transcripts were validated by RT-PCR and Sanger sequencing (data not shown). The D transcript was also validated using long read cDNA sequencing. The expression levels of these transcripts varied according to HSD17B13 rs72613567 genotype; levels of transcripts A and B decreased, while those of transcripts C and D increased in an allele dosage-dependent manner with each TA allele (see, FIG. 18, Panels A and B). Transcript A, which encodes the full-length 300 amino acid protein, was the predominant transcript in T/T homozygotes, while transcript D, which encodes the prematurely truncated protein, was the predominant transcript in TA/TA homozygotes. In human liver biopsy tissue, the truncated isoform D protein was minimally present in heterozygotes and TA/TA homozygotes, and isoform A protein abundance was reduced in an allele dosage-dependent manner (see, FIG. 18, Panels C and D). These data are consistent with HSD17B13 rs72613567 altering mRNA splicing, resulting in the synthesis of a truncated form of the protein with substantially reduced expression in human liver.

Referring to FIG. 18, expression, subcellular localization, and enzymatic activity of a novel HSD17B13 transcript is shown. Expression of HSD17B13 transcripts A and D in homozygous reference (T/T), heterozygous (T/TA), and homozyous alternate (TA/TA) carriers of the HSD17B13 rs72613567 splice variant (see, Panel A). Coding regions in HSD17B13 gene are indicated in red, untranslated regions as thick black lines, and introns as thin black lines. The asterisk in transcript D indicates the A insertion from rs72613567. mRNA expression is displayed in FPKM units (Fragments Per Kilobase of transcript per Million mapped reads). Western blot from HepG2 cells overexpressing HSD17B13 transcripts A and D. HSD17B13 transcript D was translated to a truncated protein with lower molecular weight compared to HSD17B13 transcript A (see, Panel B). HSD17B13 western blot from fresh frozen human liver and HEK293 cell samples (see, Panel C). Human liver samples are from homozygous reference (T/T), heterozygous (T/TA), and homozygous alternate (TA/TA) carriers of the HSD17B13 rs72613567 splice variant. Cell samples are from HEK293 cells overexpressing non-tagged HSD17B13 transcripts A and D. HSD17B13 transcript D was translated to a truncated protein IsoD with lower molecular weight than HSD17B13 IsoA. HSD17B13 IsoD protein levels were lower than IsoA protein levels from both human liver (left) and cell (right) samples (see, Panel D). Protein level normalized to Actin was shown in the bar columns; **P<0.001, *P<0.05. Both HSD17B13 isoforms A and D were localized on lipid droplet membrane (see, Panel E). HepG2 stably overexpressing HSD17B13 transcripts A or D were labelled with BODIPY to show lipid droplets and anti-Myc to show HSD17B13 localization. All figures are magnified to the same extent. Scale bar indicates 10 μm. Insets represent 4× amplification of the original images. Enzymatic activity of HSD17B13 isoforms A and D to 17-beta estradiol (estradiol), leukotriene B4 (LTB4), and 13-Hydroxyoctadecadienoic acid (13 (S)-HODE (see, Panel F). HSD17B13 isoform D show <10% enzymatic activity of the corresponding values for isoform A. G, HSD17B13 isoform D when overexpressed in HEK293 cells did not show much conversion of estradiol (substrate) to estrone (product) when measured in the culture media, while overexpressed HSD17B13 isoform A showed robust conversion.

### Expression of HSD17B13 in Human Liver Cells

HSD17B13 is expressed primarily in the liver (Liu et al., Acta Biochim. Pol., 2007, 54, 213-8), where it localizes to lipid droplets (Su et al., Proc. Natl. Acad. Sci. USA, 2014, 111, 11437-42), consistent with a role in the pathogenesis of fatty liver disease. The expression of HSD171B3 and its localization was evaluated in an immortalized human liver cell line stably transduced with lentivirus expressing HSD17B13 transcripts A and D. HSD17B13 isoform A was mainly detected on membranes surrounding BODIPY-labeled lipid droplets (see, FIG. 18, Panel E). Similar subcellular localization was observed for HSD17B13 isoform D at the lipid droplet surface (see, FIG. 18, Panel F).

Effect of rs72613567: TA on HSD17B13 Activity in vitro and in Cellular Models

To understand the functional consequences of premature truncation of HSD17B13 protein due to rs72613567: TA, the enzymatic activity of isoforms A and D was evaluated in vitro using recombinant protein. Greater than 300 putative substrates were examined, of which estradiol, leukotriene B4, and 13-Hydroxyoctadecadienoic acid were enzymatically converted by HSD17B13, resulting in oxidation of a hydroxyl to a ketone group. HSD17B13 isoform D showed greatly reduced activity towards the 3 substrates (see, FIG. 18, Panel F).

Compared to GFP control, HSD17B13 transcript A overexpressing cells had lower concentration of estradiol as well as higher concentration of estrone in the cell culture medium, suggesting enzyme activity against estradiol (see, FIG. 18, Panel G). HSD17B13 transcript D overexpressing cells had similar ratio of estrone/estadiol to GFP control cells, suggesting that HSD17B13 transcript D has significant loss of function. The mass spec analysis revealed rapid conversion of estrone into hydroxyestrone and other products accounting for the low accumulation of estrone compared to consumed estradiol.

Through large-scale exome sequencing, a novel association was identified between a splice variant in HSD17B13 and decreased serum transaminase levels, as well as reduced risk of nonalcoholic and alcoholic forms of liver disease, including advanced cirrhotic forms of liver disease and HCC. To our knowledge, this is the first report of a protein-altering variant that has a protective association with liver disease. The HSD17B13 rs72613567: TA allele was not associated with simple steatosis, but reduced the risk of progression to NASH. The consistency of the dosage-dependent protective associations in four independent cohorts (DiscovEHR, an independent bariatric surgery cohort in DiscovEHR, DLS, and DPLS) across several different liver disease categories and ethnicities support the notion that the reported HSD17B13 variant protects from progression to more clinically advanced stages of chronic liver disease. The observed allele dosage-dependence also argues that more profound regulation of HSD17B13 function may result in more profound effects on disease risk and progression. The HSD17B13 rs72613567: TA allele also mitigated the risk of liver injury in individuals genetically predisposed to steatotic liver disease by the variant PNPLA3 p.I148M variant. This finding may suggest an important subpopulation for therapeutic modulation of HSD17B13-individuals heterozygous or homozygous for the variant PNPLA3 148M allele.

The association findings described herein were primarily based on observations in European and Hispanic Americans who have elevated BMI. HSD17B13 is in close proximity with HSD17B11, a member of the same gene family with high sequence similarity to HSD17B13 but broader tissue distribution. Overall, the data presented herein support the position that HSD17B13 is a potential therapeutic target for prevention and treatment of fatty liver disease in humans. The data presented herein indicate that targeting of HSD17B13 could reduce progression of liver disease from steatosis to later stages of NASH, fibrosis, and cirrhosis, which are associated with significant morbidity and mortality, and for which there are currently no effective treatments.

### Example 4: HSD17B13 rs72613567: TA Mitigates the Risk of Alcoholic and Nonalcoholic Liver Disease Associated with PNPLA3 1148M

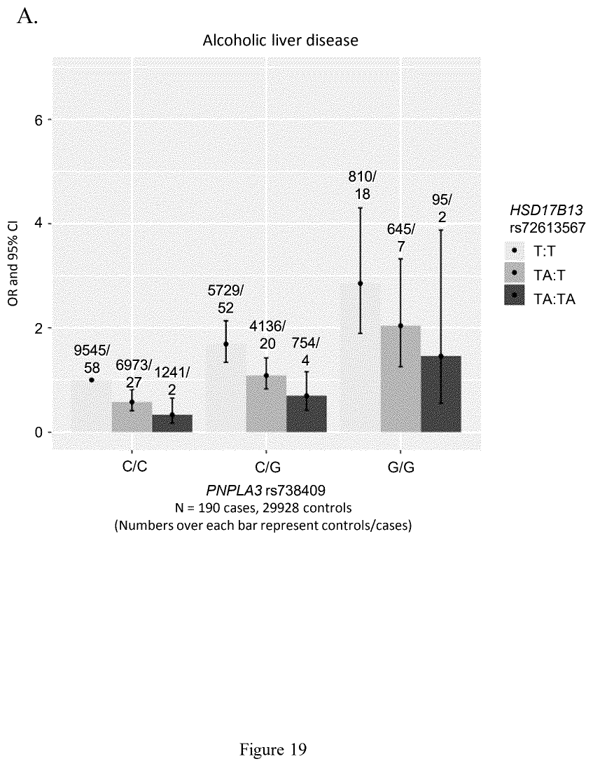

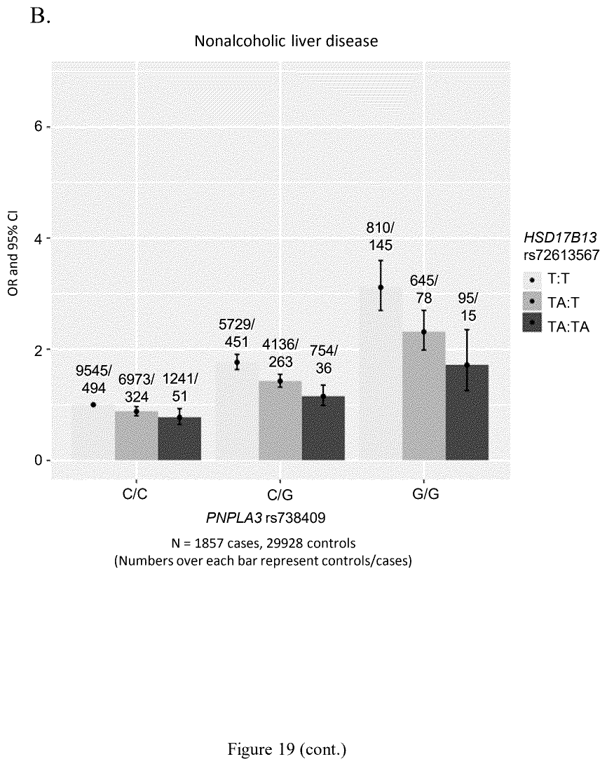

Association of HSD17B13 and PNPLA3 genotypes with liver disease was analyzed by comparing HSD17B13 and PNPLA3 genotypes of 29,928 human liver samples from control donors without steatosis, lobular inflammation, or fibrosis with either 190 samples from patients having alcoholic liver disease, or with 1857 patients having nonalcoholic liver disease. The odds ratio was calculated by the equation of (incidence rate of a group having disease)/(incidence rate of the control group) for each combination of HSD17B13 and PNPLA3 genotype with 95% confidence intervals. Referring to FIG. 19, panel A shows the association of HSD17B13 rs72613567 with alcoholic liver disease in individuals with each PNPLA3 p.I148M genotype, and panel B shows the association of HSD17B13 rs72613567 with nonalcoholic liver disease in individuals with each PNPLA3 p.I148M genotype. The data demonstrate that PNPLA3 p.I148M is associated with higher incidence of both alcoholic and nonalcoholic liver disease in a dosage-dependent manner. The HSD17B13 rs72613567: TA genotype was associated with a reduced risk for both alcoholic and nonalcoholic liver disease in an allele dosage-dependent manner.

1. A method for identifying a human subject as a candidate for treating or inhibiting a liver disease with a hydroxysteroid 17-beta dehydrogenase 13 (HSD17B13) inhibitor, the method comprising:
detecting in a sample from the subject:
i) a nucleic acid encoding a patatin like phospholipase domain containing 3 (PNPLA3) protein comprising an I148M variation; and/or
ii) a PNPLA3 protein comprising an I148M variation.

2.-6. (canceled)

7. The method according to claim 1, further comprising determining whether the subject is heterozygous for the nucleic acid molecule.

8.-12. (canceled)

13. The method according to claim 1, further comprising administering an HSD17B13 inhibitor to the subject.

14. The method according to claim 1, wherein the liver disease comprises an alcoholic liver disease.

15. The method according to claim 14, wherein the alcoholic liver disease comprises cirrhosis, steatosis, or hepatocellular carcinoma resulting from alcohol consumption.

16. The method according to claim 1, wherein the liver disease comprises a non-alcoholic liver disease.

17. The method according to claim 16, wherein the non-alcoholic liver disease comprises nonalcoholic fatty liver disease (NAFLD) or non-alcoholic steatohepatitis (NASH).

18. The method according to claim 16, wherein the non-alcoholic liver disease comprises cirrhosis, steatosis, or hepatocellular carcinoma not caused by alcohol consumption.

19. The method according to claim 13, wherein the HSD17B13 inhibitor comprises an antisense RNA.

20. The method according to claim 13, wherein the HSD17B13 inhibitor comprises a small interfering RNA (siRNA).

21. The method according to claim 13, wherein the HSD17B13 inhibitor comprises a short hairpin RNA (shRNA).

22. A method for treating a subject having a nonalcoholic liver disease, the method comprising administering an inhibitor of hydroxysteroid 17-beta dehydrogenase 13 (HSD17B13) to the subject,
wherein the subject has a first nucleic acid molecule encoding a patatin like phospholipase domain containing 3 (PNPLA3) protein comprising an I148M variation.

23. The method according to claim 22, wherein the HSD17B13 inhibitor comprises an antisense RNA.

24. The method according to claim 22, wherein the HSD17B13 inhibitor comprises a small interfering RNA (siRNA).

25. The method according to claim 22, wherein HSD17B13 inhibitor comprises a short hairpin RNA (shRNA).

26. The method according to claim 22, wherein the subject is heterozygous for the nucleic acid molecule.

27. The method according to claim 22, wherein the non-alcoholic liver disease comprises nonalcoholic fatty liver disease (NAFLD) or non-alcoholic steatohepatitis (NASH).

28. The method according to claim 22, wherein the nonalcoholic liver disease comprises cirrhosis, steatosis, or hepatocarcinoma not caused by alcohol consumption.

29. The method according to claim 22, wherein the nonalcoholic liver disease does not comprise liver fibrosis.


SEQUENCE LISTING (66 sequences)

SEQ ID NO: 1 - DNA, 19118 bp, Homo sapiens
  source 1..19118: mol_type = genomic DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtaatgtatacatcttccaactttttaaagtcacagagtaagatatgtattttaagaattatttgacttaccatctacttatctttgtatttttgtttttcaaagtttgataaattccctggtcccttagtctgtatatgtgtcaggttagttagatgaagggaatgtaattaagaactaagcagcgatttttatgacatggtgtgcaggttgatagaaagactcaggagccagtctccttccaagctgctaaatgaggcaagtcacatattatctctcagcctgttttcttggctctgaagtggggataataacttaggggatgggcaagaacgggatctgaaaattacagctacaaacaaaagtcaaacgaagaacttgcaacagaaacctttagtgcctcccctcatgcacaagcaacacagttctaaaatatttactgtctgaccctttacagaaaatgtttgccagtccgtagtcaaaaggattaaataagtaatattttcagcacttagcatatgataaacgatacgtggcacatgataaacaataactgtgttaaataaaatatgtgcgcagtgagtcaggcttttccttggacattagtatttttcctgtgttcttacttgtaaacactacattaacaaccccaaataaaactgaaggaactgaaatcttgtatcattttctctaaacttgtaaattctggtaaggccatgaaaatatatgcagagaagtgtttacaggattttaggattggaaaaattgtgaagtactccttgagaatcacattttctgcaaattacagtggttttaattaccattatattattactttctcatgttctttgctgtcatgtttagttgaaacctaaaatgtctcttacacttagagaactaattcttttctgttttttttctgaatagtgaagaatactatacaaaaaagctactacatttttatttaacagatatgagcatttatataatagaggagttgatgtatataaaaatgatttgccatctttttggtctttgaagaaattcgaatgaactttctggaagatagcaagaatttacaaatagagaaaattgttgcctgctgttctcaggcatttgtccaaaaatataaataagtataaatctatgaaaagggcttgatgaaatctaaccttcaaatctctttccagatgtgtatttttggggaaagggctatatttattaagttttttttaaattttaaaatttccagagacaagagaaaagtaaattagaaggaagtcgtattaaaaatgacttaagggcgggtgcagtggctcacacctgtaatcccagcactttgggagacggaggtgggcagattgctggagcccaggagttcaagaccagcctgggcagcacagcaaaacccccaactctacaaaaaatacaaaaattagctgggtgcgggggtgcacacccgtagtcccagctactcgggaggctgaggtgggaggatcgtttcagttcaggaagccaaggctgcaatgagctatgatggcatcattgcactccaagctgggcaatagagccaggctctgtctcaaaaaaaataaaaaaagacttaagaaaaataggtaacccaacctcaaaaattctctttgaatcattaaatttcatggttaaacatttaagctactgaatgattcactctaaggctgtaatgtaactcagatctcctttaggcgaggaagatgctggctgagttttcatcataactggctccttttgccctgtgagatgagagacacagtagcagtttggctcttatgcaatctaaactgttgcgttgggaatacggttcaaaaaacacattggagtttaagctaaagcaagtgttttgctaacaaaaagacaaggcatcacattttgcaattgtctagctcagttataaaacagaagaataggccggacgcggtggctcacgcctgtaatcccagcactttgggaggccgagacgggcggatcacgaggtcaggagatcgagaccatcctggataacacagtgaaaccccgtctctactaaaaatacaaaaaaattagccaggcgtagtggcgggcgcctgtagtcccagctactcgggaggctgaggcaggagaatggtgtgaacccgggaggcggagcttgcagtgagccgagatgacgccactgcactccagcctgggcgacagagcgagactccgtctcaaaaaaaaaaaaaaaaaaaaaactgaagaataattaattcttcaatcaaaacatctgatgaatgctctggtaacttatgctctctactgacctagaaacaaatgagagagtatggtgtggtttgtgcaatctggcagtgagcaagctaccaactaaatcagtgaaagactctcctattctttttttactcttctgcaatcccacaaaaggctatttgaggggatactgactttgagactgggtcctaacatccatgtttggggagttcaggctgctgctccagggtttagcctacagtagcgaaatacaaaggacccagagaccactcattcaaggtttgccctaaatagcagcaacaccactgtcatctcaatacacgaagaatagggcttttcaggtatccttgcctctttgtcacagagaagagtttacagattgtgagacggaaaagtataatttttaaaaccttataatattttctataaaagtcacctgaggtgaaaacttgaaaagaattataattttccagaatgtgagtcaagaaacattagagcaattttatcttaggaaagaggtctttgaatttaggctgaaagtaaattgctctgtctccatgtcctatggttatgggcaagtttggtacataaatgagaaatccatccagtggccttgcccatctcactcccaaacacctgaagaatgtaatgttatatctcctagagtagcagcatggtctccctatgaaagtccttcttctttaaggagacttctttcccttccctcctaggaggatgagtcagaatcatcaagaaaaatatgatgggcagaggcatacagtttaccattaccactagtttagaattactacttagcactttactgcctattacatagttggtgctcaacaaatgtatgataaattaatggttgagtttttctttcttctccatattcatcttccatgacaccacgaagagcaatgtttttcaagaatgttcttcaaggtttgaaagtagcctgctttagagaaactgcctactgtacagcctccaaccaagaggaaaagctgaaaaaagcatgaagggattttgttttgttttgtttgttttggttttaatatgagcattccctggcagaaaagccaggggtaatctcattgcaactaggcaatcactctcaagaaattttctaacaaataaggaggccaatttttattttattttgagacgaagtcccactctgtcacccaggttggagtgcaatggaatgatttcagctcactgcaacctccgcctcccgggttcaagtgattctcctgtctaaacttcccgagtagctgggattacaggctcccaccaccacgcccagctaattttttgtatttttagtagagatggggtttcaccattttggccagactggtctcaaactcctgacctcaagtgatccaccctcctcggcctcctaaagtgctgggattacaggcgtgagccaccacacctgacccaggaggccaatttttaaaaggttaactaatcttcatgtccaaaatgaatgttaattgttcattttggacatgaatgttaattttttttttttttttttttgagacagagtctcactctgttgcccaggctggagtccagtggcactatctccactcactgcaacttcctcctcccaggttcaagcaattatcctgcctcagcctcccaagtagctgggattacaggcccacaccatcaggcctggctaatttttgtatttttagtagagacggggtttcaccatgttggccaggctggtcttgaactcctgacctcgtgatccgccctcctcggccaaccaaagtgctgggattacaggcgtgagccaccgcgcctagccgaatgttaattgtctaaaaatttttcttctccaatgtcttctcctccacttttttcggaatttgtttcttcctaattacagcgcggtgtggaggaaactgcagctgagtgccgaaaactaggcgtcactgcgcatgcgtatgtggtagactgcagcaacagagaagagatctatcgctctctaaatcaggtgagactgcaggttcacaaatttcttcagattattttgtttcctaggacgctgacgtggaaaatgagaaaggtctttatgactgcctgatttaaattggattttagctgctaactgaagtagttatgtcaccaaggaaggatatatactttttttcttgtatgtaatccactcagctctgcccattattattgttcatattattaatcaatttcattctgatcagaagtgtgagcagtggcacagagtgactgacaaaagatttatcatcagggaatatggatcacttcctagttttgttttagtcctattaactttgcagtaattccagcttctctttaattatttcccttgtgagattttattttggtgttaatgtagtcttctgtagaaaatgtaatattaataattattatcacaattattttaaaagagtaaataccaaataatcacaatgaactaagcactctaacaaactttacattttttaattcaatccctacaataactctgtaaacttcattttacagataagcaaattatgactcagagaggttaagccagacccaggtcatgtagttattaggttatgaaaccaggatttctcaaccagcactttagaccaggtgcggtggttcacacatgtaatcccagcactttgtgaggccaaggtggaaggatcacatgagaccaagagttcaagaccagcccaggcaacatagtgagaccctatctctaaaaaaaaaaaaaaaaaaaaaaaaaaaagtttaaagaaaaacacatttttaaaaaatgaacactttaaaaatatttggtcagaatttatataggaatttatcaacataaatgttaatttcactttactgataaacttgcaaaacatgatgtgctgggtactgaaatttagatgttaaaagaacagtttatcccacctttatgacagtgttcccttggcctccacgatttgagctcaacagtctgtcttgcctgaactctgagagacctcatacaatagaagaaagactctcatctttggattatattggtcccaaaactttgagtttgaataatacacccagtgaaagtgttctttcaatttcaaaaggtgaagaaagaagtgggtgatgtaacaatcgtggtgaataatgctgggacagtatatccagccgatcttctcagcaccaaggatgaagagattaccaagacatttgaggtcaacatcctaggacatttttgggtgagtgtgagtcagaaacatttctgatttgtgcaccttctcttaagatacatgaaacttataacggagttcacatacttctggacaggaaactggccagatctttgccttaatcaagaatcattaaatttgtttgagtagaagagccacagagtctctgacacaaggacacagaattcaagtggacacaacacaccagaatgtaagctacttggtctgtcttgtccaccagtatctgacacaaagcttggcatgtaccaggagctcaacaaatgtttgtggaggtttgttaagggttgtcagtgtacatcttttcaatgctgtcacttgtgacttcatttttttccctccacaccatgattttgtaatgtgtcctcattttgtggaattttagaatggaaaggacatcagaagtaattacttggatgtatataggatcgaggacacttttggacgagactctgaggcaagtgttctagatccatggggtgctggaactgagaaatgcagctatacagacctcatataattggttagttttgtgggagatggaaatatcaacttcaactgcctttgtatagaaatttttatgattaatcttccagtgcctcaatattagtgtagaatctagggcagatctggattctagaagaaagaagaaaaaaaagagatgtgtcccccttacctttaccagctcttcacatatgtgaattggctcccatgcccaccaaactacacggagacctcatacattagctacctatagctgcataacaaattatacaaaacttagtggtttaaagcaacaatgtatgttcactatcctctcacagtttctatgggttgggaatttggaggtagcttgggttgggagttctagttctatgaatttgcataggatttattaaattcttataaaattttattgatgtttctcacaaaagaggtttttggaaaaaaagaaagacttgttttctgtaacatcaacatataatatacaatattacaaatagggagatagtgaattcaatcatgattcattagtgtggtgtagaactctcagcttacactactcaactgtcttaatacagttacacaagatttcactcttttaattagaatgataaagccccaaaccaaaaaattatatgacaccaaattatcataaggaataattttagttctgaaaactctgaatttttcccttaatattgtttagatgacatatccaaaaaaggatctatttgattccttctgaagggaaggagggggagtactgagattagtgttggcatggggcttaccataccaataaatttgtatctttatttctatcatttgtaaagaattaatcatggaatgcttggaagtattttatttcattgtataagttctctcaaatgcctttctgtcttaacaaaaataaaactacctgatttggaaacctaacgtctatgtcattgtctttcttctttctgcaatgatccttaagatcacaaaagcacttcttccatcgatgatggagagaaatcatggccacatcgtcacagtggcttcagtgtgcggccacgaagggattccttacctcatcccatattggtaagtatcacatgccagccatgttatatatttttatactttgaagggagcattacacttcaaattgttaccactggagagtcctggttcttggcatcttgaacaaagaattggacaaaactcaccaacaaagccaggaaagaatgaagcaacaaaagcagagatttattgaaaatgaaagtacgctttacagggtgggagtgggcccaagcacaggggctcaagagccaattacagaattttctggggtttaaataccccctagaggtttccactggttacttggtgtacgccctatgtaaatgaagaggatgaattaaagttacagagtcgtttactcagtgtacaccatatgtaaatggagaggatatttcctgtcatagctggagtgtttccatttgatttagttctaggaagtcagcatgaatcggccttatgttccctgcctccagaccctgttctcctgcctcaagattacaatgctgagagcagagtgatttggatttacagaatttaaatttatagtagtttagaatgattttttaaatgactttttctaaaacaatgaaaccaggttgtaattatatttaagatatttttagatttctgcaaactcctctgtagaacaatgagagaaaacagtaatgccaagcatgtttccattgtttcctggaataagaaacagaaaccccacagactgagaagcaaaacctacagaagctaaaatgaacacatgtctatgtcatggccttggtgcccaagataagacaatcagagtggtccctggatcaaaacattttacagtgtgcttgtgccatgaaagtgtgtgtgtgtgtgtgtgtgtgtgtgtgagagagagagagagagagaaaacgactctacctgactaaaagttgcagataccacactccatgcaccaccaaagacataaagggaaggaggtgagaggcgttaaggatgtactgctgtatttgccaaatatcctttcctgtaaactcttctccagatcctcataataaaattaagaggccaaagtggcaaccattgtcaagagaaaaactatcaaccattgtcaagagaataactcagttattgagagagagaggagaaatgagcagagtcctacagaagtctgtcaacacagataccagttttgtagaatttctaaatgtatttttcctgattcatatttttcaaaataaaagcagcaataaaaactgattagaaaacagtttgaagattcaatggaaaaaccttacatgtaggatggaaaactgaacattaagccaatcaatagagttatttttgttcttttgttatcattgttgtttaagaaatgagatacgttcacaattctgcttaatcatgtaagaaaatgaaaatgaattgccatttatactctcagaaaaatcacaagtggctgatttttggcttccacttgttcttaagccaaatgataccgccttctcacagaaagctgaggattggtttcactctcccttagctaacaatgcttaataattctcttacagttccagcaaatttgccgctgttggctttcacagaggtctgacatcagaacttcaggccttgggaaaaactggtatcaaaacctcatgtctctgcccagtttttgtgaatactgggttcaccaaaaatccaagcacaaggtaaggtcaaaatcaagttagaatgggtatgtggtatgataaattgatatgaaaactaatgagaaatgtttaggcaggccaactaatagaagaaaatgaagaaggaaaaataatttttcttattattattattatcttgaaattaaaggaataaagggggaaaacacattagggactagcaggaatgatcagccaccgatgaactgggatatttatttgtgtccgggagaaagcacatacatttgatcaccgttaccaccctgtctttaaaatgcaaatgttccaaggaccagcaaataaattgagtatctagctccttagtcaaggtgaatttctgcaagaactcttgtctctggtgagacaggatttgagaccacaagagaagaaaaattagtcctgaaaggagaagaaaaaagcaggaaggtgtggataagaacccgaaaattaagccatctgcttaacaaatttttctaatcctagtatatattctgctgcaggttaacaaaatatactaagcttaatgattcgaaaccaattttttactggaagggaattaatcctaaatatattcattcaaaagaactaaacaattctctgttgagtgccgcctcatttgaggatactgactcttacagcctgagttagctatgtggtctctgcagctggaatcactccctgccactggagtccttcatggtgttagaccataggtactgttgactaaagaaaaaaaaaagtttttgtttttatttttgttttttttgagacagagtctcactctgtcacccaggctggagtacagtggcgcgatctcagctcaccgcaacctccgcctttctgggttcaagcaattctccttcctcagcctcctgagtatttggattacaggcgcccaccaccacgcctggctaatttttgtatttttagtagagacggggtttcaccatgttggccaggctggtctcaaactcctgacctcaggtgtcctacctgccttggcctcctaaaatgctgggattacaggagtgagccaccatgcccggccaaaaaaataagtttttaaagaattaaaggtcatcctggctaacacagtgaaaccccgtctctactaaaaaacacaaaaaaattagccgggcgtggtggcgggcgcctgtagtcccagctgcgcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaaaaaaaagaattaaaggtgttaattttatttagaagccttactgaagactacagtcggaggcctatagcctgagagcagccctttagagaggttcagttgaactgttctgatagtgggggccatgtgctctatcctgtattgtcttcaaagcatctttccagagagctgcacattgtcacagagtcagggactttgtgaaattatgctgacaaccagaagtgagtaaacgtggcttcttacatttgctacgttgtctcacagtacttaataagtatgcaatatgtaagtaaatactatagtactattgcaactcctgattgttttcttagacaaggaattgggcccaataaaaaccctcttggtaggcattcaggcttcgtgtaccatgagctttcctaagggtatcctgccactcttggggaaggcatgatagatgaggggagtaaggataatggaactctgggtacagggttcctgggggctaacttagaggtagacacaggcaatgctaaatatttgggattgattttatagaggttgctagattgtgaatttccttagtaagggctaaggcattgatatgtaatgtcacacttggctccgaggctgggttgttggatccatgtagatgaaatcagggagagaaagggcagaacggagtaatttagaaatgtattgatttgtattactctctgttggcttgctattcaaggcagtggagaactcaatcacataataatctgcagcaaaccacagatcatcccagggaatgaagttttaacattcgctggctccctaactcctcacccagcctttacattcactggctgttcagtccatgcctggacatcttaatttgaatacaacattttaaatccatttttctgtcatcatcttgcactaacagacaattctacactaagcctatgtttatgaatatttctcaagagtacatgtacacagccttcagtataaggaaaactggaagtatgacatacctccagttgtcatactccttgggcccctcttaaattctcattaaactgcaggataggcaagtcagaggtgaatctcaaatacgaaattcttaccggaaaggggttccaatccagaccccaagagagggttcttagatttctcgcaagaaataattcggggcaaggccacagtgcaaagcaaaagcaagtttattaggaaagtaaaggagtagagaacagctactccatggagaagaatggcttgagctgctccaccaagggtatttagagttacttcttgattatatgctaaacaaggggtggattattcatgagttttccgggaaaagggtgagcaattcccagaactgagatttcctcccctttttaggccatatagggtaacttcctgccattgccatggtatttgtaaactgtcatagtgctggtggaagtgtctcttagcttgctaatgtattatagttagcttataatgagcagtgaggacaaccagaggtcactttcatcaccatcttggttttggtgggttttggccggcttctttactgcaccctattttatcaacaaggtctttatgacctgaatcttgtgccaacctcctatctcatcctgtgacaaagaatgccttaacttcctgggaatgcagcccagtaggtgtcagccttattttacccagaccctattcaagatggagttgctctgatttaaacgcctctgacaaaatgacgacctcaaaacaatccagctttatggaatacctccacaagaaagaaagtatacttagctatagaattttctccttgcatccaacaggctttgagatgtcagatgtttccttcctgtcccatgattaatcctagccattcctctttcttgtctggctccactactccttaccatctaatgcctcgccaccattttgatattttgactaagtgagctatgaaacacacctactggatatgaaagtataagtttctgataacaaaacatcaacatgggatgtggaggaagtgggtagggtggcattaatgcagcaaatcctggaatattttaaatcttcattctaaatttagtaaaaatataggataattttcctgccatcatttacttataaaattaaaattttagaaaataaaaataatattttcctctttttaatcacagattatggcctgtattggagacagatgaagtcgtaagaagtctgatagatggaatacttaccaataagaaaatgatttttgttccatcgtatatcaatatctttctgagactacagaagtaagtacagcacagaacacccaaatactaaaacaccaatagagcttttttttttgctttttttttttttagacagagtctcactctgtcaccctggctggattgcggtggttgcagtggcatgatcttggctcactgcaacctccgcctcctgggttcaagcaattctcatgcctcagacccccaagtaactgggattataggtgtgtgctgccacactacacccagctaatttttgtattttttgatagagacaggtttccccatgttggccaggctggactcgaactcctgacctcaagttatcctcctgtctcggcctcccaaagtgctgggattacagtcatgagccaccatgcctggcccaatagagctattattatggagcatctttcagttgtgaaaattggcatggaaactctccatccctggggagaacagttatttcctctgttattttcctacccagtctataaaaagagagtgattcattttctctaccaaatctactgtctctgcccaaactttgctgaagactattctaactaaaggaaacacagtttaaaaagaatgcaatatagtgaagtagttaataataaagactccatttttaaaagtctgctggaagtttggttgggattgcactgaatctatagagcaattggggagtattgacatatcaacaatattgagttttctaatccaagaacataatatctatttttaaaatcttcttcaaaatctttaaatctttaaattgtattttgtagtttttggtgtttaagtcttgcacatattttgtcagatttattccaaagtatttcacgggttcttttttttttttttttttttttttttgagacagagtttcacccttgttgcccaggctggagtgcagtggcgtgatcttggctcactgcagcttctgcctcctggcttcaagtgattctcctgcctcagcctcccaagtagctgggattacaggcacctgccccctcgcccaactaactttttgtgtttgtagtagagacagggtttcaccatgttggccaggctggtctcgaactcctgacctcatgtgatccacctgcctcagcctcccaaagtgctgggattacaggcatgagccatcatgcccagccctatttgacggtttttgacgctaatgcaagtggcattttaaaaaattttatatttcccattgtttgttgtcagtatatattggatttttgtaatttgatctcatattttgcagtcttgctaaattgctaaacctctttttgctaaactcgataagcttttttttttttggtagattcctgggcctctaattttctttatgggaaagtttttaattacaaatttaatttctttaatagctacatggctattcaatttacttattaattcttggtaatgtgtgtctttcaaggaatttgtccatttcatctaagttgtagaatttctttggcataaatttgtacataacattcccttattatccttttaatgtctttagaatgtcttatttatttatttatttatttttattatatttttttgagacagagtctcgctctgttgcccaggctggagtgcagtggcacaatcttggctcactgcaagctccgccttctgggttcatgccattctcctgcctcagcctccctagttgctgggactacaggcgcctgcaaccatgcccagcttattttttttttttttttttttttttttttttttttttttttttttttagtagagacggggtttcaccctgttagccaggatggtctcgatctcctgacctggtgatccgcccgcctcagcctcccaaagtgctgggattacaggcgtgagccaccaagcccagcctatttatttatttagtagagacagtctcactttgctgcccaggcaacaaaggttttgaatgcctggcctcaagcagtcctcctgccttggcctcccaaagtgctgggattacaggcatgagccactgcacctggccaaatgaatatgctgataatatcttctttataaggatgacataagaataaaataatgtaatacaaacaaagcccctgtcactgaaaatgtatagacttcaaatgttaaagtcttagagaacagaatttatatgaaatagcaacagcaacaatttcccagaggaaatactctctcagctttcttctgaggagcagtttctaaattgaaattgtatcagtgagaagataactatactaacttcataagccttgggcctttttgaaacaaatccatataaactatgaacaaacttgaaatagaacaatttgagaacagggtacaaactgcattggtgtatcaatttcagtatttggttttagcttaaatagactgacttgagataacataaggagaaccttgacccccaagcaacatcatctcgcgagttgactaggccgggtgtggtgtctcacgcctgtaattccagcactttgggaggccacagcaggcagatcacttgaggtcaggcattcgagaccagcctggccaacatggtgaaacctcagctctactaaagatacgaaaattagcaggcatagtggcctgcacctgtaataccaggcactcgcaggagaatcccttgaacccggaaggcggagattgcagtaaaccatgattgtgccactgcactccagcctgggcaacaggagactctgtctcggaaaaataaattttttaaaaaaatgaaaaaaaataaaagttgactaaattagtgtcttggtactaagcactgtaggaagtgagtttcatggaaccccaactctcttggggcccaaagcaagtcatattaatattgaaaattacatgcatatacatgcatatgaccaaggtgataaaaacaattattctgcctgagttggagaatagtatcccagtaaaataaacaagagtctcaaagtcttttgtatcctttgaagctgtcatggtggtttgtaactaggcaacaggtatatattgttaatcttctttgcatttaattccttttatagagagacacaattttacgagcagatgcaattactagcatgaaggtttctttgtgagggtagttaaaaggcccacatgagctctcttcttatccttgtccttctttcagccagatcttccctgcccctttgctcattccatctttcacccacctacccccaaaacaaggaagtaaatcttgcattagtcaacaataccaaagtgattttcaatatgactttctctgcagaatgttattatttctgcctctttacattcacatactgtcttcctttttttttttttttttttttttttttttagattgggtctcactctgttgcccaggctggagtgcagtggcttgatctcagctcactgtaacctccacctcctgagttcaagcaattctcctgcctcagcctcctgagtagctgggattacaggcatgtgccaccacacctggctagtttttttgtatttttagtagagacagggtttcaccatgttggtcaagctggtctcgaactcctgacctcatgatctgaccacctgtgcctctcaaagtgctgggattacaggcgtgagccaccgggccagccactctcttcctttcagttgcctactcatctcttatgcattcctggacatcagttgtccttttgaagctttcctccactatcccagcccatgtgaatcctccttccagttatagcccttaattctagatggctgatatttttcaataattgttttaagatgaccattttagcctatcagctaaacaatatcaaagacaatagctatttttcaagtactttagtttaccttattatagagtgcataatagatattcagtaaatagtaaaggagaggtgaaggcttgcatagaatggattctggtggtgtctcttggtgagcttttagcatcaagattaatcagcagtttcagcaatgagctcagaccttcagttttagatctttactcatatcagataagagagtgagaagagtggtatgtatcagtgctttatttatatttgcatccaatttgaactatgaatattacaaaggtgcacacataggttcagacagattgatttaaaatgaccaaagatgacctgtcgtaagcaacctgggtatcttaagatgcactccttggagagggaatgttcctaaaaacattttcagagggacgaactgtatgaaattcagtaaaacataaatcatgaggaaaactgattactctctttttgacatgaaatgagagttttaatgcatggttacgattattaacgtactccgctgcaagacgttaataaagttactgttttgcaggctagaatgtcttgatgctgtaatcagaacacactttttcccctttcttccagcttcaaatgcagattcataattgggctgacttctaataactgcaatgttttctgccttgggcttgcagcagaagcctgacaaaatagtgtttgtttaggcaataatttatttatttatttattgagatggagtttcattcttgtcgcccaggctggagtgcaatggcgtgatctcggctcactgcaacctctgtgttcaggcaataatttagactttaccttacttgtgattactatagcaattactatagccacaaggcataattttactgtctcatttcaattttatgaatttgaatgtttttacacttttcctaatgaagtccactatgaagttatgtcaaaaaaaaaaaagaaaaagaaagatgcacacgtaaaagagaggtggttgcaagagaagaaaagaacggaggaaagttaaacgcaaaccagataactctcagcgtattctaaatgaccaaaaacagaactctgttgtcaaagattttaaatggaaaatttttcaatttttttttcttttttgtacaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 2 - DNA, 19119 bp, Homo sapiens
  source 1..19119: mol_type = genomic DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtaatgtatacatcttccaactttttaaagtcacagagtaagatatgtattttaagaattatttgacttaccatctacttatctttgtatttttgtttttcaaagtttgataaattccctggtcccttagtctgtatatgtgtcaggttagttagatgaagggaatgtaattaagaactaagcagcgatttttatgacatggtgtgcaggttgatagaaagactcaggagccagtctccttccaagctgctaaatgaggcaagtcacatattatctctcagcctgttttcttggctctgaagtggggataataacttaggggatgggcaagaacgggatctgaaaattacagctacaaacaaaagtcaaacgaagaacttgcaacagaaacctttagtgcctcccctcatgcacaagcaacacagttctaaaatatttactgtctgaccctttacagaaaatgtttgccagtccgtagtcaaaaggattaaataagtaatattttcagcacttagcatatgataaacgatacgtggcacatgataaacaataactgtgttaaataaaatatgtgcgcagtgagtcaggcttttccttggacattagtatttttcctgtgttcttacttgtaaacactacattaacaaccccaaataaaactgaaggaactgaaatcttgtatcattttctctaaacttgtaaattctggtaaggccatgaaaatatatgcagagaagtgtttacaggattttaggattggaaaaattgtgaagtactccttgagaatcacattttctgcaaattacagtggttttaattaccattatattattactttctcatgttctttgctgtcatgtttagttgaaacctaaaatgtctcttacacttagagaactaattcttttctgttttttttctgaatagtgaagaatactatacaaaaaagctactacatttttatttaacagatatgagcatttatataatagaggagttgatgtatataaaaatgatttgccatctttttggtctttgaagaaattcgaatgaactttctggaagatagcaagaatttacaaatagagaaaattgttgcctgctgttctcaggcatttgtccaaaaatataaataagtataaatctatgaaaagggcttgatgaaatctaaccttcaaatctctttccagatgtgtatttttggggaaagggctatatttattaagttttttttaaattttaaaatttccagagacaagagaaaagtaaattagaaggaagtcgtattaaaaatgacttaagggcgggtgcagtggctcacacctgtaatcccagcactttgggagacggaggtgggcagattgctggagcccaggagttcaagaccagcctgggcagcacagcaaaacccccaactctacaaaaaatacaaaaattagctgggtgcgggggtgcacacccgtagtcccagctactcgggaggctgaggtgggaggatcgtttcagttcaggaagccaaggctgcaatgagctatgatggcatcattgcactccaagctgggcaatagagccaggctctgtctcaaaaaaaataaaaaaagacttaagaaaaataggtaacccaacctcaaaaattctctttgaatcattaaatttcatggttaaacatttaagctactgaatgattcactctaaggctgtaatgtaactcagatctcctttaggcgaggaagatgctggctgagttttcatcataactggctccttttgccctgtgagatgagagacacagtagcagtttggctcttatgcaatctaaactgttgcgttgggaatacggttcaaaaaacacattggagtttaagctaaagcaagtgttttgctaacaaaaagacaaggcatcacattttgcaattgtctagctcagttataaaacagaagaataggccggacgcggtggctcacgcctgtaatcccagcactttgggaggccgagacgggcggatcacgaggtcaggagatcgagaccatcctggataacacagtgaaaccccgtctctactaaaaatacaaaaaaattagccaggcgtagtggcgggcgcctgtagtcccagctactcgggaggctgaggcaggagaatggtgtgaacccgggaggcggagcttgcagtgagccgagatgacgccactgcactccagcctgggcgacagagcgagactccgtctcaaaaaaaaaaaaaaaaaaaaaactgaagaataattaattcttcaatcaaaacatctgatgaatgctctggtaacttatgctctctactgacctagaaacaaatgagagagtatggtgtggtttgtgcaatctggcagtgagcaagctaccaactaaatcagtgaaagactctcctattctttttttactcttctgcaatcccacaaaaggctatttgaggggatactgactttgagactgggtcctaacatccatgtttggggagttcaggctgctgctccagggtttagcctacagtagcgaaatacaaaggacccagagaccactcattcaaggtttgccctaaatagcagcaacaccactgtcatctcaatacacgaagaatagggcttttcaggtatccttgcctctttgtcacagagaagagtttacagattgtgagacggaaaagtataatttttaaaaccttataatattttctataaaagtcacctgaggtgaaaacttgaaaagaattataattttccagaatgtgagtcaagaaacattagagcaattttatcttaggaaagaggtctttgaatttaggctgaaagtaaattgctctgtctccatgtcctatggttatgggcaagtttggtacataaatgagaaatccatccagtggccttgcccatctcactcccaaacacctgaagaatgtaatgttatatctcctagagtagcagcatggtctccctatgaaagtccttcttctttaaggagacttctttcccttccctcctaggaggatgagtcagaatcatcaagaaaaatatgatgggcagaggcatacagtttaccattaccactagtttagaattactacttagcactttactgcctattacatagttggtgctcaacaaatgtatgataaattaatggttgagtttttctttcttctccatattcatcttccatgacaccacgaagagcaatgtttttcaagaatgttcttcaaggtttgaaagtagcctgctttagagaaactgcctactgtacagcctccaaccaagaggaaaagctgaaaaaagcatgaagggattttgttttgttttgtttgttttggttttaatatgagcattccctggcagaaaagccaggggtaatctcattgcaactaggcaatcactctcaagaaattttctaacaaataaggaggccaatttttattttattttgagacgaagtcccactctgtcacccaggttggagtgcaatggaatgatttcagctcactgcaacctccgcctcccgggttcaagtgattctcctgtctaaacttcccgagtagctgggattacaggctcccaccaccacgcccagctaattttttgtatttttagtagagatggggtttcaccattttggccagactggtctcaaactcctgacctcaagtgatccaccctcctcggcctcctaaagtgctgggattacaggcgtgagccaccacacctgacccaggaggccaatttttaaaaggttaactaatcttcatgtccaaaatgaatgttaattgttcattttggacatgaatgttaattttttttttttttttttttgagacagagtctcactctgttgcccaggctggagtccagtggcactatctccactcactgcaacttcctcctcccaggttcaagcaattatcctgcctcagcctcccaagtagctgggattacaggcccacaccatcaggcctggctaatttttgtatttttagtagagacggggtttcaccatgttggccaggctggtcttgaactcctgacctcgtgatccgccctcctcggccaaccaaagtgctgggattacaggcgtgagccaccgcgcctagccgaatgttaattgtctaaaaatttttcttctccaatgtcttctcctccacttttttcggaatttgtttcttcctaattacagcgcggtgtggaggaaactgcagctgagtgccgaaaactaggcgtcactgcgcatgcgtatgtggtagactgcagcaacagagaagagatctatcgctctctaaatcaggtgagactgcaggttcacaaatttcttcagattattttgtttcctaggacgctgacgtggaaaatgagaaaggtctttatgactgcctgatttaaattggattttagctgctaactgaagtagttatgtcaccaaggaaggatatatactttttttcttgtatgtaatccactcagctctgcccattattattgttcatattattaatcaatttcattctgatcagaagtgtgagcagtggcacagagtgactgacaaaagatttatcatcagggaatatggatcacttcctagttttgttttagtcctattaactttgcagtaattccagcttctctttaattatttcccttgtgagattttattttggtgttaatgtagtcttctgtagaaaatgtaatattaataattattatcacaattattttaaaagagtaaataccaaataatcacaatgaactaagcactctaacaaactttacattttttaattcaatccctacaataactctgtaaacttcattttacagataagcaaattatgactcagagaggttaagccagacccaggtcatgtagttattaggttatgaaaccaggatttctcaaccagcactttagaccaggtgcggtggttcacacatgtaatcccagcactttgtgaggccaaggtggaaggatcacatgagaccaagagttcaagaccagcccaggcaacatagtgagaccctatctctaaaaaaaaaaaaaaaaaaaaaaaaaaaagtttaaagaaaaacacatttttaaaaaatgaacactttaaaaatatttggtcagaatttatataggaatttatcaacataaatgttaatttcactttactgataaacttgcaaaacatgatgtgctgggtactgaaatttagatgttaaaagaacagtttatcccacctttatgacagtgttcccttggcctccacgatttgagctcaacagtctgtcttgcctgaactctgagagacctcatacaatagaagaaagactctcatctttggattatattggtcccaaaactttgagtttgaataatacacccagtgaaagtgttctttcaatttcaaaaggtgaagaaagaagtgggtgatgtaacaatcgtggtgaataatgctgggacagtatatccagccgatcttctcagcaccaaggatgaagagattaccaagacatttgaggtcaacatcctaggacatttttgggtgagtgtgagtcagaaacatttctgatttgtgcaccttctcttaagatacatgaaacttataacggagttcacatacttctggacaggaaactggccagatctttgccttaatcaagaatcattaaatttgtttgagtagaagagccacagagtctctgacacaaggacacagaattcaagtggacacaacacaccagaatgtaagctacttggtctgtcttgtccaccagtatctgacacaaagcttggcatgtaccaggagctcaacaaatgtttgtggaggtttgttaagggttgtcagtgtacatcttttcaatgctgtcacttgtgacttcatttttttccctccacaccatgattttgtaatgtgtcctcattttgtggaattttagaatggaaaggacatcagaagtaattacttggatgtatataggatcgaggacacttttggacgagactctgaggcaagtgttctagatccatggggtgctggaactgagaaatgcagctatacagacctcatataattggttagttttgtgggagatggaaatatcaacttcaactgcctttgtatagaaatttttatgattaatcttccagtgcctcaatattagtgtagaatctagggcagatctggattctagaagaaagaagaaaaaaaagagatgtgtcccccttacctttaccagctcttcacatatgtgaattggctcccatgcccaccaaactacacggagacctcatacattagctacctatagctgcataacaaattatacaaaacttagtggtttaaagcaacaatgtatgttcactatcctctcacagtttctatgggttgggaatttggaggtagcttgggttgggagttctagttctatgaatttgcataggatttattaaattcttataaaattttattgatgtttctcacaaaagaggtttttggaaaaaaagaaagacttgttttctgtaacatcaacatataatatacaatattacaaatagggagatagtgaattcaatcatgattcattagtgtggtgtagaactctcagcttacactactcaactgtcttaatacagttacacaagatttcactcttttaattagaatgataaagccccaaaccaaaaaattatatgacaccaaattatcataaggaataattttagttctgaaaactctgaatttttcccttaatattgtttagatgacatatccaaaaaaggatctatttgattccttctgaagggaaggagggggagtactgagattagtgttggcatggggcttaccataccaataaatttgtatctttatttctatcatttgtaaagaattaatcatggaatgcttggaagtattttatttcattgtataagttctctcaaatgcctttctgtcttaacaaaaataaaactacctgatttggaaacctaacgtctatgtcattgtctttcttctttctgcaatgatccttaagatcacaaaagcacttcttccatcgatgatggagagaaatcatggccacatcgtcacagtggcttcagtgtgcggccacgaagggattccttacctcatcccatattggtaagtatcacatgccagccatgttatatatttttatactttgaagggagcattacacttcaaattgttaccactggagagtcctggttcttggcatcttgaacaaagaattggacaaaactcaccaacaaagccaggaaagaatgaagcaacaaaagcagagatttattgaaaatgaaagtacgctttacagggtgggagtgggcccaagcacaggggctcaagagccaattacagaattttctggggtttaaataccccctagaggtttccactggttacttggtgtacgccctatgtaaatgaagaggatgaattaaagttacagagtcgtttactcagtgtacaccatatgtaaatggagaggatatttcctgtcatagctggagtgtttccatttgatttagttctaggaagtcagcatgaatcggccttatgttccctgcctccagaccctgttctcctgcctcaagattacaatgctgagagcagagtgatttggatttacagaatttaaatttatagtagtttagaatgattttttaaatgactttttctaaaacaatgaaaccaggttgtaattatatttaagatatttttagatttctgcaaactcctctgtagaacaatgagagaaaacagtaatgccaagcatgtttccattgtttcctggaataagaaacagaaaccccacagactgagaagcaaaacctacagaagctaaaatgaacacatgtctatgtcatggccttggtgcccaagataagacaatcagagtggtccctggatcaaaacattttacagtgtgcttgtgccatgaaagtgtgtgtgtgtgtgtgtgtgtgtgtgtgagagagagagagagagagaaaacgactctacctgactaaaagttgcagataccacactccatgcaccaccaaagacataaagggaaggaggtgagaggcgttaaggatgtactgctgtatttgccaaatatcctttcctgtaaactcttctccagatcctcataataaaattaagaggccaaagtggcaaccattgtcaagagaaaaactatcaaccattgtcaagagaataactcagttattgagagagagaggagaaatgagcagagtcctacagaagtctgtcaacacagataccagttttgtagaatttctaaatgtatttttcctgattcatatttttcaaaataaaagcagcaataaaaactgattagaaaacagtttgaagattcaatggaaaaaccttacatgtaggatggaaaactgaacattaagccaatcaatagagttatttttgttcttttgttatcattgttgtttaagaaatgagatacgttcacaattctgcttaatcatgtaagaaaatgaaaatgaattgccatttatactctcagaaaaatcacaagtggctgatttttggcttccacttgttcttaagccaaatgataccgccttctcacagaaagctgaggattggtttcactctcccttagctaacaatgcttaataattctcttacagttccagcaaatttgccgctgttggctttcacagaggtctgacatcagaacttcaggccttgggaaaaactggtatcaaaacctcatgtctctgcccagtttttgtgaatactgggttcaccaaaaatccaagcacaaggtaaggtcaaaatcaagttagaatgggtatgtggtatgataaattgatatgaaaactaatgagaaatgtttaggcaggccaactaatagaagaaaatgaagaaggaaaaataatttttcttattattattattatcttgaaattaaaggaataaagggggaaaacacattagggactagcaggaatgatcagccaccgatgaactgggatatttatttgtgtccgggagaaagcacatacatttgatcaccgttaccaccctgtctttaaaatgcaaatgttccaaggaccagcaaataaattgagtatctagctccttagtcaaggtgaatttctgcaagaactcttgtctctggtgagacaggatttgagaccacaagagaagaaaaattagtcctgaaaggagaagaaaaaagcaggaaggtgtggataagaacccgaaaattaagccatctgcttaacaaatttttctaatcctagtatatattctgctgcaggttaacaaaatatactaagcttaatgattcgaaaccaattttttactggaagggaattaatcctaaatatattcattcaaaagaactaaacaattctctgttgagtgccgcctcatttgaggatactgactcttacagcctgagttagctatgtggtctctgcagctggaatcactccctgccactggagtccttcatggtgttagaccataggtactgttgactaaagaaaaaaaaaagtttttgtttttatttttgttttttttgagacagagtctcactctgtcacccaggctggagtacagtggcgcgatctcagctcaccgcaacctccgcctttctgggttcaagcaattctccttcctcagcctcctgagtatttggattacaggcgcccaccaccacgcctggctaatttttgtatttttagtagagacggggtttcaccatgttggccaggctggtctcaaactcctgacctcaggtgtcctacctgccttggcctcctaaaatgctgggattacaggagtgagccaccatgcccggccaaaaaaataagtttttaaagaattaaaggtcatcctggctaacacagtgaaaccccgtctctactaaaaaacacaaaaaaattagccgggcgtggtggcgggcgcctgtagtcccagctgcgcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaaaaaaaagaattaaaggtgttaattttatttagaagccttactgaagactacagtcggaggcctatagcctgagagcagccctttagagaggttcagttgaactgttctgatagtgggggccatgtgctctatcctgtattgtcttcaaagcatctttccagagagctgcacattgtcacagagtcagggactttgtgaaattatgctgacaaccagaagtgagtaaacgtggcttcttacatttgctacgttgtctcacagtacttaataagtatgcaatatgtaagtaaatactatagtactattgcaactcctgattgttttcttagacaaggaattgggcccaataaaaaccctcttggtaggcattcaggcttcgtgtaccatgagctttcctaagggtatcctgccactcttggggaaggcatgatagatgaggggagtaaggataatggaactctgggtacagggttcctgggggctaacttagaggtagacacaggcaatgctaaatatttgggattgattttatagaggttgctagattgtgaatttccttagtaagggctaaggcattgatatgtaatgtcacacttggctccgaggctgggttgttggatccatgtagatgaaatcagggagagaaagggcagaacggagtaatttagaaatgtattgatttgtattactctctgttggcttgctattcaaggcagtggagaactcaatcacataataatctgcagcaaaccacagatcatcccagggaatgaagttttaacattcgctggctccctaactcctcacccagcctttacattcactggctgttcagtccatgcctggacatcttaatttgaatacaacattttaaatccatttttctgtcatcatcttgcactaacagacaattctacactaagcctatgtttatgaatatttctcaagagtacatgtacacagccttcagtataaggaaaactggaagtatgacatacctccagttgtcatactccttgggcccctcttaaattctcattaaactgcaggataggcaagtcagaggtgaatctcaaatacgaaattcttaccggaaaggggttccaatccagaccccaagagagggttcttagatttctcgcaagaaataattcggggcaaggccacagtgcaaagcaaaagcaagtttattaggaaagtaaaggagtagagaacagctactccatggagaagaatggcttgagctgctccaccaagggtatttagagttacttcttgattatatgctaaacaaggggtggattattcatgagttttccgggaaaagggtgagcaattcccagaactgagatttcctcccctttttaggccatatagggtaacttcctgccattgccatggtatttgtaaactgtcatagtgctggtggaagtgtctcttagcttgctaatgtattatagttagcttataatgagcagtgaggacaaccagaggtcactttcatcaccatcttggttttggtgggttttggccggcttctttactgcaccctattttatcaacaaggtctttatgacctgaatcttgtgccaacctcctatctcatcctgtgacaaagaatgccttaacttcctgggaatgcagcccagtaggtgtcagccttattttacccagaccctattcaagatggagttgctctgatttaaacgcctctgacaaaatgacgacctcaaaacaatccagctttatggaatacctccacaagaaagaaagtatacttagctatagaattttctccttgcatccaacaggctttgagatgtcagatgtttccttcctgtcccatgattaatcctagccattcctctttcttgtctggctccactactccttaccatctaatgcctcgccaccattttgatattttgactaagtgagctatgaaacacacctactggatatgaaagtataagtttctgataacaaaacatcaacatgggatgtggaggaagtgggtagggtggcattaatgcagcaaatcctggaatattttaaatcttcattctaaatttagtaaaaatataggataattttcctgccatcatttacttataaaattaaaattttagaaaataaaaataatattttcctctttttaatcacagattatggcctgtattggagacagatgaagtcgtaagaagtctgatagatggaatacttaccaataagaaaatgatttttgttccatcgtatatcaatatctttctgagactacagaagttaagtacagcacagaacacccaaatactaaaacaccaatagagcttttttttttgctttttttttttttagacagagtctcactctgtcaccctggctggattgcggtggttgcagtggcatgatcttggctcactgcaacctccgcctcctgggttcaagcaattctcatgcctcagacccccaagtaactgggattataggtgtgtgctgccacactacacccagctaatttttgtattttttgatagagacaggtttccccatgttggccaggctggactcgaactcctgacctcaagttatcctcctgtctcggcctcccaaagtgctgggattacagtcatgagccaccatgcctggcccaatagagctattattatggagcatctttcagttgtgaaaattggcatggaaactctccatccctggggagaacagttatttcctctgttattttcctacccagtctataaaaagagagtgattcattttctctaccaaatctactgtctctgcccaaactttgctgaagactattctaactaaaggaaacacagtttaaaaagaatgcaatatagtgaagtagttaataataaagactccatttttaaaagtctgctggaagtttggttgggattgcactgaatctatagagcaattggggagtattgacatatcaacaatattgagttttctaatccaagaacataatatctatttttaaaatcttcttcaaaatctttaaatctttaaattgtattttgtagtttttggtgtttaagtcttgcacatattttgtcagatttattccaaagtatttcacgggttcttttttttttttttttttttttttttgagacagagtttcacccttgttgcccaggctggagtgcagtggcgtgatcttggctcactgcagcttctgcctcctggcttcaagtgattctcctgcctcagcctcccaagtagctgggattacaggcacctgccccctcgcccaactaactttttgtgtttgtagtagagacagggtttcaccatgttggccaggctggtctcgaactcctgacctcatgtgatccacctgcctcagcctcccaaagtgctgggattacaggcatgagccatcatgcccagccctatttgacggtttttgacgctaatgcaagtggcattttaaaaaattttatatttcccattgtttgttgtcagtatatattggatttttgtaatttgatctcatattttgcagtcttgctaaattgctaaacctctttttgctaaactcgataagcttttttttttttggtagattcctgggcctctaattttctttatgggaaagtttttaattacaaatttaatttctttaatagctacatggctattcaatttacttattaattcttggtaatgtgtgtctttcaaggaatttgtccatttcatctaagttgtagaatttctttggcataaatttgtacataacattcccttattatccttttaatgtctttagaatgtcttatttatttatttatttatttttattatatttttttgagacagagtctcgctctgttgcccaggctggagtgcagtggcacaatcttggctcactgcaagctccgccttctgggttcatgccattctcctgcctcagcctccctagttgctgggactacaggcgcctgcaaccatgcccagcttattttttttttttttttttttttttttttttttttttttttttttttagtagagacggggtttcaccctgttagccaggatggtctcgatctcctgacctggtgatccgcccgcctcagcctcccaaagtgctgggattacaggcgtgagccaccaagcccagcctatttatttatttagtagagacagtctcactttgctgcccaggcaacaaaggttttgaatgcctggcctcaagcagtcctcctgccttggcctcccaaagtgctgggattacaggcatgagccactgcacctggccaaatgaatatgctgataatatcttctttataaggatgacataagaataaaataatgtaatacaaacaaagcccctgtcactgaaaatgtatagacttcaaatgttaaagtcttagagaacagaatttatatgaaatagcaacagcaacaatttcccagaggaaatactctctcagctttcttctgaggagcagtttctaaattgaaattgtatcagtgagaagataactatactaacttcataagccttgggcctttttgaaacaaatccatataaactatgaacaaacttgaaatagaacaatttgagaacagggtacaaactgcattggtgtatcaatttcagtatttggttttagcttaaatagactgacttgagataacataaggagaaccttgacccccaagcaacatcatctcgcgagttgactaggccgggtgtggtgtctcacgcctgtaattccagcactttgggaggccacagcaggcagatcacttgaggtcaggcattcgagaccagcctggccaacatggtgaaacctcagctctactaaagatacgaaaattagcaggcatagtggcctgcacctgtaataccaggcactcgcaggagaatcccttgaacccggaaggcggagattgcagtaaaccatgattgtgccactgcactccagcctgggcaacaggagactctgtctcggaaaaataaattttttaaaaaaatgaaaaaaaataaaagttgactaaattagtgtcttggtactaagcactgtaggaagtgagtttcatggaaccccaactctcttggggcccaaagcaagtcatattaatattgaaaattacatgcatatacatgcatatgaccaaggtgataaaaacaattattctgcctgagttggagaatagtatcccagtaaaataaacaagagtctcaaagtcttttgtatcctttgaagctgtcatggtggtttgtaactaggcaacaggtatatattgttaatcttctttgcatttaattccttttatagagagacacaattttacgagcagatgcaattactagcatgaaggtttctttgtgagggtagttaaaaggcccacatgagctctcttcttatccttgtccttctttcagccagatcttccctgcccctttgctcattccatctttcacccacctacccccaaaacaaggaagtaaatcttgcattagtcaacaataccaaagtgattttcaatatgactttctctgcagaatgttattatttctgcctctttacattcacatactgtcttcctttttttttttttttttttttttttttttagattgggtctcactctgttgcccaggctggagtgcagtggcttgatctcagctcactgtaacctccacctcctgagttcaagcaattctcctgcctcagcctcctgagtagctgggattacaggcatgtgccaccacacctggctagtttttttgtatttttagtagagacagggtttcaccatgttggtcaagctggtctcgaactcctgacctcatgatctgaccacctgtgcctctcaaagtgctgggattacaggcgtgagccaccgggccagccactctcttcctttcagttgcctactcatctcttatgcattcctggacatcagttgtccttttgaagctttcctccactatcccagcccatgtgaatcctccttccagttatagcccttaattctagatggctgatatttttcaataattgttttaagatgaccattttagcctatcagctaaacaatatcaaagacaatagctatttttcaagtactttagtttaccttattatagagtgcataatagatattcagtaaatagtaaaggagaggtgaaggcttgcatagaatggattctggtggtgtctcttggtgagcttttagcatcaagattaatcagcagtttcagcaatgagctcagaccttcagttttagatctttactcatatcagataagagagtgagaagagtggtatgtatcagtgctttatttatatttgcatccaatttgaactatgaatattacaaaggtgcacacataggttcagacagattgatttaaaatgaccaaagatgacctgtcgtaagcaacctgggtatcttaagatgcactccttggagagggaatgttcctaaaaacattttcagagggacgaactgtatgaaattcagtaaaacataaatcatgaggaaaactgattactctctttttgacatgaaatgagagttttaatgcatggttacgattattaacgtactccgctgcaagacgttaataaagttactgttttgcaggctagaatgtcttgatgctgtaatcagaacacactttttcccctttcttccagcttcaaatgcagattcataattgggctgacttctaataactgcaatgttttctgccttgggcttgcagcagaagcctgacaaaatagtgtttgtttaggcaataatttatttatttatttattgagatggagtttcattcttgtcgcccaggctggagtgcaatggcgtgatctcggctcactgcaacctctgtgttcaggcaataatttagactttaccttacttgtgattactatagcaattactatagccacaaggcataattttactgtctcatttcaattttatgaatttgaatgtttttacacttttcctaatgaagtccactatgaagttatgtcaaaaaaaaaaaagaaaaagaaagatgcacacgtaaaagagaggtggttgcaagagaagaaaagaacggaggaaagttaaacgcaaaccagataactctcagcgtattctaaatgaccaaaaacagaactctgttgtcaaagattttaaatggaaaatttttcaatttttttttcttttttgtacaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 3 - RNA, 900 nt, Homo sapiens
  source 1..900: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaa

SEQ ID NO: 4 - RNA, 792 nt, Homo sapiens
  source 1..792: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaa

SEQ ID NO: 5 - RNA, 783 nt, Homo sapiens
  source 1..783: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaaggtttcttcctgaacgcgcctcagcgattttaaatcgtatgcagaatattcaatttgaagcagtggttggccacaaaatcaaaatgaaa

SEQ ID NO: 6 - RNA, 822 nt, Homo sapiens
  source 1..822: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcc

SEQ ID NO: 7 - RNA, 972 nt, Homo sapiens
  source 1..972: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggaatggaaaggacatcagaagtaattacttggatgtatataggatcgaggacacttttggacgagactctgagatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaa

SEQ ID NO: 8 - RNA, 852 nt, Homo sapiens
  source 1..852: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagttaagtacagcacagaacacccaaatactaaaacaccaa

SEQ ID NO: 9 - RNA, 714 nt, Homo sapiens
  source 1..714: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcc

SEQ ID NO: 10 - RNA, 894 nt, Homo sapiens
  source 1..894: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggaatggaaaggacatcagaagtaattacttggatgtatataggatcgaggacacttttggacgagactctgagatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcc

SEQ ID NO: 11 - RNA, 813 nt, Homo sapiens
  source 1..813: mol_type = mRNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaag

SEQ ID NO: 12 - DNA, 900 bp, Homo sapiens
  source 1..900: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaa

SEQ ID NO: 13 - DNA, 792 bp, Homo sapiens
  source 1..792: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaa

SEQ ID NO: 14 - DNA, 783 bp, Homo sapiens
  source 1..783: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaaggtttcttcctgaacgcgcctcagcgattttaaatcgtatgcagaatattcaatttgaagcagtggttggccacaaaatcaaaatgaaa

SEQ ID NO: 15 - DNA, 822 bp, Homo sapiens
  source 1..822: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcc

SEQ ID NO: 16 - DNA, 972 bp, Homo sapiens
  source 1..972: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggaatggaaaggacatcagaagtaattacttggatgtatataggatcgaggacacttttggacgagactctgagatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaa

SEQ ID NO: 17 - DNA, 852 bp, Homo sapiens
  source 1..852: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagttaagtacagcacagaacacccaaatactaaaacaccaa

SEQ ID NO: 18 - DNA, 714 bp, Homo sapiens
  source 1..714: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcc

SEQ ID NO: 19 - DNA, 894 bp, Homo sapiens
  source 1..894: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggaatggaaaggacatcagaagtaattacttggatgtatataggatcgaggacacttttggacgagactctgagatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcc

SEQ ID NO: 20 - DNA, 813 bp, Homo sapiens
  source 1..813: mol_type = other DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaag

SEQ ID NO: 21 - protein, 300 aa, Homo sapiens
MNIILEILLLLITIIYSYLESLVKFFIPQRRKSVAGEIVLITGAGHGIGRQTTYEFAKRQSILVLWDINKRGVEETAAECRKLGVTAHAYVVDCSNREEIYRSLNQVKKEVGDVTIVVNNAGTVYPADLLSTKDEEITKTFEVNILGHFWITKALLPSMMERNHGHIVTVASVCGHEGIPYLIPYCSSKFAAVGFHRGLTSELQALGKTGIKTSCLCPVFVNTGFTKNPSTRLWPVLETDEVVRSLIDGILTNKKMIFVPSYINIFLRLQKFLPERASAILNRMQNIQFEAVVGHKIKMK

SEQ ID NO: 22 - protein, 264 aa, Homo sapiens
MNIILEILLLLITIIYSYLESLVKFFIPQRRKSVAGEIVLITGAGHGIGRQTTYEFAKRQSILVLWDINKVKKEVGDVTIVVNNAGTVYPADLLSTKDEEITKTFEVNILGHFWITKALLPSMMERNHGHIVTVASVCGHEGIPYLIPYCSSKFAAVGFHRGLTSELQALGKTGIKTSCLCPVFVNTGFTKNPSTRLWPVLETDEVVRSLIDGILTNKKMIFVPSYINIFLRLQKFLPERASAILNRMQNIQFEAVVGHKIKMK

SEQ ID NO: 23 - protein, 261 aa, Homo sapiens
MNIILEILLLLITIIYSYLESLVKFFIPQRRKSVAGEIVLITGAGHGIGRQTTYEFAKRQSILVLWDINKRGVEETAAECRKLGVTAHAYVVDCSNREEIYRSLNQVKKEVGDVTIVVNNAGTVYPADLLSTKDEEITKTFEVNILGHFWITKALLPSMMERNHGHIVTVASVCGHEGIPYLIPYCSSKFAAVGFHRGLTSELQALGKTGIKTSCLCPVFVNTGFTKNPSTRFLPERASAILNRMQNIQFEAVVGHKIKMK

SEQ ID NO: 24 - protein, 274 aa, Homo sapiens
MNIILEILLLLITIIYSYLESLVKFFIPQRRKSVAGEIVLITGAGHGIGRQTTYEFAKRQSILVLWDINKRGVEETAAECRKLGVTAHAYVVDCSNREEIYRSLNQVKKEVGDVTIVVNNAGTVYPADLLSTKDEEITKTFEVNILGHFWITKALLPSMMERNHGHIVTVASVCGHEGIPYLIPYCSSKFAAVGFHRGLTSELQALGKTGIKTSCLCPVFVNTGFTKNPSTRLWPVLETDEVVRSLIDGILTNKKMIFVPSYINIFLRLQKVSS

SEQ ID NO: 25 - protein, 324 aa, Homo sapiens
MNIILEILLLLITIIYSYLESLVKFFIPQRRKSVAGEIVLITGAGHGIGRQTTYEFAKRQSILVLWDINKRGVEETAAECRKLGVTAHAYVVDCSNREEIYRSLNQVKKEVGDVTIVVNNAGTVYPADLLSTKDEEITKTFEVNILGHFWNGKDIRSNYLDVYRIEDTFGRDSEITKALLPSMMERNHGHIVTVASVCGHEGIPYLIPYCSSKFAAVGFHRGLTSELQALGKTGIKTSCLCPVFVNTGFTKNPSTRLWPVLETDEVVRSLIDGILTNKKMIFVPSYINIFLRLQKFLPERASAILNRMQNIQFEAVVGHKIKMK

SEQ ID NO: 26 - protein, 284 aa, Homo sapiens
MNIILEILLLLITIIYSYLESLVKFFIPQRRKSVAGEIVLITGAGHGIGRQTTYEFAKRQSILVLWDINKRGVEETAAECRKLGVTAHAYVVDCSNREEIYRSLNQVKKEVGDVTIVVNNAGTVYPADLLSTKDEEITKTFEVNILGHFWITKALLPSMMERNHGHIVTVASVCGHEGIPYLIPYCSSKFAAVGFHRGLTSELQALGKTGIKTSCLCPVFVNTGFTKNPSTRLWPVLETDEVVRSLIDGILTNKKMIFVPSYINIFLRLQKLSTAQNTQILKHQ

SEQ ID NO: 27 - protein, 238 aa, Homo sapiens
MNIILEILLLLITIIYSYLESLVKFFIPQRRKSVAGEIVLITGAGHGIGRQTTYEFAKRQSILVLWDINKVKKEVGDVTIVVNNAGTVYPADLLSTKDEEITKTFEVNILGHFWITKALLPSMMERNHGHIVTVASVCGHEGIPYLIPYCSSKFAAVGFHRGLTSELQALGKTGIKTSCLCPVFVNTGFTKNPSTRLWPVLETDEVVRSLIDGILTNKKMIFVPSYINIFLRLQKVSS

SEQ ID NO: 28 - protein, 298 aa, Homo sapiens
MNIILEILLLLITIIYSYLESLVKFFIPQRRKSVAGEIVLITGAGHGIGRQTTYEFAKRQSILVLWDINKRGVEETAAECRKLGVTAHAYVVDCSNREEIYRSLNQVKKEVGDVTIVVNNAGTVYPADLLSTKDEEITKTFEVNILGHFWNGKDIRSNYLDVYRIEDTFGRDSEITKALLPSMMERNHGHIVTVASVCGHEGIPYLIPYCSSKFAAVGFHRGLTSELQALGKTGIKTSCLCPVFVNTGFTKNPSTRLWPVLETDEVVRSLIDGILTNKKMIFVPSYINIFLRLQKVSS

SEQ ID NO: 29 - protein, 271 aa, Homo sapiens
MNIILEILLLLITIIYSYLESLVKFFIPQRRKSVAGEIVLITGAGHGIGRQTTYEFAKRQSILVLWDINKRGVEETAAECRKLGVTAHAYVVDCSNREEIYRSLNQVKKEVGDVTIVVNNAGTVYPADLLSTKDEEITKTFEVNILGHFWITKALLPSMMERNHGHIVTVASVCGHEGIPYLIPYCSSKFAAVGFHRGLTSELQALGKTGIKTSCLCPVFVNTGFTKNPSTRLWPVLETDEVVRSLIDGILTNKKMIFVPSYINIFLRLQK

SEQ ID NO: 30 - DNA, 23830 bp, Homo sapiens
  source 1..23830: mol_type = genomic DNA
atggtccgaggggggcggggctgacgtcgcgctgggaatgccctggccgagacactgaggcagggtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctgggtgcgtctggggacgctgcccgggctccacgtgcggagtgggtgccccctaggccggggagcgggggatccccaggggtcgcggggccctggaggagcgggcatcggacgcggacacggcggggtgcatcccgagggccccctccgaggcagatgcttcctgcgggggcgctgttcctgggcccgggaagggggcgttggaaccccgagcggtccgggccgaagcctgggactctcgtgcgtccccacccctacccccatcaggcgcccgtgcatgaagggagaccctcacctccggactgagagtcggagcgtctcggagcgacggggagtagggagcgggacccggggcggagggtagtgctggcccctgcggactccgggtcccctgtgtcctctcgggaggggctggacgggctgagctgccgaggggccgatttgccctgggccggacaaagagtggggctttggccggtcccccacggtgggctccttccctctggggattgagggactcaagacaccccgcgcctgcgcttttcttttctttttttctttttttttttttgagacggagtttcgctcagtcgcccaggctggagtgcagtggcgtgatctcaactcactgcaagctccacctcccaggttcacgccattctcctgcctcagcctcccgagtagctgggactacaggcgccagccaccaagcccggctaattttttgtattttttagtagagacggggtttcaccgtgttagccaggatggtctcgatctcctgacctcgtgatctgcccacctcggcctcccagaatgctggggttacaggcgtgagccactgctccctgctgcctacgctctctgggtcgcagcccagccttctgggggctgggtagcctcccagaagggcaaccctgggcatcctccagggcaggctaactggagtctagtggggaggggtaccttgaaagaggaaagttgtttcctcctcctcctcctcctccagtgtttgggacccttcctgggggctggagtgcatccctggacaccccccaatcccatcctcttctctagtttccactgacctaggcccaccctcccctctccggctcagtactcctggaaatgagattccgtacatttgaatcttgtcctaatgaaatatttgtccatgtgggtacctgtgtgtgtgtggtgggggtgcagacggagggtttgtttctcactagctggaactactggggtgtggtatgcttcctgggaatttgtgtgccacagtcctggaggcgaggagggggttgtgagccagtaggcaggggctggggcaagtagcattgtgaagctattgacacccagacgtccccaggcaggagattatgcccccattagcccccttttatctgggcttccttaacaatggactctttgccctgcctgccagagccagcagggagtgactgttcagtggtgaggaagcgggcagaggaagccctgccattgggtaggagcagtgggcagcccctgggctgactgggaggtggggattagggattagacagtcctggctgtctgccttcccctaagccagggggagaggagcaaagggcacgaaatgtggcctccaggaggattagaccgccacatgatcatttgcacaccctggggtttagcaacaataaaagtcagcttttttgtatcccaaggtggcctgtggacacccacatggacaaatgtttacactgggacagaattcaaatgcagaggtcccaggagcctaaagtacactcactctggtatagaaaggattccttactgggcagaggacaggtgcagcctggggctttcccaggcaggacacagggaggctcaggaaccaccaagtccctggaaggtggatctggaggcgttggcaggagccactccctgggttccagggctccaggttcctgctttaaccccctgtctcacagagggctgtgcacttgggggctgctgagcatgtcccagaggctgcatcctggacacagcacctcagtgcatctgagctgaggctaacttggcaggagggacaggcagaacctgccagccacgtgcaattccacccctctggccactcagggaaggagagctgtgagtcaagatcagatttgggtcaggacaggctggggcctgcctgtccctgtgcatcccaagatttatggctggccaggggttgggctgggaggggtggtcttgcatgccaggagagtgcagatcagcctgagaggccaggccagtaagtgaggtcagatctcctgcacctgatagcattaaggccatctacaccaaagctctaatgctgatatgttcctggcctctatgtggggcatggaggtggggcatggaggtgaggcctgctcgcctgggcttctggaagtgggagactcattcctgtggctgaggcctacagcagtgctgtgtggtaggaatacactggaagccatgatgtcattgtgcattttctagaagccacattgaataaagtaaaagacacaggtagaattaatttcattgagcccaatatatccaaaataatatcattttcacatctattcaatataaaaatttactaatgagatatttcatactaagccactgaaatccagtttgtatcttacacatctcagttttgacgagccacatttcaagggcgtgatagccacatgtggctcccatagtagacagtactggtctagagaaatgttggtggcatccttgctgtctggtttctggccttgccaaaagtattaccatcccagtgtggtacattctttcatgtatttgtctcctgtccccagagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgtaagcagtttgcttatctggacgttgtcaagttagaaaagctgttttgggatgggtgtggtggctcatgcctgtcatcccggcactttgggaggccgaagcgggtgggttgcttgagcccaggagctcgagaccaacatgatgaaacccagtctctacaaaaattacagaaaaattagctaggcatggtgttgtgggcccatagtcccagctactagggaggctgaggcaggagaattgcttgagcctgggaggtggaggttgcagtaagtcatgatcatgccactgtactccagcccgggtgacagtgagatgctgtctggaaaaaaaaaaaaaagaaagactgttttgttttggaagcaacacaggcagttgtaggccccctgtgccagagtgacataaactctgtacacctccagtgatttggtccatgtttgtaaaccctgaatgttccagggcagtttcttttcttcactttttatctcttttttttgggtgggggggcggggtacagagtcttgctctgtctcccaggctggagtgcagtggcgcaatctcaacctcccgaggagctgggactacaggcacaggccatcacaccttgctaatgtttgtactttttgtagagacggggttttgccctgttgcccaggctggtcccaaactcctgcacccaagtaatctgcccacctctgcctggcagttacaatttcaaataattcctccctttccttcaacacttggctcatgaccgtccagtccaaggaacctgtcctgcaggtgtgcctctcccgagcttcctctatgcatcttccataatgaagatgccttctcactggaaaccctacaagggtgggaacgtgccttatttgcctgtatcctcagggtctagcagagagaagataatttgtaataccaaaacaccattaaattcagctgatgctttcataagcgctccttggaggaaggactccatttacttgacagatctgtgcaagacagcagcctggcgcgtctaacctgcagccagttgcatcctctgtttaaccttgtttgcggaagctttctctaaacagccagcacttgtctgttcccacatgggtccgttctcccagtgaatcaccgtggtgcctgctgactgctctgtagcacagtgcttcgcaaagtgtgatcctgggaccagcagagcagcagctcctttgagcttattggaatggcagaccctcaggtcccacctctgacctgctgcatgggaattctggggagggacgcagaatctctggttccacaggctctccggtgatgctaatgaataccggcatttgaacagcaccgatctagcccctttcagtccatgagccaacaacccttggtcctgtctgtggtgacccagtgtgactctcatggggagcaaggagaggaagttgaagttcactgacagggttgttaaggggattatgcaatagatgagacccatgggcctgaagtccgagggtgtatgttagttccccgttcttttgacccatggattaacctactctgtgcaaagggcattttcaagtttgttgccctgctcacttggagaaagcttatgaaggatcaggaaaattaaaagggtgctctcgcctataacttctctctcctttgctttcacaggccttggtatgttcctgcttcatccccttctacagtggccttatccctccttccttcagaggcgtggtaagtcggctttctctgctagcgctgagtcctgggggcctctgaagtgtgctcacacatctcctgcctgcagggcactggtgtcgggcacctcagggtctgtcccatggtggagccccatgcctcactgcctttcagacagagtagccacagctggccctatttccaggctacccgggcagcaaaacttactgcatgtgtaattaattatttggctatctgtaaggtaaactggctggttcacttaatctgcaccttaagcatcagatagcttctcagtgatctagttaaactatatgatgttggccaggcgcggtggctcatgtctgtaatcccagcactttgggagcctgaagcaggcagatcacttgaggtcaggagttcgagaccagcctggccaacagtgtgaaactctgtctctcctaaaaatacaaaaattagctgggcatggtggtgtgcacctgtaatcccagctgctcgggaggctgaggcaggagaattgcttgaacttgggaggcggaagttgcagtgagccaagatcgcaccactgcactccatcctgggtgacagagcgagactctatctcaaaaagaaaaaaaaaaaaaaggtaaataaagtatatgacactgaagaatctgttacccctggaaggtggagctttactcttagggggaactataacagtcatatatatatatttttttcttttctttttttttttttttgagatggagtctggctctgtcgcccaggctggagtgcagtggtgcaatctcggctcactgcaacctccacctcacaggttcaggcaattctcctgcctcaacctcccgagtagctgggattacaggtgcctgccgtcacgccaagctaatttttgtatttttagtagagacagggtttcatcatattggccaggctggtctccaactcctgacctcaggtgatccgcccgccttggcctcccaaagtgctgagattacaggcgtgagccatggtgcccggccaacaatcacatgtgttgtaaacaacaacaaaaatctgtcagcctggtctaacctagatttgtgctttgttttgttttgccactttgtgatgcacaggaggaagtttaggctgtaaaatactagccttttagggtaatttttgaactcacaagagcagcagcggaacctttgatgcaatcctgtatgtagcaccagcagagccacgtggcagagggactcgcattaggagcctcccattacagactacgtgctcctgtgcgttatcttatagggtccccacaaccaaggggagatgtgattattcatcctgtgtggctgtggggaacttgagagtcatacttgcccaaagagcacggccagcgagcttgcacccaggtcactctctgctcctctgtcagaacagggcatgtcttggttcactgcagggcggctcttctcattctctgtagtttggggtccaggatagtggtccacggagccactggagtgcccagctactgagtgaccaaagcatattttggatttccgacattgccacagcatggttgggcatcagcaggaccccaaccccttgttatgctggtggctttatgtggttatttgatcttccccagaactcagcaggagtgcacccagcagcaccgtagtgatgctctctggctccccagtgcacggttctggctttccttcctggtcgagagtttcaagccctctgggtcctactctgtccttttcagcccatagctttgttcaaaagctgctggcagtgttcagatttggctgagttcagtgaatatgtgcattggctgatttctgagccatgccagggggatggagaagccgaagcaggagtgtttgttctgcaggctctggagtaggcattgggtctgtgccggctcacttgctagtcttgcatccttccccaaccccctctggggatgtctggccacatcagaagacagtttgggttgtcagaactgggggagtaccaggccgaggtgggtggatcatgaggtcaggagatcgagaccatcctggctaacacagtgaaacctcatctctactaaacatacgaaaaaaattagctgggcgtggtggcgggcgcctgtagtcccagctactcgggaggctgaggcaggagaatggtgtgaacccggggggcggagcttgcagtgagctgagatcctgccactgcactccagcctgggcaacaaagcgagactccgtctcacaaacaaaacaaaacaaaacaaaacaaaatctgggggagtgccactggcatctgatgtatagaggcccgagatgctgtgtcatcacccgttgagtgcgctcataggcatcttcctgacaattagaacccattattcttcaaattcaatgcaagcaaattcaaagcattactgtgtacataccgcatgctaatcaattgcaccactggagctcctaaattcaaaacattactataaaaaagttcaaaatgcatggaaaagttgtacatggcaggagaatatttgggcttctgactaccccttgaatgaagatgatccaccagccgccttcctccttggtcttcactccagattcctagcatttcattctgtgtctctttatgcagtgaggtttttgtttgttttttgagacagagtctcactgtatcacctaggcctggagtgcagtggcgcgatctcagctcactgcaaccctcggctcctgggtttaagcgattctcctgcctcagcctcccgagcagctgagattacaagcacacatccccatgcccagctaatttttgtatttttagcagagacagggtttcaccatgttgcccaggctggtctcgaactcctggcctcaagtgatccatgtgcctcagccttccaaagtgctgggattacaggcgtgagccaccatgcccagctcctagtgaggtttttgatgccttgctacatctgccctagaaattgtgtgactacgattttggaaatgttgctgtgtaaacttgtgatcatttctggactccaggcaagaatcttgatggctaaggtgtggctgaacatgtctgattctctcctggacctgttttaggccaaactctgctctgaaattcctccgtgtggaagggcgggctggggagagcctcccagctggaatcttttggatgcctttctctgtgggtatctgatggctggctctgatggctggctgtgatggctgtggctggaaatcattgttgacatgagtttcacagatgcaggctctgtccaaattgtagcaaaagctgcctgccccagccgagctatgggcaataaggtggtttaaggatatagatgaaggaaaactcacccttagaataatttatccaaaatgctgctgtgttgtgggttagaggacattttctgaggtcccaggttcattgtttcatttaagtctcaaaagtccctccaggtgttggttctaattgtcaaagcatggggggagatgggctcatgggttaaaggtcttatcccagatttctgtatcctccttgcaagcagcaaaggggtctggatttgaatccatgaccatgtttctcctttgggtttccatcacactctgtccccgtgcactgagcaccctttagttcatatgacccccttaggcatgttacatgggcactcctataggtgcccatctggccctaggacttggccaacacaacatggactccagtttccatctgcctctttgccaggcacttttgtgcagtgcacacactgtacaacagtagacggcaaccctgagagccagagtagagcctgtcctagcaccggaatgctcggtaaggatttgtcgcaggagtgattccaaagccaatgtcctccctccatatcagcctgtttgtggctctgagaagctctgcccacatgtgaaagcttgttaagcacttaagcactaacccagagcttcagacagtgccagtcctttttccccttctttaaaagcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgagttggtggtgagggggcaggtgttctggggtgcagctcttctttgcctccctgattgccaggagctaccagttactgtctgcacaatcaaacagaaatagacctgtccttgatggttaacggaaataaaaggcgcttgtcccagaagctcaggtgaggcaccaccctgattatgggaatcacctgggaacatatacccagacctaaaactcagatccacttcccaggctgtggttatatagtcaggggggtgcagtatgggtattaggattttttattttttagttataaagatttttttttggtttgtttttgagacagggtcttgctctgccgcttaggctggagtgcagtggtgcaatcatagctcactgaagcctcagactcctgggttcaagcagtcctcccacctcagcctcctaaggagctgggacccacaggcatgcagcaccacacctggctaatttttaaaaattttgtggagtgttgcccaggctggtctcacactcctggcctcaagcgatcctcccaccccagcctcccaatgtgttgggattacaggcatgagccattgtacccagccactaagatgattcttatttggaaacacggtcaagaacaactgcgttcggtagtttaaccttttttgattgtggtggttttagtatgccttaccactctaccatagtaagaaatttgcagaccatgtacaccaacctttggtgctcctggggagaaagaaagaaggctatgcaatgcaatgcatgctcacagtccaagggagagggaaagctgtctaacaggattggttttcccgtgtgctttataagcagatgagtagaggagacagctcttattgtcctagtggcaattgggataggctgcaaagtttgttagggtggaggcttattccgggaccaagggagcccaaagaaacaagctcctgccaggcgcggtggctcacgcctgtaatcccagcactttgggaggctgaggcaggtggatcacctgaggtcaggagtttgagaccagcctggccaacatggtgaaaccccgtctccatgaaaaatacaaaaattacccgggcatggtggcgggcacctgtaatcccagctactagggaggctgaggcaggaaaatggcttgaacctcggaagtggaggtggccgttagccgagatcacgccactgcactccagcctgggcaacagagcaagactctgccttaaaaaaaaaaaaaaaaaaaagaaaagtaaaaggaaaaaaaagaggctctggcctgctggggtgcctgcaaagtctccgtggaagggtgacattcaagccgagacctccagggaactgtctcctgggagcacagagccctttgctcagcccccaggtggctcagtgcccccagccagcagactcagagcttgcatgattctttggtgctctctgcggtcttccaatgatgctgaaataaatggtgcttggtgtctccctgctgtagtccccttgcttgctttgctcacaggtgctgggagagatatgccttcgaggatatttggatgcattcaggttcttggaagagaagggtatgtatgggctgggaggatcagccatgcccttttgacaagcatttactagcggtcttggtaaagacttgagatttgccttagttctaacacttagtgcccaacgccttccttgtgttgctcaacctactcatgagcccaggagataggaaatctccgtcccattgtacagatggggaaacagaattttggaaaggagagccaagcagcacacacccctccctgaggggcagagccgagatttgaactgggatgtcatgactccagggccctctccctccccagggtccccttatctgaaggcggtttttctttccagctcgacctcttgtgacccttagtttaacaagggccgaagttaaagagtttctgcgcctggaccccaaatgaagcaatcagatttctcatctccagtcaggtgtgggtccaagcccactagacaagtttgctcttcccagagcacatttctgccttcaagtcatcctggcttgtcagggctgggggagttctgctgtagaaatattagagtggaaggaaaaagatgtgttgggagctatttttctttaatactaaaagttggttgatgaatttgtcgttggccaagaccaaggagactgcatttttaaggacatatgtgtatttatctgctcagaaaatgttcattgctgtgtgctagggatactgcagtgaacacagaggtgtgacccttgccagccttgtgagagaagtgagcagataagtaagcagaagggtgatgctgtgtcgatgggaaagtacaggtgccaatgagaaggcacaggtgtcaaggagaagacacaggatgctggaggctcatgcaggatggatctccaaggcccaggggaagaagggcctctcggaggacgtgaatccacattaagactttggggataagtaggagcgccttaggcatggggacccatggatgcgaggcctgtaggacacagacaggatggcatgaaggcctgtgcaactggaggggtggggatggggacactaagagatggctggaagtgtgggggtggggacactaagagatgactggagaagagggggtcaggagtggtgaaaaatgggagaggagggcaggctgggccttttggatacagggggattgcatcctgcagtggtagggagccactgagggctgctgcagtaggagtgaggggatcagaggagagctttggaagccccctggatgcgggacaggaagcgagataccagtgtctaggaggccagtgaggcagccacaggctccaccaggatcagggctgcgagggtcatgaggaggaaaccaatttgaaggagtccaggggaataggacttggaaatgaccgatgggacatttgggaagaggaagacagaagagcgcagtcccggcttctggctttagcagttgggcaaggggagatggggagatgtgcccatgggttgagggttgaggacattaggagggagccggtatggcaggaagagctggtgtgccagagatgctggaagcagcatctgcctgagaacagatacctggcaatattcctaagggaaagtgacatctcggagggtgaggagggcatctgatagggcctggaaagagccggggcaagcatgaatgtgaggttatcttggggggcaaggctcaggcgttgaggagcagcccctggtctcttcagcctgaagttggaagccagagttgggccaggtgcagctgtggttgtctgaagtccccctcccccagcccagtgtgccaatgctgtaagagcaagggccgctcactggtgctggtggctgagtcccagcacccaggacagggcctggcacatactggtgcccaatcctcccttctgggtgcttcttccaaggccttgtgatggaagtgagtaccctcttcgacatcagacccagcttcaaatcctggctctgctatgtattggctgcgtggctttagacaagtcttttaaccttgctgtgcttctgatttctcagctgaaaaatggagatgatgatagtggtttctgtaaggccttatggtgaagcacctagctcagggcctggaaggcaggtgtaaccagtggttcagttgttataaaccaacactaaccctcgcctttgcacctcatgaaaccagatatgtagatggagcccacaaagctagcaggagccaagctcacgtgtgtcctgctttaaagccccatacccctttctccgggtgacaaacacctgtgctcgttctcttcccttcccctcttccccttgcatttggctaataacaggccagctgcctgcctccctgcagtttggtagatgggtgggtaacgaccaccactcccacgttcgcctgatgggcttgttttccgtgcccttcacaggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacaggtacccactcctcggggtgagcacgggcagcaccttgttttctttcttgtgcattatggaggaagatggtactgccacatgggagcgatagggtgaggcaaccatgacaggtggttgggaacatctccttccatgtgtacagcctgggctgctgccatcactcccagcacagcccccaacccccccaatcctggaaccttgccaagtctcccttcccatggggtcatgaccaggaggaaaacaaactccagctgagccccttggggttccccatataggctcctgcctgtggcagctgggccctctgtacccctttccaactctgtctccctaacatggcacctgagctcctgccatcctggatttcatggaccccaaggatgggggtcctgcatctgggacttggcctattactcggagctccttttcagccgcctccctccacctgtccacccacctcaaggctcctttcttgagacctctcctaatttctcccttcccctaaacccacaattttgaacctccatcgaatggtgctgtattttataatgtcatcaaatatcaaatggagacagtgctatggtccaaatgattgtgtaccccccagaatttgtcttttgaaatcctaacccccaacatgatggtcttaggaggtggggcctttgggaggagattaggtcatgaggaaagggctgtcatgaatgggattggtgcccttattaaacagacccaagagaggtcccttgtcccttctactgtgtgaggactcagaaggtggtgtctatgaagaagcaggccctcaccagacaccaacatgtctgctgccccttgatctgggaccttgcagcctctagaactctgaaaaatcgatgtttgttgttttataagccactcagttggtggcattttgttagagtagcctgaacacggactaagtcaaacagaagaacccacaaaccagctacagagttgggcatttggagaaattcaaaaatgagtcagacataactccttattcttgaggtgccctaagagatgggacacagcagctgcccaggtgcattagtttgttctcacattgctataaagaaatacctgagactgggtaactcataaagaaagaggttgaattggctcacagttgcacaggctggacaggaagcatggtgctggcatctgctcagcttctggggaggcctcaggaaacttacaatcatggcagaaggtgaacgggaagcatgcacatcccatgactggagcaggagtgagagagagagggaaatagagggaaggtgccatacacttttaaacaaccagatctcatgagaacacattcactatcaagagaacagcaccagtggggaaatccgcccccatgatccaatcacctcccatcaggctccgcctccaacactgggaattacaatttgacatgagatgtgggcagggacacagatccaaaccatatgaccagattaatacgatttgaggcatcacgaggtcattaaagagagggaataaaagactggggctccaggaagaaggctctggaatccagcagagggtcaaggaccagcttgtaaagctggtggtgcctgagaagtacctaggagaacatagatgctgtgacgtttgatgtagctgttttttgttttgtgttttggtttttgagacagagtctcactctgttgcccaggctggagtgtgcagtggcgtgatcttggctcactggagcctccatctcccaggttcaaatgatcctcatgcctcagcctcctgagttgctgggattacaggtgcacaccaccacgcctggctaatttttgtgttttcagtagagacagggtttcaccatgttggccaggctggtcttgaactcctgacctcaagtgatccaacaacttcagcctcccaaagtgctgggatgacaggcatgagccaccatgcccagcctgatgtagctgtttctgtgcacattatttgctgtggggtatattcagatttcttaatacaagatgattctttgcctcatgacttacacaccattttctatttaatttcagctatgatattggaaatggacatgtcttttcaaggaaaataaaagcaggctttctggaatggcgacttccaaacatatttgtcaatttaaaggagctgggagtggggaccctatgctccgtaagcactctcttagctgttcttggctgtgctccccgcttcagcttcacactgcccttgctgtgaagggagcagcctgggccgggcgcggtggcttacacctgtaatcctagcactttgggaggccgaggtgggtggatcacctgaggtcaggagttcaagaccagcctggccaacatggtgaaactccatctctactaaaaatacaaaaaattagctgggcatggtggcaggtgcctgtaatcccagctacttgggaggctgaggcagaagaatcgcttgaacccaggaggcggaggttgcagtgagccgagattgcgccattgcactccagcctgggggcaacaagagcaaaactctgtctggaaaaaaaagaaaggagcagcttggcaaaccccaccttgtcgcttttgtgagtgcctctgaccctttggctgccaggacgggcgtattttatggaaatgctaagcaccaacagagtaaagtggtttggtttttcacagtggtgggagataatagctccaaattgtctttttcagcactgagtgaagaaatgaaagacaaaggtggatacatgagcaagatttgcaacttgctacccattaggataatgtcttatgtaatgctgccctgtaccctgcctgtggaatctgccattgcgattgtccagaggtgagcattttaggtggctccgtgtcttcctcacagggttgatatgaggatgaaacaagatgatagatcatggtggcatgtagtctgggacctggattgtcgtgccacagatcacagctcacagtctatgtgcaatgcccctgaatgttgcccacctgtcctcaagccacacatgcacctgtaactcagtgcaagcccagaaactccccgtggggactcctagagctgtcagtggcctcacatagcagctggtccagtctcttgtgattgcccaaggaaactgaggcctggagagcttggggtcactgctctgaggccatagagatgcctagtagaagggccaggcctagaagcaggatccttgctgcccctctgagctgtttccatttaaaatcacatgaaggccggcgccgtggctcacggctgtaatcccagcattttgggaggccaaggtgggtggatcatgtgaggtcaggagtttgagaccagcttggccaacatggtgaaatgccatctgtactaaaaatacaaaaattagtggagcatggtggcacgtgcctgtactcccagctacttggaaggctggggcagaagaatcgcttgagcctgggaggcagaggttgtagtgagccaagattgtaccactgcactccagcctgggtgacaggagagaaaccctatctcaaaataaaatgaaaggtaatgaaatgaataaaataataaatcaagtcacggccgggcacggtggctcacacctgtaatcccagcgctttgggaggccgaggtgggtggataatgaggtcaggagttcaagaccagcctggccaacatggtgaaaccatgtctctactaaaaatacaaaaattagctgggcatggtggtgcatgcctgtaatcccagctactccggaggctaaggcaggagaattgcttgaagcaggacctaggaggcggaggttggttgcagtgagccgagatcatgccactgcactctagcctgggctacagagcgaaactccgactcaaaaaaaaaaaaaaaaaaaaatcaaatcacatgaaagtagaacatagggaattccatctttcgttctaggcatagtttgttaatatgattcagagccagcagttaggagaacacagtgtgactctcctagaacttcttgattgggcttcctctgattgggtttcctctgattgggcttcctctgaaagtgggggggatggggggtggggagcagaatggtcagagcttggctcagcagtcagactgctcttcttcaaatcctggctgcattgcttactacagctgtgtgactccagatgactgaatccacctctctgtgctgcagcttcccgtctagagagatcacctggagcagagggtggtcaggagactcaatctggttactgactcacagtgcaggagtactcatcccatagtaagcatccagctagagatgttgatttctattttcaggtaataatgatgatcgtaaaattagagacagataaaaggtatgggcattaggccagggcactgcaatttctaagctgtgtgacctcaggcaagttactcgacttctctgagcctcagcggtttcatccgcaatatatggataggaaaaccgacctcagtgggttgtctgacagtggagggcacttgattaaaaaaaaaaaaattaccctggtctgaatattaccctggactgaaagaaaaatattgagctaatacaggcatcaggaatggggctgcagggagtccagggaagggagaacgaagagcctgaaggtgtgaggaggtgcgagtgctgatctgtctgctacaaagaggctgctgagcctcctgtggatgtggccctggacttggcagtttaatacctgagctgttaaaataacctcagatgctgtgttctttaaggggtaggattcagattcctgctgaaatgcttctgaaagggagggaatgagccagcccatccccagttgctttttaagatcattgggaagttctggtcttgccatttgtccctggaccactcttaggtcctcctgccccacttccatctgggtgtgtgccctgggctgtccaccacacagctacatcctgccatcttccctcctggagccactgtgccatgcatggatctgtagcttcatttttcttggcttttccctggtttttctggagcagagtctctagtaaactcccaaggaagaaaacgtttgactttatgtgtgttgggaaacgtgctttttttctattacatctcagtgataggttggccatgtctagaattgcaggttgaaaatcatttcctctcagtatattggttagtgagaagcctgggactgagacagtcacattctcacttctttgcaggtgagtgctcttaggactgtctttttatcccttatactctgaaatgtcatatgtcttggtgtaagtccttatttcagttattgagctggacaagtactggagaccccttcagtcaaagccttctgtcattctccagctctaggaaattatcttctattgttatttctgttattccttcccttccattttcttttttctttttttttttttttttttgagacagggtcttactctggtgcccaggctggaatgcagtgacctgatcatggtacactgcagcctgaacctcccagactcaagtgatcctcccacctcaacctcctaagtagctgggactgcaagcacacatcaccacacccaacaaatattttttaaaaattttgtaagatgggatcttactatgttgcccagactttttcttcctcttcctggggctcttattaggaagatgtttgacttcctgggttggattcctgtctccgtgtctgactttctctctttgtcatatttttcatcactcgttgtctttttgcgtctgctctgacagatttcctcaaattttgtcttctagtcctatcctacagtttttactttcagcaaatataatttaatctccaagagtactctcttgttcttttttcttagcattctgttcttgttttatggatgtaacattctcttggaatatttgctgtcctctagatcatcccttctccatttcttcttgggctagtttttctgtttcttcatctttctcttttatgctacttattctgggcgtgttcttggtgggttttttcccatatagcaacagaggacttggagctcagggagaaaagggtaggtgcatcacctggcagagctcccagacagtgacaggcaggctgcgggaaggatgtctacttggcggtgctaccgctttcctagaaaccctttccctggagctggttgaactgttgggttttgccctggtggtgaacgctggctccccgtgctctgcctgtttcatcaccagccccctccccttctgcctggggtccagtaatctgttgaaatatatatcttgctcattggtgagctcctgctccttcctcgttgctcttgcagatttatcacttctcgtaaggctgcgcttgtacttcgggattttctctgtgccacactgggaaacatagggtggttgcatgctgcagtcctgagcacttatttcactcacatctttacacgaagatttggtgggtgtttactttgtttttagtaagttagtctgtcatgtcctttgatcctttttttttgttttttgagatggagtctctctgtgtcctccaggctggagtgcaatgtcgcgatctcagctcactgcaacctccacctcctgggctcaagagattctcctgcttcagtctcctgagtagctgggattacaggcatgtgccaccacacctggctaatttttgtatttttagtagaggtggggtttggcatgttggccagcctggtctcaaactcctgacctcctgacctgcctgccttggcctcccaaagtgctgggattacaggtgtgagccaccacacctggccctgattaatcttttaatgcccagtctctccttcaaaagccggctcctttctctccctcgccttcctagattccttctccactccccaggatcagcctcctcctccccaccccaccactgccggggggatgtctgtggtcaggcatttatcagagaccctgaggtgggggtcctttatgtgtctgggggatggagagtctagaggaggtagcgttcagacctctccatggtgcctctgctgggctcacatgtgaccaagcacagcaaaccatgaggcaggggatggtcttgaccatgagagcccttgcagcagctgccatgggcctcagctcctctccaagctgggaagagccctgaaaagccaaggtgtttttttttccctctttatttcagtgtaagtcccttgagctttcttgaaccagaagtgggctcattttgctttagagatttcaggtgggcttgtccttgtcctagcatcccagatccaccttctgggaagtcatcagattggaggtgatgttggcagcttttgtaaacaaagggtagtgttgtaagctgttgtgtctgcctatgtgtgtgtttgtgtacttggtctcatctctgcagactggtgacatggcttccagatatgcccgacgatgtcctgtggttgcagtgggtgacctcacaggtgttcactcgagtgctgatgtgtctgctccccgcctccaggtaaatactttggctgtgggtgtgtgggccggacgggcacctctctcatctgatgaggcctcacacgacattctagaaacagctggctgaacaccaagcaaggagcttgcccttgggtgtggggaccctgtctcatgggaggcagctgagtcagtcagaggtcctggcacacctgctgagagctgccacccaggccaacctgaaccggagcctgggaagacttcccgtcggatgagtctctttgagtgcagcattgatggtggaagagcagagaggccccagataagcagggaaaggtgcttcagacagagtggctgggatgaggactggggagtgtcagatagcgctggcgtgtctgagcgaaggagctctggcacccatggcacaggaaggaggtgggaccctggaggggcagggctagcagagctcctcggagcgtgtggctaggtgcctggtaatgcaagccccctgtcctccaccctctgttgtactgagtcacagtctccggggtgaagcctagcagtctgcgttgacaggccccaggggatgccgctacttcctgaattctgaattctggaaactgagccggagttcagggcctggctcccattaccagggttgggcgttatcctgaaaatcataggccttggtttcctcacttggctaacaggggtgatccccatcccctcaatgggtttccgtgagctcctgagagcccgtagcatggtacttggcacatgctgggcatcaggaggtatggcctctcttgctattgttgttattggtagacacagaaggatttaaaagtaggggaatgcaaagatccgatttgctagggaagagggcagtagtggccaagtagagggtggatcctgggccctggctggcagcaggcagcaaggggggctgccagggcccaggcagggacgatctgtagaccgagaggcttcctaaggctcttggacaggaggaggtgtcggttccaagcctaaggagtggggcagccctggtgactggtggtcagtggtgccaggcggtgggtggtaggacaccctggcaggcaagtaggtttgtgtgggggaaactgataggcccctccagggattcgttggtggacaacacctgtgatgtccagtgggaggtgtccaggtagctgggagggccacaggcttggaagacctaggtggtgacatcagcccagcactgagggctagaagaagctgtgtctctggctgtgacggcaccctagagtgtgtgtggtgccctctactggccggcaatgtgggtccaccgtagctcagactgcacactgcagcagcgggaacggcctctaagccaacttcctccatgtgtttcaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagac

SEQ ID NO: 31 - DNA, 23830 bp, Homo sapiens
  source 1..23830: mol_type = genomic DNA
atggtccgaggggggcggggctgacgtcgcgctgggaatgccctggccgagacactgaggcagggtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctgggtgcgtctggggacgctgcccgggctccacgtgcggagtgggtgccccctaggccggggagcgggggatccccaggggtcgcggggccctggaggagcgggcatcggacgcggacacggcggggtgcatcccgagggccccctccgaggcagatgcttcctgcgggggcgctgttcctgggcccgggaagggggcgttggaaccccgagcggtccgggccgaagcctgggactctcgtgcgtccccacccctacccccatcaggcgcccgtgcatgaagggagaccctcacctccggactgagagtcggagcgtctcggagcgacggggagtagggagcgggacccggggcggagggtagtgctggcccctgcggactccgggtcccctgtgtcctctcgggaggggctggacgggctgagctgccgaggggccgatttgccctgggccggacaaagagtggggctttggccggtcccccacggtgggctccttccctctggggattgagggactcaagacaccccgcgcctgcgcttttcttttctttttttctttttttttttttgagacggagtttcgctcagtcgcccaggctggagtgcagtggcgtgatctcaactcactgcaagctccacctcccaggttcacgccattctcctgcctcagcctcccgagtagctgggactacaggcgccagccaccaagcccggctaattttttgtattttttagtagagacggggtttcaccgtgttagccaggatggtctcgatctcctgacctcgtgatctgcccacctcggcctcccagaatgctggggttacaggcgtgagccactgctccctgctgcctacgctctctgggtcgcagcccagccttctgggggctgggtagcctcccagaagggcaaccctgggcatcctccagggcaggctaactggagtctagtggggaggggtaccttgaaagaggaaagttgtttcctcctcctcctcctcctccagtgtttgggacccttcctgggggctggagtgcatccctggacaccccccaatcccatcctcttctctagtttccactgacctaggcccaccctcccctctccggctcagtactcctggaaatgagattccgtacatttgaatcttgtcctaatgaaatatttgtccatgtgggtacctgtgtgtgtgtggtgggggtgcagacggagggtttgtttctcactagctggaactactggggtgtggtatgcttcctgggaatttgtgtgccacagtcctggaggcgaggagggggttgtgagccagtaggcaggggctggggcaagtagcattgtgaagctattgacacccagacgtccccaggcaggagattatgcccccattagcccccttttatctgggcttccttaacaatggactctttgccctgcctgccagagccagcagggagtgactgttcagtggtgaggaagcgggcagaggaagccctgccattgggtaggagcagtgggcagcccctgggctgactgggaggtggggattagggattagacagtcctggctgtctgccttcccctaagccagggggagaggagcaaagggcacgaaatgtggcctccaggaggattagaccgccacatgatcatttgcacaccctggggtttagcaacaataaaagtcagcttttttgtatcccaaggtggcctgtggacacccacatggacaaatgtttacactgggacagaattcaaatgcagaggtcccaggagcctaaagtacactcactctggtatagaaaggattccttactgggcagaggacaggtgcagcctggggctttcccaggcaggacacagggaggctcaggaaccaccaagtccctggaaggtggatctggaggcgttggcaggagccactccctgggttccagggctccaggttcctgctttaaccccctgtctcacagagggctgtgcacttgggggctgctgagcatgtcccagaggctgcatcctggacacagcacctcagtgcatctgagctgaggctaacttggcaggagggacaggcagaacctgccagccacgtgcaattccacccctctggccactcagggaaggagagctgtgagtcaagatcagatttgggtcaggacaggctggggcctgcctgtccctgtgcatcccaagatttatggctggccaggggttgggctgggaggggtggtcttgcatgccaggagagtgcagatcagcctgagaggccaggccagtaagtgaggtcagatctcctgcacctgatagcattaaggccatctacaccaaagctctaatgctgatatgttcctggcctctatgtggggcatggaggtggggcatggaggtgaggcctgctcgcctgggcttctggaagtgggagactcattcctgtggctgaggcctacagcagtgctgtgtggtaggaatacactggaagccatgatgtcattgtgcattttctagaagccacattgaataaagtaaaagacacaggtagaattaatttcattgagcccaatatatccaaaataatatcattttcacatctattcaatataaaaatttactaatgagatatttcatactaagccactgaaatccagtttgtatcttacacatctcagttttgacgagccacatttcaagggcgtgatagccacatgtggctcccatagtagacagtactggtctagagaaatgttggtggcatccttgctgtctggtttctggccttgccaaaagtattaccatcccagtgtggtacattctttcatgtatttgtctcctgtccccagagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgtaagcagtttgcttatctggacgttgtcaagttagaaaagctgttttgggatgggtgtggtggctcatgcctgtcatcccggcactttgggaggccgaagcgggtgggttgcttgagcccaggagctcgagaccaacatgatgaaacccagtctctacaaaaattacagaaaaattagctaggcatggtgttgtgggcccatagtcccagctactagggaggctgaggcaggagaattgcttgagcctgggaggtggaggttgcagtaagtcatgatcatgccactgtactccagcccgggtgacagtgagatgctgtctggaaaaaaaaaaaaaagaaagactgttttgttttggaagcaacacaggcagttgtaggccccctgtgccagagtgacataaactctgtacacctccagtgatttggtccatgtttgtaaaccctgaatgttccagggcagtttcttttcttcactttttatctcttttttttgggtgggggggcggggtacagagtcttgctctgtctcccaggctggagtgcagtggcgcaatctcaacctcccgaggagctgggactacaggcacaggccatcacaccttgctaatgtttgtactttttgtagagacggggttttgccctgttgcccaggctggtcccaaactcctgcacccaagtaatctgcccacctctgcctggcagttacaatttcaaataattcctccctttccttcaacacttggctcatgaccgtccagtccaaggaacctgtcctgcaggtgtgcctctcccgagcttcctctatgcatcttccataatgaagatgccttctcactggaaaccctacaagggtgggaacgtgccttatttgcctgtatcctcagggtctagcagagagaagataatttgtaataccaaaacaccattaaattcagctgatgctttcataagcgctccttggaggaaggactccatttacttgacagatctgtgcaagacagcagcctggcgcgtctaacctgcagccagttgcatcctctgtttaaccttgtttgcggaagctttctctaaacagccagcacttgtctgttcccacatgggtccgttctcccagtgaatcaccgtggtgcctgctgactgctctgtagcacagtgcttcgcaaagtgtgatcctgggaccagcagagcagcagctcctttgagcttattggaatggcagaccctcaggtcccacctctgacctgctgcatgggaattctggggagggacgcagaatctctggttccacaggctctccggtgatgctaatgaataccggcatttgaacagcaccgatctagcccctttcagtccatgagccaacaacccttggtcctgtctgtggtgacccagtgtgactctcatggggagcaaggagaggaagttgaagttcactgacagggttgttaaggggattatgcaatagatgagacccatgggcctgaagtccgagggtgtatgttagttccccgttcttttgacccatggattaacctactctgtgcaaagggcattttcaagtttgttgccctgctcacttggagaaagcttatgaaggatcaggaaaattaaaagggtgctctcgcctataacttctctctcctttgctttcacaggccttggtatgttcctgcttcatgcccttctacagtggccttatccctccttccttcagaggcgtggtaagtcggctttctctgctagcgctgagtcctgggggcctctgaagtgtgctcacacatctcctgcctgcagggcactggtgtcgggcacctcagggtctgtcccatggtggagccccatgcctcactgcctttcagacagagtagccacagctggccctatttccaggctacccgggcagcaaaacttactgcatgtgtaattaattatttggctatctgtaaggtaaactggctggttcacttaatctgcaccttaagcatcagatagcttctcagtgatctagttaaactatatgatgttggccaggcgcggtggctcatgtctgtaatcccagcactttgggagcctgaagcaggcagatcacttgaggtcaggagttcgagaccagcctggccaacagtgtgaaactctgtctctcctaaaaatacaaaaattagctgggcatggtggtgtgcacctgtaatcccagctgctcgggaggctgaggcaggagaattgcttgaacttgggaggcggaagttgcagtgagccaagatcgcaccactgcactccatcctgggtgacagagcgagactctatctcaaaaagaaaaaaaaaaaaaaggtaaataaagtatatgacactgaagaatctgttacccctggaaggtggagctttactcttagggggaactataacagtcatatatatatatttttttcttttctttttttttttttttgagatggagtctggctctgtcgcccaggctggagtgcagtggtgcaatctcggctcactgcaacctccacctcacaggttcaggcaattctcctgcctcaacctcccgagtagctgggattacaggtgcctgccgtcacgccaagctaatttttgtatttttagtagagacagggtttcatcatattggccaggctggtctccaactcctgacctcaggtgatccgcccgccttggcctcccaaagtgctgagattacaggcgtgagccatggtgcccggccaacaatcacatgtgttgtaaacaacaacaaaaatctgtcagcctggtctaacctagatttgtgctttgttttgttttgccactttgtgatgcacaggaggaagtttaggctgtaaaatactagccttttagggtaatttttgaactcacaagagcagcagcggaacctttgatgcaatcctgtatgtagcaccagcagagccacgtggcagagggactcgcattaggagcctcccattacagactacgtgctcctgtgcgttatcttatagggtccccacaaccaaggggagatgtgattattcatcctgtgtggctgtggggaacttgagagtcatacttgcccaaagagcacggccagcgagcttgcacccaggtcactctctgctcctctgtcagaacagggcatgtcttggttcactgcagggcggctcttctcattctctgtagtttggggtccaggatagtggtccacggagccactggagtgcccagctactgagtgaccaaagcatattttggatttccgacattgccacagcatggttgggcatcagcaggaccccaaccccttgttatgctggtggctttatgtggttatttgatcttccccagaactcagcaggagtgcacccagcagcaccgtagtgatgctctctggctccccagtgcacggttctggctttccttcctggtcgagagtttcaagccctctgggtcctactctgtccttttcagcccatagctttgttcaaaagctgctggcagtgttcagatttggctgagttcagtgaatatgtgcattggctgatttctgagccatgccagggggatggagaagccgaagcaggagtgtttgttctgcaggctctggagtaggcattgggtctgtgccggctcacttgctagtcttgcatccttccccaaccccctctggggatgtctggccacatcagaagacagtttgggttgtcagaactgggggagtaccaggccgaggtgggtggatcatgaggtcaggagatcgagaccatcctggctaacacagtgaaacctcatctctactaaacatacgaaaaaaattagctgggcgtggtggcgggcgcctgtagtcccagctactcgggaggctgaggcaggagaatggtgtgaacccggggggcggagcttgcagtgagctgagatcctgccactgcactccagcctgggcaacaaagcgagactccgtctcacaaacaaaacaaaacaaaacaaaacaaaatctgggggagtgccactggcatctgatgtatagaggcccgagatgctgtgtcatcacccgttgagtgcgctcataggcatcttcctgacaattagaacccattattcttcaaattcaatgcaagcaaattcaaagcattactgtgtacataccgcatgctaatcaattgcaccactggagctcctaaattcaaaacattactataaaaaagttcaaaatgcatggaaaagttgtacatggcaggagaatatttgggcttctgactaccccttgaatgaagatgatccaccagccgccttcctccttggtcttcactccagattcctagcatttcattctgtgtctctttatgcagtgaggtttttgtttgttttttgagacagagtctcactgtatcacctaggcctggagtgcagtggcgcgatctcagctcactgcaaccctcggctcctgggtttaagcgattctcctgcctcagcctcccgagcagctgagattacaagcacacatccccatgcccagctaatttttgtatttttagcagagacagggtttcaccatgttgcccaggctggtctcgaactcctggcctcaagtgatccatgtgcctcagccttccaaagtgctgggattacaggcgtgagccaccatgcccagctcctagtgaggtttttgatgccttgctacatctgccctagaaattgtgtgactacgattttggaaatgttgctgtgtaaacttgtgatcatttctggactccaggcaagaatcttgatggctaaggtgtggctgaacatgtctgattctctcctggacctgttttaggccaaactctgctctgaaattcctccgtgtggaagggcgggctggggagagcctcccagctggaatcttttggatgcctttctctgtgggtatctgatggctggctctgatggctggctgtgatggctgtggctggaaatcattgttgacatgagtttcacagatgcaggctctgtccaaattgtagcaaaagctgcctgccccagccgagctatgggcaataaggtggtttaaggatatagatgaaggaaaactcacccttagaataatttatccaaaatgctgctgtgttgtgggttagaggacattttctgaggtcccaggttcattgtttcatttaagtctcaaaagtccctccaggtgttggttctaattgtcaaagcatggggggagatgggctcatgggttaaaggtcttatcccagatttctgtatcctccttgcaagcagcaaaggggtctggatttgaatccatgaccatgtttctcctttgggtttccatcacactctgtccccgtgcactgagcaccctttagttcatatgacccccttaggcatgttacatgggcactcctataggtgcccatctggccctaggacttggccaacacaacatggactccagtttccatctgcctctttgccaggcacttttgtgcagtgcacacactgtacaacagtagacggcaaccctgagagccagagtagagcctgtcctagcaccggaatgctcggtaaggatttgtcgcaggagtgattccaaagccaatgtcctccctccatatcagcctgtttgtggctctgagaagctctgcccacatgtgaaagcttgttaagcacttaagcactaacccagagcttcagacagtgccagtcctttttccccttctttaaaagcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgagttggtggtgagggggcaggtgttctggggtgcagctcttctttgcctccctgattgccaggagctaccagttactgtctgcacaatcaaacagaaatagacctgtccttgatggttaacggaaataaaaggcgcttgtcccagaagctcaggtgaggcaccaccctgattatgggaatcacctgggaacatatacccagacctaaaactcagatccacttcccaggctgtggttatatagtcaggggggtgcagtatgggtattaggattttttattttttagttataaagatttttttttggtttgtttttgagacagggtcttgctctgccgcttaggctggagtgcagtggtgcaatcatagctcactgaagcctcagactcctgggttcaagcagtcctcccacctcagcctcctaaggagctgggacccacaggcatgcagcaccacacctggctaatttttaaaaattttgtggagtgttgcccaggctggtctcacactcctggcctcaagcgatcctcccaccccagcctcccaatgtgttgggattacaggcatgagccattgtacccagccactaagatgattcttatttggaaacacggtcaagaacaactgcgttcggtagtttaaccttttttgattgtggtggttttagtatgccttaccactctaccatagtaagaaatttgcagaccatgtacaccaacctttggtgctcctggggagaaagaaagaaggctatgcaatgcaatgcatgctcacagtccaagggagagggaaagctgtctaacaggattggttttcccgtgtgctttataagcagatgagtagaggagacagctcttattgtcctagtggcaattgggataggctgcaaagtttgttagggtggaggcttattccgggaccaagggagcccaaagaaacaagctcctgccaggcgcggtggctcacgcctgtaatcccagcactttgggaggctgaggcaggtggatcacctgaggtcaggagtttgagaccagcctggccaacatggtgaaaccccgtctccatgaaaaatacaaaaattacccgggcatggtggcgggcacctgtaatcccagctactagggaggctgaggcaggaaaatggcttgaacctcggaagtggaggtggccgttagccgagatcacgccactgcactccagcctgggcaacagagcaagactctgccttaaaaaaaaaaaaaaaaaaaagaaaagtaaaaggaaaaaaaagaggctctggcctgctggggtgcctgcaaagtctccgtggaagggtgacattcaagccgagacctccagggaactgtctcctgggagcacagagccctttgctcagcccccaggtggctcagtgcccccagccagcagactcagagcttgcatgattctttggtgctctctgcggtcttccaatgatgctgaaataaatggtgcttggtgtctccctgctgtagtccccttgcttgctttgctcacaggtgctgggagagatatgccttcgaggatatttggatgcattcaggttcttggaagagaagggtatgtatgggctgggaggatcagccatgcccttttgacaagcatttactagcggtcttggtaaagacttgagatttgccttagttctaacacttagtgcccaacgccttccttgtgttgctcaacctactcatgagcccaggagataggaaatctccgtcccattgtacagatggggaaacagaattttggaaaggagagccaagcagcacacacccctccctgaggggcagagccgagatttgaactgggatgtcatgactccagggccctctccctccccagggtccccttatctgaaggcggtttttctttccagctcgacctcttgtgacccttagtttaacaagggccgaagttaaagagtttctgcgcctggaccccaaatgaagcaatcagatttctcatctccagtcaggtgtgggtccaagcccactagacaagtttgctcttcccagagcacatttctgccttcaagtcatcctggcttgtcagggctgggggagttctgctgtagaaatattagagtggaaggaaaaagatgtgttgggagctatttttctttaatactaaaagttggttgatgaatttgtcgttggccaagaccaaggagactgcatttttaaggacatatgtgtatttatctgctcagaaaatgttcattgctgtgtgctagggatactgcagtgaacacagaggtgtgacccttgccagccttgtgagagaagtgagcagataagtaagcagaagggtgatgctgtgtcgatgggaaagtacaggtgccaatgagaaggcacaggtgtcaaggagaagacacaggatgctggaggctcatgcaggatggatctccaaggcccaggggaagaagggcctctcggaggacgtgaatccacattaagactttggggataagtaggagcgccttaggcatggggacccatggatgcgaggcctgtaggacacagacaggatggcatgaaggcctgtgcaactggaggggtggggatggggacactaagagatggctggaagtgtgggggtggggacactaagagatgactggagaagagggggtcaggagtggtgaaaaatgggagaggagggcaggctgggccttttggatacagggggattgcatcctgcagtggtagggagccactgagggctgctgcagtaggagtgaggggatcagaggagagctttggaagccccctggatgcgggacaggaagcgagataccagtgtctaggaggccagtgaggcagccacaggctccaccaggatcagggctgcgagggtcatgaggaggaaaccaatttgaaggagtccaggggaataggacttggaaatgaccgatgggacatttgggaagaggaagacagaagagcgcagtcccggcttctggctttagcagttgggcaaggggagatggggagatgtgcccatgggttgagggttgaggacattaggagggagccggtatggcaggaagagctggtgtgccagagatgctggaagcagcatctgcctgagaacagatacctggcaatattcctaagggaaagtgacatctcggagggtgaggagggcatctgatagggcctggaaagagccggggcaagcatgaatgtgaggttatcttggggggcaaggctcaggcgttgaggagcagcccctggtctcttcagcctgaagttggaagccagagttgggccaggtgcagctgtggttgtctgaagtccccctcccccagcccagtgtgccaatgctgtaagagcaagggccgctcactggtgctggtggctgagtcccagcacccaggacagggcctggcacatactggtgcccaatcctcccttctgggtgcttcttccaaggccttgtgatggaagtgagtaccctcttcgacatcagacccagcttcaaatcctggctctgctatgtattggctgcgtggctttagacaagtcttttaaccttgctgtgcttctgatttctcagctgaaaaatggagatgatgatagtggtttctgtaaggccttatggtgaagcacctagctcagggcctggaaggcaggtgtaaccagtggttcagttgttataaaccaacactaaccctcgcctttgcacctcatgaaaccagatatgtagatggagcccacaaagctagcaggagccaagctcacgtgtgtcctgctttaaagccccatacccctttctccgggtgacaaacacctgtgctcgttctcttcccttcccctcttccccttgcatttggctaataacaggccagctgcctgcctccctgcagtttggtagatgggtgggtaacgaccaccactcccacgttcgcctgatgggcttgttttccgtgcccttcacaggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacaggtacccactcctcggggtgagcacgggcagcaccttgttttctttcttgtgcattatggaggaagatggtactgccacatgggagcgatagggtgaggcaaccatgacaggtggttgggaacatctccttccatgtgtacagcctgggctgctgccatcactcccagcacagcccccaacccccccaatcctggaaccttgccaagtctcccttcccatggggtcatgaccaggaggaaaacaaactccagctgagccccttggggttccccatataggctcctgcctgtggcagctgggccctctgtacccctttccaactctgtctccctaacatggcacctgagctcctgccatcctggatttcatggaccccaaggatgggggtcctgcatctgggacttggcctattactcggagctccttttcagccgcctccctccacctgtccacccacctcaaggctcctttcttgagacctctcctaatttctcccttcccctaaacccacaattttgaacctccatcgaatggtgctgtattttataatgtcatcaaatatcaaatggagacagtgctatggtccaaatgattgtgtaccccccagaatttgtcttttgaaatcctaacccccaacatgatggtcttaggaggtggggcctttgggaggagattaggtcatgaggaaagggctgtcatgaatgggattggtgcccttattaaacagacccaagagaggtcccttgtcccttctactgtgtgaggactcagaaggtggtgtctatgaagaagcaggccctcaccagacaccaacatgtctgctgccccttgatctgggaccttgcagcctctagaactctgaaaaatcgatgtttgttgttttataagccactcagttggtggcattttgttagagtagcctgaacacggactaagtcaaacagaagaacccacaaaccagctacagagttgggcatttggagaaattcaaaaatgagtcagacataactccttattcttgaggtgccctaagagatgggacacagcagctgcccaggtgcattagtttgttctcacattgctataaagaaatacctgagactgggtaactcataaagaaagaggttgaattggctcacagttgcacaggctggacaggaagcatggtgctggcatctgctcagcttctggggaggcctcaggaaacttacaatcatggcagaaggtgaacgggaagcatgcacatcccatgactggagcaggagtgagagagagagggaaatagagggaaggtgccatacacttttaaacaaccagatctcatgagaacacattcactatcaagagaacagcaccagtggggaaatccgcccccatgatccaatcacctcccatcaggctccgcctccaacactgggaattacaatttgacatgagatgtgggcagggacacagatccaaaccatatgaccagattaatacgatttgaggcatcacgaggtcattaaagagagggaataaaagactggggctccaggaagaaggctctggaatccagcagagggtcaaggaccagcttgtaaagctggtggtgcctgagaagtacctaggagaacatagatgctgtgacgtttgatgtagctgttttttgttttgtgttttggtttttgagacagagtctcactctgttgcccaggctggagtgtgcagtggcgtgatcttggctcactggagcctccatctcccaggttcaaatgatcctcatgcctcagcctcctgagttgctgggattacaggtgcacaccaccacgcctggctaatttttgtgttttcagtagagacagggtttcaccatgttggccaggctggtcttgaactcctgacctcaagtgatccaacaacttcagcctcccaaagtgctgggatgacaggcatgagccaccatgcccagcctgatgtagctgtttctgtgcacattatttgctgtggggtatattcagatttcttaatacaagatgattctttgcctcatgacttacacaccattttctatttaatttcagctatgatattggaaatggacatgtcttttcaaggaaaataaaagcaggctttctggaatggcgacttccaaacatatttgtcaatttaaaggagctgggagtggggaccctatgctccgtaagcactctcttagctgttcttggctgtgctccccgcttcagcttcacactgcccttgctgtgaagggagcagcctgggccgggcgcggtggcttacacctgtaatcctagcactttgggaggccgaggtgggtggatcacctgaggtcaggagttcaagaccagcctggccaacatggtgaaactccatctctactaaaaatacaaaaaattagctgggcatggtggcaggtgcctgtaatcccagctacttgggaggctgaggcagaagaatcgcttgaacccaggaggcggaggttgcagtgagccgagattgcgccattgcactccagcctgggggcaacaagagcaaaactctgtctggaaaaaaaagaaaggagcagcttggcaaaccccaccttgtcgcttttgtgagtgcctctgaccctttggctgccaggacgggcgtattttatggaaatgctaagcaccaacagagtaaagtggtttggtttttcacagtggtgggagataatagctccaaattgtctttttcagcactgagtgaagaaatgaaagacaaaggtggatacatgagcaagatttgcaacttgctacccattaggataatgtcttatgtaatgctgccctgtaccctgcctgtggaatctgccattgcgattgtccagaggtgagcattttaggtggctccgtgtcttcctcacagggttgatatgaggatgaaacaagatgatagatcatggtggcatgtagtctgggacctggattgtcgtgccacagatcacagctcacagtctatgtgcaatgcccctgaatgttgcccacctgtcctcaagccacacatgcacctgtaactcagtgcaagcccagaaactccccgtggggactcctagagctgtcagtggcctcacatagcagctggtccagtctcttgtgattgcccaaggaaactgaggcctggagagcttggggtcactgctctgaggccatagagatgcctagtagaagggccaggcctagaagcaggatccttgctgcccctctgagctgtttccatttaaaatcacatgaaggccggcgccgtggctcacggctgtaatcccagcattttgggaggccaaggtgggtggatcatgtgaggtcaggagtttgagaccagcttggccaacatggtgaaatgccatctgtactaaaaatacaaaaattagtggagcatggtggcacgtgcctgtactcccagctacttggaaggctggggcagaagaatcgcttgagcctgggaggcagaggttgtagtgagccaagattgtaccactgcactccagcctgggtgacaggagagaaaccctatctcaaaataaaatgaaaggtaatgaaatgaataaaataataaatcaagtcacggccgggcacggtggctcacacctgtaatcccagcgctttgggaggccgaggtgggtggataatgaggtcaggagttcaagaccagcctggccaacatggtgaaaccatgtctctactaaaaatacaaaaattagctgggcatggtggtgcatgcctgtaatcccagctactccggaggctaaggcaggagaattgcttgaagcaggacctaggaggcggaggttggttgcagtgagccgagatcatgccactgcactctagcctgggctacagagcgaaactccgactcaaaaaaaaaaaaaaaaaaaaatcaaatcacatgaaagtagaacatagggaattccatctttcgttctaggcatagtttgttaatatgattcagagccagcagttaggagaacacagtgtgactctcctagaacttcttgattgggcttcctctgattgggtttcctctgattgggcttcctctgaaagtgggggggatggggggtggggagcagaatggtcagagcttggctcagcagtcagactgctcttcttcaaatcctggctgcattgcttactacagctgtgtgactccagatgactgaatccacctctctgtgctgcagcttcccgtctagagagatcacctggagcagagggtggtcaggagactcaatctggttactgactcacagtgcaggagtactcatcccatagtaagcatccagctagagatgttgatttctattttcaggtaataatgatgatcgtaaaattagagacagataaaaggtatgggcattaggccagggcactgcaatttctaagctgtgtgacctcaggcaagttactcgacttctctgagcctcagcggtttcatccgcaatatatggataggaaaaccgacctcagtgggttgtctgacagtggagggcacttgattaaaaaaaaaaaaattaccctggtctgaatattaccctggactgaaagaaaaatattgagctaatacaggcatcaggaatggggctgcagggagtccagggaagggagaacgaagagcctgaaggtgtgaggaggtgcgagtgctgatctgtctgctacaaagaggctgctgagcctcctgtggatgtggccctggacttggcagtttaatacctgagctgttaaaataacctcagatgctgtgttctttaaggggtaggattcagattcctgctgaaatgcttctgaaagggagggaatgagccagcccatccccagttgctttttaagatcattgggaagttctggtcttgccatttgtccctggaccactcttaggtcctcctgccccacttccatctgggtgtgtgccctgggctgtccaccacacagctacatcctgccatcttccctcctggagccactgtgccatgcatggatctgtagcttcatttttcttggcttttccctggtttttctggagcagagtctctagtaaactcccaaggaagaaaacgtttgactttatgtgtgttgggaaacgtgctttttttctattacatctcagtgataggttggccatgtctagaattgcaggttgaaaatcatttcctctcagtatattggttagtgagaagcctgggactgagacagtcacattctcacttctttgcaggtgagtgctcttaggactgtctttttatcccttatactctgaaatgtcatatgtcttggtgtaagtccttatttcagttattgagctggacaagtactggagaccccttcagtcaaagccttctgtcattctccagctctaggaaattatcttctattgttatttctgttattccttcccttccattttcttttttctttttttttttttttttttgagacagggtcttactctggtgcccaggctggaatgcagtgacctgatcatggtacactgcagcctgaacctcccagactcaagtgatcctcccacctcaacctcctaagtagctgggactgcaagcacacatcaccacacccaacaaatattttttaaaaattttgtaagatgggatcttactatgttgcccagactttttcttcctcttcctggggctcttattaggaagatgtttgacttcctgggttggattcctgtctccgtgtctgactttctctctttgtcatatttttcatcactcgttgtctttttgcgtctgctctgacagatttcctcaaattttgtcttctagtcctatcctacagtttttactttcagcaaatataatttaatctccaagagtactctcttgttcttttttcttagcattctgttcttgttttatggatgtaacattctcttggaatatttgctgtcctctagatcatcccttctccatttcttcttgggctagtttttctgtttcttcatctttctcttttatgctacttattctgggcgtgttcttggtgggttttttcccatatagcaacagaggacttggagctcagggagaaaagggtaggtgcatcacctggcagagctcccagacagtgacaggcaggctgcgggaaggatgtctacttggcggtgctaccgctttcctagaaaccctttccctggagctggttgaactgttgggttttgccctggtggtgaacgctggctccccgtgctctgcctgtttcatcaccagccccctccccttctgcctggggtccagtaatctgttgaaatatatatcttgctcattggtgagctcctgctccttcctcgttgctcttgcagatttatcacttctcgtaaggctgcgcttgtacttcgggattttctctgtgccacactgggaaacatagggtggttgcatgctgcagtcctgagcacttatttcactcacatctttacacgaagatttggtgggtgtttactttgtttttagtaagttagtctgtcatgtcctttgatcctttttttttgttttttgagatggagtctctctgtgtcctccaggctggagtgcaatgtcgcgatctcagctcactgcaacctccacctcctgggctcaagagattctcctgcttcagtctcctgagtagctgggattacaggcatgtgccaccacacctggctaatttttgtatttttagtagaggtggggtttggcatgttggccagcctggtctcaaactcctgacctcctgacctgcctgccttggcctcccaaagtgctgggattacaggtgtgagccaccacacctggccctgattaatcttttaatgcccagtctctccttcaaaagccggctcctttctctccctcgccttcctagattccttctccactccccaggatcagcctcctcctccccaccccaccactgccggggggatgtctgtggtcaggcatttatcagagaccctgaggtgggggtcctttatgtgtctgggggatggagagtctagaggaggtagcgttcagacctctccatggtgcctctgctgggctcacatgtgaccaagcacagcaaaccatgaggcaggggatggtcttgaccatgagagcccttgcagcagctgccatgggcctcagctcctctccaagctgggaagagccctgaaaagccaaggtgtttttttttccctctttatttcagtgtaagtcccttgagctttcttgaaccagaagtgggctcattttgctttagagatttcaggtgggcttgtccttgtcctagcatcccagatccaccttctgggaagtcatcagattggaggtgatgttggcagcttttgtaaacaaagggtagtgttgtaagctgttgtgtctgcctatgtgtgtgtttgtgtacttggtctcatctctgcagactggtgacatggcttccagatatgcccgacgatgtcctgtggttgcagtgggtgacctcacaggtgttcactcgagtgctgatgtgtctgctccccgcctccaggtaaatactttggctgtgggtgtgtgggccggacgggcacctctctcatctgatgaggcctcacacgacattctagaaacagctggctgaacaccaagcaaggagcttgcccttgggtgtggggaccctgtctcatgggaggcagctgagtcagtcagaggtcctggcacacctgctgagagctgccacccaggccaacctgaaccggagcctgggaagacttcccgtcggatgagtctctttgagtgcagcattgatggtggaagagcagagaggccccagataagcagggaaaggtgcttcagacagagtggctgggatgaggactggggagtgtcagatagcgctggcgtgtctgagcgaaggagctctggcacccatggcacaggaaggaggtgggaccctggaggggcagggctagcagagctcctcggagcgtgtggctaggtgcctggtaatgcaagccccctgtcctccaccctctgttgtactgagtcacagtctccggggtgaagcctagcagtctgcgttgacaggccccaggggatgccgctacttcctgaattctgaattctggaaactgagccggagttcagggcctggctcccattaccagggttgggcgttatcctgaaaatcataggccttggtttcctcacttggctaacaggggtgatccccatcccctcaatgggtttccgtgagctcctgagagcccgtagcatggtacttggcacatgctgggcatcaggaggtatggcctctcttgctattgttgttattggtagacacagaaggatttaaaagtaggggaatgcaaagatccgatttgctagggaagagggcagtagtggccaagtagagggtggatcctgggccctggctggcagcaggcagcaaggggggctgccagggcccaggcagggacgatctgtagaccgagaggcttcctaaggctcttggacaggaggaggtgtcggttccaagcctaaggagtggggcagccctggtgactggtggtcagtggtgccaggcggtgggtggtaggacaccctggcaggcaagtaggtttgtgtgggggaaactgataggcccctccagggattcgttggtggacaacacctgtgatgtccagtgggaggtgtccaggtagctgggagggccacaggcttggaagacctaggtggtgacatcagcccagcactgagggctagaagaagctgtgtctctggctgtgacggcaccctagagtgtgtgtggtgccctctactggccggcaatgtgggtccaccgtagctcagactgcacactgcagcagcgggaacggcctctaagccaacttcctccatgtgtttcaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagac

SEQ ID NO: 32 - RNA, 1443 nt, Homo sapiens
  source 1..1443: mol_type = mRNA
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctg

SEQ ID NO: 33 - RNA, 1431 nt, Homo sapiens
  source 1..1431: mol_type = mRNA
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccgagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctg

SEQ ID NO: 34 - RNA, 1443 nt, Homo sapiens
  source 1..1443: mol_type = mRNA
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatgcccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctg

SEQ ID NO: 35 - RNA, 1431 nt, Homo sapiens
  source 1..1431: mol_type = mRNA
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccgagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatgcccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctg

SEQ ID NO: 36 - DNA, 1443 bp, Homo sapiens
  source 1..1443: mol_type = other DNA
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctg

SEQ ID NO: 37 - DNA, 1431 bp, Homo sapiens
  source 1..1431: mol_type = other DNA
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccgagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctg

SEQ ID NO: 38 - DNA, 1443 bp, Homo sapiens
  source 1..1443: mol_type = other DNA
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatgcccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctg

SEQ ID NO: 39 - DNA, 1431 bp, Homo sapiens
  source 1..1431: mol_type = other DNA
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccgagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatgcccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctg

SEQ ID NO: 40 - protein, 481 aa, Homo sapiens
MYDAERGWSLSFAGCGFLGFYHVGATRCLSEHAPHLLRDARMLFGASAGALHCVGVLSGIPLEQTLQVLSDLVRKARSRNIGIFHPSFNLSKFLRQGLCKCLPANVHQLISGKIGISLTRVSDGENVLVSDFRSKDEVVDALVCSCFIPFYSGLIPPSFRGVRYVDGGVSDNVPFIDAKTTITVSPFYGEYDICPKVKSTNFLHVDITKLSLRLCTGNLYLLSRAFVPPDLKVLGEICLRGYLDAFRFLEEKGICNRPQPGLKSSSEGMDPEVAMPSWANMSLDSSPESAALAVRLEGDELLDHLRLSILPWDESILDTLSPRLATALSEEMKDKGGYMSKICNLLPIRIMSYVMLPCTLPVESAIAIVQRLVTWLPDMPDDVLWLQWVTSQVFTRVLMCLLPASRSQMPVSSQQASPCTPEQDWPCWTPCSPKGCPAETKAEATPRSILRSSLNFFLGNKVPAGAEGLSTFPSFSLEKSL

SEQ ID NO: 41 - protein, 477 aa, Homo sapiens
MYDAERGWSLSFAGCGFLGFYHVGATRCLSEHAPHLLRDARMLFGASAGALHCVGVLSEQTLQVLSDLVRKARSRNIGIFHPSFNLSKFLRQGLCKCLPANVHQLISGKIGISLTRVSDGENVLVSDFRSKDEVVDALVCSCFIPFYSGLIPPSFRGVRYVDGGVSDNVPFIDAKTTITVSPFYGEYDICPKVKSTNFLHVDITKLSLRLCTGNLYLLSRAFVPPDLKVLGEICLRGYLDAFRFLEEKGICNRPQPGLKSSSEGMDPEVAMPSWANMSLDSSPESAALAVRLEGDELLDHLRLSILPWDESILDTLSPRLATALSEEMKDKGGYMSKICNLLPIRIMSYVMLPCTLPVESAIAIVQRLVTWLPDMPDDVLWLQWVTSQVFTRVLMCLLPASRSQMPVSSQQASPCTPEQDWPCWTPCSPKGCPAETKAEATPRSILRSSLNFFLGNKVPAGAEGLSTFPSFSLEKSL

SEQ ID NO: 42 - protein, 481 aa, Homo sapiens [sequence omitted]

SEQ ID NO: 43 - protein, 477 aa, Homo sapiens
MYDAERGWSLSFAGCGFLGFYHVGATRCLSEHAPHLLRDARMLFGASAGALHCVGVLSEQTLQVLSDLVRKARSRNIGIFHPSFNLSKFLRQGLCKCLPANVHQLISGKIGISLTRVSDGENVLVSDFRSKDEVVDALVCSCFMPFYSGLIPPSFRGVRYVDGGVSDNVPFIDAKTTITVSPFYGEYDICPKVKSTNFLHVDITKLSLRLCTGNLYLLSRAFVPPDLKVLGEICLRGYLDAFRFLEEKGICNRPQPGLKSSSEGMDPEVAMPSWANMSLDSSPESAALAVRLEGDELLDHLRLSILPWDESILDTLSPRLATALSEEMKDKGGYMSKICNLLPIRIMSYVMLPCTLPVESAIAIVQRLVTWLPDMPDDVLWLQWVTSQVFTRVLMCLLPASRSQMPVSSQQASPCTPEQDWPCWTPCSPKGCPAETKAEATPRSILRSSLNFFLGNKVPAGAEGLSTFPSFSLEKSL

SEQ ID NO: 44 - RNA, 2397 nt, Homo sapiens
  source 1..2397: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 45 - RNA, 2289 nt, Homo sapiens
  source 1..2289: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 46 - RNA, 2280 nt, Homo sapiens
  source 1..2280: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 47 - RNA, 2398 nt, Homo sapiens
  source 1..2398: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 48 - RNA, 2469 nt, Homo sapiens
  source 1..2469: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggaatggaaaggacatcagaagtaattacttggatgtatataggatcgaggacacttttggacgagactctgagatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 49 - RNA, 1715 nt, Homo sapiens
  source 1..1715: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagttaagtacagcacagaacacccaaatactaaaacaccaatagagcttttttttttgctttttttttttttagacagagtctcactctgtcaccctggctggattgcggtggttgcagtggcatgatcttggctcactgcaacctccgcctcctgggttcaagcaattctcatgcctcagacccccaagtaactgggattataggtgtgtgctgccacactacacccagctaatttttgtattttttgatagagacaggtttccccatgttggccaggctggactcgaactcctgacctcaagttatcctcctgtctcggcctcccaaagtgctgggattacagtcatgagccaccatgcctggcccaatagagctattattatggagcatctttcagttgtgaaaattggcatggaaactctccatccctggggagaacagttatttcctctgttattttcctacccagtctataaaaagagagtgattcattttctctaccaaatctactgtctctgcccaaactttgctgaagactattctaactaaaggaaacacagtttaaaaagaatgcaatatagtgaagtagttaataataaagactccatttttaaaagtctgctggaagtttggttgggattgcactgaatctatagagcaattggggagtattgacatatcaacaatattgagttttctaatccaagaacataatatctatttttaaaatcttcttcaaaatctttaaatctttaaattgtattttgtagtttttggtgtttaagtcttgcacatattttgtcagatttattccaaagtatttcacgggttcttttt

SEQ ID NO: 50 - RNA, 2290 nt, Homo sapiens
  source 1..2290: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 51 - RNA, 2470 nt, Homo sapiens
  source 1..2470: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggaatggaaaggacatcagaagtaattacttggatgtatataggatcgaggacacttttggacgagactctgagatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 52 - RNA, 1714 nt, Homo sapiens
  source 1..1714: mol_type = mRNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtaagtacagcacagaacacccaaatactaaaacaccaatagagcttttttttttgctttttttttttttagacagagtctcactctgtcaccctggctggattgcggtggttgcagtggcatgatcttggctcactgcaacctccgcctcctgggttcaagcaattctcatgcctcagacccccaagtaactgggattataggtgtgtgctgccacactacacccagctaatttttgtattttttgatagagacaggtttccccatgttggccaggctggactcgaactcctgacctcaagttatcctcctgtctcggcctcccaaagtgctgggattacagtcatgagccaccatgcctggcccaatagagctattattatggagcatctttcagttgtgaaaattggcatggaaactctccatccctggggagaacagttatttcctctgttattttcctacccagtctataaaaagagagtgattcattttctctaccaaatctactgtctctgcccaaactttgctgaagactattctaactaaaggaaacacagtttaaaaagaatgcaatatagtgaagtagttaataataaagactccatttttaaaagtctgctggaagtttggttgggattgcactgaatctatagagcaattggggagtattgacatatcaacaatattgagttttctaatccaagaacataatatctatttttaaaatcttcttcaaaatctttaaatctttaaattgtattttgtagtttttggtgtttaagtcttgcacatattttgtcagatttattccaaagtatttcacgggttcttttt

SEQ ID NO: 53 - DNA, 2397 bp, Homo sapiens
  source 1..2397: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 54 - DNA, 2289 bp, Homo sapiens
  source 1..2289: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 55 - DNA, 2280 bp, Homo sapiens
  source 1..2280: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 56 - DNA, 2398 bp, Homo sapiens
  source 1..2398: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 57 - DNA, 2469 bp, Homo sapiens
  source 1..2469: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggaatggaaaggacatcagaagtaattacttggatgtatataggatcgaggacacttttggacgagactctgagatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 58 - DNA, 1715 bp, Homo sapiens
  source 1..1715: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagttaagtacagcacagaacacccaaatactaaaacaccaatagagcttttttttttgctttttttttttttagacagagtctcactctgtcaccctggctggattgcggtggttgcagtggcatgatcttggctcactgcaacctccgcctcctgggttcaagcaattctcatgcctcagacccccaagtaactgggattataggtgtgtgctgccacactacacccagctaatttttgtattttttgatagagacaggtttccccatgttggccaggctggactcgaactcctgacctcaagttatcctcctgtctcggcctcccaaagtgctgggattacagtcatgagccaccatgcctggcccaatagagctattattatggagcatctttcagttgtgaaaattggcatggaaactctccatccctggggagaacagttatttcctctgttattttcctacccagtctataaaaagagagtgattcattttctctaccaaatctactgtctctgcccaaactttgctgaagactattctaactaaaggaaacacagtttaaaaagaatgcaatatagtgaagtagttaataataaagactccatttttaaaagtctgctggaagtttggttgggattgcactgaatctatagagcaattggggagtattgacatatcaacaatattgagttttctaatccaagaacataatatctatttttaaaatcttcttcaaaatctttaaatctttaaattgtattttgtagtttttggtgtttaagtcttgcacatattttgtcagatttattccaaagtatttcacgggttcttttt

SEQ ID NO: 59 - DNA, 2290 bp, Homo sapiens
  source 1..2290: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 60 - DNA, 2470 bp, Homo sapiens
  source 1..2470: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggaatggaaaggacatcagaagtaattacttggatgtatataggatcgaggacacttttggacgagactctgagatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaaggtttcttcctgaacgcgcctcagcgattttaaatcgtatgcagaatattcaatttgaagcagtggttggccacaaaatcaaaatgaaatgaataaataagctccagccagagatgtatgcatgataatgatatgaatagtttcgaatcaatgctgcaaagctttatttcacattttttcagtcctgataatattaaaaacattggtttggcactagcagcagtcaaacgaacaagattaattacctgtcttcctgtttctcaagaatatttacgtagtttttcataggtctgtttttcctttcatgcctcttaaaaacttctgtgcttacataaacatacttaaaaggttttctttaagatattttatttttccatttaaaggtggacaaaagctacctccctaaaagtaaatacaaagagaacttatttacacagggaaggtttaagactgttcaagtagcattccaatctgtagccatgccacagaatatcaacaagaacacagaatgagtgcacagctaagagatcaagtttcagcaggcagctttatctcaacctggacatattttaagattcagcatttgaaagatttccctagcctcttcctttttcattagcccaaaacggtgcaactctattctggactttattacttgattctgtcttctgtataactctgaagtccaccaaaagtggaccctctatatttcctccctttttatagtcttataagatacattatgaaaggtgaccgactctattttaaatctcagaattttaagttctagccccatgataacctttttctttgtaatttatgctttcatatatccttggtcccagagatgtttagacaattttaggctcaaaaattaaagctaacacaggaaaaggaactgtactggctattacataagaaacaatggacccaagagaagaaaaggaagaaagaaaggttttttggtttttgttttgttttgttttgttttttgtttttttgagatggagtctcactctttcgcccaggctggagtgcagtggtatgatctcagctcactgcaagctccacctcccgggttcacgccattctcctgcctcagcctcctgagtagctgggactacaggcgcccgccaccacacccggctaattttttgtattttttgtagagacggggtttcaccatgttagccaagatggtctcgatctcctgacctcgtgatccacctgcctcggcctcccaaagtgctgggattacgggtgtgagccaccgtgcccagcctttttttttttaatagaaaaaataatccgactcccactacatcaagactaatcttgttttgtgtgtttttcacatgtattatagaatgcttttgcatggactatcctcttgtttttattaaaaacaaatgatttttttaaaagtcacaaaaacaattcactaaaaataaatatgtcattgtgctttaaaaaaataacctcttgtagttataaaataaaacgtttgacttctaaactctg

SEQ ID NO: 61 - DNA, 1714 bp, Homo sapiens
  source 1..1714: mol_type = other DNA
agacagtacctcctccctaggactacacaaggactgaaccagaaggaagaggacagagcaaagccatgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtaagtacagcacagaacacccaaatactaaaacaccaatagagcttttttttttgctttttttttttttagacagagtctcactctgtcaccctggctggattgcggtggttgcagtggcatgatcttggctcactgcaacctccgcctcctgggttcaagcaattctcatgcctcagacccccaagtaactgggattataggtgtgtgctgccacactacacccagctaatttttgtattttttgatagagacaggtttccccatgttggccaggctggactcgaactcctgacctcaagttatcctcctgtctcggcctcccaaagtgctgggattacagtcatgagccaccatgcctggcccaatagagctattattatggagcatctttcagttgtgaaaattggcatggaaactctccatccctggggagaacagttatttcctctgttattttcctacccagtctataaaaagagagtgattcattttctctaccaaatctactgtctctgcccaaactttgctgaagactattctaactaaaggaaacacagtttaaaaagaatgcaatatagtgaagtagttaataataaagactccatttttaaaagtctgctggaagtttggttgggattgcactgaatctatagagcaattggggagtattgacatatcaacaatattgagttttctaatccaagaacataatatctatttttaaaatcttcttcaaaatctttaaatctttaaattgtattttgtagtttttggtgtttaagtcttgcacatattttgtcagatttattccaaagtatttcacgggttcttttt

SEQ ID NO: 62 - DNA, 25 bp, synthetic construct
  misc_feature 1..25: note = forward primer
  source 1..25: mol_type = other DNA
atgaacatcatcctagaaatccttc

SEQ ID NO: 63 - DNA, 25 bp, synthetic construct
  misc_feature 1..25: note = reverse primer
  source 1..25: mol_type = other DNA
atcatgcatacatctctggctggag

SEQ ID NO: 64 - DNA, 20 bp, synthetic construct
  misc_feature 1..20: note = primer
  source 1..20: mol_type = other DNA
atcagaacttcaggccttgg

SEQ ID NO: 65 - DNA, 21 bp, synthetic construct
  misc_feature 1..21: note = first exon
  source 1..21: mol_type = other DNA
gcaaagccatgaacatcatcc

SEQ ID NO: 66 - DNA, 24 bp, synthetic construct
  misc_feature 1..24: note = last exon
  source 1..24: mol_type = other DNA
tcttgatgtagtgggagtcggatt